(19) United States
(12) Patent Application Publication
(10) Pub. No.: US 2024/0016943 A1
(43) Pub. Date: Jan. 18, 2024

(54) SYNTHETIC DNA BINDING DOMAINS AND USES THEREOF

(71) Applicant: The University of Chicago, Chicago, IL (US)

(72) Inventors: Raymond E. MOELLERING, Chicago, IL (US); Thomas E. Speltz, Chicago, IL (US); Sean SHANGGUAN, Chicago, IL (US)

(73) Assignee: The University of Chicago, Chicago, IL (US)

(21) Appl. No.: 18/038,633

(22) PCT Filed: Nov. 24, 2021
(86) PCT No.: PCT/US2021/060808

§ 371 Date: May 24, 2023

Related U.S. Application Data
(60) Provisional application No. 63/117,710, filed on Nov. 24, 2020.

(51) Int. Cl. A61K 47/64; C07K 7/08; C07K 14/00; A61K 47/54

(52) U.S. Cl. CPC A61K 47/64; C07K 7/08; C07K 14/001; A61K 47/545

(57) ABSTRACT

In aspects, the invention provides a polypeptide construct comprising (a) a first polypeptide comprising an amino acid sequence derived from a basic helix of a transcription factor protein that comprises a basic helix-loop-helix domain; and (b) a second polypeptide comprising an amino acid sequence derived from a helix that extends in the C-terminal direction from the end of the loop of a basic helix-loop-helix domain of a transcription factor protein that comprises a basic helix-loop-helix domain; wherein the first polypeptide and the second polypeptide are linked through an interpolypeptide covalent linkage. Additional aspects of the invention are as described herein.

### CROSS-REFERENCE TO RELATED APPLICATIONS

This patent application is a U.S. National Phase of International Patent Application No. PCT/US2021/060808, filed Nov. 24, 2021, which claims the benefit of co-pending U.S. Provisional Patent Application No. 63/117,710, filed Nov. 24, 2020, each of which is incorporated by reference in its entirety herein.

### STATEMENT REGARDING FEDERALLY SPONSORED RESEARCH OR DEVELOPMENT

This invention was made with government support under DP2GM128199-01 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide sequence listing submitted concurrently herewith and identified as follows: One 281,989 Byte ASCII (Text) file named “767465_ST25” created on May 23, 2023.

### BACKGROUND

Transcription factors (TFs) regulate cellular state by binding specific DNA promoter and enhancer sequences, thereby recruiting transcriptional machinery to activate or repress gene expression. DNA binding is carried out by modular domains that are shared by large families of transcription factors, and conserved across evolution. Aberrant TF activity is widely and unambiguously implicated in human disease. For example, many cancers are hallmarked by direct genetic alteration of TFs by amplification, deletion, translocation, or mutation. Cancers that do not harbor these direct alterations to TFs invariably rely on dysregulated upstream signaling pathways that ultimately impinge on TF function and gene expression programs. There is a need in the art for synthetic DNA binding domains (sDBDs), including those that are synthetic transcriptional regulators (STRs), including to treat or prevent cancer.

### BRIEF SUMMARY

In aspects, the disclosure provides a polypeptide construct comprising (a) a first polypeptide comprising an amino acid sequence derived from a basic helix of a transcription factor protein that comprises a basic helix-loop-helix domain; and (b) a second polypeptide comprising an amino acid sequence derived from a helix that extends in the C-terminal direction from the end of the loop of a basic helix-loop-helix domain of a transcription factor protein that comprises a basic helix-loop-helix domain; wherein the first polypeptide and the second polypeptide are linked through an interpolypeptide covalent linkage.

In aspects, the disclosure provides a polypeptide construct comprising (a) the polypeptide construct as described above; (b) a third polypeptide comprising an amino acid sequence derived from a basic helix of a transcription factor protein that comprises a basic helix-loop-helix domain; and (c) a fourth polypeptide comprising an amino acid sequence derived from a helix that extends in the C-terminal direction from the end of the loop of a basic helix-loop-helix domain of a transcription factor protein that comprises a basic helix-loop-helix domain; wherein the third polypeptide and the fourth polypeptide are linked through an interpolypeptide covalent linkage.

In aspects, the disclosure provides pharmaceutical compositions and methods of treating disease using the polypeptide constructs and polypeptides as described herein.

Additional aspects of the disclosure are as described herein.

### BRIEF DESCRIPTION OF THE DRAWINGS

FIG. 1 shows exemplary positions of residues within exemplary polypeptides, as described herein, that can be covalently linked, in accordance with aspects of the disclosure.

FIG. 2A shows a schematic of synthesis of a branched zipper helix, in accordance with aspects of the disclosure.

FIG. 2B shows a schematic of homodimerization, in accordance with aspects of the disclosure.

FIG. 2C shows schematics of heterodimerization with orthogonal chemistry for the synthesis of asymmetric tetrahelical peptide conjugate, in accordance with aspects of the disclosure.

FIG. 2D shows schematics of heterodimerization by switching the order of conjugation chemistry, in accordance with aspects of the disclosure.

FIG. 3A presents a schematic depiction, in accordance with aspects of the disclosure, of the basic helix-loop-helix (bHLH) domains of MYC and MAX. Individual monomers form hetero- or homodimeric complexes of MYC/MAX or MAX/MAX with duplex DNA. Cross-dimer ligation of B and Z helices from opposing monomers results in non-natural mimics that could assemble the tetrahelix bundle in a ‘sandwich’ dimer ([B-Z]₂) and recognize specific DNA sequences.

FIG. 3B presents convergent synthesis, in accordance with aspects of the disclosure, of STRs containing secondary and tertiary domain stabilizing groups. B and Z helices are synthesized on-resin with bisalkylated, terminal olefin containing ‘S₅’ amino acids at defined positions for on-resin ring closing metathesis. ‘Stapled’ B helices harbor an orthogonal Lys(Mmt) at a defined C-terminal position for deprotection and acylation with a maleimide linker. Stapled helices (Z2 and B1 shown here) are ligated in aqueous solution and readily purified to yield STRs of approximately 6 kDa.

FIG. 4A is a structural representation of a MAX-STR, in accordance with aspects of the disclosure.

FIG. 4B presents sequences of individual basic and zipper peptides containing helix stabilizing amino acids, mutations and interhelix ligation sites, in accordance with aspects of the disclosure. S₅, (S)-5-pentenyl alanine; N_{L}, norleucine; A_{ib}, α-amino isobutyric acid; k, D-lysine. Interhelix ligation sites (final K of basic helices; the C of zipper helices) here represent a glycylmaleimide modified lysine in the basic helix, ligated via a thioether with the corresponding zipper helix cysteine in elaborated STRs.

FIG. 4C presents graphs showing quantified band intensity from DNA competition EMSA gels containing constant levels of STR116, STR118, and MAX/MAX binding to E-box oligonucleotide probe in the presence of increasing doses of the listed unlabeled competitor DN, in accordance with aspects of the disclosure.

FIG. 5 presents petal plots showing activity features for indicated STRs, in accordance with aspects of the disclosure.

FIG. 6A is a graph showing per-cell total fluorescence intensity divided by nuclear region from HeLa cells treated with FITC-STR or DMSO, in accordance with aspects of the disclosure. Per-cell values (n=60 cells) with mean and s.d. shown as solid bars.

FIG. 6B is a graph showing ChTP-qPCR quantification of endogenous MYC occupancy at control and E-box-containing target genes in HeLa cells, in accordance with aspects of the disclosure. IgG is represented by the bar to the left of each x-axis tick mark, and Myc is represented by the bar to the right of each x-axis tick mark. Mean and s.e.m. of two independent biological replicates. Statistical analyses are by unpaired, two-sided t test. ns: not significant; *, p<0.05.

FIG. 6C is a graph showing photo-ChIP-qPCR quantification of P-BioSTR118 occupancy at control and E-box-containing target genes in P493-6 cells, in accordance with aspects of the disclosure. Biotin-Block is represented by the bar to the left of each x-axis tick mark, and STR118 is represented by the bar to the right of each x-axis tick mark. Mean and s.e.m. of three independent biological replicates. Statistical analyses are by unpaired, two-sided t test. ns: not significant; *, p<0.05; **, p<0.01.

FIG. 6D is a graph showing firefly luciferase activity in HCT116 E-box reporter cells measured after STR treatment (24 hr, 20 μM), in accordance with aspects of the disclosure. Mean and s.d. for 3 independent biological replicates. Statistical analyses are by unpaired, two-sided t test. *, p<0.05; ****, p<0.0001.

FIG. 6E is a graph showing relative viability of P493-6 cells treated with tetracycline (+Tet), or with vehicle, STR116, or STR118 for each time point shown, in accordance with aspects of the disclosure. Mean and s.d. from two biological replicates. For each day, from left to right, is Myc-ON, Myc-OFF (+Tet), STR116 at 10 μM, STR118 at 10 μM.

FIG. 6F is a graph showing 72-Hour viability of P493-6 cells treated with STR116 under conditions of low (left) or high MYC expression (right), in accordance with aspects of the disclosure. Mean and s.d. from two biological replicates.

FIG. 7 presents aschematic of contacts between an individual B-Z monomer and one half-site of the E-box containing oligonucleotide, in accordance with aspects of the disclosure. Dashes denote sequence-specific and backbone contacts, respectively; double wedges denote Van der Waals interactions.

FIG. 8A presents a schematic depicting the modular reprogramming of the MAX-STR scaffold to generate OLIG2-STR and TFAP4-STRs with altered sequence specificities, in accordance with aspects of the disclosure.

FIG. 8B presents sequences of B-Z (MAX-derived), STR69, and STR640, in accordance with aspects of the disclosure, and also the sequences of duplex DNA probes E1, E2, and E3 (antisense complement strand not shown) as used in Example 1.

FIG. 8C presents graphs showing dose-dependent target selectivity curves from quantified EMSA gels for MAX-, TFAP4- and OLIG2-derived STRs binding to indicated target sequences E1, E2, and E3 of FIG. 8B, in accordance with aspects of the disclosure.

FIG. 9 presents the chemical structure of P-BioSTR118, in accordance with aspects of the disclosure.

FIG. 10 presents sequences and representative models, in accordance with aspects of the disclosure, for STRs that contain the basic sequence of OLIG2 grafted onto MAX ‘B-Z’ structure (MAX-OLIG2-STR) and an STR mimetic developed from the complete primary sequence of OLIG2 (OLIG2-STR).

FIG. 11 is a graph showing activity of a c-myc responsive luciferase reporter gene with increasing concentrations of STR1180, in accordance with aspects of the disclosure.

FIG. 12 is a graph showing results of the helical tetramers of STR116 (STR116T) and STR118 (STR118T) tested in a luciferase assay, in accordance with aspects of the disclosure.

FIG. 13 is a graph showing binding data for STR116T and STR118T, in accordance with aspects of the disclosure.

FIG. 14 presents the chemical structure of STR116T, in accordance with aspects of the disclosure.

FIG. 15 presents the chemical structure of STR118T, in accordance with aspects of the disclosure.

### DETAILED DESCRIPTION

In aspects, the disclosure provides a polypeptide construct comprising (a) a first polypeptide comprising an amino acid sequence derived from a basic helix of a transcription factor protein that comprises a basic helix-loop-helix domain; and (b) a second polypeptide comprising an amino acid sequence derived from a helix that extends in the C-terminal direction from the end of the loop of a basic helix-loop-helix domain of a transcription factor protein that comprises a basic helix-loop-helix domain; wherein the first polypeptide and the second polypeptide are linked through an interpolypeptide covalent linkage.

In aspects of the disclosure, synthetic transcription factors bind to DNA with comparable affinity and specify when compared to native proteins. In aspects, synthetic transcription factors comprise a covalent helix cross-dimer, wherein two defined helices that comprise a DNA-binding helix (basic helix, B) and a structure-orienting zipper helix (Z), each derived from bHLH protein family proteins or derivatives thereof, are chemically connected, e.g., via intermolecular side chain-to-side chain linkers. Ligation positions on the helices for the intermolecular connection can be chosen for opposing helices of the tetrahelix bundle normally formed by two bHLH proteins that have bound to one another. In the case of synthetic DNA binding domains herein, the B-Z helices can be chosen from opposing monomers, and chemically linked such that they can self-assemble in a “sandwich-like” fashion to bind DNA. Therefore, the monomeric sDBDs described herein can be chemically and structurally defined but completely non-natural in structure. The fully synthetic di-helix monomer can dimerize with an additional synthetic transcription factor to form a tertiary structure that mimics the natural transcription factor DNA binding bHLH domain. Due to the cross-dimer linkage, synthetic transcription factors described herein may not form productive binding interactions with native bHLH domains. Synthesis of sDBDs derived from the bHLH transcription factors can be modular in nature.

The amino sequences of bHLH domains from 105 human proteins are identified in Tables 1A and 1B. Clustal Omega (https://www.ebi.ac.uk/Tools/msa/clustalo/) was used to perform a multiple sequence alignment and the aligned sequences are presented in Table 2. Approximately positions 1-29 represent the “B” helix, approximately positions 30-43 represent the loop, and approximately positions 44-60 represent the “Z” helix. To generate a synthetic transcription factor, a covalent chemical crosslink is formed between a “B” and “Z” helix derived from any bHLH domain. The chemical crosslink can be, e.g., between position 23 of the “B” helix and position 51 of the “Z” helix, or a different position that maintains the defined binding orientation of synthetic transcription factors. Altering the non-natural amino acid positions and helix-stabilization strategies can modulate the resulting STF's binding activity and proteolytic stability. N-terminal extension or truncation of amino acids to the “B” helix can be used to modulate DNA binding affinity and specificity and C-terminal amino acid extension or truncation to the “Z” helix can be used to modulate DNA binding affinity and specificity.

TABLE 1A

| | | | SEQ ID |
|---|---|---|---|
| Protein | Name | B Sequence (e.g., 1-36) | No |
| Generic sequence | | --\hat{ }---\hat{ }---\hat{ }---\hat{ }---\hat{ }---\hat{ }-----\hat{ }---\hat{ }--- | |
| cyclizing positions | | | |
| Generic sequence | | ----------------------*--**--*---*-* | |
| Interhelix ligation | | | |
| positions | | | |
| Generic sequence | | --#---#---#---#---#---#-----#---#--- | |
| sites for stabilizing | | | |
| mutations | | | |
| Max-derived | B | --------KRAHHNALERKRRDHIKDSFH*LRDSVP | 1 |
| Max-derived | BLI1 | PRFQSAADKRAHHNALERKRRDHIKDSFH*LRDSVP | 2 |
| Max-derived | B1 | --------KR\hat{ }HHN\hat{ }LERKRRDHIKDSFH*LRDSVP | 3 |
| Max-derived | B6 | --------KR\hat{ }HHN\hat{ }LERkRRDHIKDSFH*LRDSVP | 4 |
| Max-derived | B11 | PRFQSA\hat{ }DKR\hat{ }HHNALERKRRDHIKDSFH*LRDSVP | 5 |
| Aryl hydrocarbon | 1B | QKTVKPIPAEGIKSNPSKRHRDRLNTELDRLASLLP | 6 |
| receptor | | | |
| Neuronal PAS domain- | 2B | --------MYRSTKGASKARRDQINAEIRNLKELLP | 7 |
| containing protein 4 | | | |
| Aryl hydrocarbon | 3B | PLQKQRPAVGAEKSNPSKRHRDRLNAELDHLASLLP | 8 |
| receptor repressor | | | |
| Neuronal PAS domain- | 4B | SGPCLQAQRKEKSRNAARSRRGKENLEFFELAKLLP | 9 |
| containing protein 1 | | | |
| Neuronal PAS domain- | 5B | TYQNLQALRKEKSRDAARSRRGKENFEFYELAKLLP | 10 |
| containing protein 3 | | | |
| Single-minded homolog | 6B | --------MKEKSKNAARTRREKENSEFYELAKLLP | 11 |
| 1 | | | |
| Single-minded | 7B | --------MKEKSKNAAKTRREKENGEFYELAKLLP | 12 |
| homolog 2 | | | |
| Endothelial PAS | 8B | KKRSSSERRKEKSRDAARCRRSKETEVFYELAHELP | 13 |
| domain-containing | | | |
| protein 1 | | | |
| Hypoxia-inducible | 9B | KKKISSERRKEKSRDAARSRRSKESEVFYELAHQLP | 14 |
| factor 1-alpha | | | |
| Hypoxia-inducible | 10B | RARSTTELRKEKSRDAARSRRSQETEVLYQLAHTLP | 15 |
| factor 3-alpha | | | |
| Nuclear receptor | 11B | SHKRKGSPCDTLASSTEKRRREQENKYLEELAELLS | 16 |
| coactivator 1 | | | |
| Nuclear receptor | 12B | RKECPDQLGPSPKRNTEKRNREQENKYIEELAELIF | 17 |
| coactivator 2 | | | |
| Nuclear receptor | 13B | RKLPCDTPGQGLTCSGEKRRREQESKYIEELAELIS | 18 |
| coactivator 3 | | | |
| Spermatogenesis-and | 14B | TVAEGPSSCLRRNVISERERRKRMSLSCERLRALLP | 19 |
| oogenesis-specific | | | |
| basic helix-loop- | | | |
| helix-containing | | | |
| protein 1 | | | |
| Transcription factor- | 15B | GPQGGRSQRRERHNRMERDRRRRIRICCDELNLLVP | 20 |
| like 5 protein | | | |
| Spermatogenesis-and | 16B | SEFEKNKKISLLHSSKEKLRRERIKYCCEQLRTLLP | 21 |
| oogenesis-specific | | | |
| basic helix-loop- | | | |
| helix-containing | | | |
| protein 2 | | | |
| Hairy and enhancer of | 17B | DKLKERKRTPVSHKVIEKRRRDRINRCLNELGKTVP | 22 |
| split-related protein | | | |
| HELT | | | |
| Hairy/enhancer-of- | 18B | PSSSQMQARKKHRGIIEKRRRDRINSSLSELRRLVP | 23 |
| split related with | | | |
| YRPW motif-like | | | |
| protein | | | |
| Hairy/enhancer-of- | 19B | TTSSQILARKRRRGIIEKRRRDRINNSLSELRRLVP | 24 |
| split related with | | | |
| YRPW motif protein 1 | | | |
| Hairy/enhancer-of- | 20B | TTTSQIMARKKRRGIIEKRRRDRINNSLSELRRLVP | 25 |
| split related with | | | |
| YRPW motif protein 2 | | | |
| Class E basic helix- | 21B | RSEDSKETYKLPHRLIEKKRRDRINECIAQLKDLLP | 26 |
| loop-helix protein 40 | | | |
| Class E basic helix- | 22B | KRDDTKDTYKLPHRLIEKKRRDRINECIAQLKDLLP | 27 |
| loop-helix protein 41 | | | |
| Transcription factor | 23B | DRAENRDGPKMLKPLVEKRRRDRINRSLEELRLLLL | 28 |
| HES-7 | | | |
| Transcription | 24B | DGWETRGDRKARKPLVEKKRRARINESLQELRLLLA | 29 |
| cofactor HES-6 | | | |
| Transcription factor | 25B | ELLSPKEKNRLRKPVVEKMRRDRINSSIEQLKLLLE | 30 |
| HES-5 | | | |
| Transcription factor | 26B | ---------------MEKKRRARINVSLEQLKSLLE | 31 |
| HES-3 | | | |
| Transcription factor | 27B | RAGDAAELRKSLKPLLEKRRRARINQSLSQLKGLIL | 32 |
| HES-2 | | | |
| Transcription factor | 28B | KPKTASEHRKSSKPIMEKRRRARINESLSQLKTLIL | 33 |
| HES-1 | | | |
| Transcription factor | 29B | KPRSAAEHRKSSKPVMEKRRRARINESLAQLKTLIL | 34 |
| HES-4 | | | |
| Circadian locomoter | 30B | EEDDKDKAKRVSRNKSEKKRRDQFNVLIKELGSMLP | 35 |
| output cycles protein | | | |
| kaput | | | |
| Neuronal PAS domain- | 31B | DEDEKDRAKRASRNKSEKKRRDQFNVLIKELSSMLP | 36 |
| containing protein 2 | | | |
| Aryl hydrocarbon | 32B | SSADKERLARENHSEIERRRRNKMTAYITELSDMVP | 37 |
| receptor nuclear | | | |
| translocator | | | |
| Aryl hydrocarbon | 33B | EHQVKMKAFREAHSQTEKRRRDKMNNLIEELSAMIP | 38 |
| receptor nuclear | | | |
| translocator-like | | | |
| protein 2 | | | |
| Basic helix-loop- | 34B | TKKQHRKKNRETHNAVERHRKKKINAGINRIGELIP | 39 |
| helix domain- | | | |
| containing protein | | | |
| USF3 | | | |
| Upstream stimulatory | 35B | PRTTRDEKRRAQHNEVERRRRDKINNWIVQLSKIIP | 40 |
| factor 1 | | | |
| Upstream stimulatory | 36B | TRTPRDERRRAQHNEVERRRRDKINNWIVQLSKIIP | 41 |
| factor 2 | | | |
| Transcription factor | 37B | RALAKERQKKDNHNLIERRRRFNINDRIKELGMLIP | 42 |
| EB | | | |
| Transcription factor | 38B | RALAKERQKKDNHNLIERRRRYNINYRIKELGTLIP | 43 |
| EC | | | |
| Microphthalmia- | 39B | RALAKERQKKDNHNLIERRRRFNINDRIKELGTLIP | 44 |
| associated | | | |
| transcription factor | | | |
| Transcription factor | 40B | KALLKERQKKDNHNLIERRRRFNINDRIKELGTLIP | 45 |
| E3 | | | |
| Sterol regulatory | 41B | ASAQSRGEKRTAHNAIEKRYRSSINDKIIELKDLVV | 46 |
| element-binding | | | |
| protein 1 | | | |
| Sterol regulatory | 42B | LEPPKEGERRTTHNIIEKRYRSSINDKIIELKDLVM | 47 |
| element-binding | | | |
| protein 2 | | | |
| Max-binding protein | 43B | KKRPGGIGTREVHNKLEKNRRAHLKECFETLKRNIP | 48 |
| MNT | | | |
| Max dimerization | 44B | QAPGAQDSGRSVHNELEKRRRAQLKRCLERLKQQMP | 49 |
| protein 3 | | | |
| Max dimerization | 45B | GLVRKAPNNRSSHNELEKHRRAKLRLYLEQLKQLVP | 50 |
| protein 4 | | | |
| Max dimerization | 46B | KSKKNNSSSRSTHNEMEKNRRAHLRLCLEKLKGLVP | 51 |
| protein 1 | | | |
| Max-interacting | 47B | GSSNTSTANRSTHNELEKNRRAHLRLCLERLKVLIP | 52 |
| protein 1 | | | |
| Max-like protein X | 48B | YKESYKDRRRRAHTQAEQKRRDAIKRGYDDLQTIVP | 53 |
| MLX-interacting | 49B | KNVAALKNRQMKHISAEQKRRFNIKMCFDMLNSLIS | 54 |
| protein | | | |
| Carbohydrate- | 50B | PDSNKTENRRITHISAEQKRRFNIKLGFDTLHGLVS | 55 |
| responsive element- | | | |
| binding protein | | | |
| Factor in the | 51B | ENLQLVLERRRVANAKERERIKNLNRGFARLKALVP | 56 |
| germline alpha | | | |
| Protein max | 52B | PRFQSAADKRAHHNALERKRRDHIKDSFHSLRDSVP | 57 |
| Protein L-Myc | 53B | SSDTEDVTKRKNHNFLERKRRNDLRSRFLALRDQVP | 58 |
| Myc proto-oncogene | 54B | SSDTEENVKRRTHNVLERQRRNELKRSFFALRDQIP | 59 |
| protein | | | |
| N-myc proto-oncogene | 55B | NSDSEDSERRRNHNILERQRRNDLRSSFLTLRDHVP | 60 |
| protein | | | |
| Transcription factor | 56B | QKAEREKERRVANNARERLRVRDINEAFKELGRMCQ | 61 |
| E2-alpha | | | |
| Transcription factor | 57B | QKIEREKERRMANNARERLRVRDINEAFKELGRMCQ | 62 |
| 12 | | | |
| Transcription factor | 58B | QKAEREKERRMANNARERLRVRDINEAFKELGRMVQ | 63 |
| 4 | | | |
| Achaete-scute homolog | 59B | SLPQQQPAAVARRNERERNRVKLVNLGFATLREHVP | 64 |
| 1 | | | |
| Achaete-scute homolog | 60B | AETGGGAAAVARRNERERNRVKLVNLGFQALRQHVP | 65 |
| 2 | | | |
| Achaete-scute homolog | 61B | CEYSYGPAFTRKRNERERQRVKCVNEGYAQLRHHLP | 66 |
| 3 | | | |
| Achaete-scute homolog | 62B | LDSAFEPAFLRKRNERERQRVRCVNEGYARLRDHLP | 67 |
| 4 | | | |
| Achaete-scute homolog | 63B | YEYPFEPAFIQKRNERERQRVKCVNEGYARLRGHLP | 68 |
| 5 | | | |
| Transcription factor | 64B | QRDOERRIRREIANSNERRRMQSINAGFQSLKTLIP | 69 |
| AP-4 | | | |
| Mesoderm posterior | 65B | RSSRLGSGQRQSASEREKLRMRTLARALHELRRFLP | 70 |
| protein 1 | | | |
| Mesoderm posterior | 66B | ARTGPAGGQROSASEREKLRMRTLARALHELRRFLP | 71 |
| protein 2 | | | |
| Mesogenin-1 | 67B | TKVRMSVQRRRKASEREKLRMRTLADALHTLRNYLP | 72 |
| Oligodendrocyte | 68B | AKEEQQQQLRRKINSRERKRMQDLNLAMDALREVIL | 73 |
| transcription factor | | | |
| 1 | | | |
| Oligodendrocyte | 69B | MTEPELQQLRLKINSRERKRMHDLNIAMDGLREVMP | 74 |
| transcription factor | | | |
| 2 | | | |
| Oligodendrocyte | 70B | LSEQDLQQLRLKINGRERKRMHDLNLAMDGLREVMP | 75 |
| transcription factor | | | |
| 3 | | | |
| Class E basic helix- | 71B | KKSKEQKALRLNINARERRRMHDLNDALDELRAVIP | 76 |
| loop-helix protein 22 | | | |
| Class E basic helix- | 72B | RRPREQRSLRLSINARERRRMHDLNDALDGLRAVIP | 77 |
| loop-helix protein 23 | | | |
| Class A basic helix- | 73B | GRRDSSIQRRLESNERERQRMHKLNNAFQALREVIP | 78 |
| loop-helix protein 15 | | | |
| Protein atonal | 74B | QVNGVQKQRRLAANARERRRMHGLNHAFDQLRNVIP | 79 |
| homolog 1 | | | |
| Protein atonal | 75B | RLESAARRRLAANARERRRMQGLNTAFDRLRRVVP | 80 |
| homolog 7 | | | |
| Protein atonal | 76B | EIKALQQTRRLLANARERTRVHTISAAFEALRKQVP | 81 |
| homolog 8 | | | |
| Neurogenin-2 | 77B | TVQRIKKTRRLKANNRERNRMHNLNAALDALREVLP | 82 |
| Neurogenin-1 | 78B | LLHSLRRSRRVKANDRERNRMHNLNAALDALRSVLP | 83 |
| Neurogenin-3 | 79B | ALSKORRSRRKKANDRERNRMHNLNSALDALRGVLP | 84 |
| Neurogenic | 80B | ARLERFRARRVKANARERTRMHGLNDALDNLRRVMP | 85 |
| differentiation | | | |
| factor 4 | | | |
| Neurogenic | 81B | ARLERFKLRRMKANARERNRMHGLNAALDNLRKVVP | 86 |
| differentiation | | | |
| factor 1 | | | |
| Neurogenic | 82B | ARLERSKLRRQKANARERNRMHDLNAALDNLRKVVP | 87 |
| differentiation | | | |
| factor 2 | | | |
| Neurogenic | 83B | LRLERVKFRRQEANARERNRMHGLNDALDNLRKVVP | 88 |
| differentiation | | | |
| factor 6 | | | |
| Myogenic factor 5 | 84B | KRKSTTMDRRKAATMRERRRLKKVNQAFETLKRCTT | 89 |
| Myoblast | 85B | KRKTTNADRRKAATMRERRRLSKVNEAFETLKRCTS | 90 |
| determination protein | | | |
| 1 | | | |
| Myogenic factor 6 | 86B | KRKSAPTDRRKAATLRERRRLKKINEAFEALKRRTV | 91 |
| Myogenin | 87B | KRKSVSVDRRRAATLREKRRLKKVNEAFEALKRSTL | 92 |
| Class A basic helix- | 88B | ARPVRSKARRMAANVRERKRILDYNEAFNALRRALR | 93 |
| loop-helix protein 9 | | | |
| Fer3-like protein | 89B | RKRVITYAQRQAANIRERKRMFNLNEAFDQLRRKVP | 94 |
| Pancreas | 90B | RSEAELQQLRQAANVRERRRMQSINDAFEGLRSHIP | 95 |
| transcription factor | | | |
| 1 subunit alpha | | | |
| Helix-loop-helix | 91B | RRRRATAKYRTAHATRERIRVEAFNLAFAELRKLLP | 96 |
| protein 1 | | | |
| Helix-loop-helix | 92B | RRRRATAKYRSAHATRERIRVEAFNLAFAELRKLLP | 97 |
| protein 2 | | | |
| T-cell acute | 93B | ------MTRKIFTNTRERWRQQNVNSAFAKLRKLIP | 98 |
| lymphocytic leukemia | | | |
| protein 2 | | | |
| Protein lyl-1 | 94B | GHQPQKVARRVFTNSRERWRQQNVNGAFAELRKLLP | 99 |
| T-cell acute | 95B | DGPHTKVVRRIFTNSRERWRQQNVNGAFAELRKLIP | 100 |
| lymphocytic leukemia | | | |
| protein 1 | | | |
| Heart-and neural | 96B | ALGGRLGRRKGSGPKKERRRTESINSAFAELRECIP | 101 |
| crest derivatives- | | | |
| expressed protein 1 | | | |
| Heart-and neural | 97B | LGGPRPVKRRGTANRKERRRTOSINSAFAELRECIP | 102 |
| crest derivatives- | | | |
| expressed protein 2 | | | |
| Transcription factor | 98B | GLALGRSEASPENAARERSRVRTLRQAFLALQAALP | 103 |
| 23 | | | |
| Transcription factor | 99B | GSRSGSGRPAAANAARERSRVQTLRHAFLELORTLP | 104 |
| 24 | | | |
| Musculin | 100B | SAAECKQSQRNAANARERARMRVLSKAFSRLKTSLP | 105 |
| Transcription factor | 101B | VSQEGKQVORNAANARERARMRVLSKAFSRLKTTLP | 106 |
| 21 | | | |
| Basic helix-loop- | 102B | GRPGREPRORHTANARERDRTNSVNTAFTALRTLIP | 107 |
| helix transcription | | | |
| factor scleraxis | | | |
| Transcription factor | 103B | AGPVVVVRQRQAANARERDRTQSVNTAFTALRTLIP | 108 |
| 15 | | | |
| Twist-related protein | 104B | QSYEELQTQRVMANVRERQRTQSLNEAFAALRKIIP | 109 |
| 1 | | | |
| Twist-related protein | 105B | QSFEELQSQRILANVRERQRTQSLNEAFAALRKIIP | 110 |
| 2 | | | |

TABLE 1B

| | | | SEQ ID |
|---|---|---|---|
| Protein | Name | Z Sequence (e.g., 1-31) | NO |
| Generic sequence | | ---\hat{ }---\hat{ }------\hat{ }--\hat{ }--\hat{ }\hat{ }--------- | |
| cyclizing positions | | | |
| Generic sequence | | --**--**--*--*----------------- | |
| Interhelix ligation | | | |
| positions | | | |
| Generic sequence | | --##---#------##-##--##-------- | |
| sites for stabilizing | | | |
| mutations | | | |
| Max-derived | Z | SRAQIL*KATEYIQY#RRKN----------- | 111 |
| Max-derived | ZL1 | SRAQIL*KATEYIQY#RRKNHTHQQDIDDLK | 112 |
| Max-derived | Z2 | SRAQIL*KATEYIQ\hat{ }HRR\hat{ }N----------- | 113 |
| Max-derived | Z4 | SR#QIL*QATEYIQ\hat{ }HRR\hat{ }N----------- | 114 |
| Max-derived | Z6 | SR#QIL*QATEYIQ\hat{ }HRR\hat{ }LHTHE------- | 115 |
| Aryl hydrocarbon | 1Z | DKLSVLRLSVSYLRAKSFFDVALKSSPTERN | 116 |
| receptor | | | |
| Neuronal PAS domain- | 2Z | SYLHIMSLACIYTRKGVFFAGGTPLAGPTGL | 117 |
| containing protein 4 | | | |
| Aryl hydrocarbon | 3Z | DKLSVLRLSVSYLRVKSFFQVVQEQSSRQPA | 118 |
| receptor repressor | | | |
| Neuronal PAS domain- | 4Z | DKASIVRLSVTYLRLRRFAALGAPPWGLRAA | 119 |
| containing protein 1 | | | |
| Neuronal PAS domain- | 5Z | DKASIIRLTISYLKMRDFANQGDPPWNLRME | 120 |
| containing protein 3 | | | |
| Single-minded homolog | 6Z | DKASIIRLTTSYLKMRVVFPEGLGEAWGHSS | 121 |
| 1 | | | |
| Single-minded | 7Z | DKASIIRLTTSYLKMRAVFPEGLGDAWGQPS | 122 |
| homolog 2 | | | |
| Endothelial PAS | 8Z | DKASIMRLAISFLRTHKLLSSVCSENESEAE | 123 |
| domain-containing | | | |
| protein 1 | | | |
| Hypoxia-inducible | 9Z | DKASVMRLTISYLRVRKLLDAGDLDIEDDMK | 124 |
| factor 1-alpha | | | |
| Hypoxia-inducible | 10Z | DKASIMRLTISYLRMHRLCAAGEWNQVGAGG | 125 |
| factor 3-alpha | | | |
| Nuclear receptor | 11Z | DKCKILKKTVDQIQLMKRMEQEKSTTDDDVQ | 126 |
| coactivator 1 | | | |
| Nuclear receptor | 12Z | DKCAILKETVKQIRQIKEQEKAAAANIDEVQ | 127 |
| coactivator 2 | | | |
| Nuclear receptor | 13Z | DKCAILKETVRQIRQIKEQGKTISNDDDVQK | 128 |
| coactivator 3 | | | |
| Spermatogenesis-and | 14Z | DMASVLEMSVQFLRLASALGPSQEQHAILAS | 129 |
| oogenesis-specific | | | |
| basic helix-loop- | | | |
| helix-containing | | | |
| protein 1 | | | |
| Transcription factor- | 15Z | DKATTLQWTTAFLKYIQERHGDSLKKEFESV | 130 |
| like 5 protein | | | |
| Spermatogenesis-and | 16Z | DAASVLEATVDYVKYIREKISPAVMAQITEA | 131 |
| oogenesis-specific | | | |
| basic helix-loop- | | | |
| helix-containing | | | |
| protein 2 | | | |
| Hairy and enhancer of | 17Z | EKAEILEMTVQYLRALHSADFPRGREKAELL | 132 |
| split-related protein | | | |
| HELT | | | |
| Hairy/enhancer-of- | 18Z | EKAEVLQMTVDHLKMLHATGGTGFFDARALA | 133 |
| split related with | | | |
| YRPW motif-like | | | |
| protein | | | |
| Hairy/enhancer-of- | 19Z | EKAEILQMTVDHLKMLHTAGGKGYFDAHALA | 134 |
| split related with | | | |
| YRPW motif protein 1 | | | |
| Hairy/enhancer-of- | 20Z | EKAEILQMTVDHLKMLQATGGKGYFDAHALA | 135 |
| split related with | | | |
| YRPW motif protein 2 | | | |
| Class E basic helix- | 21Z | EKAVVLELTLKHVKALTNLIDQQQQKIIALQ | 136 |
| loop-helix protein 40 | | | |
| Class E basic helix- | 22Z | EKAVVLELTLKHLKALTALTEQQHQKIIALQ | 137 |
| loop-helix protein 41 | | | |
| Transcription factor | 23Z | EKAEILEFAVGYLRERSRVEPPGVPRSPVQD | 138 |
| HES-7 | | | |
| Transcription | 24Z | ENAEVLELTVRRVQGVLRGRAREREQLQAEA | 139 |
| cofactor HES-6 | | | |
| Transcription factor | 25Z | EKADILEMAVSYLKHSKAFVAAAGPKSLHQD | 140 |
| HES-5 | | | |
| Transcription factor | 26Z | EKADILELSVKYMRSLONSLQGLWPVPRGAE | 141 |
| HES-3 | | | |
| Transcription factor | 27Z | EKADVLEMTVRFLQELPASSWPTAAPLPCDS | 142 |
| HES-2 | | | |
| Transcription factor | 28Z | EKADILEMTVKHLRNLQRAQMTAALSTDPSV | 143 |
| HES-1 | | | |
| Transcription factor | 29Z | EKADILEMTVRHLRSLRRVQVTAALSADPAV | 144 |
| HES-4 | | | |
| Circadian locomoter | 30Z | DKSTVLQKSIDFLRKHKEITAQSDASEIRQD | 145 |
| output cycles protein | | | |
| kaput | | | |
| Neuronal PAS domain- | 31Z | DKTTVLEKVIGFLQKHNEVSAQTEICDIQQD | 146 |
| containing protein 2 | | | |
| Aryl hydrocarbon | 32Z | DKLTILRMAVSHMKSLRGTGNTSTDGSYKPS | 147 |
| receptor nuclear | | | |
| translocator | | | |
| Aryl hydrocarbon | 33Z | DKLTVLRMAVQHLRSLKGLTNSYVGSNYRPS | 148 |
| receptor nuclear | | | |
| translocator-like | | | |
| protein 2 | | | |
| Basic helix-loop- | 34Z | SKNMILDQAFKYITELKRONDELLLNGGNNE | 149 |
| helix domain- | | | |
| containing protein | | | |
| USF3 | | | |
| Upstream stimulatory | 35Z | SKGGILSKACDYIQELRQSNHRLSEELQGLD | 150 |
| factor 1 | | | |
| Upstream stimulatory | 36Z | SKGGILSKACDYIRELRQTNQRMQETFKEAE | 151 |
| factor 2 | | | |
| Transcription factor | 37Z | NKGTILKASVDYIRRMQKDLQKSRELENHSR | 152 |
| EB | | | |
| Transcription factor | 38Z | NKGTILKASVEYIKWLQKEQQRARELEHRQK | 153 |
| EC | | | |
| Microphthalmia- | 39Z | NKGTILKASVDYIRKLQREQQRAKELENRQK | 154 |
| associated | | | |
| transcription factor | | | |
| Transcription factor | 40Z | NKGTILKASVDYIRKLQKEQQRSKDLESRQR | 155 |
| E3 | | | |
| Sterol regulatory | 41Z | NKSAVLRKAIDYIRFLQHSNQKLKQENLSLR | 156 |
| element-binding | | | |
| protein 1 | | | |
| Sterol regulatory | 42Z | HKSGVLRKAIDYIKYLQQVNHKLRQENMVLK | 157 |
| element-binding | | | |
| protein 2 | | | |
| Max-binding protein | 43Z | SNLSVLRTALRYIQSLKRKEKEYEHEMERLA | 158 |
| MNT | | | |
| Max dimerization | 44Z | TTLSLLRRARMHIQKLEDQEQRARQLKERLR | 159 |
| protein 3 | | | |
| Max dimerization | 45Z | TTLSLLKRAKVHIKKLEEQDRRALSIKEQLQ | 160 |
| protein 4 | | | |
| Max dimerization | 46Z | TTLSLLTKAKLHIKKLEDCDRKAVHQIDQLQ | 161 |
| protein 1 | | | |
| Max-interacting | 47Z | TTLGLLNKAKAHIKKLEEAERKSQHOLENLE | 162 |
| protein 1 | | | |
| Max-like protein X | 48Z | SKAIVLQKTIDYIQFLHKEKKKQEEEVSTLR | 163 |
| MLX-interacting | 49Z | SHAITLQKTVEYITKLQQERGOMQEEARRLR | 164 |
| protein | | | |
| Carbohydrate- | 50Z | SKATTLQKTAEYILMLQQERAGLQEEAQQLR | 165 |
| responsive element- | | | |
| binding protein | | | |
| Factor in the | 51Z | SKVDILKGATEYIQVLSDLLEGAKDSKKQDP | 166 |
| germline alpha | | | |
| Protein max | 52Z | SRAQILDKATEYIQYMRRKNHTHQQDIDDLK | 167 |
| Protein L-Myc | 53Z | PKVVILSKALEYLQALVGAEKRMATEKRQLR | 168 |
| Myc proto-oncogene | 54Z | PKVVILKKATAYILSVQAEEQKLISEEDLLR | 169 |
| protein | | | |
| N-myc proto-oncogene | 55Z | AKVVILKKATEYVHSLQAEEHQLLLEKEKLQ | 170 |
| protein | | | |
| Transcription factor | 56Z | TKLLILHQAVSVILNLEQQVRERNLNPKAAC | 171 |
| E2-alpha | | | |
| Transcription factor | 57Z | TKLLILHQAVAVILSLEQQVRERNLNPKAAC | 172 |
| 12 | | | |
| Transcription factor | 58Z | TKLLILHQAVAVILSLEQQVRERNLNPKAAC | 173 |
| 4 | | | |
| Achaete-scute homolog | 59Z | SKVETLRSAVEYIRALQQLLDEHDAVSAAFQ | 174 |
| 1 | | | |
| Achaete-scute homolog | 60Z | SKVETLRSAVEYIRALQRLLAEHDAVRNALA | 175 |
| 2 | | | |
| Achaete-scute homolog | 61Z | SKVETLRAAIKYINYLQSLLYPDKAETKNNP | 176 |
| 3 | | | |
| Achaete-scute homolog | 62Z | SKVETLRAAIDYIKHLQELLERQAWGLEGAA | 177 |
| 4 | | | |
| Achaete-scute homolog | 63Z | SKVETLRAAIRYIKYLQELLSSAPDGSTPPA | 178 |
| 5 | | | |
| Transcription factor | 64Z | SKAAILQQTAEYIFSLEQEKTRLLQQNTQLK | 179 |
| AP-4 | | | |
| Mesoderm posterior | 65Z | TKIETLRLAIRYIGHLSAVLGLSEESLQRRC | 180 |
| protein 1 | | | |
| Mesoderm posterior | 66Z | TKIETLRLAIRYIGHLSAVLGLSEESLQCRR | 181 |
| protein 2 | | | |
| Mesogenin-1 | 67Z | TKIQTLKYTIKYIGELTDLLNRGREPRAQSA | 182 |
| Oligodendrocyte | 68Z | SKIATLLLARNYILLLGSSLQELRRALGEGA | 183 |
| transcription factor | | | |
| 1 | | | |
| Oligodendrocyte | 69Z | SKIATLLLARNYILMLTNSLEEMKRLVSEIY | 184 |
| transcription factor | | | |
| 2 | | | |
| Oligodendrocyte | 70Z | SKIATLLLARNYILMLTSSLEEMKRLVGEIY | 185 |
| transcription factor | | | |
| 3 | | | |
| Class E basic helix- | 71Z | SKIATLLLAKNYILMQAQALEEMRRLVAYLN | 186 |
| loop-helix protein 22 | | | |
| Class E basic helix- | 72Z | SKIATLLLAKNYILMQAQALDEMRRLVAFLN | 187 |
| loop-helix protein 23 | | | |
| Class A basic helix- | 73Z | SKIETLTLAKNYIKSLTATILTMSSSRLPGL | 188 |
| loop-helix protein 15 | | | |
| Protein atonal | 74Z | SKYETLQMAQIYINALSELLQTPSGGEQPPP | 189 |
| homolog 1 | | | |
| Protein atonal | 75Z | SKYETLQMALSYIMALTRILAEAERFGSERD | 190 |
| homolog 7 | | | |
| Protein atonal | 76Z | SKLAILRIACNYILSLARLADLDYSADHSNL | 191 |
| homolog 8 | | | |
| Neurogenin-2 | 77Z | TKIETLRFAHNYIWALTETLRLADHCGGGGG | 192 |
| Neurogenin-1 | 78Z | TKIETLRFAYNYIWALAETLRLADQGLPGGG | 193 |
| Neurogenin-3 | 79Z | TKIETLRFAHNYIWALTQTLRIADHSLYALE | 194 |
| Neurogenic | 80Z | SKIETLRLARNYIWALSEVLETGOTPEGKGF | 195 |
| differentiation | | | |
| factor 4 | | | |
| Neurogenic | 81Z | SKIETLRLAKNYIWALSEILRSGKSPDLVSF | 196 |
| differentiation | | | |
| factor 1 | | | |
| Neurogenic | 82Z | SKIETLRLAKNYIWALSEILRSGKRPDLVSY | 197 |
| differentiation | | | |
| factor 2 | | | |
| Neurogenic | 83Z | SKIETLRLAKNYIWALSEILRIGKRPDLLTF | 198 |
| differentiation | | | |
| factor 6 | | | |
| Myogenic factor 5 | 84Z | PKVEILRNAIRYIESLQELLREQVENYYSLP | 199 |
| Myoblast | 85Z | PKVEILRNAIRYIEGLQALLRDQDAAPPGAA | 200 |
| determination protein | | | |
| 1 | | | |
| Myogenic factor 6 | 86Z | PKVEILRSAISYIERLQDLLHRLDQQEKMQE | 201 |
| Myogenin | 87Z | PKVEILRSAIQYIERLQALLSSLNQEERDLR | 202 |
| Class A basic helix- | 88Z | SKIATLRRAIHRIAALSLVLRASPAPRGPCG | 203 |
| loop-helix protein 9 | | | |
| Fer3-like protein | 89Z | SRIETLRLAIVYISFMTELLESCEKKESG | 204 |
| Pancreas | 90Z | SKVDTLRLAIGYINFLSELVQADLPLRGGGA | 205 |
| transcription factor | | | |
| 1 subunit alpha | | | |
| Helix-loop-helix | 91Z | SKIEILRLAICYISYLNHVLDV | 206 |
| protein 1 | | | |
| Helix-loop-helix | 92Z | SKIEILRLAICYISYLNHVLDV | 207 |
| protein 2 | | | |
| T-cell acute | 93Z | SKNETLRLAMRYINFLVKVLGEQSLQQTGVA | 208 |
| lymphocytic leukemia | | | |
| protein 2 | | | |
| Protein lyl-1 | 94Z | SKNEVLRLAMKYIGFLVRLLRDQAAALAAGP | 209 |
| T-cell acute | 95Z | SKNEILRLAMKYINFLAKLLNDQEEEGTQRA | 210 |
| lymphocytic leukemia | | | |
| protein 1 | | | |
| Heart-and neural | 96Z | SKIKTLRLATSYIAYLMDVLAKDAQSGDPEA | 211 |
| crest derivatives- | | | |
| expressed protein 1 | | | |
| Heart-and neural | 97Z | SKIKTLRLATSYIAYLMDLLAKDDQNGEAEA | 212 |
| crest derivatives- | | | |
| expressed protein 2 | | | |
| Transcription factor | 98Z | SKLDVLVLAASYIAHLTRTLGHELPGPAWPP | 213 |
| 23 | | | |
| Transcription factor | 99Z | SKLDVLLLATTYIAHLTRSLQDDAEAPADAG | 214 |
| 24 | | | |
| Musculin | 100Z | SKLDTLRLASSYIAHLROLLQEDRYENGYVH | 215 |
| Transcription factor | 101Z | SKLDTLRLASSYIAHLRQILANDKYENGYIH | 216 |
| 21 | | | |
| Basic helix-loop- | 102Z | SKIETLRLASSYISHLGNVLLAGEACGDGQP | 217 |
| helix transcription | | | |
| factor scleraxis | | | |
| Transcription factor | 103Z | SKIETVRLASSYIAHLANVLLLGDSADDGQP | 218 |
| 15 | | | |
| Twist-related protein | 104Z | SKIQTLKLAARYIDFLYQVLQSDELDSKMAS | 219 |
| 1 | | | |
| Twist-related protein | 105Z | SKIQTLKLAARYIDFLYQVLQSDEMDNKMTS | 220 |
| 2 | | | |

Thus, the basic helix of a protein is defined as the region of the protein amino acid sequence that aligns with the basic helices of the amino acid sequences as shown in Table 2. The zipper helix of a protein is defined as the region of the protein amino acid sequence that aligns with the zipper helices of the amino acid sequences as shown in Table 2. Such alignment can be achieved using an alignment program, such as, e.g., Clustal Omega, and performing a global alignment of a new protein sequence against the sequences as shown in Table 2 or the full length amino acid sequences of the proteins listed in Table 2.

TABLE 2

| | | | | | SEQ |
|---|---|---|---|---|---|
| | | | | | ID |
| | | COV | pid | 1[ . . . . : ]60 | NO |
| 1 | sp\|Q8IUM7\| | 100.0% | 100.0% | -MYRSTKGASKARRDQINAEIRNLKELLPLAE------ADKVRLSYLHIMSLACIYTRKG | 221 |
| 2 | sp\|P35869 | 100.0% | 27.8% | PAEGIKSNPSKRHRDRINTELDRLASLLPFPQ------DVINKLDKLSVLRLSVSYLRAK | 222 |
| 3 | sp\|A9YTQ3 | 100.0% | 29.6% | AVGAEKSNPSKRHRDRLNAELDHLASLLPFPP------DIISKLDKLSVLRLSVSYLRVK | 223 |
| 4 | sp\|Q99742\| | 100.0% | 29.6% | QRKEKSRNAARSRRGKENLEFFELAKLLPLPG------AISSQLDKASIVRLSVTYLRLR | 224 |
| 5 | sp\|Q8IXF0\| | 100.0% | 27.8% | LRKEKSRDAARSRRGKENFEFYELAKLLPLPA------AITSQLDKASIIRLTISYLKMR | 225 |
| 6 | sp\|P81133\| | 100.0% | 32.1% | -MKEKSKNAARTRREKENSEFYELAKLLPLPS------AITSQLDKASIIRLTTSYLKMR | 226 |
| 7 | sp\|Q14190\| | 100.0% | 34.0% | -MKEKSKNAAKTRREKENGEFYELAKLLPLPS------AITSQLDKASIIRLTTSYLKMR | 227 |
| 8 | sp\|Q99814\| | 100.0% | 24.1% | RRKEKSRDAARCRRSKETEVFYELAHELPLPH------SVSSHLDKASIMRLAISFLRTH | 228 |
| 9 | sp\|Q16665\| | 100.0% | 22.2% | RRKEKSRDAARSRRSKESEVFYELAHQLPLPH------NVSSHLDKASVMRLTISYLRVR | 229 |
| 10 | sp\|Q9Y2N7\| | 100.0% | 25.9% | LRKEKSRDAARSRRSQETEVLYQLAHTLPFAR------GVSAHLDKASIMRLTISYLRMH | 230 |
| 11 | sp\|Q15788\| | 98.1% | 18.6% | PCDTLASSTEKRRREQENKYLEELAELLSAN--ISDIDSLSVKPDKCKILKKTVDQIQLM | 231 |
| 12 | sp\|Q15596\| | 98.1% | 16.9% | LGPSPKRNTEKRNREQENKYIEELAELIFAN--FNDIDNFNFKPDKCAILKETVKQIRQI | 232 |
| 13 | sp\|Q9Y6Q9\| | 98.1% | 20.3% | PGQGLTCSGEKRRREQESKYIEELAELISAN--LSDIDNFNVKPDKCAILKETVRQIRQI | 233 |
| 14 | sp\|Q5JUK2\| | 94.3% | 16.4% | SCLRRNVISERERRKRMSLSCERLRALLPQF--D------GRREDMASVLEMSVQFLRLA | 234 |
| 15 | sp\|Q9UL49\| | 94.3% | 11.1% | QRRERHNRMERDRRRRIRICCDELNLLVPFC---------NAETDKATTLQWTTAFLKYI | 235 |
| 16 | sp\|Q9NX45\| | 94.3% | 16.4% | KISLLHSSKEKLRRERIKYCCEQLRTLLPYV--K------GRKNDAASVLEATVDYVKYI | 236 |
| 17 | sp\|A6NFD8\| | 98.1% | 26.3% | RTPVSHKVIEKRRRDRINRCLNELGKTVPMA--LAK--QSSGKLEKAEILEMTVQYLRAL | 237 |
| 18 | sp\|Q9NQ87\| | 98.1% | 19.3% | ARKKHRGIIEKRRRDRINSSLSELRRLVPTA--FEK--QGSSKLEKAEVLQMTVDHLKML | 238 |
| 19 | sp\|Q9Y5J3\| | 98.1% | 22.8% | ARKRRRGIIEKRRRDRINNSLSELRRLVPSA--FEK--QGSAKLEKAEILQMTVDHLKML | 239 |
| 20 | sp\|Q9UBP5\| | 98.1% | 21.1% | ARKKRRGIIEKRRRDRINNSLSELRRLVPTA--FEK--QGSAKLEKAEILQMTVDHLKML | 240 |
| 21 | sp\|014503\| | 98.1% | 24.6% | TYKLPHRLIEKKRRDRINECIAQLKDLLPEH--LKL--TTLGHLEKAVVLELTLKHVKAL | 241 |
| 22 | sp\|Q9C0J9\| | 98.1% | 24.6% | TYKLPHRLIEKKRRDRINECIAQLKDLLPEH--LKL--TTLGHLEKAVVLELTLKHLKAL | 242 |
| 23 | sp\|Q9BYE0\| | 98.1% | 23.7% | GPKMLKPLVEKRRRDRINRSLEELRLLLLER--TRDQNLRNPKLEKAEILEFAVGYLRER | 243 |
| 24 | sp\|Q96HZ4\| | 98.1% | 20.4% | DRKARKPLVEKKRRARINESLQELRLLLAGA-------EVQAKLENAEVLELTVRRVQGV | 244 |
| 25 | sp\|Q5TA89\| | 98.1% | 25.9% | KNRLRKPVVEKMRRDRINSSIEQLKLLLEQE--FA-RHQPNSKLEKADILEMAVSYLKHS | 245 |
| 26 | sp\|Q5TGS1\| | 84.9% | 30.0% | --------MEKKRRARINVSLEQLKSLLEKH---YSHQIRKRKLEKADILELSVKYMRSL | 246 |
| 27 | sp\|Q9Y543\| | 98.1% | 15.3% | LRKSLKPLLEKRRRARINQSLSQLKGLILPL--LGRENSNCSKLEKADVLEMTVRFLQEL | 247 |
| 28 | sp\|Q14469\| | 98.1% | 22.0% | HRKSSKPIMEKRRRARINESLSQLKTLILDA--LKKDSSRHSKLEKADILEMTVKHLRNL | 248 |
| 29 | sp\|Q9HCC6\| | 98.1% | 22.0% | HRKSSKPVMEKRRRARINESLAQLKTLILDA--LRKESSRHSKLEKADILEMTVRHLRSL | 249 |
| 30 | sp\|015516\| | 94.3% | 24.1% | AKRVSRNKSEKKRRDQFNVLIKELGSMLPGN---------ARKMDKSTVLQKSIDFLRKH | 250 |
| 31 | sp\|Q99743\| | 94.3% | 22.2% | AKRASRNKSEKKRRDQFNVLIKELSSMLPGN---------TRKMDKTTVLEKVIGFLQKH | 251 |
| 32 | sp\|P27540 | 98.1% | 16.4% | LARENHSEIERRRRNKMTAYITELSDMVPTCSA------LARKPDKLTILRMAVSHMKSL | 252 |
| 33 | sp\|Q8WYA1 | 98.1% | 21.8% | AFREAHSQTEKRRRDKMNNLIEELSAMIPQCNP------MARKLDKLTVLRMAVQHLRSL | 253 |
| 34 | sp\|Q68DE3\| | 96.2% | 22.2% | KNRETHNAVERHRKKKINAGINRIGELIPCSP--------ALKQSKNMILDQAFKYITEL | 254 |
| 35 | sp\|P22415\| | 100.0% | 26.8% | KRRAQHNEVERRRRDKINNWIVQLSKIIPDCSME----STKSGQSKGGILSKACDYIQEL | 255 |
| 36 | sp\|Q15853\| | 100.0% | 28.6% | RRRAQHNEVERRRRDKINNWIVQLSKIIPDCNAD----NSKTGASKGGILSKACDYIREL | 256 |
| 37 | sp\|P19484\| | 98.1% | 25.5% | QKKDNHNLIERRRRFNINDRIKELGMLIPKAND------LDVRWNKGTILKASVDYIRRM | 257 |
| 38 | sp\|014948\| | 98.1% | 20.0% | QKKDNHNLIERRRRYNINYRIKELGTLIPKSND------PDMRWNKGTILKASVEYIKWL | 258 |
| 39 | sp\|075030\| | 98.1% | 23.6% | QKKDNHNLIERRRRFNINDRIKELGTLIPKSND------PDMRWNKGTILKASVDYIRKL | 259 |
| 40 | sp\|P19532\| | 98.1% | 23.6% | QKKDNHNLIERRRRFNINDRIKELGTLIPKSSD------PEMRWNKGTILKASVDYIRKL | 260 |
| 41 | sp\|P36956\| | 94.3% | 22.2% | EKRTAHNAIEKRYRSSINDKIIELKDLVVGT---------EAKLNKSAVLRKAIDYIRFL | 261 |
| 42 | sp\|Q12772\| | 94.3% | 18.5% | ERRTTHNIIEKRYRSSINDKIIELKDLVMGT---------DAKMHKSGVLRKAIDYIKYL | 262 |
| 43 | sp\|Q99583\| | 96.2% | 18.5% | GTREVHNKLEKNRRAHLKECFETLKRNIPNV-------D-DKKTSNLSVLRTALRYIQSL | 263 |
| 44 | sp\|Q9BW11\| | 98.1% | 25.9% | SGRSVHNELEKRRRAQLKRCLERLKQQMPLG-------ADCARYTTLSLLRRARMHIQKL | 264 |
| 45 | sp\|Q14582\| | 98.1% | 25.9% | NNRSSHNELEKHRRAKLRLYLEQLKQLVPLG-------PDSTRHTTLSLLKRAKVHIKKL | 265 |
| 46 | sp\|Q05195\| | 98.1% | 22.2% | SSRSTHNEMEKNRRAHLRLCLEKLKGLVPLG-------PESSRHTTLSLLTKAKLHIKKL | 266 |
| 47 | sp\|P50539\| | 98.1% | 24.1% | ANRSTHNELEKNRRAHLRLCLERLKVLIPLG-------PDCTRHTTLGLLNKAKAHIKKL | 267 |
| 48 | sp\|Q9UH92\| | 100.0% | 18.6% | RRRRAHTQAEQKRRDAIKRGYDDLQTIVPTCQ-QQDFSIGSQKLSKAIVLQKTIDYIQFL | 268 |
| 49 | sp\|Q9HAP2\| | 94.3% | 16.7% | NRQMKHISAEQKRRFNIKMCFDMLNSLISNN---------SKLTSHAITLQKTVEYITKL | 269 |
| 50 | sp\|Q9NP71\| | 100.0% | 14.5% | NRRITHISAEQKRRFNIKLGFDTLHGLVSTLS-A----QPSLKVSKATTLQKTAEYILML | 270 |
| 51 | sp\|Q6QHK4\| | 98.1% | 20.4% | ERRRVANAKERERIKNLNRGFARLKALVPFL-------PQSRKPSKVDILKGATEYIQVL | 271 |
| 52 | sp\|P61244\| | 96.2% | 18.5% | DKRAHHNALERKRRDHIKDSFHSLRDSVPSL-------Q-GEKASRAQILDKATEYIQYM | 272 |
| 53 | sp\|P12524\| | 98.1% | 16.7% | TKRKNHNFLERKRRNDLRSRFLALRDQVPTL-------ASCSKAPKVVILSKALEYLQAL | 273 |
| 54 | sp\|P01106\| | 98.1% | 14.8% | VKRRTHNVLERQRRNELKRSFFALRDQIPEL-------ENNEKAPKVVILKKATAYILSV | 274 |
| 55 | sp\|P04198\| | 98.1% | 14.8% | ERRRNHNILERQRRNDLRSSFLTLRDHVPEL-------VKNEKAAKVVILKKATEYVHSL | 275 |
| 56 | sp\|P15923\| | 98.1% | 14.5% | ERRVANNARERLRVRDINEAFKELGRMCQLH------LNSEKPQTKLLILHQAVSVILNL | 276 |
| 57 | sp\|Q99081\| | 98.1% | 14.5% | ERRMANNARERLRVRDINEAFKELGRMCQLH------LKSEKPQTKLLILHQAVAVILSL | 277 |
| 58 | sp\|P15884\| | 98.1% | 14.5% | ERRMANNARERLRVRDINEAFKELGRMVQLH------LKSDKPQTKLLILHQAVAVILSL | 278 |
| 59 | sp\|P50553\| | 98.1% | 18.5% | AAVARRNERERNRVKLVNLGFATLREHVPNG-------AANKKMSKVETLRSAVEYIRAL | 279 |
| 60 | sp\|Q99929 | 98.1% | 16.7% | AAVARRNERERNRVKLVNLGFQALRQHVPHG-------GASKKLSKVETLRSAVEYIRAL | 280 |
| 61 | sp\|Q9NQ33 | 98.1% | 20.4% | AFTRKRNERERQRVKCVNEGYAQLRHHLPEE-------YLEKRLSKVETLRAAIKYINYL | 281 |
| 62 | sp\|Q6XD76\| | 98.1% | 20.4% | AFLRKRNERERQRVRCVNEGYARLRDHLPRE-------LADKRLSKVETLRAAIDYIKHL | 282 |
| 63 | sp\|Q7RTU5\| | 98.1% | 20.4% | AFIQKRNERERQRVKCVNEGYARLRGHLPGA-------LAEKRLSKVETLRAAIRYIKYL | 283 |
| 64 | sp\|Q01664\| | 96.2% | 24.1% | IRREIANSNERRRMQSINAGFQSLKTLIPHT--------DGEKLSKAAILQQTAEYIFSL | 284 |
| 65 | sp\|Q9BRJ9\| | 98.1% | 17.9% | GQRQSASEREKLRMRTLARALHELRRFLPPS-----VAPAGQSLTKIETLRLAIRYIGHL | 285 |
| 66 | sp\|Q0VG99\| | 98.1% | 17.9% | GQRQSASEREKLRMRTLARALHELRRFLPPS-----LAPAGQSLTKIETLRLAIRYIGHL | 286 |
| 67 | sp\|A6NI15\| | 98.1% | 14.3% | QRRRKASEREKLRMRTLADALHTLRNYLPPV-----YSQRGQPLTKIQTLKYTIKYIGEL | 287 |
| 68 | sp\|Q8TAK6\| | 100.0% | 18.3% | QLRRKINSRERKRMQDLNLAMDALREVILPYSAAHCQGAPGRKLSKIATLLLARNYILLL | 288 |
| 69 | sp\|Q13516\| | 98.1% | 19.6% | QLRLKINSRERKRMHDLNIAMDGLREVMPYA-----HGPSVRKLSKIATLLLARNYILML | 289 |
| 70 | sp\|Q7RTU3\| | 98.1% | 21.4% | QLRLKINGRERKRMHDLNLAMDGLREVMPYA-----HGPSVRKLSKIATLLLARNYILML | 290 |
| 71 | sp\|Q8NFJ8\| | 98.1% | 17.9% | ALRLNINARERRRMHDLNDALDELRAVIPYA-----HSPSVRKLSKIATLLLAKNYILMQ | 291 |
| 72 | sp\|Q8NDY6\| | 98.1% | 19.6% | SLRLSINARERRRMHDLNDALDGLRAVIPYA-----HSPSVRKLSKIATLLLAKNYILMQ | 292 |
| 73 | sp\|Q7RTS1\| | 98.1% | 18.5% | QRRLESNERERQRMHKLNNAFQALREVIPHV-------RADKKLSKIETLTLAKNYIKSL | 293 |
| 74 | sp\|Q92858\| | 98.1% | 16.7% | QRRLAANARERRRMHGLNHAFDQLRNVIPSF-------NNDKKLSKYETLQMAQIYINAL | 294 |
| 75 | sp\|Q8N100\| | 98.1% | 14.8% | RRRLAANARERRRMQGLNTAFDRLRRVVPQW-------GQDKKLSKYETLQMALSYIMAL | 295 |
| 76 | sp\|Q96SQ7\| | 98.1% | 22.2% | TRRLLANARERTRVHTISAAFEALRKQVPCY-------SYGQKLSKLAILRIACNYILSL | 296 |
| 77 | sp\|Q9H2A3\| | 98.1% | 18.5% | TRRLKANNRERNRMHNLNAALDALREVLPTF-------PEDAKLTKIETLRFAHNYIWAL | 297 |
| 78 | sp\|Q92886\| | 98.1% | 18.5% | SRRVKANDRERNRMHNLNAALDALRSVLPSF-------PDDTKLTKIETLRFAYNYIWAL | 298 |
| 79 | sp\|Q9Y4Z2\| | 98.1% | 16.7% | SRRKKANDRERNRMHNLNSALDALRGVLPTF-------PDDAKLTKIETLRFAHNYIWAL | 299 |
| 80 | sp\|Q9HD90\| | 98.1% | 18.5% | ARRVKANARERTRMHGLNDALDNLRRVMPCY-------SKTQKLSKIETLRLARNYIWAL | 300 |
| 81 | sp\|Q13562\| | 98.1% | 20.4% | LRRMKANARERNRMHGLNAALDNLRKVVPCY-------SKTQKLSKIETLRLAKNYIWAL | 301 |
| 82 | sp\|Q15784\| | 98.1% | 20.4% | LRRQKANARERNRMHDLNAALDNLRKVVPCY-------SKTQKLSKIETLRLAKNYIWAL | 302 |
| 83 | sp\|Q96NK8\| | 98.1% | 18.5% | FRRQEANARERNRMHGLNDALDNLRKVVPCY-------SKTQKLSKIETLRLAKNYIWAL | 303 |
| 84 | sp\|P13349\| | 96.2% | 16.7% | DRRKAATMRERRRLKKVNQAFETLKRCTTTN-------P-NQRLPKVEILRNAIRYIESL | 304 |
| 85 | sp\|P15172\| | 96.2% | 16.7% | DRRKAATMRERRRLSKVNEAFETLKRCTSSN-------P-NQRLPKVEILRNAIRYIEGL | 305 |
| 86 | sp\|P23409\| | 96.2% | 18.5% | DRRKAATLRERRRLKKINEAFEALKRRTVAN-------P-NQRLPKVEILRSAISYIERL | 306 |
| 87 | sp\|P15173\| | 96.2% | 22.2% | DRRRAATLREKRRLKKVNEAFEALKRSTLLN-------P-NQRLPKVEILRSAIQYIERL | 307 |
| 88 | sp\|Q7RTU4\| | 98.1% | 14.8% | ARRMAANVRERKRILDYNEAFNALRRALRHD-------LGGKRLSKIATLRRAIHRIAAL | 308 |
| 89 | sp\|Q96RJ6\| | 98.1% | 20.4% | AQRQAANIRERKRMFNLNEAFDQLRRKVPTF-------AYEKRLSRIETLRLAIVYISFM | 309 |
| 90 | sp\|Q7RTS3\| | 98.1% | 20.4% | QLRQAANVRERRRMQSINDAFEGLRSHIPTL-------PYEKRLSKVDTLRLAIGYINFL | 310 |
| 91 | sp\|Q02575\| | 98.1% | 22.2% | KYRTAHATRERIRVEAFNLAFAELRKLLPTL-------PPDKKLSKIEILRLAICYISYL | 311 |
| 92 | sp\|Q02577\| | 98.1% | 22.2% | KYRSAHATRERIRVEAFNLAFAELRKLLPTL-------PPDKKLSKIEILRLAICYISYL | 312 |
| 93 | sp\|Q16559\| | 98.1% | 20.4% | TRKIFTNTRERWRQQNVNSAFAKLRKLIPTH-------PPDKKLSKNETLRLAMRYINFL | 313 |
| 94 | sp\|P12980\| | 98.1% | 22.2% | ARRVFTNSRERWRQQNVNGAFAELRKLLPTH-------PPDRKLSKNEVLRLAMKYIGFL | 314 |
| 95 | sp\|P17542\| | 98.1% | 22.2% | VRRIFTNSRERWRQQNVNGAFAELRKLIPTH-------PPDKKLSKNEILRLAMKYINFL | 315 |
| 96 | sp\|096004\| | 98.1% | 22.2% | RRKGSGPKKERRRTESINSAFAELRECIPNV-------PADTKLSKIKTLRLATSYIAYL | 316 |
| 97 | sp\|P61296\| | 98.1% | 20.4% | KRRGTANRKERRRTQSINSAFAELRECIPNV-------PADTKLSKIKTLRLATSYIAYL | 317 |
| 98 | sp\|Q7RTU1\| | 98.1% | 18.5% | EASPENAARERSRVRTLRQAFLALQAALPAV-------PPDTKLSKLDVLVLAASYIAHL | 318 |
| 99 | sp\|Q7RTUO\| | 98.1% | 18.5% | RPAAANAARERSRVQTLRHAFLELQRTLPSV-------PPDTKLSKLDVLLLATTYIAHL | 319 |
| 100 | sp\|060682\| | 98.1% | 22.2% | SQRNAANARERARMRVLSKAFSRLKTSLPWV-------PPDTKLSKLDTLRLASSYIAHL | 320 |
| 101 | sp\|043680\| | 98.1% | 22.2% | VQRNAANARERARMRVLSKAFSRLKTTLPWV-------PPDTKLSKLDTLRLASSYIAHL | 321 |
| 102 | sp\|Q7RTU7\| | 98.1% | 18.5% | RQRHTANARERDRTNSVNTAFTALRTLIPTE-------PADRKLSKIETLRLASSYISHL | 322 |
| 103 | sp\|Q12870\| | 98.1% | 18.5% | RQRQAANARERDRTQSVNTAFTALRTLIPTE-------PVDRKLSKIETVRLASSYIAHL | 323 |
| 104 | sp\|Q15672\| | 96.2% | 16.7% | TQRVMANVRERQRTQSLNEAFAALRKIIPTL-------PS-DKLSKIQTLKLAARYIDFL | 324 |
| 105 | sp\|Q8WVJ9\| | 96.2% | 16.7% | SQRILANVRERQRTQSLNEAFAALRKIIPTL-------PS-DKLSKIQTLKLAARYIDFL | 325 |

| | | |
|---|---|---|
| consensus/100% | ..t..t..hE+.Rhtthp..h.tLtthlt.............p.p+h.hLp.shtal..h | 327/335 |
| consensus/90% | ..t..t..hE+.Rhtthp..h.tLtthlt.............p.p+h.hLp.shtal..h | 327/335 |
| consensus/80% | th+.tts.hE+pRhpplstthttLtphls...........st+hsKhthLphshpalt.l | 328/336 |
| consensus/70% | tp+.ttsthE+pRhpplNtshtpLpphlPhh.........sp+hsKhphLchuhpYlthL | 333/337 |

The alignment can be enhanced using principles taken from the alignments of the sequences of Table 2. Based on the alignments shown in Table 2, “consensus” sequences emerge, wherein the basic helix can have the amino acid sequence of

(SEQ ID NO: 330)
xxxxxxxxxtpxxxxxxtxxhxxltxxhxxx

wherein the representations of the symbols are found below in Table 3.

TABLE 3

| | Key (lower | |
|---|---|---|
| Class | case letters) | Residues (upper case letters) |
| aliphatic | l | I, L, V |
| any | x | A, C, D, E, F, G, H, I, K, L, M, N, P, |
| | | Q, R, S, T, V, W, Y |
| aromatic | a | F, H, W, Y |
| charged | c | D, E, H, K, R |
| hydrophobic | h | A, C, F, G, H, I, K, L, M, R, T, V, W, Y |
| polar | p | C, D, E, H, K, N, Q, R, S, T |
| positive | + | H, K, R |
| small | s | A, C, D, G, N, P, S, T, V |
| tiny | u | A, G, S |
| turnlike | t | A, C, D, E, G, H, K, N, Q, R, S, T |

Some variations among sequences are possible, and there is a 90% likelihood that the basic helix can have a sequence of

(SEQ ID NO: 331)
xxtxxtxxhE+xRhtthpxxhxtLtthlxxx

an 80% likelihood that the basic helix can have a sequence of

(SEQ ID NO: 332)
th+xttsxhE+pRhpplstthttLtphlsxx

a 70% likelihood that the basic helix can have a sequence of

(SEQ ID NO: 333)
tp+xttsthE+pRhpplNtshtpLpph1Phh.

The zipper helix can have the amino acid sequence of

(SEQ ID NO: 334)
xxxxxxxxhhxxxxxxhxxx

a 90% likelihood that the zipper helix can have a sequence of

(SEQ ID NO: 335)
txpxp+hxhLpxshtalxxh

an 80% likelihood that the zipper helix can have a sequence of

(SEQ ID NO: 336)
st+hsKhthLphshpaltxl

a 70% likelihood that the zipper helix can have a sequence of

(SEQ ID NO: 337)
sp+hsKhphLchuhpYlthL.

The sequences of Table 2 can be used without adding the new sequences to the list of sequences in Table 2, where the consensus criteria are maintained for each new sequence, as the criteria are given above. As additional alignments are performed with new sequences, the new sequences can be added to those sequences in Table 2 to update the list of sequences and the consensus criteria. As understood by those in the art, the consensus criteria may evolve with the addition of any new sequences.

In aspects, the basic helix of the first polypeptide comprises the amino acid sequence extending 36 residues in the N-terminal direction from the start of the loop of the basic helix-loop-helix domain. In aspects, the second polypeptide comprises the amino acid sequence extending 31 residues in the C-terminal direction from the end of the loop of the basic helix-loop-helix domain. In aspects, the second polypeptide comprises a leucine zipper helix.

In aspects, the amino acid sequence of the first polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the first polypeptide. In aspects, the amino acid sequence of the second polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the second polypeptide.

As used herein, “covalent cross-link is internal to the polypeptide” and the like means that the cross-link starts at a residue within the polypeptide chain and ends at a residue within the same polypeptide chain.

In aspects of the disclosure, a polypeptide according to the disclosure can include one or more non-natural amino acids. A first non-natural amino acid can be cross-linked to a second non-natural amino acid that is substituted or inserted at a position in the polypeptide which is four residues away. The relative positions of the first and second non-natural amino acids in this stapled polypeptide are designated as (i, i+4). In aspects of the disclosure, the first non-natural amino acid can be cross-linked to a second non-natural amino acid located seven residues away (i, i+7) in the polypeptide. In aspects of the disclosure, the first non-natural amino acid can be cross-linked to a second non-natural amino acid located three residues away (i, i+3) in the polypeptide.

In aspects, each set of non-natural amino acids of the first and second polypeptides are capable of undergoing a Diels-Alder reaction, a Huisgen reaction, or an olefin metathesis reaction.

In aspects, in the first or second polypeptides, one non-natural amino acid within a set is Xaa^{A1} and the other non-natural amino acid within the set is Xaa^{B1}
wherein

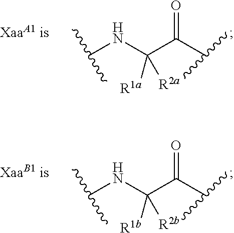

R^{1a} and R^{1b} are independently H, alkyl, alkenyl, alkynyl, arylalkyl, cycloalkylalkyl, heteroarylalkyl, or heterocyclylalkyl;
R^{2a} and R^{2b} are (i) independently alkenyl, alkynyl, azido, amino, carboxylic acid, or sulfide or (ii) taken together to form alkylene, alkenylene, alkynylene, or [R^{3a}—X—R^{3b}]ₙ, each of which is substituted with 0-6 R⁴;
each R^{3a} and R^{3b} are independently alkylene, alkenylene or alkynylene;
each R⁴ is independently halo, alkyl, OR⁵, N(R⁵)₂, SR⁵, SOR⁵, SO₂R⁵, CO₂R⁵, R⁵;
each X is independently O, S, SO, SO₂, CO, CO₂, CONR⁵ or

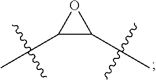 [SMILES: *C1OC1*]

each R⁵ is independently H or alkyl; and
n is an integer 1-4.

In the aspects of the disclosure, halo includes any halogen, e.g., F, Cl, Br, I.

As used herein, unless otherwise specified, the term “alkyl” means a saturated straight chain or branched non-cyclic hydrocarbon having an indicated number of carbon atoms (e.g., C₁-C₂₀, C₁-C₁₀, C₁-C₄, C₁-C₆, etc.). An alky group may have 1, 2, 3, 4, 5, 6, 7, 8, or more carbons. Representative saturated straight chain alkyls include -methyl, -ethyl, -n-propyl, -n-butyl, -n-pentyl, -n-hexyl, -n-heptyl, -n-octyl, -n-nonyl and -n-decyl; while representative saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. “Alkenyl” means an unsaturated straight chain or branched non-cyclic hydrocarbon having an indicated number of carbon atoms (e.g., C₁-C₂₀, C₁-C₁₀, C₁-C₄, C₁-C₆, etc.), where at least one carbon-carbon bond is a double bond. An alkenyl group may have 1, 2, 3, 4, 5, 6, 7, 8, or more carbons. “Alkynyl” means an unsaturated straight chain or branched non-cyclic hydrocarbon having an indicated number of carbon atoms (e.g., C₁-C₂₀, C₁-C₁₀, C₁-C₄, C₁-C₆, etc.), where at least one carbon-carbon bond is a triple bond. An alkynyl group may have 1, 2, 3, 4, 5, 6, 7, 8, or more carbons. As understood in the art, “alkylene,” “alkenylene,” and “alkynylene” are the bivalent radical forms of alkyl, alkenyl, and alkynyl, respectively.

The term “cycloalkyl,” as used herein, means a cyclic alkyl moiety containing from, for example, 3 to 6 carbon atoms, preferably from 5 to 6 carbon atoms. Examples of such moieties include cyclopropyl, cyclobutyl, cyclopentyl, cyclohexyl, and the like. “Cycloalkylalkyl” is a cycloalkyl as defined above substituted with an alkyl as defined above.

The term “heterocyclyl” means a cycloalkyl moiety having one or more heteroatoms selected from nitrogen, sulfur, and/or oxygen. Preferably, a heterocyclyl is a 5 or 6-membered monocyclic ring and contains one, two, or three heteroatoms selected from nitrogen, oxygen, and/or sulfur. The heterocyclyl can be attached to the parent structure through a carbon atom or through any heteroatom of the heterocyclyl that results in a stable structure. Examples of such heterocyclic rings are pyrrolinyl, pyranyl, piperidyl, tetrahydrofuranyl, tetrahydrothiopheneyl, and morpholinyl. “Heterocyclylalkyl” is a heterocyclyl as defined above substituted with an alkyl as defined above.

As used herein, unless otherwise specified, the term “alkylamino” means —NH(alkyl) or —N(alkyl)(alkyl), wherein alkyl is defined above. As used herein, unless otherwise specified, the term “cycloalkylamino” means —NH(cycloalkyl) or —N(cycloalkyl)(cycloalkyl), wherein cycloalkyl is defined above.

The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, preferably from 6 to 18 carbon atoms, more preferably from 6 to 14 carbon atoms and most preferably from 6 to 10 carbon atoms. It is understood that the term aryl includes carbocyclic moieties that are planar and comprise 4n+2 π electrons, according to Hückel's Rule, wherein n=1, 2, or 3. “Arylalkyl” means an aryl as defined above substituted with an alkyl as defined above.

The term “heteroaryl” refers to aromatic 4, 5, or 6 membered monocyclic groups, 9 or 10 membered bicyclic groups, and 11 to 14 membered tricyclic aryl groups having one or more heteroatoms (O, S, or N). Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The nitrogen and sulfur atoms may optionally be oxidized, and the nitrogen atoms may optionally be quaternized. Illustrative examples of heteroaryl groups are pyridinyl, pyridazinyl, pyrimidyl, pyrazinyl, triazinyl, pyrrolyl, pyrazolyl, imidazolyl, (1,2,3)- and (1,2,4)-triazolyl, pyrazinyl, pyrimidinyl, tetrazolyl, furyl, thiophenyl, isothiazolyl, thiazolyl, isoxazolyl, oxadiazolyl, oxazolyl, pyrrolo[2,3-c]pyridinyl, pyrrolo[3,2-c]pyridinyl, pyrrolo[2,3-b]pyridinyl, pyrrolo[3,2-b]pyridinyl, pyrrolo[3,2-d]pyrimidinyl, and pyrrolo[2,3-d]pyrimidinyl. “Heteroarylalkyl” is a heteroaryl as defined above substituted with an alkyl as defined above.

Whenever a range of the number of atoms in a structure is indicated (e.g., a C₁-C₈, C₁-C₆, C₁-C₄, or C₁-C₃ alkyl, haloalkyl, alkylamino, alkenyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, haloalkyl, alkylamino, alkenyl, etc.) referenced herein encompasses and specifically describes 1, 2, 3, 4, 5, 6, 7, or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, 5-6 carbon atoms, 5-7 carbon atoms, 5-8 carbon atoms, 6-7 carbon atoms, or 6-8 carbon atoms, as appropriate).

In aspects, the non-natural amino acids of the first or second polypeptide are capable of forming together a thioether, ether, amide, amine, triazole, or carbon-carbon double bond or a Diels-Alder adduct after reaction. In aspects, the non-natural amino acids are independently selected from (S)-2-(4′-pentenyl)alanine (S₅), (R)-2-(2′-propenyl)alanine (R3), and (R)-2-(7′-octenyl)alanine (R8). In aspects, the non-natural amino acids have undergone reaction to form the intrapolypeptide covalent cross-link with each other.

In aspects of the disclosure, the cross-link of the polypeptide is formed from the amino acid at position i within the polypeptide and another amino acid at position i+4 within the polypeptide, and the amino acid at position i is (S)-2-(4′-pentenyl)alanine (S5) and the amino acid at position i+4 is S5; or formed from the amino acid at position i within the polypeptide and another amino acid at position i+3 within the polypeptide, and the amino acid at position i is (R)-2-(4′-pentenyl)alanine (R5) or (R)-2-(2′-propenyl)alanine (R3) and the amino acid at position i+3 is S5; or formed from the amino acid at position i within the polypeptide and another amino acid at position i+7 within the polypeptide, and the amino acid at position i is (R)-2-(7′-octenyl)alanine (R8) and the amino acid at position i+7 is S5.

In aspects of the disclosure, (a) the cross-link of the first polypeptide is formed from the amino acid at position i within the first polypeptide and another amino acid at position i+4 within the first polypeptide, and the amino acid at position i is (S)-2-(4′-pentenyl)alanine (S5) and the amino acid at position i+4 is S5; or formed from the amino acid at position i within the first polypeptide and another amino acid at position i+3 within the first polypeptide, and the amino acid at position i is (R)-2-(4′-pentenyl)alanine (R5) or (R)-2-(2′-propenyl)alanine (R3) and the amino acid at position i+4 is S5; or formed from the amino acid at position i within the first polypeptide and another amino acid at position i+7 within the first polypeptide, and the amino acid at position i is (R)-2-(7′-octenyl)alanine (R8) and the amino acid at position i+7 is S5; and (b) the cross-link of the second polypeptide is formed from the amino acid at position i within the second polypeptide and another amino acid at position i+4 within the second polypeptide, and the amino acid at position i is (S)-2-(4′-pentenyl)alanine (S5) and the amino acid at position i+4 is S5; or formed from the amino acid at position i within the second polypeptide and another amino acid at position i+3 within the second polypeptide, and the amino acid at position i is (R)-2-(4′-pentenyl)alanine (R5) or (R)-2-(2′-propenyl)alanine (R3) and the amino acid at position i+4 is S5; or formed from the amino acid at position i within the second polypeptide and another amino acid at position i+7 within the second polypeptide, and the amino acid at position i is (R)-2-(7′-octenyl)alanine (R8) and the amino acid at position i+7 is S5.

In aspects of the disclosure, a reside other than Xaa^{A1} or Xaa^{B1} within the first polypeptide and a residue other than Xaa^{A1} and Xaa^{B1} within the second polypeptide are covalently linked, forming a ligated construct. In aspects, the linkage involves butyrlmaleimide (ButMal), glyclmaleimde (Glymal), or bismaleimidohexane:

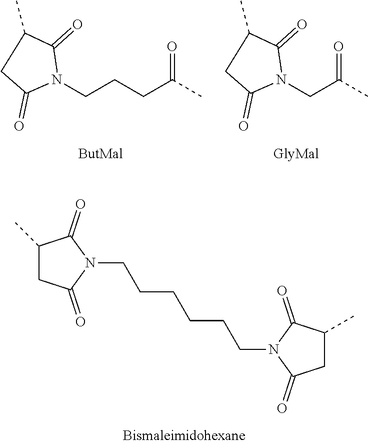

In aspects, the covalent linkage results in an adduct that forms from a Diels-Alder reaction, an olefin metathesis reaction, copper-catalyzed azide-alkyne click chemistry, cystine formation via oxidation of two cysteine residues, crosslink formation via alkylation of one or more cysteine residues, thiol-ene chemistry, or a lactam bridge formation between N- or C-termini and/or residue side chain(s). In aspects, at least one of the residues is a non-natural amino acid or an amino acid derivative. In aspects, the reactive functional groups are each independently bound to an amino acid side chain, amino acid amino group, amino acid carboxy group, or amino acid α-carbon. In aspects, the adduct is bound to a side chain, amine group, carboxy group, or α-carbon of one amino acid and to a side chain, amine group, carboxy group, or α-carbon of a different amino acid within the same polypeptide to provide a cyclic structure. In aspects, the macrocyclic polypeptide is formed in which one reactive functional group includes a diene and a different reactive functional group includes a dienophile. The complementary diene and dienophile pair can react to form a macrocyclic peptide through an intramolecular Diels-Alder reaction. The reactive functional groups may each independently be conjugated to a terminal amino acid or an internal amino acid. In aspects, the adduct is formed from a reaction between a hexadiene group and a maleimide group, a maleimide group and a furan group, a cyclopentadiene group and another cyclopentadiene group, a cyclopentadiene group and a maleimide group, or a cyclopentadiene group and an aliphatic olefin (for example, an aliphatic olefin used as a peptide staple). In aspects, the adduct is one of the Diels-Alder adducts:

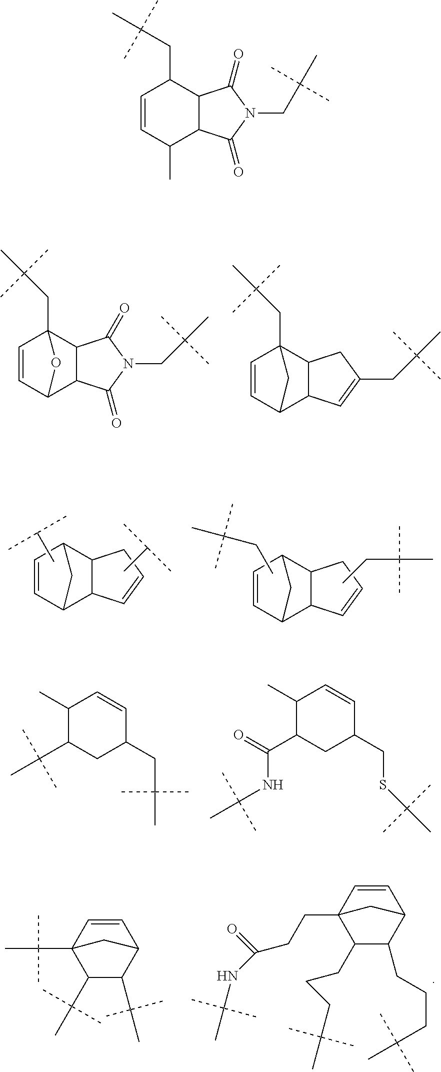

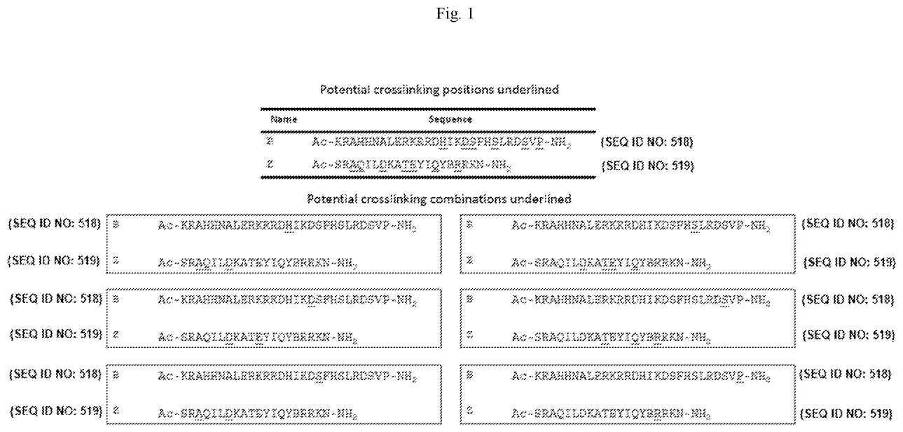

In aspects of the disclosure, exemplary positions of residues that can be covalently linked are shown in FIG. 1. In aspects, one residue is cysteine or a cysteine derivative and the other residue is lysine or a lysine derivative. In aspects, the cysteine and/or lysine are derivatized to form a diene or a dienophile. In aspects, the interpolypeptide covalent linkage between the first polypeptide and the second polypeptide is a maleimide-thiol adduct.

In aspects, the disclosure provides a polypeptide construct comprising (a) a polypeptide construct as described above; (b) a third polypeptide comprising an amino acid sequence derived from a basic helix of a transcription factor protein that comprises a basic helix-loop-helix domain; and (c) a fourth polypeptide comprising an amino acid sequence derived from a helix that extends in the C-terminal direction from the end of the loop of a basic helix-loop-helix domain of a transcription factor protein that comprises a basic helix-loop-helix domain; wherein the third polypeptide and the fourth polypeptide are linked through an interpolypeptide covalent linkage.

In aspects, the basic helix of the third polypeptide comprises the amino acid sequence extending 36 residues in the N-terminal direction from the start of the loop of the basic helix-loop-helix domain. In aspects, the fourth polypeptide comprises the amino acid sequence extending 31 residues in the C-terminal direction from the end of the loop of the basic helix-loop-helix domain. In aspects, the fourth polypeptide comprises a leucine zipper helix.

In aspects, the amino acid sequence of the third polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the third polypeptide. In aspects, the amino acid sequence of the fourth polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the fourth polypeptide.

In aspects, each set of non-natural amino acids of the third and fourth polypeptides are capable of undergoing a Diels-Alder reaction, a Huisgen reaction, or an olefin metathesis reaction.

In aspects, in the third or fourth polypeptides, one non-natural amino acid within a set is Xaa^{A1} and the other non-natural amino acid within the set is Xaa^{B1}
wherein

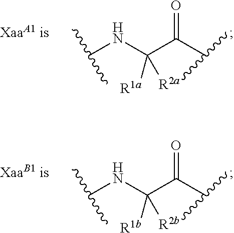

R^{1a} and R^{1b} are independently H, alkyl, alkenyl, alkynyl, arylalkyl, cycloalkylalkyl, heteroarylalkyl, or heterocyclylalkyl;
R^{2a} and R^{2b} are (i) independently alkenyl, alkynyl, azido, amino, carboxylic acid, or sulfide or (ii) taken together to form alkylene, alkenylene, alkynylene, or [R^{3a}—X—R^{3b}]ₙ, each of which is substituted with 0-6 R⁴;
each R^{3a} and R^{3b} are independently alkylene, alkenylene or alkynylene;
each R⁴ is independently halo, alkyl, OR⁵, N(R⁵)₂, SR⁵, SOR⁵, SO₂R⁵, CO₂R⁵, R⁵;
each X is independently O, S, SO, SO₂, CO, CO₂, CONR⁵ or

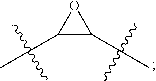 [SMILES: *C1OC1*]

each R⁵ is independently H or alkyl; and
n is an integer 1-4.

In aspects, the non-natural amino acids of the third or fourth polypeptide are capable of forming together a thioether, ether, amide, amine, triazole, or carbon-carbon double bond or a Diels-Alder adduct after reaction. In aspects, the non-natural amino acids are independently selected from (S)-2-(4′-pentenyl)alanine (S5), (R)-2-(2′-propenyl)alanine (R3), and (R)-2-(7′-octenyl)alanine (R8). In aspects, the non-natural amino acids have undergone reaction to form the intrapolypeptide covalent cross-link with each other.

In aspects of the disclosure, the cross-link of the polypeptide is formed from the amino acid at position i within the polypeptide and another amino acid at position i+4 within the polypeptide, and the amino acid at position i is (S)-2-(4′-pentenyl)alanine (S5) and the amino acid at position i+4 is S5; or formed from the amino acid at position i within the polypeptide and another amino acid at position i+3 within the polypeptide, and the amino acid at position i is (R)-2-(4′-pentenyl)alanine (R5) or (R)-2-(2′-propenyl)alanine (R3) and the amino acid at position i+3 is S5; or formed from the amino acid at position i within the polypeptide and another amino acid at position i+7 within the polypeptide, and the amino acid at position i is (R)-2-(7′-octenyl)alanine (R8) and the amino acid at position i+7 is S5.

In aspects of the disclosure, (a) the cross-link of the third polypeptide is formed from the amino acid at position i within the third polypeptide and another amino acid at position i+4 within the third polypeptide, and the amino acid at position i is (S)-2-(4′-pentenyl)alanine (S5) and the amino acid at position i+4 is S5; or formed from the amino acid at position i within the third polypeptide and another amino acid at position i+3 within the third polypeptide, and the amino acid at position i is (R)-2-(4′-pentenyl)alanine (R5) or (R)-2-(2′-propenyl)alanine (R3) and the amino acid at position i+4 is S5; or formed from the amino acid at position i within the third polypeptide and another amino acid at position i+7 within the third polypeptide, and the amino acid at position i is (R)-2-(7′-octenyl)alanine (R8) and the amino acid at position i+7 is S5; and (b) the cross-link of the fourth polypeptide is formed from the amino acid at position i within the fourth polypeptide and another amino acid at position i+4 within the fourth polypeptide, and the amino acid at position i is (S)-2-(4′-pentenyl)alanine (S5) and the amino acid at position i+4 is S5; or formed from the amino acid at position i within the fourth polypeptide and another amino acid at position i+3 within the fourth polypeptide, and the amino acid at position i is (R)-2-(4′-pentenyl)alanine (R5) or (R)-2-(2′-propenyl)alanine (R3) and the amino acid at position i+4 is S5; or formed from the amino acid at position i within the fourth polypeptide and another amino acid at position i+7 within the fourth polypeptide, and the amino acid at position i is (R)-2-(7′-octenyl)alanine (R8) and the amino acid at position i+7 is S5.

In aspects of the disclosure, a reside other than Xaa^{A1} or Xaa^{B1} within the third polypeptide and a residue other than Xaa^{A1} and Xaa^{B1} within the fourth polypeptide are covalently linked, forming a ligated construct. In aspects, the linkage involves butyrlmaleimide (ButMal), glyclmaleimde (Glymal), or bismaleimidohexane. In aspects, the covalent linkage results in an adduct that forms from a Diels-Alder reaction, an olefin metathesis reaction, copper-catalyzed azide-alkyne click chemistry, cystine formation via oxidation of two cysteine residues, crosslink formation via alkylation of one or more cysteine residues, thiol-ene chemistry, or a lactam bridge formation between N- or C-termini and/or residue side chain(s). In aspects, at least one of the residues is a non-natural amino acid or an amino acid derivative. In aspects, the reactive functional groups are each independently bound to an amino acid side chain, amino acid amino group, amino acid carboxy group, or amino acid α-carbon. In aspects, the adduct is bound to a side chain, amine group, carboxy group, or α-carbon of one amino acid and to a side chain, amine group, carboxy group, or α-carbon of a different amino acid within the same polypeptide to provide a cyclic structure. In aspects, the macrocyclic polypeptide is formed in which one reactive functional group includes a diene and a different reactive functional group includes a dienophile. The complementary diene and dienophile pair can react to form a macrocyclic peptide through an intramolecular Diels-Alder reaction. The reactive functional groups may each independently be conjugated to a terminal amino acid or an internal amino acid. In aspects, the adduct is formed from a reaction between a hexadiene group and a maleimide group, a maleimide group and a furan group, a cyclopentadiene group and another cyclopentadiene group, a cyclopentadiene group and a maleimide group, or a cyclopentadiene group and an aliphatic olefin (for example, an aliphatic olefin used as a peptide staple). In aspects, the adduct is one of the Diels-Alder adducts:

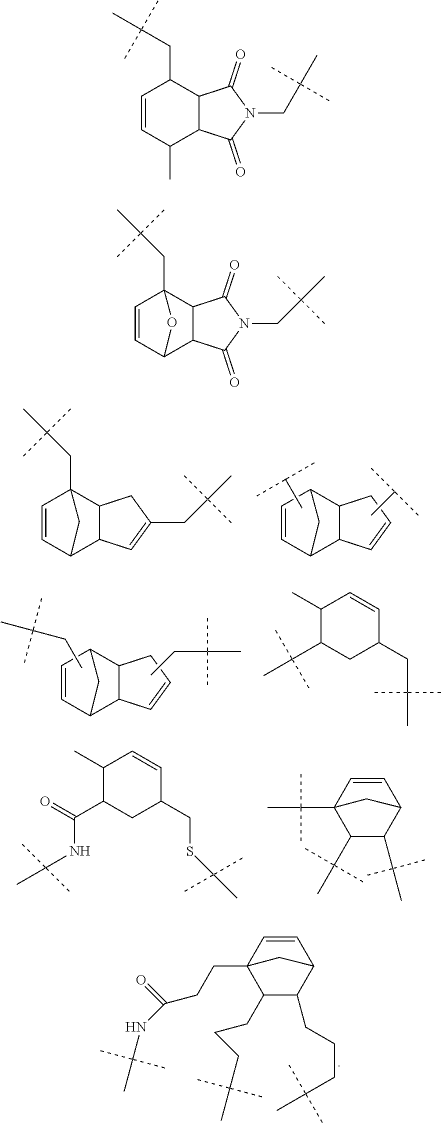

In aspects of the disclosure, exemplary positions of residues that can be covalently linked are shown in FIG. 1. In aspects, one residue is cysteine or a cysteine derivative and the other residue is lysine or a lysine derivative. In aspects, the cysteine and/or lysine are derivatized to form a diene or a dienophile. In aspects, the interpolypeptide covalent linkage between the third polypeptide and the fourth polypeptide is a maleimide-thiol adduct.

In aspects, the second polypeptide and the fourth polypeptide are linked through an interpolypeptide covalent linkage, creating what is referred to herein as a helical tetramer. In aspects, the interpolypeptide linkage is between the C-terminal amino acid of the second polypeptide and the C-terminal amino acid of the fourth polypeptide. In aspects, the interpolypeptide covalent linkage between the second polypeptide and the fourth polypeptide is a maleimide-thiol adduct. In aspects, any suitable helical dimer described herein may be covalently linked to any other suitable helical dimer described herein to create a helical tetramer.

In aspects of the disclosure, a reside other than Xaa^{A1} or Xaa^{B1} within the second polypeptide and a residue other than Xaa^{A1} and Xaa^{B1} within the fourth polypeptide are covalently linked, forming a ligated construct. In aspects, the linkage involves butyrlmaleimide (ButMal), glyclmaleimde (Glymal), or bismaleimidohexane. In aspects, the covalent linkage results in an adduct that forms from a Diels-Alder reaction, an olefin metathesis reaction, copper-catalyzed azide-alkyne click chemistry, cystine formation via oxidation of two cysteine residues, crosslink formation via alkylation of one or more cysteine residues, thiol-ene chemistry, or a lactam bridge formation between N- or C-termini and/or residue side chain(s). In aspects, at least one of the residues is a non-natural amino acid or an amino acid derivative. In aspects, the reactive functional groups are each independently bound to an amino acid side chain, amino acid amino group, amino acid carboxy group, or amino acid α-carbon. In aspects, the adduct is bound to a side chain, amine group, carboxy group, or α-carbon of one amino acid and to a side chain, amine group, carboxy group, or α-carbon of a different amino acid within the same polypeptide to provide a cyclic structure. In aspects, the macrocyclic polypeptide is formed in which one reactive functional group includes a diene and a different reactive functional group includes a dienophile. The complementary diene and dienophile pair can react to form a macrocyclic peptide through an intramolecular Diels-Alder reaction. The reactive functional groups may each independently be conjugated to a terminal amino acid or an internal amino acid. In aspects, the adduct is formed from a reaction between a hexadiene group and a maleimide group, a maleimide group and a furan group, a cyclopentadiene group and another cyclopentadiene group, a cyclopentadiene group and a maleimide group, or a cyclopentadiene group and an aliphatic olefin (for example, an aliphatic olefin used as a peptide staple). In aspects, the adduct is one of the Diels-Alder adducts:

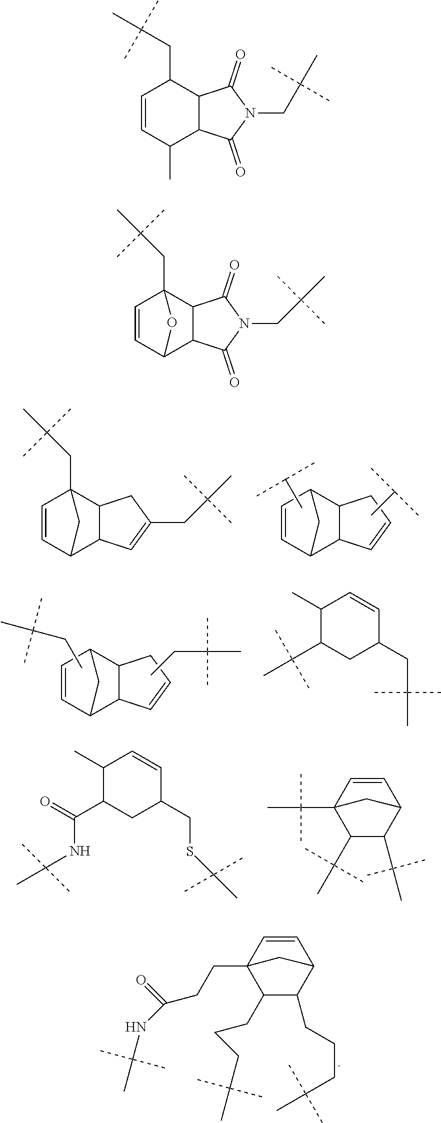

In aspects, one residue is cysteine or a cysteine derivative and the other residue is lysine or a lysine derivative. In aspects, the cysteine and/or lysine are derivatized to form a diene or a dienophile. In aspects, the interpolypeptide covalent linkage between the second polypeptide and the fourth polypeptide is a maleimide-thiol adduct.

In aspects, the N-terminus or the C-terminus of the first, second, third, or fourth polypeptide is capped. In aspects of the disclosure, the N-terminus is capped and the cap is acetyl or the C-terminus is capped and the cap is —NH₂. Exemplary N-terminal caps include:

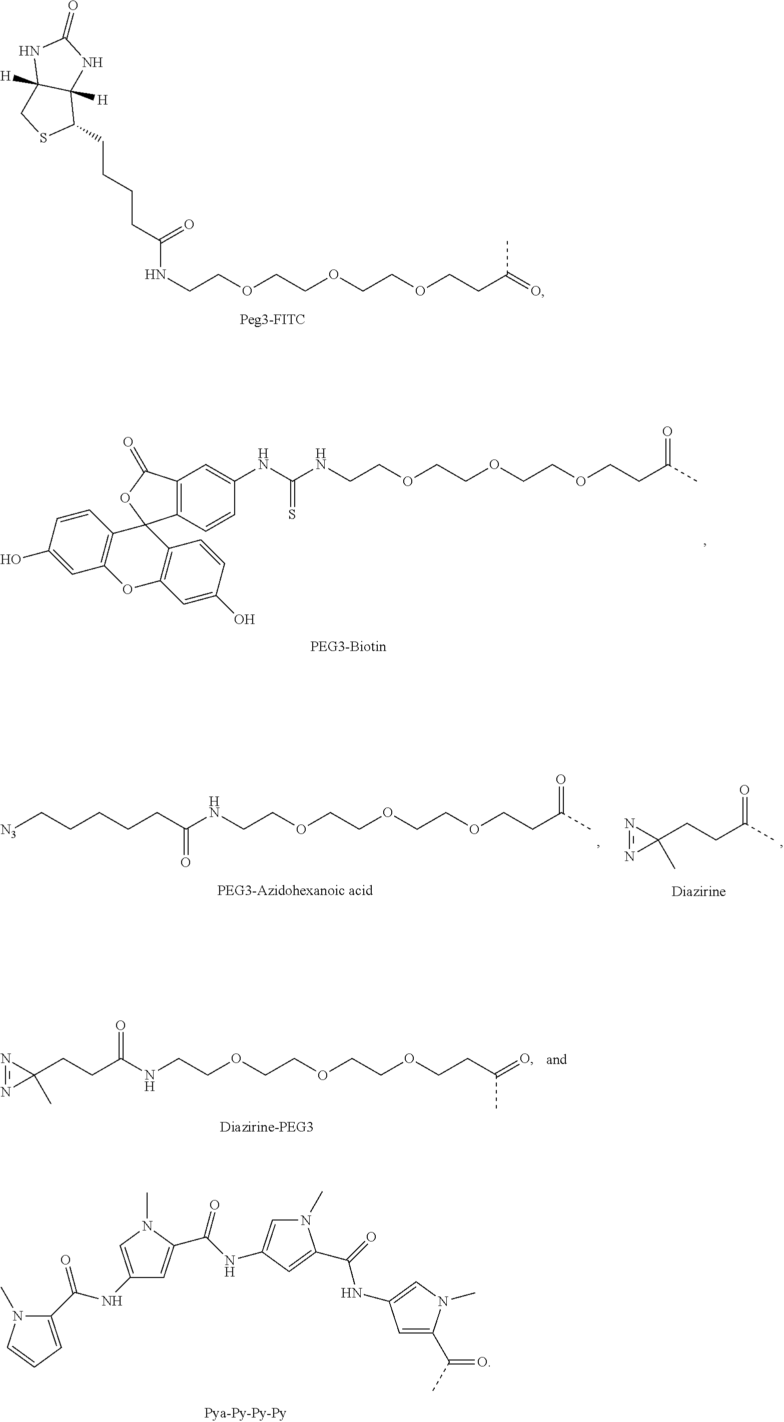

In aspects, the polypeptide construct binds to duplex DNA comprising the sequence of 5′-CANNTG-3′, wherein each N is independently any one of A, C, G, or T. In aspects, the DNA comprises the sequence of 5′-CACGTG-3′, 5′-CAGCTG-3′, 5′-CATATG-3′, 5′-CGTACG-3′, or 5′-CGCGCG-3′.

In aspects, the disclosure provides a polypeptide construct comprising (a) a first polypeptide comprising an amino acid sequence derived from a basic helix as listed in Table 2; and (b) a second polypeptide comprising an amino acid sequence derived from a helix as listed in Table 2; wherein the first polypeptide and the second polypeptide are linked through an interpolypeptide covalent linkage.

In aspects, the disclosure provides a polypeptide comprising the sequence of any of the polypetides described herein. In aspects, the disclosure provides a polypeptide comprising the sequence of any one of:

(Ac-RAQILCKATEYIQS₅MRRS₅Nβ)₂K-NH₂
(Ac-RAQILCKATEYIQS₅MRRS₅N is SEQ ID NO: 338)
(Ac-RAQILCKATEYIQYMRRKNβ)₂K-NH₂
(Ac-RAQILCKATEYIQYMRRKN is SEQ ID NO: 339)
(Ac-RAS₅ILCS₅ATEYIQYMRRKNβ)₂K-NH₂
(Ac-RAS₅ILCS₅ATEYIQYMRRKN is SEQ ID NO: 340)
(SEQ ID NO: 341)
Ac-HNALERKRRDHIKDSFHKLRDSVP
(SEQ ID NO: 342)
Ac-KRAHHNALERKRRDHIKDSFHK(GlyMal)LRDSVP-NH₂
(SEQ ID NO: 343)
Ac-KRAHHNALERKRRDHIKDSFHKLRDSVP
(SEQ ID NO: 344)
Ac-KRAHHNALERKRRDHIKDSFS₅K(GlyMal)LRS₅SVP-NH₂
(SEQ ID NO: 345)
Ac-KRAHHNALERKRRDHIKDSFS₅KLRS₅SVP
(SEQ ID NO: 346)
Ac-KRAHHNALERS₅RRDS₅IKDSFHK(GlyMal)LRDSVP-NH₂
(SEQ ID NO: 347)
AC-KRAHHNALERS₅RRDS₅IKDSFHKLRDSVP
(SEQ ID NO: 348)
Ac-KRAHHNS₅LERS₅RRDHIKDSFHK(GlyMal)LRDSVP-NH₂
(SEQ ID NO: 349)
Ac-KRAHHNS₅LERS₅RRDHIKDSFHKLRDSVP
(SEQ ID NO: 350)
Ac-KRAipHHNALERS₅RRDS₅IKDSFHKLRDSVP
(SEQ ID NO: 351)
Ac-KRAjHHNS₅LERS₅RRDHIKDSFHKLRDSVP
(SEQ ID NO: 352)
Ac-KRS₅HHNS₅LER(D-lysine)RRDHIKDSFHKLRDSVP
(SEQ ID NO: 353)
Ac-KRS₅HHNS₅LERAibRRDHIKDSFHKLRDSVP
(SEQ ID NO: 354)
Ac-KRS₅HHNS₅LERKRRDHIKDSFHK(GlyMal)LRDSVP-NH₂
(SEQ ID NO: 355)
Ac-KRS₅HHNS₅LERKRRDHIKDSFHKLRDSVP
(SEQ ID NO: 356)
Ac-KVC(StBu)ILKKATAYILS₅VQAS₅K(GlyMal)-NH₂
(SEQ ID NO: 357)
Ac-KVC(StBu)ILKKATAYILSVQAEK(GlyMal)-NH₂
(SEQ ID NO: 358)
Ac-KVCILKKATAYILS₅VQAS₅K(N3)-NH₂
(SEQ ID NO: 359)
Ac-KVCILKKATAYILSVQAEK(N3)-NH₂
(SEQ ID NO: 360)
Ac-KVS₅ILC(StBu)S₅ATAYILSVQAEK(GlyMal)-NH₂
(SEQ ID NO: 361)
Ac-KVS₅ILCS₅ATAYILSVQAEK(N3)-NH₂
(SEQ ID NO: 362)
Ac-KVVILC(StBu)KATAYILS₅VQAS₅K(GlyMal)-NH₂
(SEQ ID NO: 363)
Ac-KVVILC(StBu)KATAYILSVQAEK(GlyMal)-NH₂
(SEQ ID NO: 364)
Ac-KVVILCKATAYILS₅VQAS₅K(N3)-NH₂
(SEQ ID NO: 365)
Ac-KVVILCKATAYILSVQAEK(N3)-NH₂
(SEQ ID NO: 366)
Ac-RAC(StBu)ILDKATEYIQS₅MRRS₅C-NH₂
(SEQ ID NO: 367)
Ac-RAC(StBu)ILDKATEYIQYMRRKC-NH₂
(SEQ ID NO: 368)
Ac-RACILDKATEYIQS₅MRRS₅C(StBu)-NH₂
(SEQ ID NO: 369)
Ac-RACILDKATEYIQYMRRKC(StBu)-NH₂
(SEQ ID NO: 370)
Ac-RAQILC(StBu)KATEYIQS₅MRRS₅C-NH₂
(SEQ ID NO: 371)
Ac-RAQILC(StBu)KATEYIQS₅MRRS₅NβC-NH₂
(SEQ ID NO: 372)
Ac-RAQILC(StBu)KATEYIQYMRRKC-NH₂
(SEQ ID NO: 373)
Ac-RAQILC(StBu)KATEYIQYMRRKNβC-NH₂
(SEQ ID NO: 374)
Ac-RAQILCKATEYIQS₅MRRS₅C(StBu)-NH₂
(SEQ ID NO: 375)
Ac-RAQILCKATEYIQYMRRKC(StBu)-NH₂
(SEQ ID NO: 376)
Ac-RAS₅ILC(StBu)S₅ATEYIQYMRRKC-NH₂
(SEQ ID NO: 377)
Ac-RAS₅ILC(StBu)S₅ATEYIQYMRRKNβC-NH₂
(SEQ ID NO: 378)
Ac-RAS₅ILCS₅ATEYIQYMRRKC(StBu)-NH₂
(SEQ ID NO: 379)
Ac-SRA_{ib}QILCQATEYIQSN_{L}RRS₅N
(SEQ ID NO: 380)
Ac-SRAQILC(StBu)KATEYIQS₅N_{L}RRS₅NβC-NH₂
(SEQ ID NO: 381)
Ac-SRAQILCKATEYIQS₅N_{L}RRS₅N
(SEQ ID NO: 382)
Ac-SRAQILCKATEYIQYN_{L}R
(SEQ ID NO: 383)
Ac-SRAQILCKATEYIQYN_{L}RRKN
(SEQ ID NO: 384)
Ac-SRAQILCQATEYIQSN_{L}RRS₅N
(SEQ ID NO: 385)
Ac-SRAS₅ILC(StBu)S₅ATEYIQYN_{L}RRKNβC-NH₂
(SEQ ID NO: 386)
Ac-SRAS₅ILCS₅ATEYIQYN_{L}RRKN
(SEQ ID NO: 387)
Ac-WβADKRAHHNALERKRRDHIKDSFHK(GlyMal)LRDSV-NH₂
(SEQ ID NO: 388)
Ac-WβADKRAHHNALERKRRDHIKDSFHK(N3)LRDSV-NH₂
(SEQ ID NO: 389)
Ac-WβADKRAHHNALERKRRDHIKDSFHSLK(GlyMal)DSV-NH₂
(SEQ ID NO: 390)
Ac-WβADKRAHHNALERKRRDHIKDSFHSLK(N3)DSV-NH₂
(SEQ ID NO: 391)
Ac-WβADKRAHHNALERKRRDHIKDSFS₅K(GlyMal)LRS₅SV-NH₂
(SEQ ID NO: 392)
Ac-WβADKRAHHNALERKRRDHIKDSFS₅K(N3)LRS₅SV-NH₂
(SEQ ID NO: 393)
Ac-WβADKRAHHNALERKRRDHIKDSFS₅SLK(GlyMal)S₅SV-NH₂
(SEQ ID NO: 394)
Ac-WβADKRAHHNALERKRRDHIKDSFS₅SLK(N3)S₅SV-NH₂
(SEQ ID NO: 395)
Ac-WβADKRAHHNALERS₅RRDS₅IKDSFHK(GlyMal)LRDSV-NH₂
(SEQ ID NO: 396)
Ac-WβADKRAHHNALERS₅RRDS₅IKDSFHK(N3)LRDSV-NH₂
(SEQ ID NO: 397)
Ac-WβADKRAHHNALERS₅RRDS₅IKDSFHSLK(GlyMal)DSV-NH₂
(SEQ ID NO: 398)
Ac-WβADKRAHHNALERS₅RRDS₅IKDSFHSLK(N3)DSV-NH₂
(SEQ ID NO: 399)
Ac-WβADKRAHHNS₅LERS₅RRDHIKDSFHK(GlyMal)LRDSV-NH₂
(SEQ ID NO: 400)
Ac-WβADKRAHHNS₅LERS₅RRDHIKDSFHK(N3)LRDSV-NH₂
(SEQ ID NO: 401)
Ac-WβADKRAHHNS₅LERS₅RRDHIKDSFHSLK(GlyMal)DSV-NH₂
(SEQ ID NO: 402)
Ac-WβADKRAHHNS₅LERS₅RRDHIKDSFHSLK(N3)DSV-NH₂
(SEQ ID NO: 403)
Ac-WβADKRS₅HHNS₅LERKRRDHIKDSFHK(GlyMal)LRDSV-NH₂
(SEQ ID NO: 404)
Ac-WβADKRS₅HHNS₅LERKRRDHIKDSFHK(N3)LRDSV-NH₂
(SEQ ID NO: 405)
Ac-WβADKRS₅HHNS₅LERKRRDHIKDSFHSLK(GlyMal)DSV-NH₂
(SEQ ID NO: 406)
Ac-WβADKRS₅HHNS₅LERKRRDHIKDSFHSLK(N3)DSV-NH₂
(SEQ ID NO: 407)
Ac-WβKRAHHNALERKRRDHIKDSFHK(GlyMal)LRDSV-NH₂
(SEQ ID NO: 408)
Ac-WβKRAHHNALERKRRDHIKDSFS&K(GlyMal)LRS₅SV-NH₂
(SEQ ID NO: 409)
Ac-WβKRAHHNALERS₅RRDS₅IKDSFHK(GlyMal)LRDSV-NH₂
(SEQ ID NO: 410)
Ac-WβKRAHHNS₅LERS₅RRDHIKDSFHK(GlyMal)LRDSV-NH₂
(SEQ ID NO: 411)
Ac-WβKRS₅HHNS₅LERKRRDHIKDSFHK(GlyMal)LRDSV-NH₂
(SEQ ID NO: 412)
Ac-WβNVKRRTHNS₅LERS₅RRNELKRSFFALK(GlyMal)DQI-NH₂
(SEQ ID NO: 413)
Ac-WβNVKRRTHNS₅LERS₅RRNELKRSFFALK(N3)DQI-NH₂
(SEQ ID NO: 414)
Ac-WβNVKRRTHNS₅LERS₅RRNELKRSFFK(GlyMal)LRDQI-NH₂
(SEQ ID NO: 415)
Ac-WβNVKRRTHNS₅LERS₅RRNELKRSFFK(N3)LRDQI-NH₂
(SEQ ID NO: 416)
Ac-WβNVKRRTHNVLERQRRNELKRSFFALK(GlyMal)DQI-NH₂
(SEQ ID NO: 417)
Ac-WβNVKRRTHNVLERQRRNELKRSFFALK(N3)DQI-NH₂
(SEQ ID NO: 418)
Ac-WβNVKRRTHNVLERQRRNELKRSFFK(GlyMal)LRDQI-NH₂
(SEQ ID NO: 419)
Ac-WβNVKRRTHNVLERQRRNELKRSFFK(N3)LRDQI-NH₂
(SEQ ID NO: 420)
Ac-WβNVKRRTHNVLERQRRNELKRSFS₅ALK(GlyMal)S₅QI-NH₂
(SEQ ID NO: 421)
Ac-WβNVKRRTHNVLERQRRNELKRSFS₅ALK(N3)S₅QI-NH₂
(SEQ ID NO: 422)
Ac-WβNVKRRTHNVLERQRRNELKRSFS₅K(GlyMal)LRSQI-NH₂
(SEQ ID NO: 423)
Ac-WβNVKRRTHNVLERQRRNELKRSFS₅K(N3)LRS₅QI-NH₂
(SEQ ID NO: 424)
Ac-WβNVKRRTHNVLERS₅RRNS₅LKRSFFALK(GlyMal)DQI-NH₂
(SEQ ID NO: 425)
Ac-WβNVKRRTHNVLERS₅RRNS₅LKRSFFALK(N3)DQI-NH₂
(SEQ ID NO: 426)
Ac-WβNVKRRTHNVLERS₅RRNS₅LKRSFFK(GlyMal)LRDQI-NH₂
(SEQ ID NO: 427)
Ac-WβNVKRRTHNVLERS₅RRNS₅LKRSFFK(N3)LRDQI-NH₂
(SEQ ID NO: 428)
Ac-WβNVKRS₅THNS₅LERQRRNELKRSFFALK(GlyMal)DQI-NH₂
(SEQ ID NO: 429)
Ac-WβNVKRS₅THNS₅LERQRRNELKRSFFALK(N3)DQI-NH₂
(SEQ ID NO: 430)
Ac-WβNVKRS₅THNS₅LERQRRNELKRSFFK(GlyMal)LRDQI-NH₂
(SEQ ID NO: 431)
Ac-WβNVKRSTHNS₅LERQRRNELKRSFFK(N3)LRDQI-NH₂
(SEQ ID NO: 432)
Ac-KRAHHNALERKRRDHIKDSFHKLRDSVP-NH₂
(SEQ ID NO: 433)
Ac-SRAQILCKATEYIQYN_{L}RRKN-NH₂
(SEQ ID NO: 434)
Ac-PRFQSAADKRAHHNALERKRRDHIKDSFHKLRDSVP-NH₂
(SEQ ID NO: 435)
Ac-QSAADKRAHHNALERKRRDHIKDSFHKLRDSVP-NH₂
(SEQ ID NO: 436)
Ac-IEVESDADKRAHHNALERKRRDHIKDSFHKLRDSVP-NH₂
(SEQ ID NO: 437)
Ac-PRSSDTEENVKRAHHNALERKRRDHIKDSFHKLRDSVP-NH₂
(SEQ ID NO: 438)
Ac-TEENVKRAHHNALERKRRDHIKDSFHKLRDSVP-NH₂
(SEQ ID NO: 439)
Ac-KSKKNNSSKRAHHNALERKRRDHIKDSFHKLRDSVP-NH₂
(SEQ ID NO: 440)
Ac-KNNSSKRAHHNALERKRRDHIKDSFHKLRDSVP-NH₂
(SEQ ID NO: 441)
Ac-PRFQSAADKRS₅HHNS₅LERKRRDHIKDSFHKLRDSVP-NH₂
(SEQ ID NO: 442)
Ac-PRFQSAS₅DKRS₅HHNALERKRRDHIKDSFHKLRDSVP-NH₂
(SEQ ID NO: 443)
Ac-PRS₅FQSS₅DKRAHHNALERKRRDHIKDSFHKLRDSVP-NH₂
(SEQ ID NO: 433)
Ac-SRAQILCKATEYIQYN_{L}RRKN-NH₂
(SEQ ID NO: 444)
Ac-SRAQILCKATEYIQYN_{L}RRKNHTHQQDIDDLK-NH₂
(SEQ ID NO: 445)
Ac-SRAQILCKATEYIQYN_{L}RRKNHTLISE-NH₂
(SEQ ID NO: 446)
Ac-SRAQILCKATEYIQYN_{L}RRKLHTHE-NH₂
(SEQ ID NO: 447)
Ac-SRA_{ib}QILCQATEYIQS₅N_{L}RRS₅LHTHE-NH₂
(SEQ ID NO: 432)
Ac-KRAHHNALERKRRDHIKDSFHKLRDSVP-NH₂
(SEQ ID NO: 448)
Ac-KRS₅HHNS₅LERKRRDHIKDSFHKLRDSVP-NH₂
(SEQ ID NO: 449)
Ac-KRS₅HANS₅LERKRLDHIKDSFHKLRDSVP-NH₂
(SEQ ID NO: 450)
Ac-KRS₅HANS₅LERKRTDHIKDSFHKLRDSVP-NH₂
(SEQ ID NO: 451)
Ac-KKS₅HSNS₅LARKRRDHIKDSFHKLRDSVP-NH₂
(SEQ ID NO: 452)
Ac-KRS₅HHNS₅LNRKRRDHIKDSFHKLRDSVP-NH₂
(SEQ ID NO: 453)
Ac-PRFQSA(S5)DKR(S5)HHNALERKRRDHIKD
SFHK(GlyMal)LRDSVP-NH₂
(SEQ ID NO: 454)
Ac-SR(Aib)QILCQATEYIQ(S5)(Nle)RR(S5)LHTHE-NH₂
(SEQ ID NO: 453)
Ac-PRFQSA(S5)DKR(S5)HHNALERKRRDHIKDSFHK
(GlyMal)LRDSVP-NH₂
(SEQ ID NO: 455)
Ac-SRAQILCKATEYIQYLR(S5)KIH(S5)LE-NH₂

wherein
   Ac is acetyl;
   A_{ib} is 2-aminoisobutyric acid;
   N_{L} is norleucine;
   β prior to an amino acid represents that amino acid is a p amino acid;
   GlyMal is glycylmaleimide;
   StBu is tert-butylsulfenyl;
   K(N3) is azidolysine; and
   S₅ is (S)-2-(4′-pentenyl)alanine.

The polypeptides may be synthesized using any suitable method. The Example below provides suitable methods. Also, for example, the process may include synthesis, ring closing metathesis (RCM) and capping for a single helix and partial synthesis, RCM, hydrogenation, synthesis, RCM, and capping for two helices. In aspects of the disclosure, the side chains of non-natural amino acids, wherein each non-natural amino acid includes a moiety, wherein each moiety is capable of undergoing a reaction with a moiety of one other of the non-natural amino acids to form a covalent cross-link, can be covalently linked (e.g., R3 to S5, S5 to S5, R5 to S5, or R8 to S5) in the presence of a catalyst to produce the “staple” of the polypeptide. Methods of synthesis that may be used are described in Bird et al., Methods in Enzymology, 446: 369-386 (2008), incorporated by reference herein, and Shim et al., Chem. Biol. Drug Des., 82: 635-642 (2013), incorporated by reference herein. Specifically, methods of making hydrocarbon stapled polypeptides are known in the art and have been described (see, e.g., the Examples and Verdine et al., “Stapled Peptides for Intracellular Drug Targets” in Methods in Enzymology, 503: 3-23 (2012), which is incorporated by reference herein).

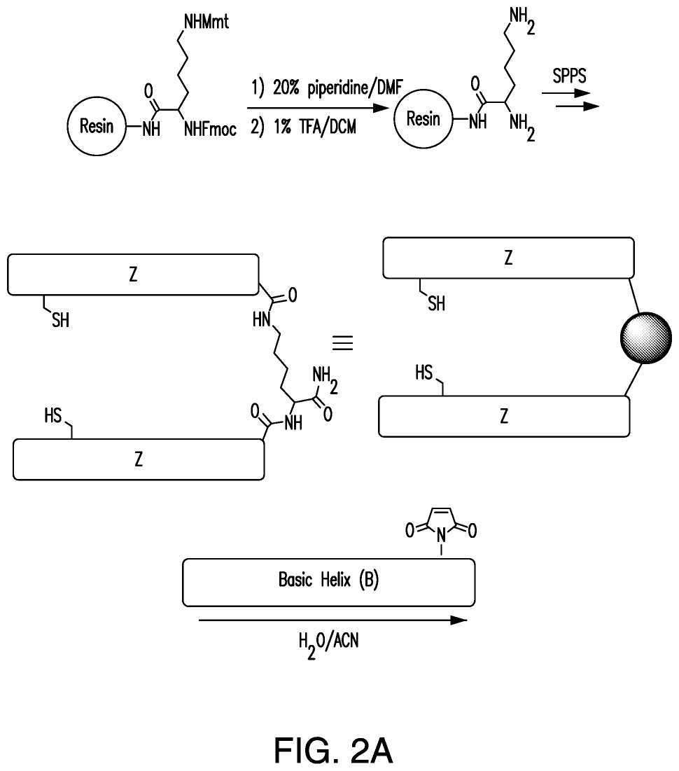

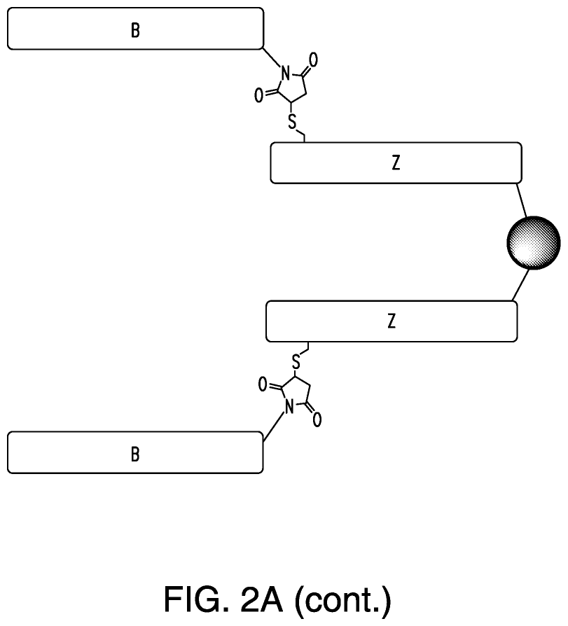

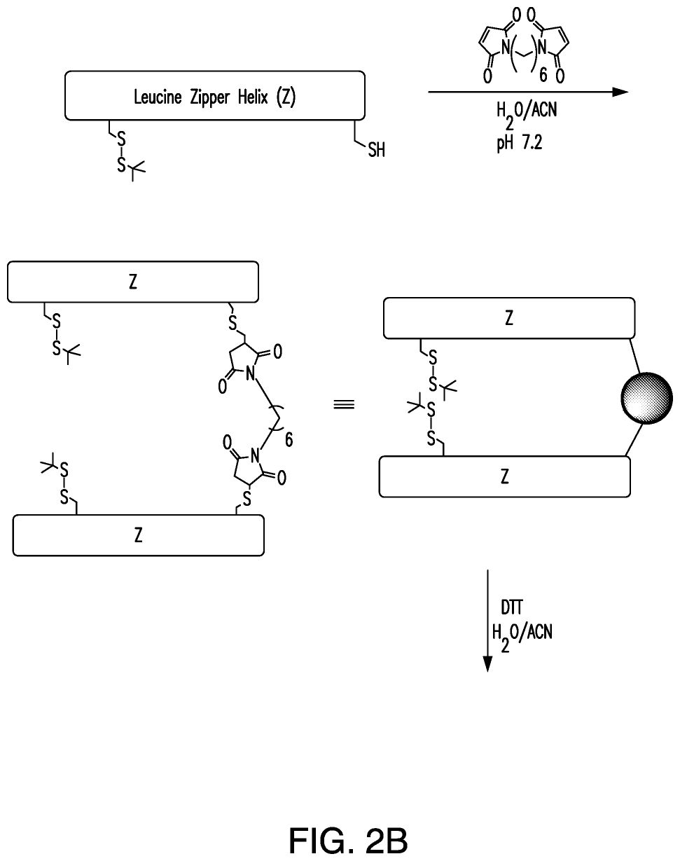

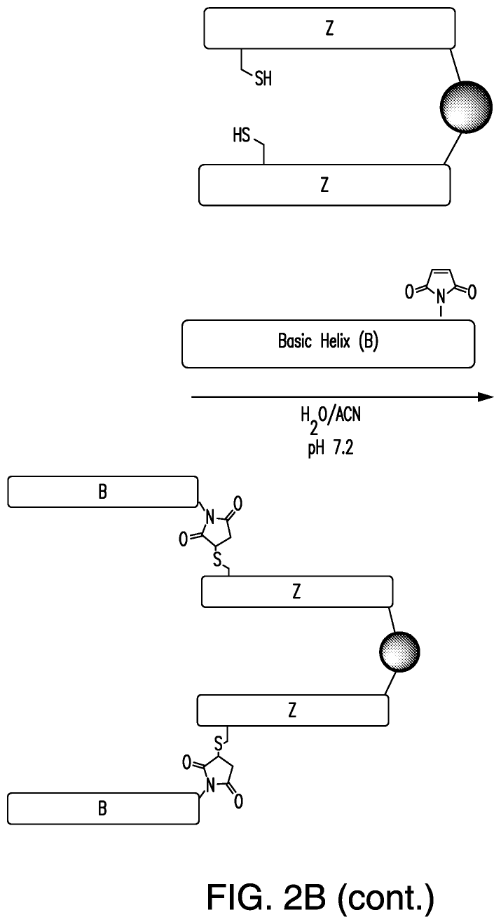

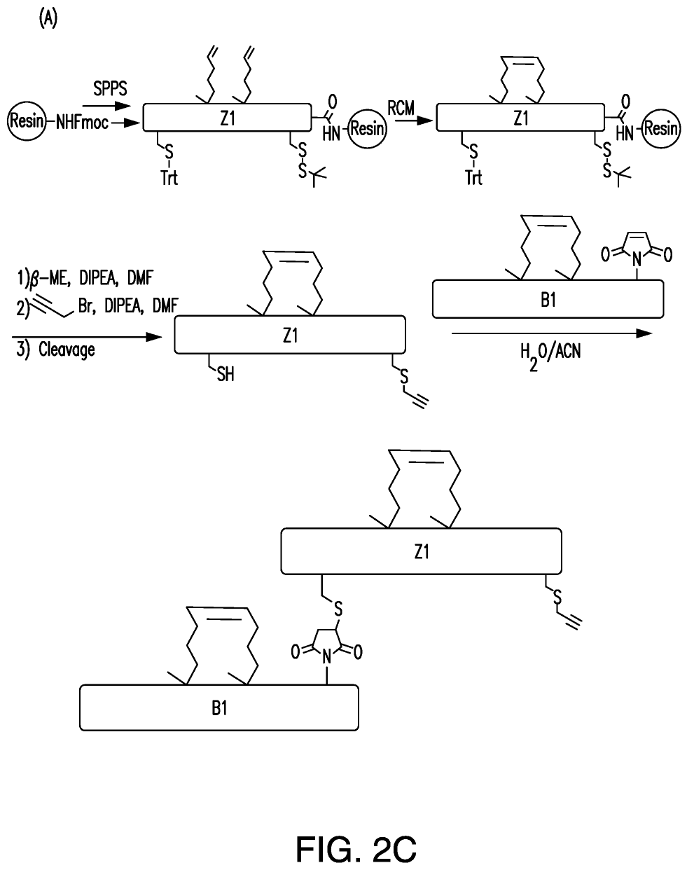

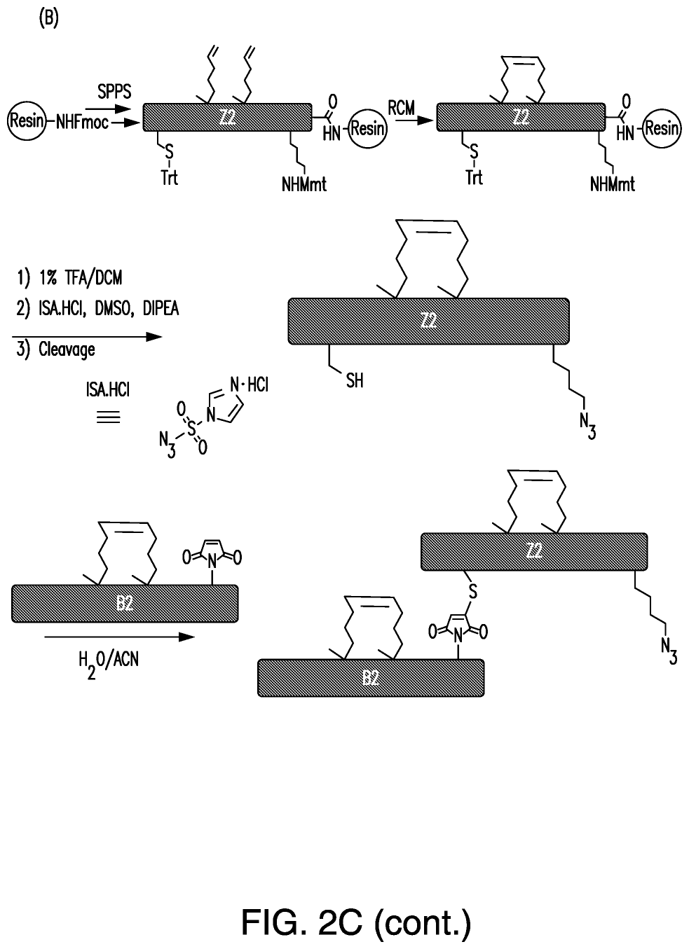

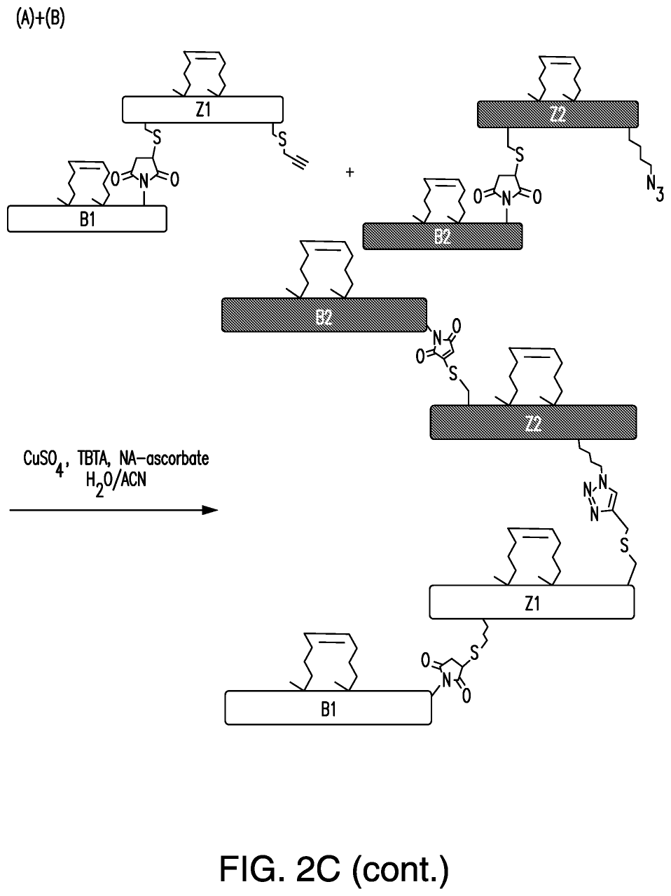

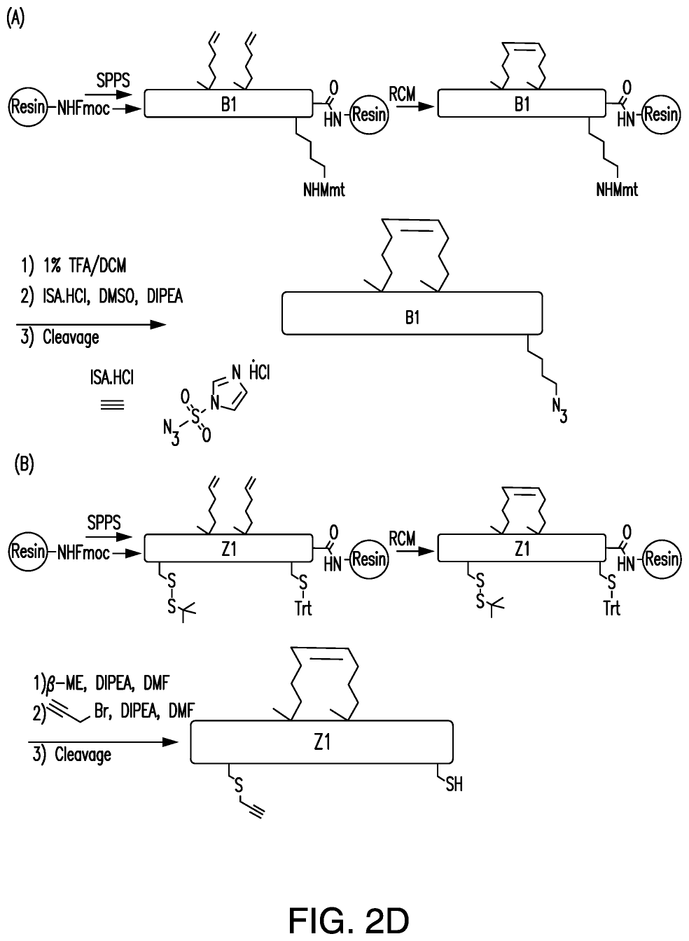

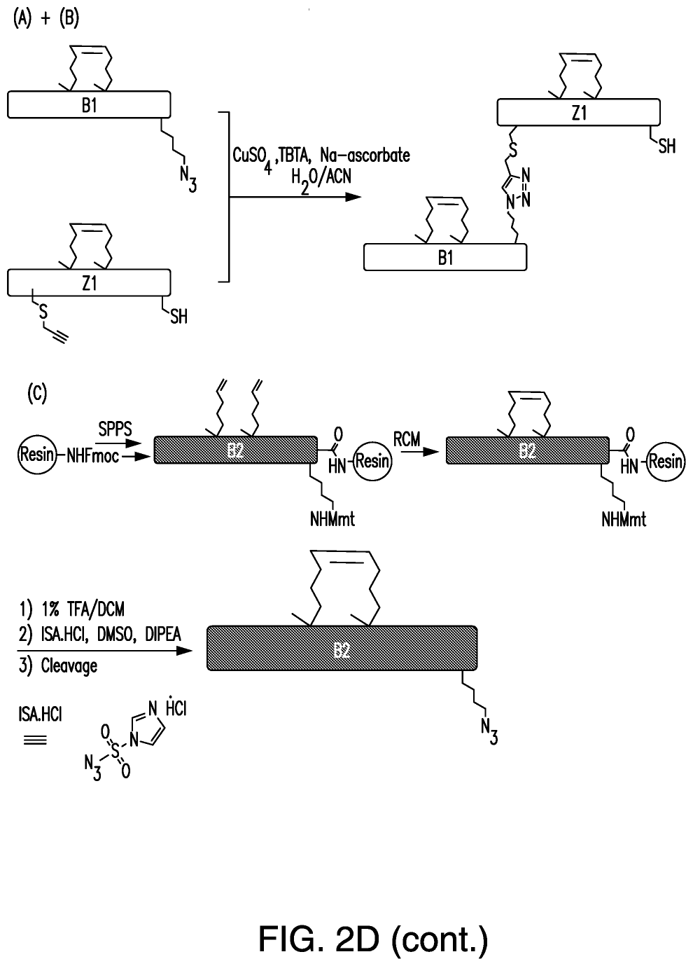

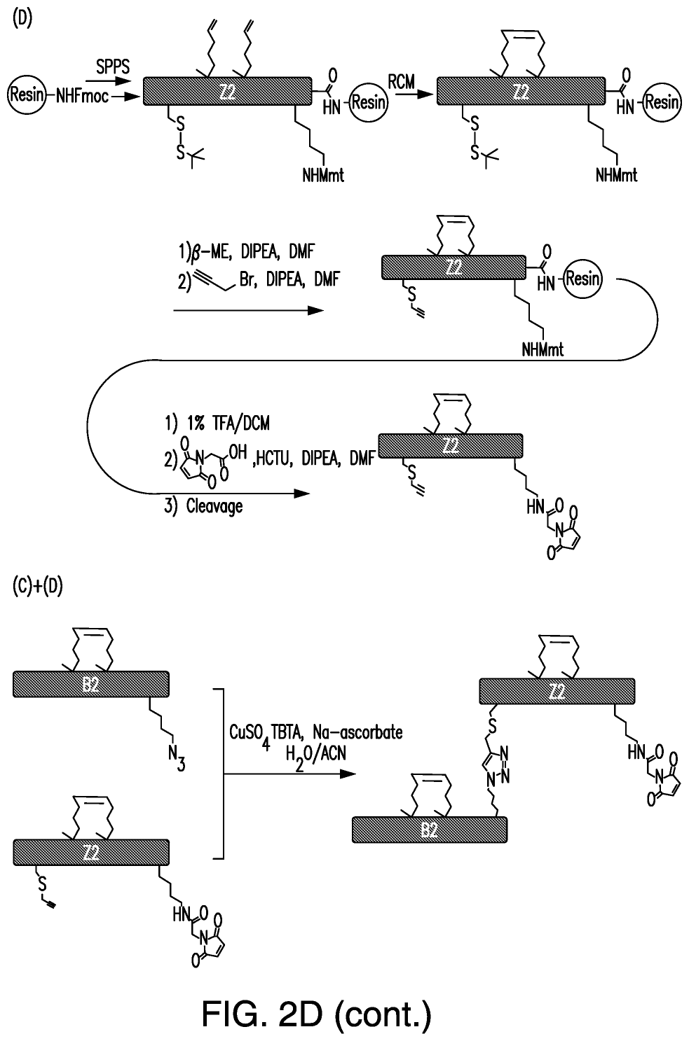

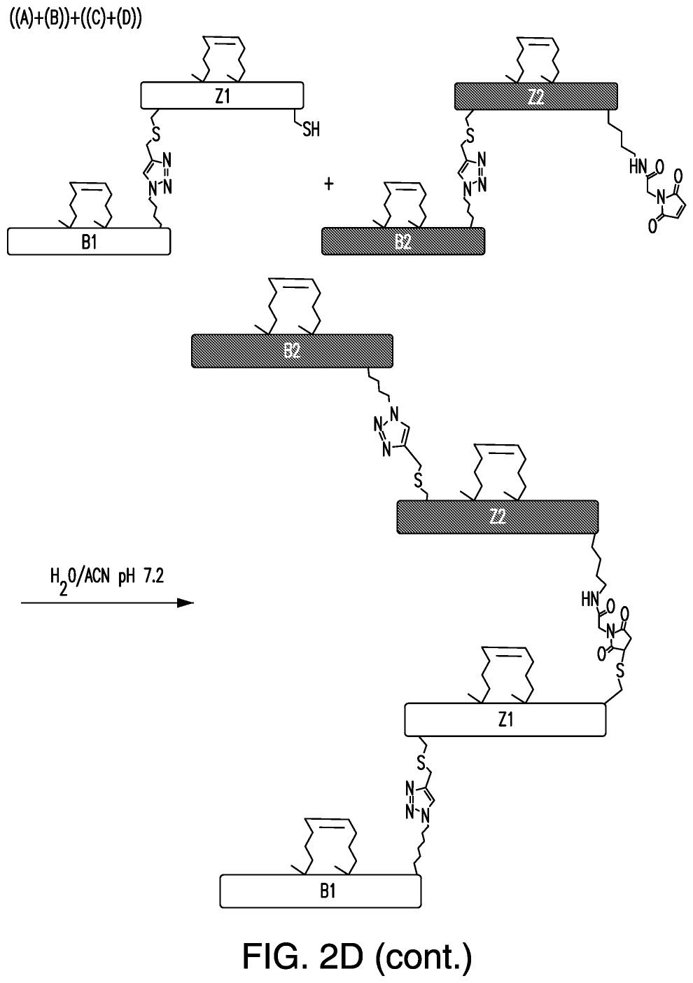

In aspects of the disclosure, the polypeptides can be synthesized as shown in FIG. 2A, which is a schematic of synthesis of a branched zipper helix. In aspects of the disclosure, the polypeptides can be synthesized as shown in FIG. 2B, which is a schematic of homodimerization. In aspects of the disclosure, the polypeptides can be synthesized as shown in FIG. 2C which is a schematic of heterodimerization with orthogonal chemistry for the synthesis of asymmetric tetrahelical peptide conjugate. In aspects of the disclosure, the polypeptides can be synthesized as shown in FIG. 2D, which is a schematic of heterodimerization by switching the order of conjugation chemistry.

In aspects of the disclosure, one or more peptide bonds may be replaced by a different bond that may increase the stability of the polypeptide. Peptide bonds can be replaced by: a retro-inverso bond (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

Amino acids of the polypeptides may be substituted using amino acid substitutions. Such substitutions may be conservative substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Gly, Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc. Also, substitution of amino acids may be accomplished using amino acids having high helical propensity, such as α-aminoisobutyric acid (A_{ib}). Substitutions that do not impact DNA binding are preferable.

The polypeptides can be any suitable length of amino acids. For example, any of the inventive sequences can have an additional 1, 2, 3, 4, 5, 6, 7, 8, 9, 10, or more amino acids on either the N-terminus or C-terminus or both.

Any of the polypeptides may be isolated. Any of the polypeptides may be purified. By “isolated” is meant the removal of a substance (e.g., a polypeptide) from its natural environment. By “purified” is meant that a given substance (e.g., a polypeptide), whether one that has been removed from nature (e.g., a protein enzymatically cleaved into polypeptides) or synthesized (e.g., by polypeptide synthesis), has been increased in purity, wherein “purity” is a relative term, not “absolute purity.” It is to be understood, however, that polypeptides may be formulated with diluents or adjuvants and still for practical purposes be isolated. For example, polypeptides can be mixed with an acceptable carrier or diluent when used for introduction into cells.

The polypeptides described herein may be provided in the form of a salt, e.g., a pharmaceutically acceptable salt. Suitable pharmaceutically acceptable acid addition salts, for example, include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

In aspects of the disclosure, the disclosure provides a pharmaceutical composition comprising a therapeutically effective amount of a polypeptide or a polypeptide composition described herein and a pharmaceutically acceptable excipient.

In aspects, the disclosure provides a pharmaceutical composition comprising a therapeutically effective amount of a polypeptide construct or a polypeptide described herein or and a pharmaceutically acceptable excipient. Thus, one or more polypeptides described herein can be administered alone or in a composition (e.g., formulated in a pharmaceutically acceptable composition). Such a composition comprises a carrier (e.g., a pharmaceutically acceptable carrier), such as those known in the art. A pharmaceutically acceptable carrier (or excipient) preferably is chemically inert to the polypeptide and has few or no detrimental side effects or toxicity under the conditions of use. The choice of carrier is determined, in part, by the particular method used to administer the composition.

Carrier formulations suitable for parenteral, oral, nasal (and otherwise inhaled), topical, and other administrations can be found in Remington's Pharmaceutical Sciences 17^{th} ed., Mack Publishing Co., Easton, PA (2000), which is incorporated by reference herein. Requirements for effective pharmaceutical carriers in parenteral and injectable compositions are well known to those of ordinary skill in the art. See, e.g., Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986). Accordingly, there is a wide variety of suitable formulations of the composition.

The composition can contain suitable buffering agents, including, for example, acetate buffer, citrate buffer, borate buffer, or a phosphate buffer. The pharmaceutical composition also, optionally, can contain suitable preservatives, such as benzalkonium chloride, chlorobutanol, parabens, and thimerosal.

The composition can be presented in unit dosage form and can be prepared by any suitable method, many of which are well known in the art of pharmacy. Such methods include the step of bringing the polypeptide into association with a carrier that constitutes one or more accessory ingredients. In general, the composition is prepared by uniformly and intimately bringing the polypeptide into association with a liquid carrier, a finely divided solid carrier, or both, and then, if necessary, shaping the product.

The composition can be administered using any suitable method including, but not limited to parenteral, oral, nasal (or otherwise inhaled), and topical administration. Delivery systems useful in the context of the disclosure include time-released, delayed-release, and sustained-release delivery systems.

A composition suitable for parenteral administration conveniently comprises a sterile aqueous preparation of the polypeptide, which may be isotonic with the blood of the recipient. This aqueous preparation can be formulated according to known methods using suitable dispersing or wetting agents and suspending agents.

Sterile powders for sterile injectable solutions can be prepared by vacuum drying and/or freeze-drying to yield a powder of the polypeptide, optionally, in association with a filler or diluent.

A composition suitable for oral administration can be formulated in discrete units such as capsules, cachets, tablets, or lozenges, each containing a predetermined amount of the polypeptide as a powder or granules. A tablet may be made by compression or molding, optionally with one or more accessory ingredients. Compressed tablets may be prepared by compressing in a suitable machine, with the polypeptide being in a free-flowing form, such as a powder or granules, which optionally is mixed with a binder, disintegrant, lubricant, inert diluent, surface polypeptide, or discharging agent. Molded tablets comprised of a mixture of the polypeptide with a suitable carrier may be made by molding in a suitable machine.

Liquid dosage forms for oral and parenteral administration include, but are not limited to, pharmaceutically acceptable emulsions, microemulsions, solutions, suspensions, syrups and elixirs. In addition to the active polypeptide, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In aspects of the disclosure for parenteral administration, the proteins, polypeptides, and polypeptides of the disclosure are mixed with solubilizing agents such a Cremophor, alcohols, oils, modified oils, glycols, polysorbates, cyclodextrins, polymers, or any combination thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles and solvents that may be employed are water, Ringer's solution, U.S.P. and isotonic sodium chloride solution, and 1,3-butanediol. In addition, sterile, fixed oils can be employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Topical formulations comprise at least one polypeptide dissolved or suspended in one or more media, such as mineral oil, petroleum, polyhydroxy alcohols, or other bases used for topical pharmaceutical formulations. Transdermal formulations may be prepared by incorporating the polypeptide in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

The amount (e.g., therapeutically effective amount) of polypeptide suitable for administration depends on the specific polypeptide used and the particular route of administration. In aspects of the disclosure, for example, polypeptide can be administered in a dose of about 0.5 ng to about 900 ng (e.g., about 1 ng, 25 ng, 50 ng, 100, ng, 200 ng, 300 ng, 400 ng, 500, ng, 600 ng, 700 ng, 800 ng, or any range bounded by any two of the aforementioned values), in a dose of about 1 μg to about 900 μg (e.g., about 1 μg, 2 μg, 5 μg, 10 μg, 15 μg, 20 μg, 25 μg, 30 μg, 40 μg, 50 μg, 60 μg, 70 μg, 80 μg, 90 μg, 100 μg, 200 μg, 300 μg, 400 μg, 500, μg, 600 μg, 700 μg, 800 μg, or any range bounded by any two of the aforementioned values), or in a dose of about 1 mg to about 200 mg (e.g., about 1 mg, 2 mg, 5 mg, 10 mg, 15 mg, 20 mg, 25 mg, 30 mg, 40 mg, 50 mg, 60 mg, 70 mg, 80 mg, 90 mg, 100 mg, 125 mg, 150 mg, 175 mg, or any range bounded by any two of the aforementioned values) per kilogram body weight of the subject. Several doses can be provided over a period of days or weeks.

In aspects, the disclosure provides a method of treating disease in a subject in need thereof comprising administering to the subject a therapeutically effective amount of a polypeptide construct or a polypeptide described herein, or a pharmaceutical composition described herein.

The terms “treat,” “treating,” “treatment,” “therapeutically effective,” “inhibit,” etc. used herein do not necessarily imply 100% or complete treatment/inhibition/reduction. Rather, there are varying degrees, which one of ordinary skill in the art recognizes as having a potential benefit or therapeutic effect. In this respect, the polypeptides and methods can provide any amount of any level of treatment/inhibition/reduction. Furthermore, the treatment provided by the inventive method can include the treatment of one or more conditions or symptoms of the disease being treated.

The terms “co-administering,” “co-administration” and “co-administered” used herein refer to the administration of an polypeptide described herein and one or more additional therapeutic agents sufficiently close in time to (i) enhance the effectiveness of the polypeptide or the one or more additional therapeutic agents and/or (ii) reduce an undesirable side effect of the polypeptide or the one or more additional therapeutic agents. In this regard, the polypeptide can be administered first, and the one or more additional therapeutic agents can be administered second, or vice versa. Alternatively, the polypeptide and the one or more additional therapeutic agents can be co-administered simultaneously.

The term “subject” is used herein to refer to human or animal subjects (e.g., mammals).

In aspects of the disclosure, the disclosure provides a method of treating disease in a subject in need thereof comprising administering to the subject a therapeutically effective amount of a polypeptide, a polypeptide composition, or a pharmaceutical composition described herein.

The following includes certain aspects of the disclosure.
1. A polypeptide construct comprising:
(a) a first polypeptide comprising an amino acid sequence derived from a basic helix of a transcription factor protein that comprises a basic helix-loop-helix domain; and
(b) a second polypeptide comprising an amino acid sequence derived from a helix that extends in the C-terminal direction from the end of the loop of a basic helix-loop-helix domain of a transcription factor protein that comprises a basic helix-loop-helix domain;
wherein the first polypeptide and the second polypeptide are linked through an interpolypeptide covalent linkage.
2. The polypeptide construct of aspect 1, wherein the basic helix of the first polypeptide comprises the amino acid sequence extending 36 residues in the N-terminal direction from the start of the loop of the basic helix-loop-helix domain.
3. The polypeptide construct of aspect 1 or 2, wherein the helix of the second polypeptide comprises the amino acid sequence extending 31 residues in the C-terminal direction from the end of the loop of the basic helix-loop-helix domain.
4. The polypeptide construct of any one of aspects 1-3, wherein the amino acid sequence of the first polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the first polypeptide.
5. The polypeptide construct of any one of aspects 1-4, wherein the amino acid sequence of the second polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the second polypeptide.
6. The polypeptide construct of aspect 4 or 5, wherein each set of non-natural amino acids are capable of undergoing a Diels-Alder reaction, a Huisgen reaction, or an olefin metathesis reaction.
7. The polypeptide construct of aspect 4 or 5, wherein one non-natural amino acid within a set is Xaa^{A1} and the other non-natural amino acid within the set is Xaa^{B1},
wherein

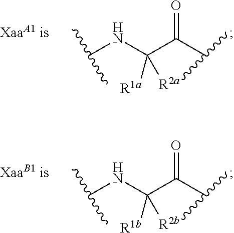

R^{1a} and R^{1b} are independently H, alkyl, alkenyl, alkynyl, arylalkyl, cycloalkylalkyl, heteroarylalkyl, or heterocyclylalkyl;
R^{2a} and R^{2b} are (i) independently alkenyl, alkynyl, azido, amino, carboxylic acid, or sulfide or (ii) taken together to form alkylene, alkenylene, alkynylene, or [R^{3a}—X—R^{3b}]ₙ, each of which is substituted with 0-6 R⁴;
each R^{3a} and R^{3b} are independently alkylene, alkenylene or alkynylene;
each R⁴ is independently halo, alkyl, OR⁵, N(R⁵)₂, SR⁵, SOR⁵, SO₂R⁵, CO₂R⁵, R⁵;
each X is independently O, S, SO, SO₂, CO, CO₂, CONR⁵ or

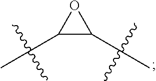 [SMILES: *C1OC1*]

each R⁵ is independently H or alkyl; and
n is an integer 1-4.
8. The polypeptide construct of aspect 4 or 5, wherein the non-natural amino acids are capable of forming together a thioether, ether, amide, amine, triazole, or carbon-carbon double bond or a Diels-Alder adduct after reaction.
9. The polypeptide construct of aspect 4 or 5, wherein the non-natural amino acids are independently selected from (S)-2-(4′-pentenyl)alanine (S5), (R)-2-(2′-propenyl)alanine (R3), and (R)-2-(7′-octenyl)alanine (R8).
10. The polypeptide construct of any one of aspects 4-9, wherein the non-natural amino acids have undergone reaction to form the intrapolypeptide covalent cross-link with each other.
11. The polypeptide construct of any one of aspects 1-10, wherein the interpolypeptide covalent linkage between the first polypeptide and the second polypeptide is a maleimide-thiol adduct.
12. A polypeptide construct comprising:
(a) the polypeptide construct of any one of aspects 1-11;
(b) a third polypeptide comprising an amino acid sequence derived from a basic helix of a transcription factor protein that comprises a basic helix-loop-helix domain; and
(c) a fourth polypeptide comprising an amino acid sequence derived from a helix that extends in the C-terminal direction from the end of the loop of a basic helix-loop-helix domain of a transcription factor protein that comprises a basic helix-loop-helix domain;
wherein the third polypeptide and the fourth polypeptide are linked through an interpolypeptide covalent linkage.
13. The polypeptide construct of aspect 12, wherein the basic helix of the third polypeptide comprises the amino acid sequence extending 36 residues in the N-terminal direction from the start of the loop of the basic helix-loop-helix domain.
14. The polypeptide construct of aspect 12 or 13, wherein the helix of the fourth polypeptide comprises the amino acid sequence extending 31 residues in the C-terminal direction from the end of the loop of the basic helix-loop-helix domain.
15. The polypeptide construct of any one of aspects 12-14, wherein the amino acid sequence of the third polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the third polypeptide.
16. The polypeptide construct of any one of aspects 12-15, wherein the amino acid sequence of the fourth polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the fourth polypeptide.
17. The polypeptide construct of aspect 15 or 16, wherein each set of non-natural amino acids are capable of undergoing a Diels-Alder reaction, a Huisgen reaction, or an olefin metathesis reaction.
18. The polypeptide construct of aspect 15 or 16, wherein one non-natural amino acid within a set is Xaa^{A1} and the other non-natural amino acid within the set is Xaa^{B1},
wherein

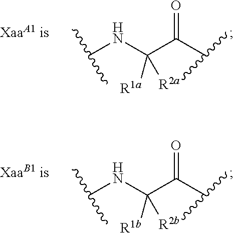

R^{1a} and R^{1b} are independently H, alkyl, alkenyl, alkynyl, arylalkyl, cycloalkylalkyl, heteroarylalkyl, or heterocyclylalkyl;
R² and R^{2b} are (i) independently alkenyl, alkynyl, azido, amino, carboxylic acid, or sulfide or (ii) taken together to form alkylene, alkenylene, alkynylene, or [R^{3a}—X—R^{3b}]ₙ, each of which is substituted with 0-6 R⁴;
each R³ and R^{3b} are independently alkylene, alkenylene or alkynylene;
each R⁴ is independently halo, alkyl, OR⁵, N(R⁵)₂, SR⁵, SOR⁵, SO₂R⁵, CO₂R⁵, R⁵;
each X is independently O, S, SO, SO₂, CO, CO, CONR⁵ or

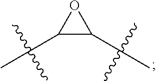 [SMILES: *C1OC1*]

each R⁵ is independently H or alkyl; and
n is an integer 1-4.
19. The polypeptide construct of aspect 15 or 16, wherein the non-natural amino acids are capable of forming together a thioether, ether, amide, amine, triazole, or carbon-carbon double bond or a Diels-Alder adduct after reaction.
20. The polypeptide construct of aspect 15 or 16, wherein the non-natural amino acids are independently selected from (S)-2-(4′-pentenyl)alanine (S5), (R)-2-(2′-propenyl)alanine (R3), and (R)-2-(7′-octenyl)alanine (R8).
21. The polypeptide construct of any one of aspects 15-20, wherein the non-natural amino acids have undergone reaction to form the intrapolypeptide covalent cross-link with each other.
22. The polypeptide construct of any one of aspects 15-21, wherein the interpolypeptide covalent linkage between the third polypeptide and the fourth polypeptide is a maleimide-thiol adduct.
23. The polypeptide construct of any one of aspects 15-22, wherein the second polypeptide and the fourth polypeptide are linked through an interpolypeptide covalent linkage.
24. The polypeptide construct of aspect 23, wherein the interpolypeptide linkage is between the C-terminal amino acid of the second polypeptide and the C-terminal amino acid of the fourth polypeptide.
25. The polypeptide construct of aspect 23 or 24, wherein the interpolypeptide covalent linkage between the second polypeptide and the fourth polypeptide is a maleimide-thiol adduct.
26. The polypeptide construct of any one of aspects 1-25, wherein the N-terminus or the C-terminus of the first, second, third, or fourth polypeptide is capped.
27. The polypeptide construct of aspect 26, wherein the N-terminus cap is acetyl or the C-terminus cap is —NH₂.
28. The polypeptide construct of any one of aspects 1-27, wherein the polypeptide construct binds to duplex DNA comprising the sequence of 5′-CANNTG-3′, wherein each N is independently any one of A, C, G, or T.
29. A polypeptide construct comprising:
(a) a first polypeptide comprising an amino acid sequence derived from a basic helix as listed in Table 2; and
(b) a second polypeptide comprising an amino acid sequence derived from a helix as listed in Table 2;
wherein the first polypeptide and the second polypeptide are linked through an interpolypeptide covalent linkage.
30. A polypeptide comprising the sequence of any one of the polypetides described herein, examples being:

(Ac-RAQILCKATEYIQS₅MRRS₅Nβ)₂K-NH₂
(Ac-RAQILCKATEYIQS₅MRRS₅N is SEQ ID NO: 338)
(Ac-RAQILCKATEYIQYMRRKNβ)2K-NH₂
(Ac-RAQILCKATEYIQYMRRKN is SEQ ID NO: 339)
(Ac-RAS₅ILCS₅ATEYIQYMRRKNβ)₂K-NH₂
(Ac-RAS₅ILCS₅ATEYIQYMRRKN is SEQ ID NO: 340)
(SEQ ID NO: 341)
Ac-HNALERKRRDHIKDSFHKLRDSVP
(SEQ ID NO: 342)
Ac-KRAHHNALERKRRDHIKDSFHK(GlyMal)LRDSVP-NH₂
(SEQ ID NO: 343)
Ac-KRAHHNALERKRRDHIKDSFHKLRDSVP
(SEQ ID NO: 344)
Ac-KRAHHNALERKRRDHIKDSFS₅K(GlyMal)LRS₅SVP-NH₂
(SEQ ID NO: 345)
Ac-KRAHHNALERKRRDHIKDSFS₅KLRS₅SVP
(SEQ ID NO: 346)
Ac-KRAHHNALERS₅RRDS₅IKDSFHK(GlyMal)LRDSVP-NH₂
(SEQ ID NO: 347)
Ac-KRAHHNALERS₅RRDS₅IKDSFHKLRDSVP
(SEQ ID NO: 348)
Ac-KRAHHNS₅LERS₅RRDHIKDSFHK(GlyMal)LRDSVP-NH₂
(SEQ ID NO: 349)
Ac-KRAHHNS₅LERS₅RRDHIKDSFHKLRDSVP
(SEQ ID NO: 350)
Ac-KRA_{ib}HHNALERS₅RRDS₅IKDSFHKLRDSVP
(SEQ ID NO: 351)
Ac-KRA_{ib}HHNS₅LERS₅RRDHIKDSFHKLRDSVP
(SEQ ID NO: 352)
Ac-KRS₅HHNS₅LER(D-lysine)RRDHIKDSFHKLRDSVP
(SEQ ID NO: 353)
Ac-KRS₅HHNS₅LERA_{ib}RRDHIKDSFHKLRDSVP
(SEQ ID NO: 354)
Ac-KRS₅HHNS₅LERKRRDHIKDSFHK(GlyMal)LRDSVP-NH₂
(SEQ ID NO: 355)
Ac-KRSHHNS₅LERKRRDHIKDSFHKLRDSVP
(SEQ ID NO: 356)
Ac-KVC(StBu)ILKKATAYILS₅VQAS₅K(GlyMal)-NH₂
(SEQ ID NO: 357)
Ac-KVC(StBu)ILKKATAYILSVQAEK(GlyMal)-NH₂
(SEQ ID NO: 358)
Ac-KVCILKKATAYILS₅VQAS₅K(N3)-NH₂
(SEQ ID NO: 359)
Ac-KVCILKKATAYILSVQAEK(N3)-NH₂
(SEQ ID NO: 360)
Ac-KVS₅ILC(StBu)S₅ATAYILSVQAEK(GlyMal)-NH₂
(SEQ ID NO: 361)
Ac-KVS₅ILCS₅ATAYILSVQAEK(N3)-NH₂
(SEQ ID NO: 362)
Ac-KVVILC(StBu)KATAYILS₅VQAS₅K(GlyMal)-NH₂
(SEQ ID NO: 363)
Ac-KVVILC(StBu)KATAYILSVQAEK(GlyMal)-NH₂
(SEQ ID NO: 364)
Ac-KVVILCKATAYILS₅VQAS₅K(N3)-NH₂
(SEQ ID NO: 365)
Ac-KVVILCKATAYILSVQAEK(N3)-NH₂
(SEQ ID NO: 366)
Ac-RAC(StBu)ILDKATEYIQS₅MRRS₅C-NH₂
(SEQ ID NO: 367)
Ac-RAC(StBu)ILDKATEYIQYMRRKC-NH₂
(SEQ ID NO: 368)
Ac-RACILDKATEYIQS₅MRRS₅C(StBu)-NH₂
(SEQ ID NO: 369)
Ac-RACILDKATEYIQYMRRKC(StBu)-NH₂
(SEQ ID NO: 370)
Ac-RAQILC(StBu)KATEYIQS₅MRRS₅C-NH₂
(SEQ ID NO: 371)
Ac-RAQILC(StBu)KATEYIQS₅MRRS₅NβC-NH₂
(SEQ ID NO: 372)
Ac-RAQILC(StBu)KATEYIQYMRRKC-NH₂
(SEQ ID NO: 373)
Ac-RAQILC(StBu)KATEYIQYMRRKNβC-NH₂
(SEQ ID NO: 374)
Ac-RAQILCKATEYIQS₅MRRS₅C(StBu)-NH₂
(SEQ ID NO: 375)
Ac-RAQILCKATEYIQYMRRKC(StBu)-NH₂
(SEQ ID NO: 376)
Ac-RAS₅ILC(StBu)SATEYIQYMRRKC-NH₂
(SEQ ID NO: 377)
Ac-RAS₅ILC(StBu)S₅ATEYIQYMRRKNβC-NH₂
(SEQ ID NO: 378)
Ac-RAS₅ILCS₅ATEYIQYMRRKC(StBu)-NH₂
(SEQ ID NO: 379)
Ac-SRA_{ib}QILCQATEYIQSNRRS₅N
(SEQ ID NO: 380)
Ac-SRAQILC(StBu)KATEYIQS₅N_{L}RRS₅NβC-NH₂
(SEQ ID NO: 381)
Ac-SRAQILCKATEYIQS₅N_{L}RRS₅N
(SEQ ID NO: 382)
Ac-SRAQILCKATEYIQYN_{L}R
(SEQ ID NO: 383)
Ac-SRAQILCKATEYIQYNRRKN
(SEQ ID NO: 384)
Ac-SRAQILCQATEYIQS₅N_{L}RRS₅N
(SEQ ID NO: 385)
Ac-SRAS₅ILC(StBu)S₅ATEYIQYN_{L}RRKNβC-NH₂
(SEQ ID NO: 386)
Ac-SRAS₅ILCS₅ATEYIQYNRRKN
(SEQ ID NO: 387)
Ac-WβADKRAHHNALERKRRDHIKDSFHK(GlyMal)LRDSV-NH₂
(SEQ ID NO: 388)
Ac-WβADKRAHHNALERKRRDHIKDSFHK(N3)LRDSV-NH₂
(SEQ ID NO: 389)
Ac-WβADKRAHHNALERKRRDHIKDSFHSLK(GlyMal)DSV-NH₂
(SEQ ID NO: 390)
Ac-WβADKRAHHNALERKRRDHIKDSFHSLK(N3)DSV-NH₂
(SEQ ID NO: 391)
Ac-WβADKRAHHNALERKRRDHIKDSFS₅K(GlyMal)LRS₅SV-NH₂
(SEQ ID NO: 392)
Ac-WβADKRAHHNALERKRRDHIKDSFS₅K(N3)LRS₅SV-NH₂
(SEQ ID NO: 393)
Ac-WβADKRAHHNALERKRRDHIKDSFS₅SLK(GlyMal)S₅SV-NH₂
(SEQ ID NO: 394)
Ac-WβADKRAHHNALERKRRDHIKDSFS₅SLK(N3)S₅SV-NH₂
(SEQ ID NO: 395)
Ac-WβADKRAHHNALERS₅RRDS₅IKDSFHK(GlyMal)LRDSV-NH₂
(SEQ ID NO: 396)
Ac-WβADKRAHHNALERS₅RRDS₅IKDSFHK(N3)LRDSV-NH₂
(SEQ ID NO: 397)
Ac-WβADKRAHHNALERS₅RRDS₅IKDSFHSLK(GlyMal)DSV-NH₂
(SEQ ID NO: 398)
Ac-WβADKRAHHNALERS₅RRDS₅IKDSFHSLK(N3)DSV-NH₂
(SEQ ID NO: 399)
Ac-WβADKRAHHNS₅LERS₅RRDHIKDSFHK(GlyMal)LRDSV-NH₂
(SEQ ID NO: 400)
Ac-WβADKRAHHNS₅LERS₅RRDHIKDSFHK(N3)LRDSV-NH₂
(SEQ ID NO: 401)
Ac-WβADKRAHHNS₅LERS₅RRDHIKDSFHSLK(GlyMal)DSV-NH₂
(SEQ ID NO: 402)
Ac-WβADKRAHHNS₅LERS₅RRDHIKDSFHSLK(N3)DSV-NH₂
(SEQ ID NO: 403)
Ac-WβADKRSHHNS₅LERKRRDHIKDSFHK(GlyMal)LRDSV-NH₂
(SEQ ID NO: 404)
Ac-WβADKRS₅HHNS₅LERKRRDHIKDSFHK(N3)LRDSV-NH₂
(SEQ ID NO: 405)
Ac-WβADKRSHHNS₅LERKRRDHIKDSFHSLK(GlyMal)DSV-NH₂
(SEQ ID NO: 406)
Ac-WβADKRS₅HHNS₅LERKRRDHIKDSFHSLK(N3)DSV-NH₂
(SEQ ID NO: 407)
Ac-WβKRAHHNALERKRRDHIKDSFHK(GlyMal)LRDSV-NH₂
(SEQ ID NO: 408)
Ac-WβKRAHHNALERKRRDHIKDSFS₅K(GlyMal)LRS₅SV-NH₂
(SEQ ID NO: 409)
Ac-WβKRAHHNALERS₅RRDS₅IKDSFHK(GlyMal)LRDSV-NH₂
(SEQ ID NO: 410)
Ac-WβKRAHHNS₅LERS₅RRDHIKDSFHK(GlyMal)LRDSV-NH₂
(SEQ ID NO: 411)
Ac-WβKRS₅HHNS₅LERKRRDHIKDSFHK(GlyMal)LRDSV-NH₂
(SEQ ID NO: 412)
Ac-WβNVKRRTHNS₅LERS₅RRNELKRSFFALK(GlyMal)DQI-NH₂
(SEQ ID NO: 413)
Ac-WβNVKRRTHNS₅LERS₅RRNELKRSFFALK(N3)DQI-NH₂
(SEQ ID NO: 414)
Ac-WβNVKRRTHNS₅LERS₅RRNELKRSFFK(GlyMal)LRDQI-NH₂
(SEQ ID NO: 415)
Ac-WβNVKRRTHNS₅LERS₅RRNELKRSFFK(N3)LRDQI-NH₂
(SEQ ID NO: 416)
Ac-WβNVKRRTHNVLERQRRNELKRSFFALK(GlyMal)DQI-NH₂
(SEQ ID NO: 417)
Ac-WβNVKRRTHNVLERQRRNELKRSFFALK(N3)DQI-NH₂
(SEQ ID NO: 418)
Ac-WβNVKRRTHNVLERQRRNELKRSFFK(GlyMal)LRDQI-NH₂
(SEQ ID NO: 419)
Ac-WβNVKRRTHNVLERQRRNELKRSFFK(N3)LRDQI-NH₂
(SEQ ID NO: 420)
Ac-WβNVKRRTHNVLERQRRNELKRSFS₅ALK(GlyMal)S₅QI-NH₂
(SEQ ID NO:421)
Ac-WβNVKRRTHNVLERQRRNELKRSFS₅ALK(N3)S₅QI-NH₂
(SEQ ID NO:422)
Ac-WβNVKRRTHNVLERQRRNELKRSFS₅K(GlyMal)LRS₅QI-NH₂
(SEQ ID NO: 423)
Ac-WβNVKRRTHNVLERQRRNELKRSFS₅K(N3)LRS₅QI-NH₂
(SEQ ID NO:424)
Ac-WβNVKRRTHNVLERS₅RRNS₅LKRSFFALK(GlyMal)DQI-NH₂
(SEQ ID NO: 425)
Ac-WβNVKRRTHNVLERS₅RRNS₅LKRSFFALK(N3)DQI-NH₂
(SEQ ID NO:426)
Ac-WβNVKRRTHNVLERS₅RRNS₅LKRSFFK(GlyMal)LRDQI-NH₂
(SEQ ID NO: 427)
Ac-WβNVKRRTHNVLERS₅RRNS₅LKRSFFK(N3)LRDQI-NH₂
(SEQ ID NO:428)
Ac-WβNVKRS₅THNS₅LERQRRNELKRSFFALK(GlyMal)DQI-NH₂
(SEQ ID NO: 429)
Ac-WβNVKRS₅THNS₅LERQRRNELKRSFFALK(N3)DQI-NH₂
(SEQ ID NO:430)
Ac-WβNVKRS₅THNS₅LERQRRNELKRSFFK(GlyMal)LRDQI-NH₂
(SEQ ID NO: 431)
Ac-WβNVKRS₅THNS₅LERQRRNELKRSFFK(N3)LRDQI-NH₂
(SEQ ID NO: 432)
Ac-KRAHHNALERKRRDHIKDSFHKLRDSVP-NH₂
(SEQ ID NO: 433)
Ac-SRAQILCKATEYIQYNRRKN-NH₂
(SEQ ID NO: 434)
Ac-PRFQSAADKRAHHNALERKRRDHIKDSFHKLRDSVP-NH₂
(SEQ ID NO: 435)
Ac-QSAADKRAHHNALERKRRDHIKDSFHKLRDSVP-NH₂
(SEQ ID NO: 436)
Ac-IEVESDADKRAHHNALERKRRDHIKDSFHKLRDSVP-NH₂
(SEQ ID NO: 437)
Ac-PRSSDTEENVKRAHHNALERKRRDHIKDSFHKLRDSVP-NH₂
(SEQ ID NO: 438)
Ac-TEENVKRAHHNALERKRRDHIKDSFHKLRDSVP-NH₂
(SEQ ID NO: 439)
Ac-KSKKNNSSKRAHHNALERKRRDHIKDSFHKLRDSVP-NH₂
(SEQ ID NO: 440)
Ac-KNNSSKRAHHNALERKRRDHIKDSFHKLRDSVP-NH₂
(SEQ ID NO:441)
Ac-PRFQSAADKRS₅HHNS₅LERKRRDHIKDSFHKLRDSVP-NH₂
(SEQ ID NO:442)
Ac-PRFQSAS₅DKRS₅HHNALERKRRDHIKDSFHKLRDSVP-NH₂
(SEQ ID NO: 443)
Ac-PRS₅FQSS₅DKRAHHNALERKRRDHIKDSFHKLRDSVP-NH₂
(SEQ ID NO: 433)
Ac-SRAQILCKATEYIQYNRRKN_{L}NH₂
(SEQ ID NO: 444)
Ac-SRAQILCKATEYIQYNRRKNHTHQQDIDDLK-NH₂
(SEQ ID NO: 445)
Ac-SRAQILCKATEYIQYNRRKNHTLISE-NH₂
(SEQ ID NO: 446)
Ac-SRAQILCKATEYIQYNRRKLHTHE-NH₂
(SEQ ID NO: 447)
Ac-SRA_{ib}pQILCQATEYIQS₅NLRRS₅LHTHE-NH₂
(SEQ ID NO: 432)
Ac-KRAHHNALERKRRDHIKDSFHKLRDSVP-NH₂
(SEQ ID NO: 448)
Ac-KRS₅HHNS₅LERKRRDHIKDSFHKLRDSVP-NH₂
(SEQ ID NO: 449)
Ac-KRSHANS₅LERKRLDHIKDSFHKLRDSVP-NH₂
(SEQ ID NO: 450)
Ac-KRS₅HANS₅LERKRTDHIKDSFHKLRDSVP-NH₂
(SEQ ID NO: 451)
Ac-KKSHSNS₅LARKRRDHIKDSFHKLRDSVP-NH₂
(SEQ ID NO: 452)
Ac-KRS₅HHNS₅LNRKRRDHIKDSFHKLRDSVP-NH₂

wherein
Ac is acetyl;
A_{ib} is 2-aminoisobutyric acid;
N_{L} is norleucine;
β prior to an amino acid represents that amino acid is a p amino acid;
GlyMal is glycylmaleimide;
StBu is tert-butylsulfenyl;
K(N3) is azidolysine; and
S₅ is (S)-2-(4′-pentenyl)alanine.
31. A pharmaceutical composition comprising a therapeutically effective amount of the polypeptide construct of any one of aspects 1-29 or the polypeptide of aspect 30 and a pharmaceutically acceptable excipient.
32. A method of treating disease in a subject in need thereof comprising administering to the subject a therapeutically effective amount of the polypeptide construct of any one of aspects 1-29, the polypeptide of aspect 30, or the pharmaceutical composition of aspect 31.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

The following examples further illustrate the disclosure but, of course, should not be construed as in any way limiting its scope.

### Example 1

This example demonstrates helical dimers, in accordance with aspects of the disclosure.

### Materials and Methods

### Cell Culture

HeLa cells were purchased from ATCC (Manassas, VA, USA). HCT116 cells were purchased from BPS Biosciences (San Diego, CA, USA). HeLa and P493-6 cells were cultured in RPMI-1640 with 10% FBS and 1% penicillin/streptomycin. HCT116 cells were cultured in McCoy's 5A medium with 10% FBS and 1% penicillin/streptomycin. All cell culture was performed under 37° C. with 5% CO₂.

### STR Synthesis and Purification

A Symphony X automated peptide synthesizer was used to prepare linear peptides on Rink amide MBHA resin. Fmoc-based solid phase chemistry, ring closing metathesis, and N-terminal modifications were carried out as previously described (Kim et al., Nature Protocols, 6: 761-771 (2011); Mitra et al., Nat. Commun., 8: 660 (2017); each of which is incorporated by reference herein). Lysine residues bearing monomethoxy trityl (Mmt) side chain protecting groups were incorporated at cross-linking positions of basic helices. On-resin Mmt deprotection was carried out for 5×2 min consecutive cycles of 1% TFA/DCM solution mixed by N2 bubbling. Deprotected lysine residues were functionalized with maleimide by 2 hr treatment with a 0.1 M solution of 2-(2,5-dioxo-2,5-dihydro-1H-pyrrol-1-yl)acetic acid (Mal-Gly-OH) (5 eq), HCTU (4.8 eq), and DIPEA (10 eq.) in DMF, with the exception STR69, which was connected with an aminobutyric acid maleimide interhelix linker. Crude peptides cleaved from resin were purified on a Waters preparatory HPLC system using an Xbridge Prep C18 5 μm OBN (19.5×150 mm) column; solvent A (0.1% TFA in H₂O); solvent B (MeOH); and a 10-min method with the following gradient (flowrate 30 mL/min): 35% B over 1 min; 35-85% B over 7 min; 95% B over 1 min; 35% B over 1 min. STR monomer ligation was performed in 50 mM sodium phosphate buffer pH 7.2+25% ACN as follows: a purified basic sequence bearing a maleimide (0.5 mL, 0.5 mM) and a purified zipper sequence with a free thiol (0.5 mL, 0.5 mM) were combined in a microcentrifuge tube and mixed by rotation for 2 hrs at room temperature. The reaction mixture was diluted into 3 mL of 50% ACN/H₂O+0.1% TFA and the ligated STR was purified using the same HPLC method as for individual monomers. STR purity and molecular weight were confirmed by LC-MS using an Agilent system equipped with a Phenomonex C18, 5 μm (5.0×50 mm) column; solvent A (95:5:0.1 H₂O/ACN/TFA) and solvent B (95:5:0.1 ACN/H₂O/TFA); 0.5 ml min flowrate, 0-2 min (0% B), 2-16 min (0-75% B), 16.5-18.5 min (100% B), 19 min (0% B). STR concentrations were quantified using 280 nm absorbance readings and compounds were stored as lyophilized powder or DMSO stocks.

### Electrophoretic Mobility Shift Assays (EMSAs)

For direct DNA binding experiments, STRs were serial diluted (3-fold increments) at 2× concentration in 20 μL of 1× binding buffer (20 mM HEPES pH 8.0, 150 mM NaCl, 5% glycerol, 1 mM EDTA, 2 mM MgCl₂, 0.5 mg/mL BSA, 1 mM DTT, 0.05% NP-40). 20 μL of 10 nM IRD700-labeled E-box probe in 1× binding buffer was added and samples were incubated for 30 min at RT followed by 15 min at 4° C. 3.5 μL of each reaction was loaded on a 6% acrylamide, 0.5× TBE gel equilibrated to 4° C. Higher affinity compounds (e.g., STR116 and STR118) were run at 10-fold lower concentration of compound and 0.5 nM final concentration of IRD-labeled oligo. Electrophoresis was carried out for 60 min at 110 V and 4° C. with 0.5× TBE+1 mM MgCl₂ running buffer. Gels were pre-run at 110 V for 60 min prior to sample loading. For specificity experiments, 25 nM STR116, 10 nM STR118, or 5 nM MAX, and 5 nM labeled E-box probe were incubated with 0, 5, 25, 125, or 625 nM unlabeled competitor oligo for 30 min at RT in binding buffer. For protein competition experiments, 1 nM TRD-Ebox oligo was added to a mixture of STR (0-1 mM) and 30 nM protein (MAX or MYC:MAX) and incubated for 30 minutes at room temperature in binding buffer. For DNA probe specificity experiments B-Z (100 nM), STR69 (1 μM), and STR640 (125 nM) and MAX (10 nM) were incubated with 1 nM IRD-labeled oligo and 0.01 mg/ml salmon sperm DNA for 30 minutes at RT in binding buffer. All samples were equilibrated to 4° C. for 15 minutes before loading onto 6% gel. Gels were imaged using an Odyssey Li-COR. ImageJ was used to quantify band intensity and fraction bound DNA was calculated by dividing band intensity of bound DNA by the band intensity of the free DNA from a vehicle treated lane. A four-parameter dose-response curve fit to a plot of normalized fraction bound DNA vs. log B-Z concentration yields an IC50 which was reported as the apparent K_{D}. The sequences of STR116, STR118, STR69, and STR640 are shown in Table 4.

TABLE 4

| | | SEQ ID |
|---|---|---|
| | | NO: |
| STR116 | Ac-PRFQSA(S5)DKR(S5)HHNALERKRRD | 453 |
| | HIKDSFHK(GlyMal)LRDSVP-NH₂ | |
| | Ac-SR(Aib)QILCQATEYIQ(S5)(Nle)RR | 454 |
| | (S5)LHTHE-NH₂ | |
| STR118 | Ac-PRFQSA(S5)DKR(S5)HHNALERKRRDHI | 453 |
| | KDSFHK(GlyMal)LRDSVP-NH₂ | |
| | Ac-SRAQILCKATEYIQYLR(S5) | 455 |
| | KIH(S5)LE-NH₂ | |
| STR69 | Ac-LRLKINSRERKRMHDLNIAMDK(ButMal) | 456 |
| | LREVMP-NH₂ | |
| | Ac-SKIATLCLARNYILMLTNSL-NH₂ | 457 |
| STR640 | Ac-RREIANSNERRRMQSINAGFQK(GlyMal) | 458 |
| | LKTLIP-NH₂ | |
| | Ac-SKAAILCQTAEYIFRLRRKIHTLE-NH₂ | 459 |

### MYC and MAX Protein Expression and Purification

Human MYC (residues 356-434) and MAX (residues 22-102) proteins was expressed with a N-terminal hexahistidine tag in Escherichia coli strain BL21(DE3) using a pET28c vector. Transformed bacteria was grown at 37° C. and induced with 0.5 mM isopropyl-β-D-thiogalactoside (IPTG) at an A₆₀₀=0.8. Cells were pelleted 14 h after induction and lysed in lysis buffer (100 mM NaH₂PO₄, 10 mM Tris, 300 mM NaCl, 8 M urea, 10 mM imidazole, pH 8.0) with Complete EDTA-free Protease Inhibitor (Roche) by sonication. The lysate was centrifuged to clear insoluble matter before loading onto Ni-NTA resin (Qiagen). After being washed once with lysis buffer and three times with wash buffer (50 mM NaH₂PO₄, 300 mM NaCl, 8 M urea, 20 mM imidazole, pH 8.0), column-bound protein was eluted using elution buffer (50 mM NaH₂PO₄, 300 mM NaCl, 8 M urea, 250 mM imidazole, pH 8.0), dialyzed into desired buffer and further concentrated by centrifugation using a 3-kDa exclusion filter. The protein concentration was determined using A₂₈₀ measurements and MAX protein was mixed with MYC at 1:1 ratio or used in homodimeric form.

### Circular Dichroism Spectroscopy

Lyophilized STR samples were resuspended in 20 mM phosphate buffer pH 7.4 and diluted to 10 μM. Circular dichroism spectra were obtained on a Jasco J-170 using a 0.1 cm quartz cuvette with the following settings: wavelength, 260-180 nm; data pitch, 1.0 nm; scan rate, 50 nm min⁻¹; accumulations, 3; temperature, 25-85° C. with 6° C. increments. Means-Movement smoothing at the lowest setting was applied to the recorded data.

### In Vitro Trypsin Stability Assay

Each STR, 30 μM, was dissolved in 330 μL of 20 mM phosphate buffer pH 7.4 in a microcentrifuge tube and heated to 37° C. on a tabletop shaker (500 rpm). 30 μL of the reaction mixture was added to 60 uL of quenching solution (ACN+3% formic acid with 500 nM fmoc-lysine-OH internal standard) for 0 s timepoint sample. Thermo-Pierce MS-Grade Trypsin was added to a final concentration of 0.5 μg/ml and additional 30 μL aliquots were quenched at indicated timepoints. Quenched samples were cooled to 4° C. and centrifuged at 20,000×G for 3 min. Sample injections were analyzed by LC-MS using an Agilent system equipped with a Phenomonex C18 5 μm (5.0×50 mm) column; solvent A (95:5:0.1 H₂O/ACN/TFA) and solvent B (95:5:0.1 ACN/H₂O/TFA); 0.5 ml min⁻¹ flowrate, 0-2 min (5% B), 2-8.8 min (5-95% B), 9-11 min (95% B), 11.1 min (5% B). Intact STR was calculated by normalizing the background subtracted integrated area under the curve for the EIC of (M+4H)/4, ±0.5 mass units, where M is mass and H is hydrogen ion, to the integrated area under the curve for the A₂₈₀ peak of the internal standard. Fraction intact STR was calculated by dividing the intact STR by the normalized STR signal in the initial 0 s sample. GraphPad Prism was used to plot fraction intact STR vs time, and the proteolytic half-life was derived using a non-linear one-phase decay with the plateau constant set equal to zero.

### Conditioned Media Binding Assay.

HeLa cells were grown to 90% confluency in a 6 cm plate and the media was collected. 10 μM STR was resuspended in 0.5 mL of the conditioned media and incubated with gentle shaking at 37° C. At 0, 24 and 48 hrs., treatment media (2 μL) was diluted into 48 μL of 1× EMSA binding buffer containing 5 nM E-box probe. DNA binding was measured using an electrophoretic mobility shift assay. Fraction bound to E-box probe was calculated by dividing the band intensity of bound DNA by the sum of the bound DNA+free DNA.

### Cellular Viability Assays.

Approximately 5,000 HeLa cells were seeded in a 96-well plate. The following day an equal volume of media containing 2× compound or DMSO vehicle were added to experimental wells and the plate was incubated for the indicated time of experiment. A 2× volume of lysis buffer was added to additional wells 45 minutes prior to the final time-point and LDH activity in treatment medium was measured using the Pierce LDH Cytotoxicity Assay Kit according to manufacturer protocol (Thermo Scientific #88953), or cell viability was measured by using the CellTiter-Glo Cell Viability Assay (Promega #G9241). Approximately 1,000 P493-6 cells were seeded into a 96-well plate and an equal volume of 2× compound, DMSO vehicle in media, or 0.2 mg/ml tetracycline was added. Media was exchanged and cells were retreated at indicated timepoints. Cell viability was measured by using the CellTiter-Glo Cell Viability Assay (Promega #G9241) at the indicated timepoints. P493-6 cells were cultured for 72 Hrs with 0.1 mg/mL tetracycline to prepare ‘MYC-OFF’ phenotype.

### Fluorescence Microscopy and Quantitative Analysis.

HeLa cells were seeded in the 12-well chamber slide with 2500 cells per well (Ibidi, #81201). Cells at 70-80% confluency were treated either with DMSO as the negative control or 5 μM FITC-labeled STR for indicated duration. Cells were washed with phosphate buffer saline (PBS) for five consecutive times, fixed by 4% formaldehyde in PBS at room temperature for 10 mins, and then washed twice with PBS. The rubber frame was then removed and slide was dried in the dark at room temperature, covered with cover glass (Fisher, #12-545 M), mounted with 5 mL In Situ Mounting Medium with DAPI (Sigma, DUO82040), and sealed with nail polish. Leica Stellaris 8 Laser Scanning Confocal with an HC PL APO CS2 40× oil objective was used to image a single focal plane to accurately detect the DAPI and FITC signal location using HyD detectors. Identical microscope acquisition parameters were set and used within experiments. Post-acquisition processing was performed using ImageJ software (NIH). The workflow was as follows: open all channels for each field of view; designate a color for each channel; adjust brightness/contrast for all channels (applying the same levels for all conditions within and between experiments to allow for direct comparison); merge the channels together; adjust the image unit from pixel to micrometer; export the processed TIFF files for quantification. For quantitative analysis, nuclear boundaries were identified manually using the DAPI image. Then the “ROI Manager” tool in ImageJ was exploited to add all the cell outlines as a collection and overlay with the FITC channel to measure per-cell nuclear fluorescence intensity. Typical quantitative comparisons were made using data from three or more independent fields of view per independent biological replicate condition.

### PAGE Gel Analysis of STR Uptake.

Approximately 0.1×10⁶ HeLa cells were seeded in each well of a 12-well plate. Cells were treated for 12 hours with 1 μM B-Z-FITC, 1 μM STR116-FITC, or 1 μM STR118-FITC. After the indicated treatment time, media was aspirated, cells were washed with PBS (2×1 mL) and treated with 0.25% trypsin (0.25 mL) for 5 min at 37° C. The trypsin was quenched with the addition of 1 mL of media and the detached cells were transferred to a microcentrifuge tube and centrifuged at 500× g for 4 min. The media was aspirated, 20 μL of RIPA buffer (50 mM Tris, pH 7.4, 150 mM NaCl, 0.25% deoxycholate, 1% NP-40, 1 mM EDTA)+Complete EDTA-free Protease Inhibitor (Roche) was added and cells were incubated in RIPA buffer for 10 min on ice. After lysis, 6.6 μL of 4× SDS loading buffer was added, samples were heated to 95° C. for 10 minutes, cooled to RT and analyzed by SDS-PAGE using a tris-glycine buffer system with an 18% acrylamide gel.

### Western Blot.

P493-6 cells were treated with DMSO, 0.1 ug/mL tetracycline, 10 mM STR116, or 10 mM STR118 for 48 Hours. Harvested cells were lysed in RIPA buffer and protein concentration was determined using the Pierce BCA Protein Assay Kit (Thermo Scientific, Cat. no. 23225). Samples were loaded at equal protein concentration, separated by SDS-PAGE, and transferred to nitrocellulose membranes (Amersham, Cat. no. 10600001). Membranes were incubated with rabbit anti-C-Myc (1:1000, Cat. no. 18583, Cell Signaling Technology), mouse anti-CCNB1 (1:1000, Cat. no. 4135, Cell Signaling Technology), rabbit anti-β-Actin (1:4000, Cat. no. 4970, Cell Signaling Technology) and rabbit anti-LDHA (1:4000, Cat. no. 3582, Cell Signaling Technology). After washing, membranes were stained with IRDye-conjugated secondary antibodies (IRDye 680LT Goat anti-mouse 1:10000, Cat. no. 926-68020, and IRDye 800CW Donkey anti-Rabbit 1:10000, Cat. no. 926-32213, Licor) and blots were visualized by LI-COR.

### Luciferase Assay.

Myc Reporter (Luc)—HCT116 cells were purchased from BPS Biosciences, Inc., San Diego (Cat. #: 60520). The assay was performed using the manufacturer procedure. 25,000 cells/well were seeded into a 96-well plate. The following day, the media was removed, and cells were treated in triplicate with the indicated treatment using Assay Medium 7B: Opti-MEM (Life Technologies #31985-062)+0.5% FBS+1% non-essential amino acids+1 mM sodium pyruvate+1% penicillin/streptomycin and a final concentration of 0.5% DMSO. Treated cells were incubated for 24 hours and luciferase activity was measured using the ONE-Step™ Luciferase Assay System (BPS Cat. #60690) and percent luciferase activity was calculated as directed in the manufacturer protocol.

### ChIP-qPCR.

MYC ChIP: HeLa cells were seeded in 200-mm dishes. After reaching 70% confluence, cells were crosslinked with 1% formaldehyde, fragmented by sonication, and incubated with c-Myc antibody (N-262, scbt) or IgG (ab171870, abcam) overnight. The mixture was then immunoprecipitated with protein A beads (Genescript, pre-treated with 1% BSA for 1 hour) for 1 hour. Immunoprecipitated complexes were successively washed with Low Salt Wash Buffer I (0.1% SDS, 1% Triton X-100, 2 mM EDTA, 20 mM Tris-HCl, 150 mM NaCl, pH 8.0), High Salt Wash Buffer (0.1% SDS, 1% Triton X-100, 2 mM EDTA, 20 mM Tris-HCl, 500 mM NaCl, pH 8.0), and LiCl Wash Buffer (250 mM LiCl, 1% NP-40, 1% Sodium Deoxycholate, 1 mM EDTA, 10 mM Tris-HCl, pH 8.0). All washes were performed at RT for 8 min on a rotator. The complexes were eluted with 1% SDS at 30° C. for 15 min, and then incubated at 65° C. overnight to reverse crosslink protein-DNA complexes. After decrosslinking, DNA was purified using QIAQuick PCR Purification Kit (Qiagen) according to the manufacturer's instructions.

### Photo-ChIP-qPCR.

P493-6 cells (1.5×10⁷) were treated with 10 mM P-BioSTR118 and incubated at 37° C. for 24 hours. Treatment media was aspirated to remove extracellular photo probe and cells were resuspend in 15 mL of RPMI, transferred to a 15 cm plate and irradiated over ice for 10 min (365 nm, Spectrolinker XL-1500a, Spectroline). After irradiation, media was removed, and cells were washed with 10 mL cold PBS. DNA was fragmented by sonication and an aliquot of input DNA was reserved. The remaining sample (˜900 uL) was diluted into binding and washing buffer (10 mM Tris-HCl pH 7.5, 1 mM EDTA, 2 M NaCl [2×]) and equally divided between Dynabeads MyOne Streptavidin C1 (ThermoFisher Scientific, Cat. No. 65001) that were prepared with or without biotin blocking (200 mM biotin in binding and washing buffer, 2×10 min pretreatment). Biotinylated DNA enrichment was performed for 30 minutes at RT by rotation and samples were washed with binding and washing buffer (4×4 min.). Biotinylated DNA was dissociated from beads by adding 100 mL of 0.1% SDS and heating for 7 min at 95 C. Eluted and input DNA was purified using QIAQuick PCR Purification Kit (Qiagen) according to the manufacturer's instructions.

qPCR

ChIP DNA from both experiments was quantified in triplicate using quantitative PCR on a LightCycler 480 (Roche). The sequences of the qPCR primers are listed in Table 5.

TABLE 5

| Primer | Sequence | SEQ ID NO: |
|---|---|---|
| intergenic | TTTTCTCACATTGCCCCTGT | 460 |
| region | | |
| forward | | |
| intergenic | TCAATGCTGTACCAGGCAAA | 461 |
| region | | |
| reverse | | |
| SNTG2 forward | GCCGCACTGGAATTTATCC | 462 |
| SNTG2 reverse | AGGAGCCTCACAGATGCAGT | 463 |
| RPL37 forward | TGACTGCTAACGTGCGAAAC | 464 |
| RPL37 reverse | GTCAAGAGGAGGATGCGGTA | 465 |
| TORIA forward | GAGTTTCCGGAAGCAAAACA | 466 |
| TORIA reverse | GCGGAGGCCATCTTTCTT | 467 |
| MRPS15 forward | TAAACGTGGGCACACAACC | 468 |
| MRPS15 reverse | TAGGTGGCGTGACTCTGATG | 469 |
| FBXW8 forward | GTGATAGGCAGCAGAGCTGA | 470 |
| FBXW8 reverse | TGTACGCACGTGGTGGTC | 471 |
| CASP8 forward | ACCCTGCAGTTCCTTCTGTG | 472 |
| CASP8 reverse | GAAAACACTTCCCTCCAGCA | 473 |

Enrichment plots for each target gene show percent input relative to non-enriched input DNA.

### X—Ray Crystallography and Structural Refinement

Purified peptide B-Z was dissolved in 50 mM HEPES pH 6.0, 200 mM NaCl and 10 mM MgCl₂ to yield a 200 μM solution. To this solution, 16-mer oligonucleotides (2.5 mM) containing E-box site in duplex buffer (100 mM potassium acetate, 30 mM HEPES, pH 7.5, Integrated DNA technologies, Lot #11-05-01-12) were added to make the final concentration of oligonucleotides 100 μM. Co-crystals were generated using hanging drop vapor diffusion where 1 μL of complex solution was mixed with 1 μL well solution. Clear rectangular crystals emerged in 50 mM Tris pH 7.0, 30% 2-Methyl-2,4-pentanediol, 50 mM NaCl and 10 mM MgCl₂. Diffraction data was collected at the Advanced Photon Source, Argonne National Laboratories, Argonne, Illinois, at the SBC 19-BM beamline (0.97 Å). Data were indexed, scaled and merged using HKL-300016 (Minor et al., Acta Crystallogr D Biol Crystallogr, 62: 859-866 (2006), incorporated by reference herein). Molecular replacement was performed in Phenix using PDB: 1HLO as the search model with ligand removed (Adams et al., Acta Crystallogr D Biol Crystallogr, 66: 213-221 (2010), incorporated by reference herein). The model was refined using iterative rounds of Phenix Refine and manual inspection with Coot (Emsley et al., Acta Crystallogr D Biol Crystallogr, 66: 486-501 (2010), incorporated by reference herein). Ligand constraints for interhelix were generated using Elbow. Final deposited structure will be released for open access in the Protein Data Bank (Accession: 7RCU). All x-ray crystal structure images were generated using Pymol. Data collection and refinement statistics are reported in Table 6.

TABLE 6
Data collection

| | |
|---|---|
| Wavelength (Å) | 1.0000 |
| Resolution range (Å) | 46.266-2.69037 |
| Space group | P1 |
| a, b, c (Å) | 25.666, 46.266, 166.017 |
| α, β, γ (º) | 90.052, 90.048, 90.021 |
| Number of Reflections (all/unique) | 18350/10194 |
| I/σ | 1.7 |
| Redundancy | 1.8 |
| Completeness (%) | 86.36 |

Refinement

| | |
|---|---|
| R-work | 0.235 |
| R-free | 0.276 |
| Number of non-hydrogen atoms | 5840 |
| macromolecules | 5718 |
| ligands | 74 |
| water | 48 |
| RMS(bonds) | 0.012 |
| RMS(angles) | 1.31 |
| Ramachandran favored (%) | 93.37 |
| Ramachandran allowed (%) | 6.63 |
| Ramachandran outliers (%) | 0 |
| Clashscore | 8 |
| Average B-factor | 54.0 |
| macromolecules | 47.0 |
| ligands | 56.3 |
| waters | 43.3 |

### Results

### Design and Synthesis of MAX-Derived Synthetic Transcriptional Repressor Mimetics.

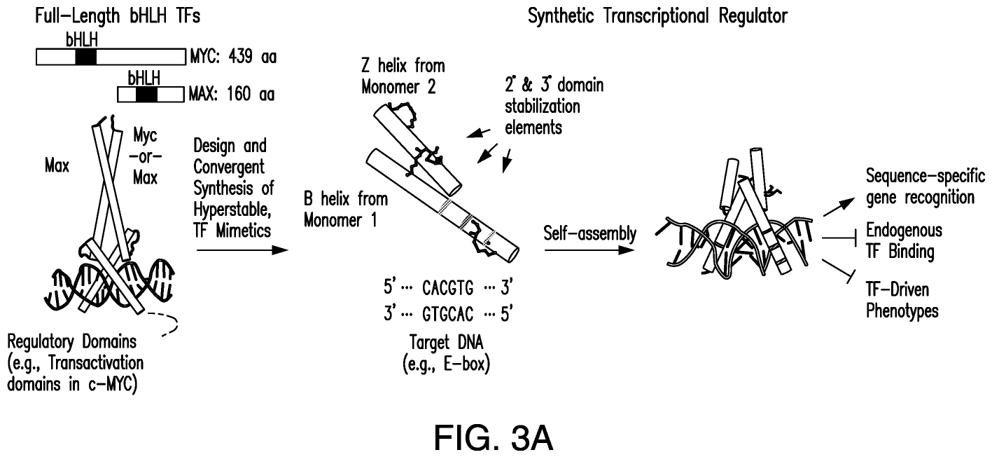

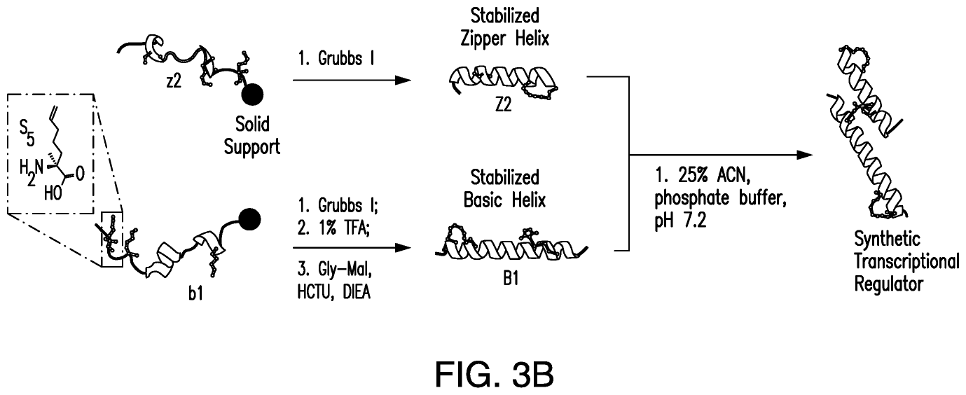

The core DNA-binding domain of basic helix-loop-helix (bHLH) TFs such as MYC and MAX, contain a leucine zipper helix connected to a basic DNA-binding helix through a flexible loop (FIG. 3A). Protein homo- or heterodimerization through the leucine zipper helices results in the formation of a stable tetrahelix core that orients two DNA-binding α-helices for interaction with the major groove of DNA (FIG. 3A). This domain architecture is conserved across hundreds of bHLH TFs and is similar for other families such as the bZIP TFs. Given the conserved and modular structure of this DBD, it was reasoned that a non-natural mimetic comprised of the minimal DNA-binding helix as well as the N-terminal portion of the leucine zipper in a bHLH protein such as MAX would be sufficient for potent and specific DNA binding. A linear peptide containing these elements, such as engineered miniproteins, would be >60 amino acids, and would therefore be synthetically challenging and likely suffer from pharmacologic limitations. Instead, a hybrid and convergent synthetic approach was considered, where shorter peptides encompassing the basic (B) and minimal leucine zipper (Z) helices are synthesized and then ligated to build the larger tertiary structure, which was hypothesized would be capable of dimerizing to form the minimal, tetrahelical bHLH structure (FIG. 3A). This approach would also permit inclusion of non-natural amino acids to augment structural stabilization. Since MAX can form DNA-binding homodimers, it was hypothesized that a non-natural approach, where ‘B’ and ‘Z’ helices from opposing monomers are connected at specific sites not involved in binding, could result in a ‘cross-dimer’ bHLH mimic that would self-associate in a sandwich fashion to recreate the tetrahelix core (FIG. 3A). Functionally, it was also hypothesized that the cross-dimer mimetic could act as a dominant-negative by blocking endogenous protein binding to DNA targets but would not interfere with protein-protein interactions within the extended MYC/MAX interaction network.

A model basic-zipper, cross-dimer STR derived from MAX was synthesized, and specific DNA binding was quantified using electrophoretic mobility shift assays (EMSA, or gel-shift) with either a consensus E-box oligonucleotide (targeted by MYC and MAX) or a control oligonucleotide containing the unrelated AP1 consensus binding site. It was found that a basic-zipper helix hybrid (B-Z) potently bound E-box containing DNA with an apparent K_{D} of 16 nM and showed no stable binding to the control AP1 oligonucleotide. By contrast, it was found that neither the complete B-helix, nor a suitable hydrocarbon ‘stapled’ version (B1) showed any appreciable binding to E-box DNA. This observation runs contrary to reports in the literature that unmodified or ‘stapled’ basic domain helices alone can specifically bind DNA, at least for MAX.

Based on these results, a modular route was devised to synthesize STRs that contained both secondary and tertiary domain stabilization elements. Among the possible strategies to achieve this, a route was elected in which zipper and basic helix peptides were synthesized containing bis-alkylated, terminal olefin containing ‘S₅’ amino acids for side-chain ‘stapling’ by ring-closing metathesis (FIG. 3B). Each helix also harbored an orthogonal ligation synthon, which in this case included a thiol on the Z helix and an orthogonally protected, C-terminal lysine on the B helix that permitted installation of a maleimide after helix stapling. Synthesis and modification of each stabilized helix followed by inter-helix ligation in aqueous solution proved to be a general and high-yielding route to produce stabilized STRs of approximately 6 kDa.

### Optimized MAX-STRs Specifically Bind E-Box DNA and Inhibit MYC/MAX Binding.

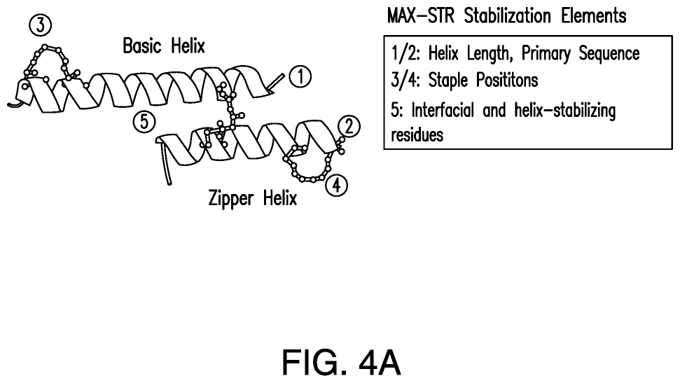

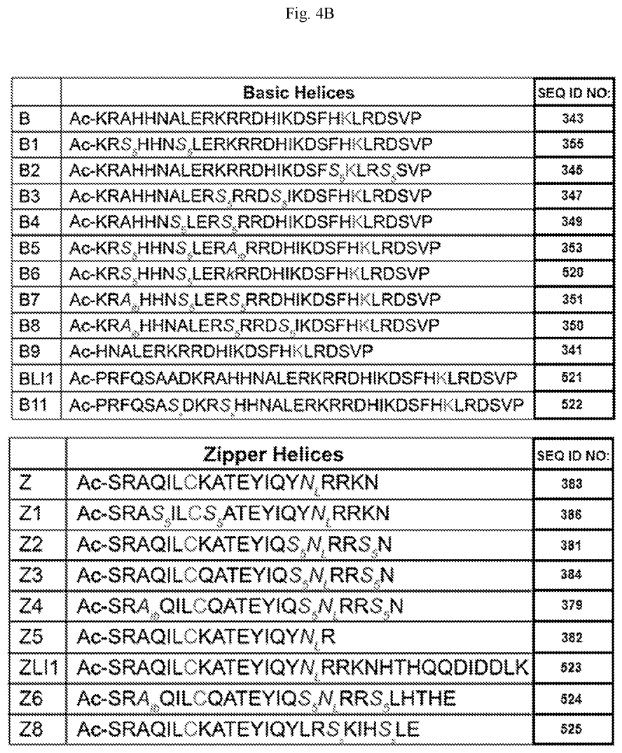

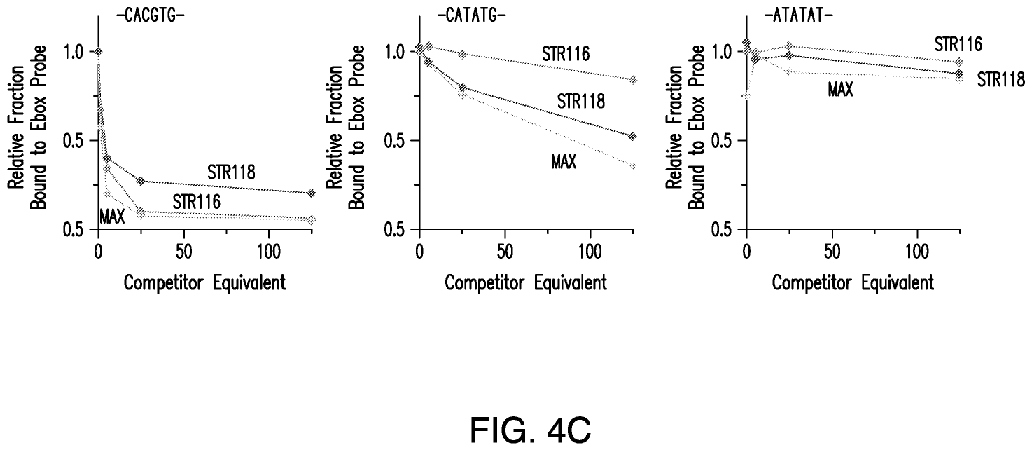

To identify MAX-STR structure-function relationships, a focused medicinal chemistry campaign was undertaken around the ‘B-Z’ progenitor. Two parallel libraries of basic and zipper helices with promising stapling positions, peptide lengths and modifications for stability (e.g., structural and metabolic; FIGS. 4A and 4B) were synthesized. Each individual modified zipper or basic helix peptide in the library was ligated with the corresponding unstapled helix partner (B or Z alone) for controlled comparison of individual structural changes and corresponding changes in DNA-binding. Within the basic helix library, it was found that truncation of even a few N-terminal residues (helix B9) abrogated DNA-binding, whereas N-terminal extension modestly improved affinity (FIG. 4B). The location of i→i+4 staples on the back face of the basic helix significantly impacted binding affinity. The most N- and C-terminal staple positions largely maintained tight binding affinity, and central staples reduced affinity by 10- to 20-fold. Within the Z helix, truncation of the C-terminal residues, or introduction of the side chain staples around the inter-helix ligation site significantly reduced affinity, whereas peptides with C-terminal extension or introduction of staples at other positions largely retained tight binding (FIG. 4B).

Using these SAR determinants as a guide, a library of MAX-STRs was synthesized that contained stabilizing modifications in both helices, and several lead compounds for further study were identified. These included B1-Z2, which was based on and had a similar binding affinity to the original unmodified B-Z scaffold. Two additional leads, STR116 and STR118, encompassed changes predicted to preserve or improve binding and augment metabolic stability, as discussed below. STR118 exhibited very high affinity for E-box DNA (K_{D}=3 nM), which is on par with measurements and reported affinities for full-length MYC/MAX and MAX/MAX. Competitive EMSA experiments demonstrated that STR116, STR118 and MAX protein binding could be effectively competed with excess unlabeled E-box oligonucleotide (FIG. 4C). DNA in which the E-box had been replaced entirely exhibited no competition with either STR or MAX protein. Competitor DNA containing a more subtle mutation of the two central nucleotides in the E-box site (CG to TA) demonstrated some competition with MAX protein and each STR at high concentrations. Collectively, these data confirmed that optimized STRs can bind E-box DNA with potency and specificity equivalent to, or even beyond, natural TF DNA-binding domains.

TABLE 7
K_{d} app, nM

| | |
|---|---|
| B1-Z | 18 |
| B2-Z | 19 |
| B3-Z | 77 |
| B4-Z | 239 |
| B7-Z | 169 |
| B8-Z | 66 |
| B9-Z | — |
| BLI1-Z | 8.3 |
| B-Z1 | 58 |
| B-Z2 | 22 |
| B-Z3 | 18 |
| B-Z4 | 20 |
| B-Z5 | 98 |
| B-ZLI1 | 20 |

TABLE 8
K_{d} app, (95% C.I.), nM

| | |
|---|---|
| B-Z | 16, (13-19) |
| BLI1-ZL1 | 25, (14-35) |
| B1-Z2 | 26, (22-29) |
| B1-Z4 | 40, (37-43) |
| B5-Z4 | 111, (98-126) |
| B6-Z4 | 57, (49-66) |
| B11-Z4 | 18, (16-20) |
| STR116 | 14, (13-16) |
| STR118 | 3.5, (3.0-4.0) |

Next tested was whether STRs were capable of competing with DNA binding by recombinant MAX/MAX and MYC/MAX. Competition EMSAs at saturating concentrations of MAX showed minimal competition by the unmodified B-Z molecule, nor the stabilized progenitor B1-Z2. STR118 caused dose-dependent inhibition of both MAX/MAX and MYC/MAX bound E-box DNA complexes accompanied by the formation of the stable STR-E-box DNA complex with IC50 values of 61 nM and 170 nM, respectively. In line with its lower affinity, STR116 inhibited MAX/MAX and MYC/MAX DNA binding with IC50 values of 400 nM and 1.0 μM, respectively. STR116 and B-Z have similar equilibrium dissociation constants, suggesting that kinetic factors may play a role in effective competition for DNA binding. These data confirm that lead STRs can directly inhibit MYC/MAX and MAX/MAX DNA binding through the formation of a dominant-negative STR-DNA complex.

### Secondary and Tertiary Domain Preorganization Promotes Thermal and Proteolytic Stability.

Beyond promoting potent and specific DNA binding, synthetic stabilization of secondary and tertiary elements should augment the structural, and therefore pharmacologic, stability of STRs. This effect has been demonstrated with diverse side chain macrocyclization of α-helical, loop and β-sheet peptides, resulting in molecules with demonstrated activity in cells, animal models and more recently in humans. Circular dichroism (CD) spectroscopy of the non-stapled B-Z progenitor confirmed that it is largely unstructured in solution. By contrast, spectra of all STRs showed strong absorbance minima at 208 and 222 nm, consistent with a predominantly α-helical structure. Temperature-dependent CD of STR118 confirmed that the structure templated by inter- and intramolecular stabilizing elements is highly thermally stable, with retention of helicity even at 85° C. Indeed, an aliquot of STR118 heated to 95° C. and then cooled showed identical binding activity compared to an aliquot kept at room temperature, reinforcing the hybrid properties of these fully synthetic ‘biologic’ structures.

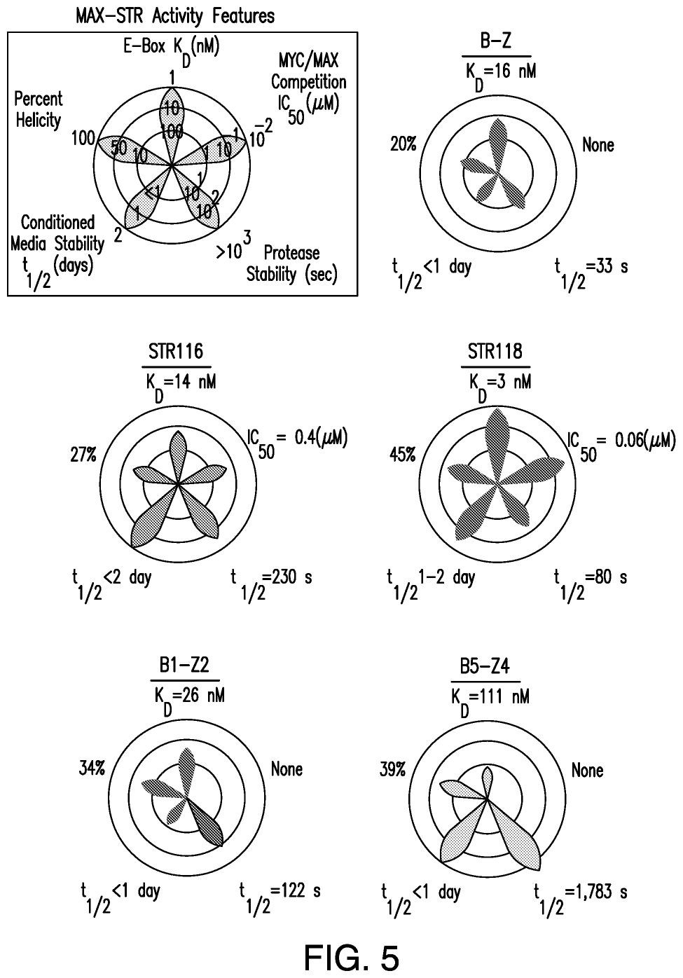

To ascertain how this structural stability impacts biological activity, a series of experiments were performed aimed at quantifying the chemical and proteolytic sensitivity of wild-type and stabilized MAX-STRs. To start, a kinetic trypsin stability assay was employed which can be very active against arginine- and lysine-rich DNA binding domains. The full-length MAX bHLH domain, which is structurally analogous to Omomyc and related polypeptide mimics of natural bHLH domains, was immediately degraded and exhibited a half-life of 20 seconds in this assay. Likewise, the wild-type B-Z molecule was rapidly proteolyzed at several positions, as measured by LC-MS. Introduction of hydrocarbon staples clearly protected internal and adjacent cleavage sites in stabilized molecules like B1-Z2. Targeted helix-stabilizing substitutions, such as aminoisobutyric acid (A_{ib}) or synonymous mutations not recognized by trypsin were introduced near those sites to further reduced proteolytic sensitivity, but typically led to losses in binding affinity (e.g., B5-Z4). Both STR116 and -118 embodied a combination of stabilizing modifications balanced with retained or improved binding affinity and exhibited significantly increased half-lives (>10-fold) relative to natural bHLH protein structures like MAX. To complement trypsin stability assays, MAX-STRs were incubated in conditioned media and assessed their functional integrity (e.g., retained capacity for DNA binding) over time by EMSA. The unmodified (B-Z) and stabilized (B1-Z2) showed >50% loss of activity within the first day. Consistent with higher stability in CD and trypsin assays, both STR118 and STR116 exhibited increased stability in conditioned media. Together, these data confirm that vigilant introduction of local and global stabilizing modifications can produce STRs with potent DNA-binding activity, hyperstable structures and improved pharmacologic features such as protease resistance (FIG. 5).

### Optimized MAX-STRs Penetrate and Distribute Throughout Cells Intact.

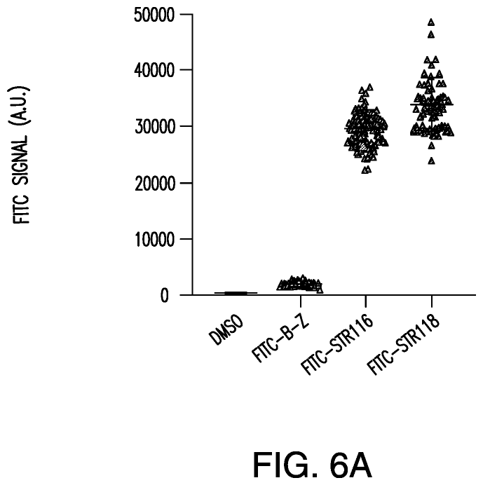

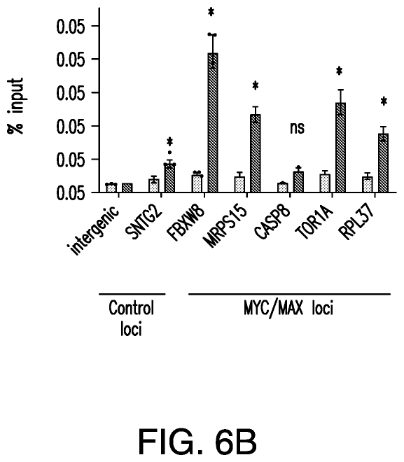

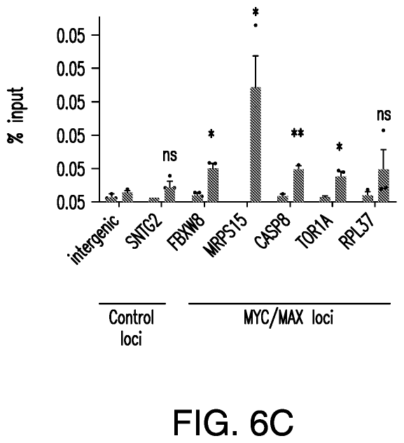

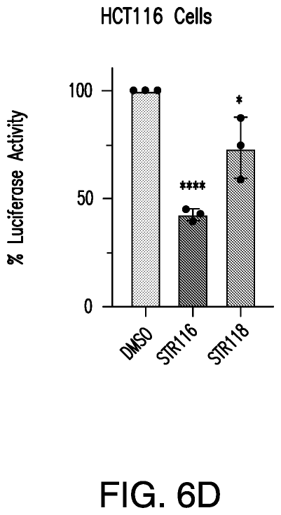

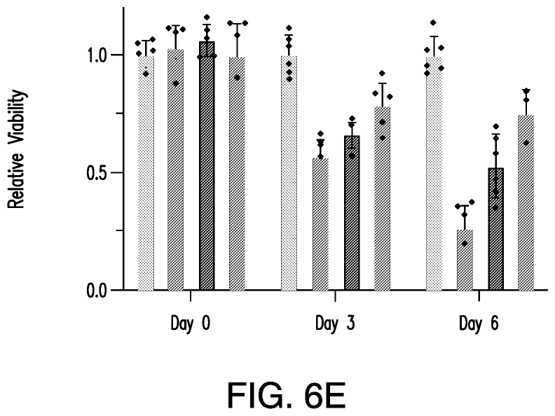

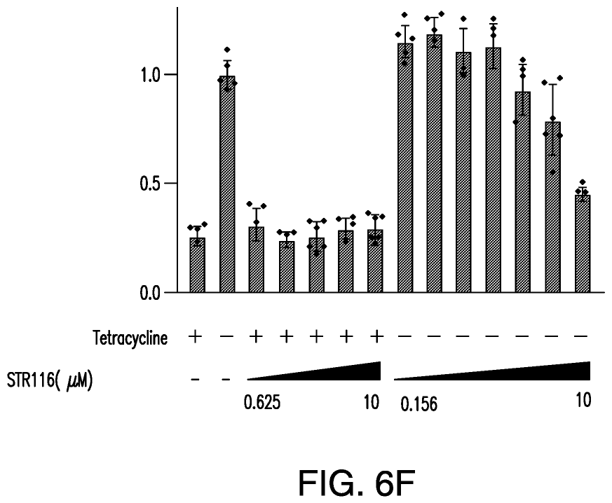

Stabilized peptides and cell-penetrating proteins interact with and enter cells via different mechanisms compared to many cell-permeable small molecules. Attributes such as secondary structure, charge, hydrophobicity, solubility and proteolytic stability have been shown to be important for productive cellular uptake and sub-cellular distribution for different classes of stabilized peptides. To determine whether STRs were capable of penetrating cells, first synthesized were fluorescein isothiocyanate (FITC) labeled versions of lead compounds, and imaged cells incubated with each molecule by confocal fluorescence microscopy. Cellular uptake of all molecules was observed, however the intensity and sub-cellular localization showed distinct patterns. The unmodified B-Z compound demonstrated weak uptake and punctate distribution relative to its stabilized counterparts STR116 and STR118. STR118 and -116 were present in cells at much higher levels and showed significant distribution in the cytosol and nucleus of cells (FIG. 6A). Significant changes were not observed in cellular morphology, membrane integrity or viability under these assay conditions.

To confirm that intact MAX-STRs are present inside of cells—and at what relative concentrations—the intracellular contents of cells were extracted after treatment with each compound for visualization of the intact molecules by gel electrophoresis. Full-length FITC-STR116 and FITC-STR118 were observed in cells after 12 hrs at much higher concentrations relative to the unstructured B-Z progenitor. These combined data confirmed that MAX-STRs can penetrate cells and access intracellular compartments intact, and that differences in uptake and sub-cellular distribution likely stem from collective differences in proteolytic stability and target binding activities.

### MAX-STRs Bind E-Box Containing Genes and Oppose MYC-Dependent Phenotypes in Cells.

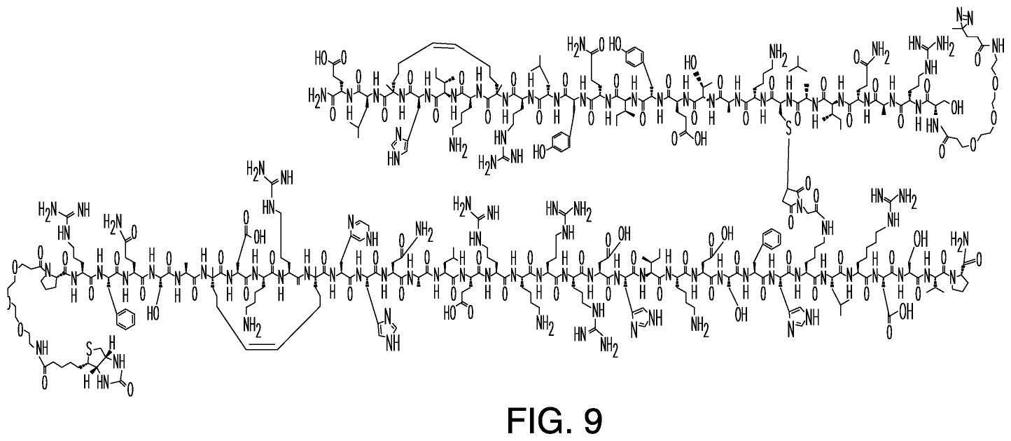

The combined biochemical and pharmacologic properties of optimized compounds suggested they should be capable of directly binding E-box sites within cells. Chromatin immunoprecipitation (ChIP) studies have validated the association of MYC and MAX proteins with specific E-box promoters and enhancers in numerous cell lines. Guided by these datasets, ChIP-qPCR assays were used to directly interrogate STR engagement with relevant target genes in cells. MYC- and MAX-dependent ChIP-qPCR confirmed that both proteins associated with several previously validated, E-box containing genes in P493-6 B-cells, including RPL37, MRPS15, TOR1A and FBXW8 (FIG. 6B). Also monitored was binding at SNTG2 and an intergenic region of the genome, which do not contain E-box sites, and confirmed that they were minimally enriched by MYC. A biotinylated analog of STR118 was synthesized for use in analogous ChIP assays, however suitable conditions for stable formaldehyde crosslinking to purified E-box containing DNA in vitro were not identified, which is consistent with the inefficient crosslinking reported for many small DNA binding proteins. Therefore, it was hypothesized that synthetic installation of a photoactive moiety proximal to phosphodiester backbone contact surface of DNA could allow for light-activated photocrosslinking to bound DNA within cells. To test this approach, a biotinylated-STR118 analog was synthesized containing a diazirine linked moiety at the N-terminus of the zipper helix (P-BioSTR118), and it was found that this molecule formed a stable, covalent complex with E-box DNA in vitro only upon exposure to 365 nm light (FIG. 9). Subsequently performed was a direct ‘photo-ChIP’ enrichment experiment with P-BioSTR118-treated, proliferating P493-6 cells. Significant binding and enrichment of P-BioSTR118 was observed at each of the MYC-bound E-box containing genes inside of cells, and no enrichment at negative control regions was observed (FIG. 6C). Additionally, none of the target loci were enriched in a control condition in which streptavidin beads were blocked with free biotin, thereby preventing enrichment STR118-DNA adducts. Collectively, these data confirm that optimized STRs can access intracellular compartments intact and specifically engage target E-box sites in the genome.

To assess whether optimized MAX-STRs can inhibit MYC-dependent function in cells, the MYC-responsive P493-6 cancer cell line was utilized. P493-6 cells grown in the absence of tetracycline constitutively express MYC (‘MYC-ON’), however, cells treated with tetracycline (‘MYC-OFF’) have low levels of MYC protein and severely reduced proliferation in cell culture (FIGS. 6E and 6F). Therefore, this cell model enables direct comparison of cellular responses in the presence and absence of oncogenic MYC signaling. Treatment of ‘MYC-ON’ P493-6 cells with STR116 resulted in dose- and time-dependent inhibition of cell proliferation, mirroring the effects of tetracycline-induced MYC blockade (FIGS. 6E and 6F). By contrast, STR116 treatment of P493-6 cells in the ‘MYC-OFF’ state had no significant effect on cell growth, confirming a MYC-dependent phenotypic response (FIG. 6F). Tetracycline treatment in these cells also blunts the expression of known MYC-target genes like CCNB1 and LDHA, which drive proliferation and anabolic growth. STR116 treatment of ‘MYC-ON’ cells significantly reduced expression of both LDHA and CCNB1 protein, although to a lesser extent than complete MYC ablation by tetracycline treatment. Treatment with STR118 resulted in less pronounced decreases in target protein expression and MYC-dependent growth, likely resulting from decreased stability and accumulation of intact molecule in cells relative to STR116. Likewise, STR116 treatment resulted in a more significant reduction in E-box regulated reporter gene activity in HCT116 reporter cells, relative to STR118 (FIG. 6D). Collectively, these data confirmed that targeting the MYC/MAX binding through cell-permeable MAX-STRs can mirror some of the effects of MYC protein depletion and inhibit MYC-dependent phenotypes.

### X-Ray Crystal Structure Confirms MAX-STRs Mimic Full-Length Transcription Factors and Specifically Bind the DNA Major Groove.

The biophysical and biochemical structure activity relationships elucidated here strongly suggest that STRs assemble and recognize E-box DNA in a manner similar to full length bHLH TFs like MYC and MAX. To directly confirm this hypothesis, the progenitor STR, B-Z, was crystallized with a 16-mer oligonucleotide containing a central 5′-CACGTG-3′ target sequence. A screen of crystallization conditions yielded reproducible rod-like crystals, permitting the 2.7 Å structure to be solved by X-ray diffraction followed by molecular replacement using a published structure of the MAX/MAX ternary complex with E-box DNA (PDB ID: 1HLO; Table 6 and methods) (Brownlie et al., Structure, 5: 509-520 (1997), incorporated by reference herein). The asymmetric unit cell consisted of four B-Z dimers, each bound to a single duplex DNA, with crystal contacts observed between inter-unit tetrahelix bundles and single base pair overhangs of adjacent oligonucleotide duplexes. The zipper and basic sequences in each B-Z monomer were completely α-helical and connected by a well-ordered glycyl-maleimide-cysteine adduct on the back face of each helix. The interhelix crosslink was closely packed by surrounding residues on each helix, effectively locking the B and Z helices into a defined register relative to one another. As predicted by the cross-dimer design, each B-Z monomer forms a ‘sandwich-like’ homodimer to form a tetrahelix bundle and orient the basic helices for sequence specific DNA binding. The interface formed between the pseudo-symmetric homodimer buries approximately 1590 Å² and is mediated by extensive contacts between residues in both the B and Z helices. Anchoring this interface is an extensive hydrophobic core in the tetrahelix interior formed by bIIe39, bLys40, bPhe43, bLeu46, bArg47, bVal50, bPro51, zArg60, zIle63, zLeu64, zAla67, zThr68, zTyr70, zIle71, zNle74 and zArg75, where ‘b’ and ‘z’ refer to the basic and zipper helix numbering from the parent MAX protein. Supporting this core is an additional layer of solvent exposed hydrophobic and polar contacts that contribute to the intermolecular tertiary and quaternary structure, including close packing between zTyr70 and zNle74 with bVal50 and bPro51.

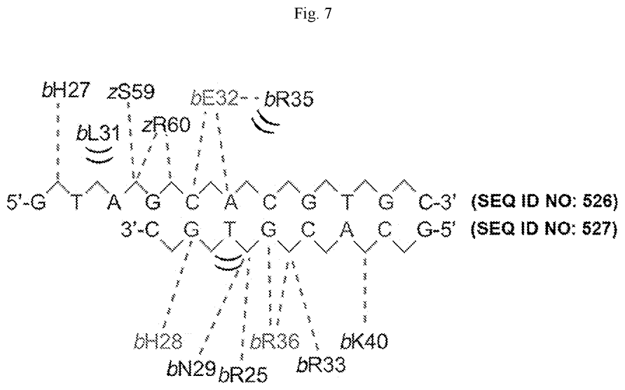

Like full-length MYC and MAX, the B-Z dimer binds the E-box target DNA with each monomer interacting with half of the 5′-CACGTG-3′ recognition sequence. Each basic helix makes numerous contacts to the phosphodiester backbone of DNA, as well as four sequence-specific contacts deep in the major groove. Backbone contacts are made by residues throughout the entire basic helix and encompass a 12-nucleotide span surrounding the E-box. These contacts include a bHis27-PO₄ contact three nucleotides outside of the E-box, and bArg25, bAsn29, bArg33, bArg36, bLys40, zSer59 and zArg60 all make contacts to phosphodiester positions within the core E-box sequence (FIG. 7). Each monomer makes hydrogen bond-mediated, sequence-specific contacts with both strands of the 5′-CAC-3′ half-site (FIG. 7). The ‘antisense’ contacts (denoted by ‘) include bHis28 and N7/C6 carbonyl of Guanosine-6′ and bArg36 with N7/C6 carbonyl of Guanosine-4′. On the ‘sense’ strand, bGlu32 makes close contact with N6 of Adenosine-2, N4 of Cytosine-3 and potentially N7 of the 5′-Guanosine outside of the E-box in this sequence. Superimposing the DNA-bound B-Z and MAX/MAX structures reveals a striking congruence between the DNA binding residues, with an overall RMSD of 0.847 Å for the backbone of the entire DNA binding domain held in common. The interface between DNA binding surface of B-Z (1781 Å²) is also comparable to that of the MAX homodimer (1726 Å²). Taken together, these data confirmed that the modular, synthetic STR architecture mimics the overall structure and sequence-specific DNA binding function of a full-length transcription factor.

Programming Alternative DNA Recognition with Modular STR Designs.

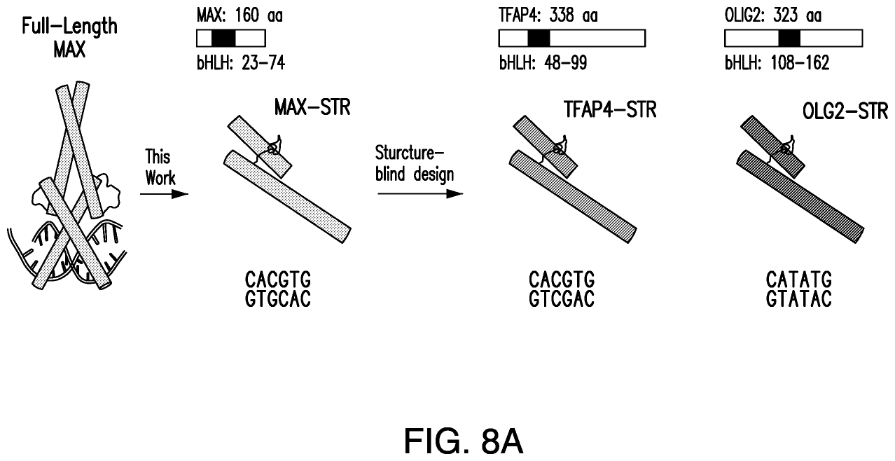

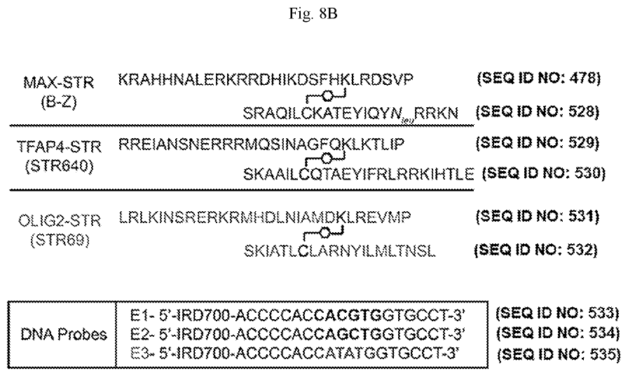

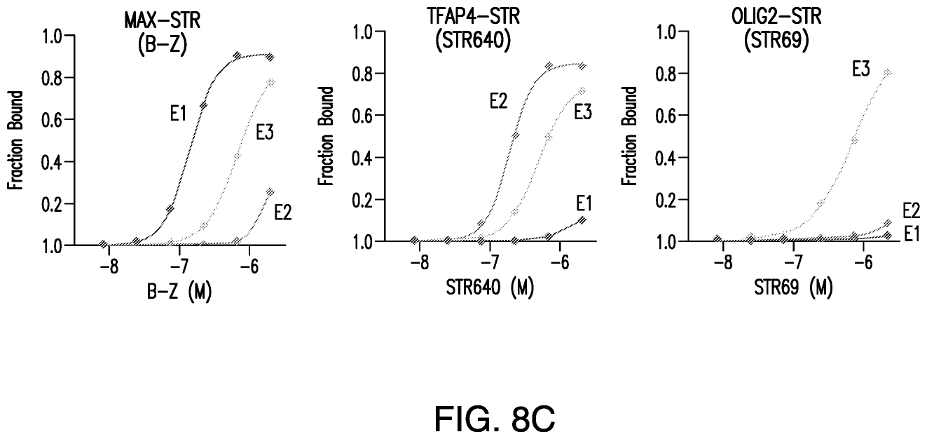

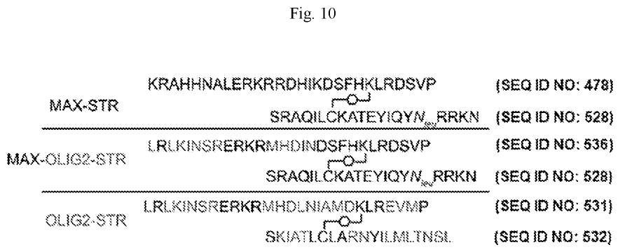

MAX homodimers exhibit sequence specificity for the E-Box sequence, whereas many of the 107 known human bHLH transcription factors bind distinct NCANNTGN motifs (where ‘N’ is any nucleotide). Given the near identical structural alignment of the DNA-binding region of MAX and a MAX-STR in crystal structures, next determined was if unique sequence-specific binding preferences could be programmed into novel STRs through incorporation of the aligned primary sequences of alternate bHLH proteins (FIG. 8A). Oligodendrocyte transcription factor 2 (OLIG2) and Transcription factor activating protein 4 (TFAP4) were chosen as model bHLH transcription factors with known/predicted DNA binding specificities that depart from MYC/MAX. A new STR was designed and synthesized for each target TF based solely on sequence alignment of the bHLH domains of OLIG2 and TFAP4 with the ‘B-Z’ progenitor STR derived from MAX (FIGS. 8A-8C). EMSA gels clearly show that the MAX derived STR B-Z retains similar specificity to the intact protein, whereas the STR69 and STR640 show specificity for the OLIG2- and TFAP4-preferred motifs (target sequences in E3 and E2, respectively) and show minimal binding to the E-box motif (FIGS. 8B, 8C, and 10). Despite the considerable differences in the primary sequences of the bHLH domains in OLIG2, TFAP4 and MAX, the direct application of the STR design strategy generated a potent and specific synthetic TF mimetic (FIG. 8C) without any advanced engineering. Intriguingly, an alternative approach of replacing the DNA-contacting residues in B-Z with those from OLIG2 did not yield a functional DNA-binding STR, reinforcing the potential importance of long-range crosstalk between the tetrahelix bundle and DNA contact surfaces in bHLH proteins and STRs alike (FIG. 10). Overall, these data confirm that the STR design strategy is a modular approach to develop bHLH TF-defined STR mimetics for other TFs and unique target DNA motifs.

### DISCUSSION

This study established design, synthesis, and structure activity relationships for a novel class of synthetic transcription factor mimetics derived from MAX. These non-natural, ‘cross-dimer’ STRs recapitulate the cooperative association of DNA binding domains, as found in hundreds of known transcription factors, however their hybrid architecture suggests they should not interfere with protein-protein interactions between endogenous bHLH proteins.

A modular, convergent synthetic route was developed that enabled introduction of multiple non-natural stabilizing elements and in the process identified the necessary structural features required for high affinity and specific DNA recognition. Basic helices alone, whether containing internal stabilizing elements or synthetically dimerized, lack significant DNA recognition capacity. Preorganization of the proximal basic-zipper helix register is sufficient to drive formation of the tetrahelix core above the basic helices, which ultimately permits high affinity and sequence-specific DNA binding. Notably, observed was comparable specificity, but not increased affinity, of several stapled STRs (e.g., B1-Z2 & B 11-Z6) relative to the non-stapled progenitor B-Z. As these molecules are considerably more helical and thermally stable than B-Z, these data support published models of disordered-to-ordered search and binding by the basic helices as being integral to forming tight, specific complexes with DNA. Preorganization of the individual helices may result in a thermodynamic trade-off between avoiding the energetic cost of folding to bind the major groove versus the restrictions on subtle conformational adjustments for optimal binding, which may be more accessible to non-stapled versions. STRs based on the minimal B-Z core were incapable of competing with MYC/MAX or MAX/MAX for E-box DNA binding. Optimized derivatives of this scaffold, including STR116 and STR118, were potent competitors with the full-length MYC/MAX and MAX/MAX complexes. This finding suggests that kinetic factors may be important determinants of effective competition, as STR116 and B-Z have identical K_{D} values for E-box binding, but only the former effectively competed for DNA binding.

In addition to improved biochemical properties, it was found that activity-guided stabilization of tertiary and secondary elements led to significant increases in structural and proteolytic stability of several STRs. Targeted modification at and around sites of proteolysis within these molecules correlated with protection from proteolysis, however these changes had to be balanced with preservation of DNA binding. The net result of this enhanced stability for optimized molecules like STR116 and STR118, at least as explored here, was enhanced protease resistance, cellular penetration, DNA binding activity and activity in cellular models. The studies also confirmed that DNA binding domains comprised of canonical amino acids alone, such as MAX and by analogs like Omomyc and other engineered proteins, are largely unstructured and highly sensitive to proteases. As such, they are likely to suffer significant degradation inside and outside of cells if used as chemical probes and therapeutics. More generally, the data support the notion that modular synthesis of secondary and tertiary domain epitopes can be used to generate pharmacologically active mimetics, such as those targeting DNA in this study, and likely other proteins and biomolecules in the future.

Molecules with improved stability and biochemical properties, such as STR116 and STR118, are cell permeable and can specifically engage E-box target genes in live cells. Treatment of B cells with STR116, which is the more stable and cell permeable of the two, results in decreased expression of known MYC target genes and antiproliferative activity only in the context of oncogenic MYC-signaling in P493-6 B cells.

The biochemical and structural findings also support the notion that the self-associating, cross-dimer STR architecture mimics full-length TF protein structure and function, and therefore should be applicable to other bHLH TFs. The platform was applied to efficiently construct STRs derived from OLIG2 and TFAP4, two bHLH-TFs that are implicated in cancer pathogenesis. The resulting molecules, STR64 and STR69, display differentiated sequence specific DNA binding from their MAX-derived B-Z ancestor and represent potential antagonists of their respective transcription factors. These findings confirm that the general approach described herein forms a basis for methodical development of sequence specific synthetic transcriptional regulators for the study and pharmacologic targeting of gene expression regulated by diverse transcription factors.

### Example 2

This example further demonstrates helical dimers, in accordance with aspects of the disclosure.

TABLE 9

| | Molecular | LCMS | LCMS Retention | |
|---|---|---|---|---|
| sDBD | weight | Method | time (min) | Observed ions |
| B-Z | 6122.02 | A | 10.21 | 1531.8, 1225.1, 1021.0, 875.5, 766.3 |
| B1-Z | 6230.2 | A | 10.23 | 1558.4, 1246.6, 1039.2, 890.8, 779.8 |
| B2-Z | 6120.13 | A | 10.57 | 1530.8, 1224.8, 1020.8, 875.2, 765.8 |
| B3-Z | 6107.04 | A | 10.93 | 1527.5, 1222.3, 1018.4, 873.5 |
| B4-Z | 6173.11 | A | 10.12 | 1543.9, 1235.5, 1029.7, 882.7, 772.5 |
| B7-Z | 6187.13 | A | 10.13 | 1574.5, 1238.3, 1031.8, 884.5, 774.0 |
| B8-Z | 6121.07 | A | 10.95 | 1530.9, 1224.9, 1020.9 |
| B9-Z | 5629.43 | A | 10.3 | 1877.7, 1408.3, 1126.8, 938.9, 805.0 |
| BLI1-Z | 6994.96 | C | 5.62 | 1399.7, 1166.8, 1000.1, 875.3 |
| B-Z1 | 6116.05 | A | 11.47 | 1529.7, 1223.8, 1020.2, 874.8 |
| B-Z2 | 6081.01 | A | 10.79 | 1520.8, 1216.9, 1014.2, 869.71 |
| B-Z3 | 6080.96 | A | 10.86 | 1520.8, 1216.8, 1014.2, 869.7, 761.0 |
| B-Z4 | 6094.99 | A | 10.95 | 1524.5, 1219.6, 1016.5, 871.5, 762.8 |
| B-Z5 | 5723.55 | A | 10.28 | 1908.4, 1431.7, 1145.3 |
| B-ZL1 | 7453.43 | C | 5.59 | 1864.2, 1491.5, 1243.0, 1065.6 |
| B1-Z1 | 6224.24 | A | 11.48 | 1556.7, 1245.6, 1038.3, 889.8 |
| B1-Z2 | 6189.19 | A | 10.78 | 1547.9, 1238.6, 1032.3, 885.0 |
| B2-Z1 | 6114.17 | A | 11.72 | 1529.2, 1223.5, 1019.3, 874.2 |
| B2-Z2 | 6079.12 | A | 11.04 | 1520.4, 1216.7, 1013.8, 869.1 |
| B1-Z4 | 6203.18 | B | 7.3 | 1551.7, 1241.6, 1034.7 |
| B2-Z4 | 6093.1 | B | 7.47 | 1523.9, 1219.2, 1016.3 |
| B5-Z4 | 6160.11 | B | 7.4 | 1540.8, 1232.8, 1027.4, 880.8 |
| B6-Z4 | 6207.2 | B | 7.76 | 1551.4, 1241.7, 1034.8, 886.9, 776.2 |
| BLI1-ZL1 | 8326.37 | C | 5.6 | 1666.1, 1388.5, 1190.3, 1041.6, |
| STR116 | 7579.67 | B | 7.28 | 1895.7, 1516.7, 1264.1, 1083.5 |
| STR118 | 7575.77 | B | 7.18 | 1894.8, 1515.8, 1263.4, 1083.0 |
| MAX-OLIG2- | 6136.1 | B | 7.08 | 1534.8, 1228.1 |
| STR | | | | |
| STR640 | 6408.48 | B | 7.41 | 1602.9, 1282.5, 1068.8 |
| STR69 | 5950.18 | B | 6.95 | 1984.2, 1488.3, 1191.0 |
| B | 3488.99 | A | 10.59 | 1744.9, 1396.3, 1163.6, 873.0, 698.8 |
| B1 | 3597.17 | A | 11.04 | 1799.3, 1439.5, 1199.8, 900.0, 720.3 |
| FITC-B-Z | 6672.6 | A | 10.677 | 1669.2, 1335.3, 1112.8, 954.2, 834.8 |
| FITC-STR116 | 8130.25 | B | 7.39 | 2033.3, 1626.7, 1355.9, 1162.2 |
| FITC-STR118 | 8126.35 | B | 7.32 | 2032.3, 1626.1, 1355.2, 1161.7 |
| P-BioSTR118 | 8234.58 | B | 7.28 | 1647.7, 1373.2, 1177.2, 1030.2 |

Method A: Solvent A (95:5:0.1% H2O/ACN/TFA) and solvent B (95:5:0.1% ACN/H2O/TFA); 0.5 ml min-1 flowrate, 0-2 min (0% B), 2-16 min (0-75% B), 16.5-18.5 min (100% B), 19 min (0% B).
Method B: Solvent A (95:5:0.1% H2O/ACN/TFA) and solvent B (95:5:0.1% ACN/H2O/TFA); 0.5 ml min-1 flowrate, 0-2 min (5% B), 2-8.8 min (5-95% B), 9-11 min (95% B), 11.1 min (5% B).
Method C: Solvent A (99.9:0.1% H2O/TFA) and solvent B (100% ACN); 0.5 ml min-1 flowrate, 0-1.4 min (5% B), 1.4-6.4 min (5-75% B), 6.5 min (95% B), 8.25 min (95% B).

### Example 3

This example further demonstrates helical dimers and helical tetramers, in accordance with aspects of the disclosure.

The following peptides in Table 10 have been synthesized. They have the core B-Z sequence with appended amino acid sequences from other transcription factors (MAX [isoforms 1 and 2], MAD, AND MYC).

TABLE 10

| | | SEQ |
|---|---|---|
| | | ID |
| Name | Sequence | NO: |
| B | Ac-KRAHHNALERKRRDHIKDSFHKLRD | 432 |
| | PSV-NH₂ | |
| Z | Ac-SRAQILCKATEYIQYBRRKN-NH₂ | 474 |
| BLI1 | Ac-PRFQSAADKRAHHNALERKRRDHIK | 434 |
| | DSFHKLRDSVP-NH₂ | |
| BLIIs | Ac-QSAADKRAHHNALERKRRDHIKDSF | 435 |
| | HKLRDSVP-NH₂ | |
| BLI2 | Ac-IEVESDADKRAHHNALERKRRDHIK | 436 |
| | DSFHKLRDSVP-NH₂ | |
| BMYC | Ac-PRSSDTEENVKRAHHNALERKRRDH | 437 |
| | IKDSFHKLRDSVP-NH₂ | |
| BMYCs | Ac-TEENVKRAHHNALERKRRDHIKDSF | 438 |
| | HKLRDSVP-NH₂ | |
| BMAD | Ac-KSKKNNSSKRAHHNALERKRRDHIK | 439 |
| | DSFHKLRDSVP-NH₂ | |
| BMADs | Ac-KNNSSKRAHHNALERKRRDHIKDSF | 440 |
| | HKLRDSVP-NH₂ | |
| B10 | Ac-PRFQSAADKRS₅HHNSSLERKRRDH | 441 |
| | IKDSFHKLRDSVP-NH₂ | |
| B11 | Ac-PRFQSAS₅DKRS₅HHNALERKRRDH | 442 |
| | IKDSFHKLRDSVP-NH₂ | |
| B12 | Ac-PRSsFQSS₅DKRAHHNALERKRRDH | 443 |
| | IKDSFHKLRDSVP-NH₂ | |

The following peptides in Table 11 also have been synthesized. The zipper helix is extended. ZL1 is a natural variant for MAX protein, whereas ZM3 and ZM4 are designer sequences. Z6 is an extension of the Z4 sequence.

TABLE 11

| | | SEQ |
|---|---|---|
| | | ID |
| Name | Sequence | NO: |
| Z | Ac-SRAQILCKATEYIQYN_{L}RRKN-NH₂ | 433 |
| ZL1 | Ac-SRAQILCKATEYIQYN_{L}RRKNHTHQ | 444 |
| | QDIDDLK-NH₂ | |
| ZM3 | Ac-SRAQILCKATEYIQYN_{L}RRKNHTLISE-NH₂ | 445 |
| ZM4 | Ac-SRAQILCKATEYIQYN_{L}RRKLHTHE-NH₂ | 446 |
| ZM5 | Ac-SRAQILCKATEYIQYLRRKIHTLE-NH₂ | 475 |
| Z6 | Ac-SRA_{ib}QILCQATEYIQS₅N_{L}RRS₅ | 447 |
| | LHTHE-NH₂ | |
| Z7 | Ac-SRA_{ib}QILCQATEYIQSN_{Leu}RRS₅ | 476 |
| | LHTS₅E-NH₂ | |
| Z8 | Ac-SRAQILCKATEYIQYLRS₅KIHSSLE-NH₂ | 477 |

Results are provided in the tables below with respect to experiments performed using polypeptides of the Examples. Tables 12 and 13 present binding data with regard to helical dimers (a first polypeptide covalently bound to a second polypeptide as described herein) that come together to from tetrahelical structures when bound and helical tetramers (a first polypeptide covalently bound to a second polypeptide, a third polypeptide covalently bound to a fourth polypeptide, and the second polypeptide covalently bound to the fourth polypeptide).

TABLE 12
EMSA

| | Helical Dimer apparent | Helical Tetramer apparent | |
|---|---|---|---|
| Name | K_{D} (nM) | K_{D} (nM) | Fold increase |
| B-Z | 16 | 2 | 8 |
| B1-Z2 | 26 | 1.8 | 14 |

TABLE 13
EMSA Competition with MAX protein

| Name | Helical Dimer | Helical Tetramer |
|---|---|---|
| B-Z | Does not compete | Does not compete |
| B1-Z2 | Does not compete | 1100 (IC₅₀ (nM)) |

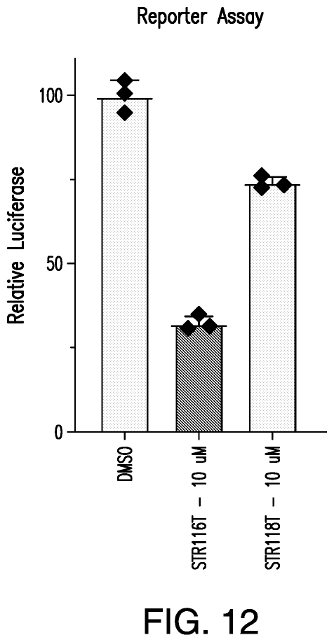

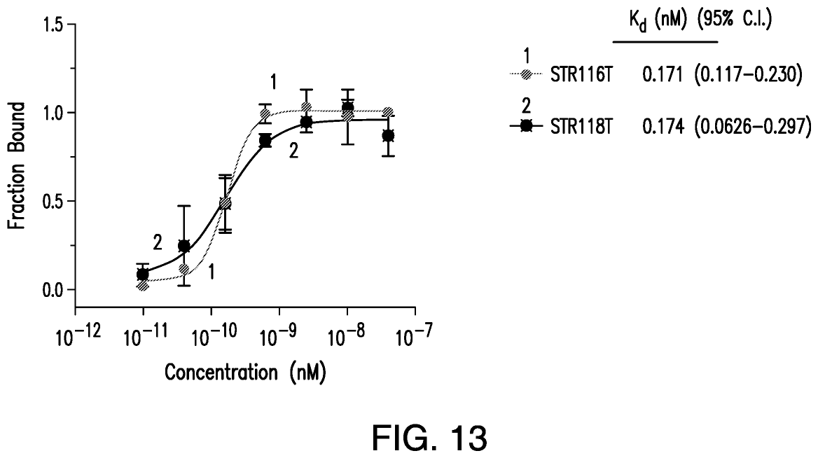

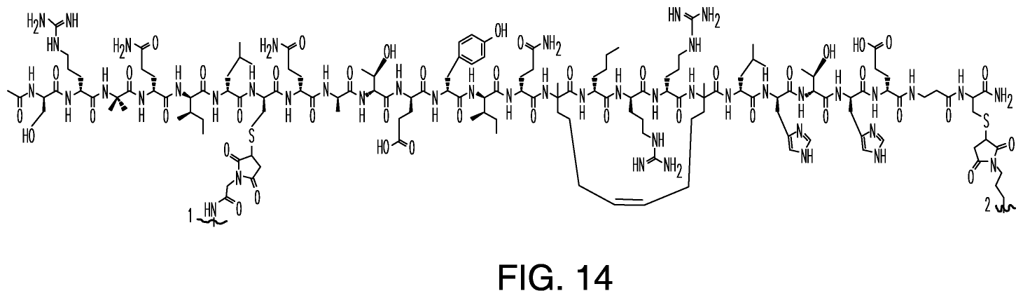

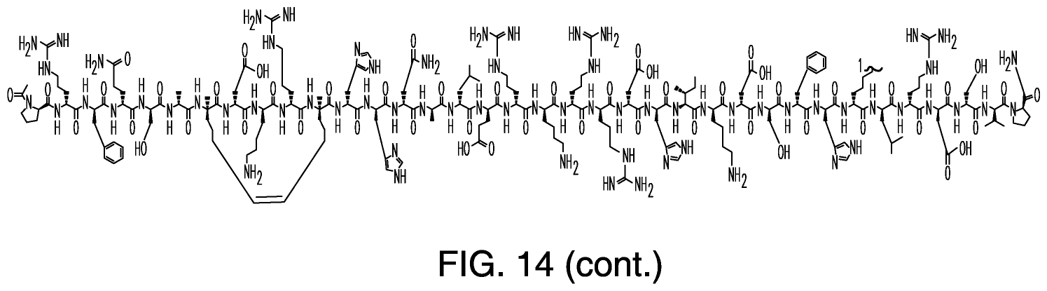

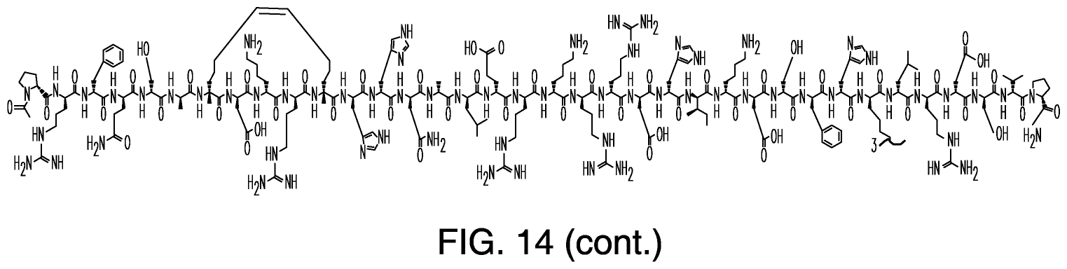

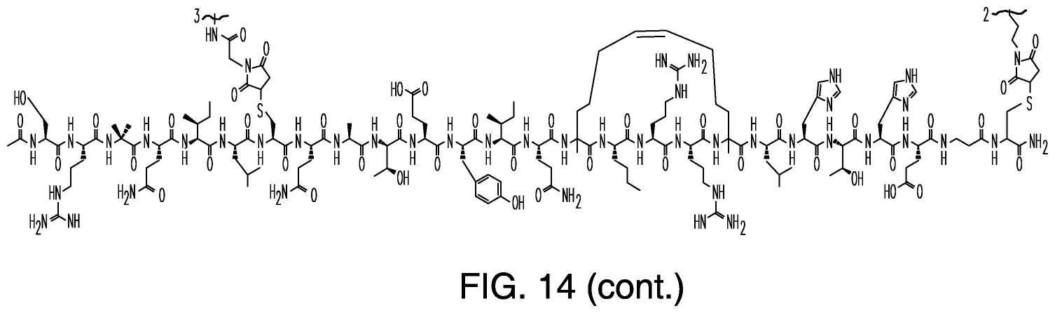

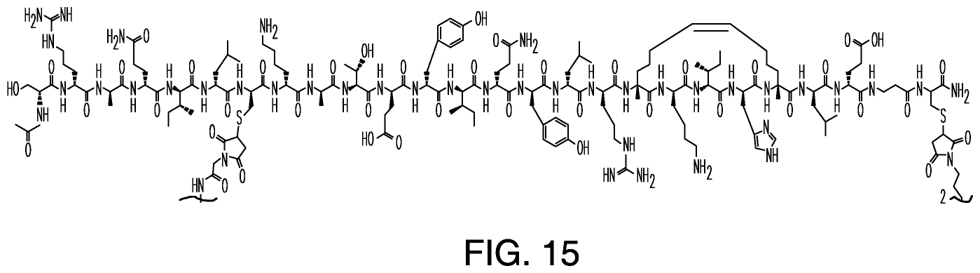

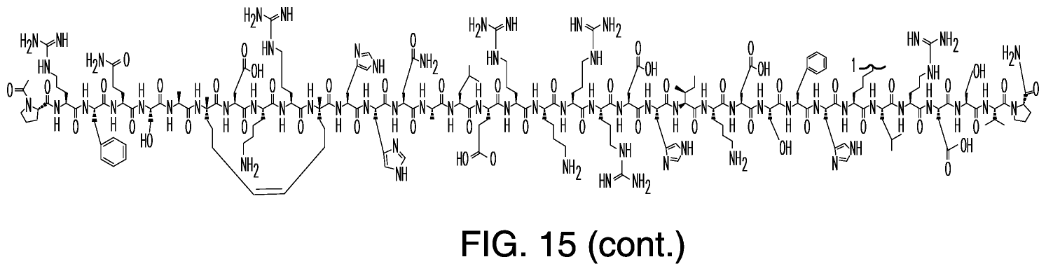

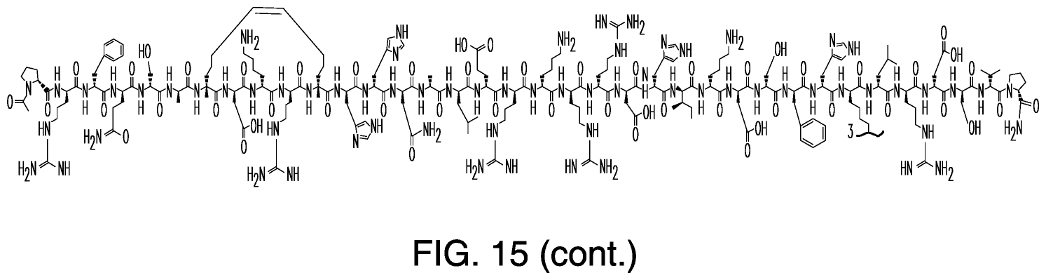

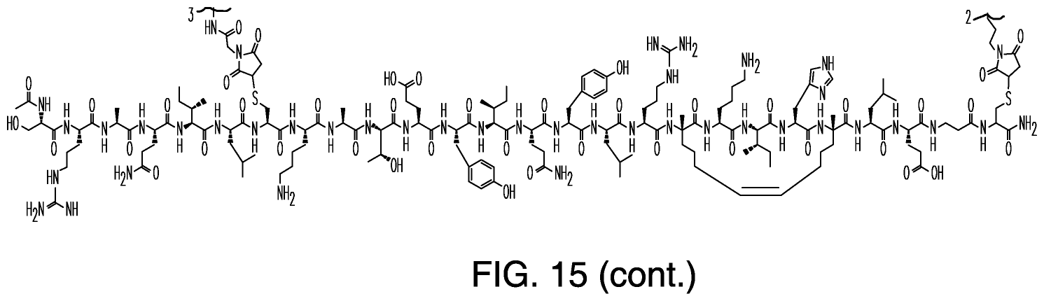

Helical tetramers of STR116 (STR116T) and STR118 (STR118T) were tested in a luciferase assay, with the results shown in FIG. 12. Binding data with Kd for STR116T and STR118T is shown in FIG. 13. The structure of STR116T is shown in FIG. 14. The structure of STR118T is shown in FIG. 15.

Results are provided in the tables below with respect to experiments performed using polypeptides of the Examples.

TABLE 14
EMSA Binding Curve

| sDBD | kd (nM) |
|---|---|
| B-Z | 16 |
| B1-Z | 17 |
| B2-Z | 15 |
| B3-Z | 76 |
| B4-Z | 25 |
| B7-Z | 173 |
| B8-Z | 672 |
| B9-Z | 242 |
| B-Z1 | 45 |
| B-Z2 | 22 |
| B-Z3 | 15 |
| B-Z4 | 14 |
| B-Z5 | 88 |
| B1-Z1 | 49 |
| B1-Z2 | 26 |
| B2-Z2 | 18 |
| B1-Z4 | 40 |
| B2-Z4 | 15 |
| B5-Z4 | 118 |
| B6-Z4 | 57 |
| B7-Z4 | 234 |
| B8-Z4 | 164 |
| B-ZL1 | 19 |
| BLI1-Z | 12 |
| BLI1-ZL1 | 19 |
| B-ZM4 | 8 |
| BLI1-ZM4 | 10 |
| BLI1-ZM5 | 2.8 |
| B10-Z6 | 16 |
| B11-ZM4 | 21 |
| B11-Z4 | 38 |
| B11-Z6 | 14 |
| B11-Z7 | 22 |
| B11-Z8 | 4.2 |
| B12-Z6 | 8 |
| B | >2000 |
| B1 | >2000 |

TABLE 15
MAX Competition Titrations

| sDBD | IC₅₀ (nM) |
|---|---|
| BLI1-Z | 905 |
| BLI1-ZL1 | 935 |
| BLI1-ZM4 | 560 |
| B10-Z6 | 326 |
| B11-ZM4 | 560 |
| B11-Z4 | 496 |
| B11-Z6 | 65 |
| B11-Z8 | 79 |
| B12-Z6 | 62 |

TABLE 16
MAX Competition 500 nM sDBD

| sDBD | |
|---|---|
| BLI1-Z | 50% |
| BLI2-Z | 16% |
| B-ZM4 | 41% |
| BLI1s-Z | 22% |
| BMYCs-Z | 7% |
| BMADS-Z | 24% |
| BMYC-Z | 9% |
| BMAD-Z | 66% |

TABLE 17
EMSA Fraction bound (sDBD = 500 nM)

| sDBD | |
|---|---|
| BLI1-Z | 100% |
| BLI2-Z | 72% |
| BLI1-ZL1 | 100% |
| BLI2-ZL1 | 54% |
| B-ZL1 | 100% |
| B-Z | 100% |

TABLE 18
EMSA Fraction bound (sDBD = 25 nM)

| sDBD | |
|---|---|
| B-ZM3 | 53% |
| B-ZM4 | 78% |
| BLI1s-Z | 50% |
| BMYCs-Z | 27% |
| BMADs-Z | 63% |
| BMYCs-Z | 19% |
| BMADs-Z | 82% |

### Example 4

This example further demonstrates aspects of the disclosure.

The sequences of Table 19 incorporate point mutations derived from homologous bHLH proteins of Table 20. The bHLH proteins have different DNA specificity. Substituting amino acids from the DNA binding groove is contemplated to alter sDBD specificity.

TABLE 19

| | | SEQ |
|---|---|---|
| | | ID |
| Name | Sequence | NO: |
| B | Ac-KRAHHNALERKRRDHIKDSFHKLRDSVP-NH₂ | 432 |
| B1 | Ac-KRS₅HHNSSLERKRRDHIKDSFHKLRDSVP-NH₂ | 448 |
| BRAEL | Ac-KRS₅HANSSLERKRLDHIKDSFHKLRDSVP-NH₂ | 449 |
| BRAET | Ac-KRS₅HANSSLERKRTDHIKDSFHKLRDSVP-NH₂ | 450 |
| BKSAR | Ac-KKSSHSNS₅LARKRRDHIKDSFHKLRDSVP-NH₂ | 451 |
| BRHNR | Ac-KRS₅HHNS₅LNRKRRDHIKDSFHKLRDSVP-NH₂ | 452 |

TABLE 20

| | | SEQ | | SEQ | | |
|---|---|---|---|---|---|---|
| | Basic | ID | | ID | Speci- | |
| Protein | Sequence | NO: | Groove | NO: | ficity | PDB |
| MAX | KRAHHNALERK | 478 | RHER | 479 | CACGTG | 1hlo |
| | RRDHIKDSFH | | | | | |
| | KLRDSVP | | | | | |
| MyoD | RRKAATMRERR | 480 | RAEL | 481 | CAGCTG | 1MDY |
| | RLSKVNEAFE | | | | | |
| | TLKRCTSSNP | | | | | |
| TWIST | QRVMANVRERQ | 482 | RAET | 483 | CATATG | N/A |
| | RTQSLNEAFA | | | | | |
| | ALRKIIP | | | | | |
| HIF | RKEKSRDAARS | 484 | KSAR | 485 | CGTACG | 4ZPR |
| | RRSKESEVFYE | | | | | |
| | LAHQLP | | | | | |
| | KRAHHNALNRK | 486 | RHNR | 487 | CGCGCG | N/A |
| | RRDHIKDSFH | | | | | |
| | KLRDSVP | | | | | |

### Example 5

This example further demonstrates helical dimers, in accordance with aspects of the disclosure.

Additional sequences are provided in Table 21 below.

TABLE 21

| | | SEQ |
|---|---|---|
| NAME | Sequence | ID NO: |
| B11s | Ac-(S5)DKR(S5)HHNALERKRRDHIKDSFHK(GlyMal)LRDSVP-NH₂ | 489 |
| B11-dk | Ac-PRFQSA(S5)DKR(S5)HHNALER(D-lysine) | 490 |
| | RRDHIKDSFHK(Gly-Mal)LRDSVP-NH₂ | |
| B11R | Ac-PRFQSA(S5)DKR(S5)HHNALERKRRDHIKDSFHCLRDSVP-NH₂ | 491 |
| B10- | Ac-PAAKRVKLDKR(S5)HHN(S5)LERKRRDHIKDSFHK(GlyMal) | 492 |
| Cmyc(NLS) | LRDSVP-NH₂ | |
| Olig4 | Ac-LRLKINSRERKRMHDINDSFHK(GlyMal)LRDSVP-NH₂ | 493 |
| TFAP4 | Ac-RREIANSNERRRMQSINDSFHK(GlyMal)LRDSVP-NH₂ | 494 |
| B69 | Ac-LRLKINSRERKRMHDLNIAMDK(GlyMal)LREVMP-NH₂ | 495 |
| B64 | Ac-RREIANSNERRRMQSINAGFQK(GlyMal)LKTLIP-NH₂ | 458 |
| B85 | Ac-RRKAATMRERRRLSKVNEAFEK(GlyMal)LKRSTS-NH₂ | 496 |
| B69G | Ac-LRLKINSRERKRMHDLNIAMDK(ButMal)LREVMP-NH₂ | 456 |
| B64G | Ac-RREIANSNERRRMQSINAGFQK(ButMal)LKTLIP-NH₂ | 497 |
| B85G | Ac-RRKAATMRERRRLSKVNEAFEK(ButMal)LKRSTS-NH₂ | 498 |
| B64L | Ac-QRDQERRIRREIANSNERRRMQSINAGFQK(GlyMal)LKTLIP-NH₂ | 499 |
| B69L | Ac-MTEPELQQLRLKINSRERKRMHDLNIAMDK(GlyMal)LREVMP-NH₂ | 500 |
| B85L | Ac-KRKTTNADRRKAATMRERRRLSKVNEAFEK(GlyMal)LKRCTS-NH₂ | 501 |
| Z7 | Ac-SR(Aib)QILCQATEYIQS(NI)RR(S5)LHT(S5)E-NH₂ | 502 |
| ZM5 | Ac-SRAQILCKATEYIQYLRRKIHTLE-NH₂ | 475 |
| Z8 | Ac-SRAQILCKATEYIQYLR(S5)KIH(S5)LE-NH₂ | 455 |
| Z8-Biotin | Biotin-PEG3-SRAQILCKATEYIQYLR(S5)KIH(S5)LE-NH₂ | 503 |
| Z9 | Ac-SR(Aib)QILCQATEYIQYLR(S5)KIH(S5)LE-NH₂ | 504 |
| Z8-DZ1 | Diazirine-PEG3-SRAQILCKATEYIQYLR(S5)KIH(S5)LE-NH₂ | 505 |
| Z8-DZ2 | Diazirine-(6-aminohexanoic acid)- | 506 |
| | SRAQILCKATEYIQYLR(S5)KIH(S5)LE-NH₂ | |
| Z8-DZ3 | Diazirine-SRAQILCKATEYIQYLR(S5)KIH(S5)LE-NH₂ | 507 |
| Z8R | Ac-SRAQILKKATEYIQYLR(S5)KIH(S5)LE-NH₂ | 508 |
| Z70 | Ac-SRA_{ib}QILCQATEYIQSLRR(S5)IHT(S5)E-NH₂ | 509 |
| Z80 | Ac-SRAQILCKATEYIQRLR(S5)KIR(S5)LE-NH₂ | 488 |
| Z10 | Ac-SRAQILCKATEYIQYLRRKI(S5)TLE(S5)-NH₂ | 510 |
| Z81 | Ac-SRAQILCKATEYIQYLR(S5)KIH(S5)LEPKKKRKV-NH₂ | 511 |
| Z82 | Ac-SRAQILCEATEYIQELR(S5)KIE(S5)LE-NH₂ | 512 |
| Z- | Pya-Py-Py-Py-PEG3-SRAQILCKATEYIQY(N1)RRKN-NH₂ | 513 |
| Polyamide | | |
| Z69 | Ac-SKIATLCLARNYILMLTNSL-NH₂ | 457 |
| Z64 | Ac-SKAAILCQTAEYIFSLEQEK-NH₂ | 514 |
| Z85 | Ac-PKVEILCNAIRYIEGLQALL-NH₂ | 515 |
| Z690 | Ac-SKIATLCLARNYILRLRRKIHTLE-NH₂ | 516 |
| Z640 | Ac-SKAAILCQTAEYIFRLRRKIHTLE-NH₂ | 459 |
| Z850 | Ac-PK VEILCNAIRYIERLRRKIHTLE-NH₂ | 517 |

Certain sequences were made and tested together for binding to an E-box probe. The Kd values are shown in Table 22.

TABLE 22

| STR | Kd (nM) |
|---|---|
| B11s-Z8 | 6.5 |
| B11-Z10 | 4.8 |
| B11-Z80 | 2.7 |
| B11-Z70 | 11 |
| B11-Z81 | >100 |

B11-Z70 and B11-Z80 have strategic substitutions to increase solubility and are soluble in RPMI media+10% FBS at concentrations >75 μM.

### Example 6

This example further demonstrates aspects of the disclosure.

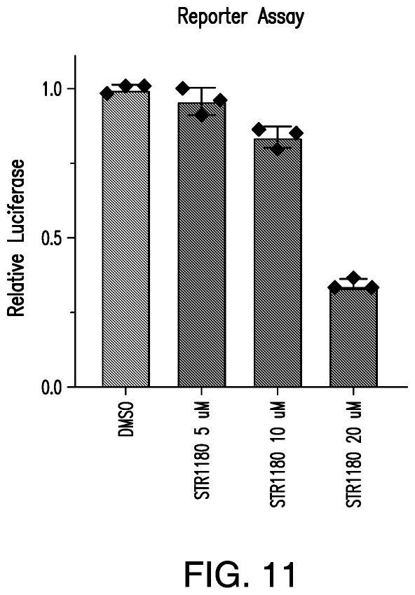

HCT116 cells expressing a c-myc responsive luciferase reporter were treated with increasing concentrations of STR1180 for 24 hours and the activity of the reporter gene was measured (FIG. 11). The assay was performed as described by the manufacturer, BPS Biosciences Inc., Catalog #60520.

The sequence of STR1180 is shown in Table 23.

TABLE 23

| | | SEQ ID |
|---|---|---|
| | | NO: |
| STR1180 | Ac-PRFQSA(S5)DKR(S5)HHNALE | 453 |
| | RKRRDHIKDSFHK(GlyMal) | |
| | LRDSVP-NH₂ | |
| | Ac-SRAQILCKATEYIQRLR(S5)KIR | 488 |
| | (S5)LE-NH₂ | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A polypeptide construct comprising:
(a) a first polypeptide comprising an amino acid sequence derived from a basic helix of a transcription factor protein that comprises a basic helix-loop-helix domain; and
(b) a second polypeptide comprising an amino acid sequence derived from a helix that extends in the C-terminal direction from the end of the loop of a basic helix-loop-helix domain of a transcription factor protein that comprises a basic helix-loop-helix domain;
wherein the first polypeptide and the second polypeptide are linked through an interpolypeptide covalent linkage.

2. The polypeptide construct of claim 1, wherein the basic helix of the first polypeptide comprises the amino acid sequence extending 36 residues in the N-terminal direction from the start of the loop of the basic helix-loop-helix domain.

3. The polypeptide construct of claim 1 or 2, wherein the helix of the second polypeptide comprises the amino acid sequence extending 31 residues in the C-terminal direction from the end of the loop of the basic helix-loop-helix domain.

4. The polypeptide construct of any one of claims 1-3, wherein the amino acid sequence of the first polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the first polypeptide.

5. The polypeptide construct of any one of claims 1-4, wherein the amino acid sequence of the second polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the second polypeptide.

6. The polypeptide construct of claim 4 or 5, wherein each set of non-natural amino acids are capable of undergoing a Diels-Alder reaction, a Huisgen reaction, or an olefin metathesis reaction.

7. The polypeptide construct of claim 4 or 5, wherein one non-natural amino acid within a set is Xaa^{A1} and the other non-natural amino acid within the set is Xaa^{B1},
wherein 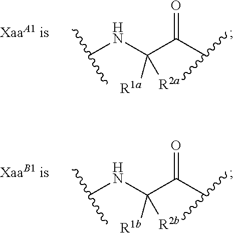
R^{1a} and R^{1b} are independently H, alkyl, alkenyl, alkynyl, arylalkyl, cycloalkylalkyl, heteroarylalkyl, or heterocyclylalkyl;
R^{2a} and R^{2b} are (i) independently alkenyl, alkynyl, azido, amino, carboxylic acid, or sulfide or (ii) taken together to form alkylene, alkenylene, alkynylene, or [R^{3a}—X—R^{3b}]ₙ, each of which is substituted with 0-6 R⁴;
each R^{3a} and R^{3b} are independently alkylene, alkenylene or alkynylene;
each R⁴ is independently halo, alkyl, OR⁵, N(R⁵)₂, SR⁵, SOR⁵, SO₂R⁵, CO₂R⁵, R⁵;
each X is independently O, S, SO, SO₂, CO, CO₂, CONR⁵ or 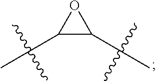 [SMILES: *C1OC1*]
each R⁵ is independently H or alkyl; and
n is an integer 1-4.

8. The polypeptide construct of claim 4 or 5, wherein the non-natural amino acids are capable of forming together a thioether, ether, amide, amine, triazole, or carbon-carbon double bond or a Diels-Alder adduct after reaction.

9. The polypeptide construct of claim 4 or 5, wherein the non-natural amino acids are independently selected from (S)-2-(4′-pentenyl)alanine (S5), (R)-2-(2′-propenyl)alanine (R3), and (R)-2-(7′-octenyl)alanine (R8).

10. The polypeptide construct of any one of claims 4-9, wherein the non-natural amino acids have undergone reaction to form the intrapolypeptide covalent cross-link with each other.

11. The polypeptide construct of any one of claims 1-10, wherein the interpolypeptide covalent linkage between the first polypeptide and the second polypeptide is a maleimide-thiol adduct.

12. A polypeptide construct comprising:
(a) the polypeptide construct of any one of claims 1-11;
(b) a third polypeptide comprising an amino acid sequence derived from a basic helix of a transcription factor protein that comprises a basic helix-loop-helix domain; and
(c) a fourth polypeptide comprising an amino acid sequence derived from a helix that extends in the C-terminal direction from the end of the loop of a basic helix-loop-helix domain of a transcription factor protein that comprises a basic helix-loop-helix domain;
wherein the third polypeptide and the fourth polypeptide are linked through an interpolypeptide covalent linkage.

13. The polypeptide construct of claim 12, wherein the basic helix of the third polypeptide comprises the amino acid sequence extending 36 residues in the N-terminal direction from the start of the loop of the basic helix-loop-helix domain.

14. The polypeptide construct of claim 12 or 13, wherein the helix of the fourth polypeptide comprises the amino acid sequence extending 31 residues in the C-terminal direction from the end of the loop of the basic helix-loop-helix domain.

15. The polypeptide construct of any one of claims 12-14, wherein the amino acid sequence of the third polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the third polypeptide.

16. The polypeptide construct of any one of claims 12-15, wherein the amino acid sequence of the fourth polypeptide comprises a set of two non-natural amino acids, wherein the non-natural amino acids are the same or different, wherein each of the non-natural amino acids includes a moiety, wherein the moieties are capable of undergoing a reaction to form an intrapolypeptide covalent cross-link with each other, wherein when formed the covalent cross-link is internal to the fourth polypeptide.

17. The polypeptide construct of claim 15 or 16, wherein each set of non-natural amino acids are capable of undergoing a Diels-Alder reaction, a Huisgen reaction, or an olefin metathesis reaction.

18. The polypeptide construct of claim 15 or 16, wherein one non-natural amino acid within a set is Xaa^{A1} and the other non-natural amino acid within the set is Xaa^{B1},
wherein 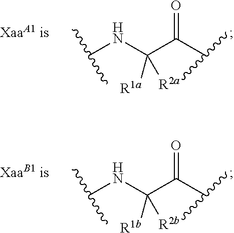
R^{1a} and R^{1b} are independently H, alkyl, alkenyl, alkynyl, arylalkyl, cycloalkylalkyl, heteroarylalkyl, or heterocyclylalkyl;
R^{2a} and R^{2b} are (i) independently alkenyl, alkynyl, azido, amino, carboxylic acid, or sulfide or (ii) taken together to form alkylene, alkenylene, alkynylene, or [R^{3a}—X—R^{3b}]ₙ, each of which is substituted with 0-6 R⁴;
each R^{3a} and R^{3b} are independently alkylene, alkenylene or alkynylene;
each R⁴ is independently halo, alkyl, OR⁵, N(R⁵)₂, SR⁵, SOR⁵, SO₂R⁵, CO₂R⁵, R⁵;
each X is independently O, S, SO, SO₂, CO, CO₂, CONR⁵ or 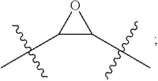 [SMILES: *C1OC1*]
each R⁵ is independently H or alkyl; and
n is an integer 1-4.

19. The polypeptide construct of claim 15 or 16, wherein the non-natural amino acids are capable of forming together a thioether, ether, amide, amine, triazole, or carbon-carbon double bond or a Diels-Alder adduct after reaction.

20. The polypeptide construct of claim 15 or 16, wherein the non-natural amino acids are independently selected from (S)-2-(4′-pentenyl)alanine (S5), (R)-2-(2′-propenyl)alanine (R3), and (R)-2-(7′-octenyl)alanine (R8).

21. The polypeptide construct of any one of claims 15-20, wherein the non-natural amino acids have undergone reaction to form the intrapolypeptide covalent cross-link with each other.

22. The polypeptide construct of any one of claims 15-21, wherein the interpolypeptide covalent linkage between the third polypeptide and the fourth polypeptide is a maleimide-thiol adduct.

23. The polypeptide construct of any one of claims 15-22, wherein the second polypeptide and the fourth polypeptide are linked through an interpolypeptide covalent linkage.

24. The polypeptide construct of claim 23, wherein the interpolypeptide linkage is between the C-terminal amino acid of the second polypeptide and the C-terminal amino acid of the fourth polypeptide.

25. The polypeptide construct of claim 23 or 24, wherein the interpolypeptide covalent linkage between the second polypeptide and the fourth polypeptide is a maleimide-thiol adduct.

26. The polypeptide construct of any one of claims 1-25, wherein the N-terminus or the C-terminus of the first, second, third, or fourth polypeptide is capped.

27. The polypeptide construct of claim 26, wherein the N-terminus cap is acetyl or the C-terminus cap is —NH₂.

28. The polypeptide construct of any one of claims 1-27, wherein the polypeptide construct binds to duplex DNA comprising the sequence of 5′-CANNTG-3′, wherein each N is independently any one of A, C, G, or T.

29. A polypeptide construct comprising:
(a) a first polypeptide comprising an amino acid sequence derived from a basic helix as listed in Table 2; and
(b) a second polypeptide comprising an amino acid sequence derived from a helix as listed in Table 2;
wherein the first polypeptide and the second polypeptide are linked through an interpolypeptide covalent linkage.

30. A polypeptide comprising the sequence of any one of
(Ac-RAQILCKATEYIQS₅MRRS₅Nβ)₂K-NH₂
(Ac-RAQILCKATEYIQYMRRKNβ)₂K-NH₂
(Ac-RAS₅ILCS₅ATEYIQYMRRKNβ)₂K-NH₂
Ac-HNALERKRRDHIKDSFHKLRDSVP
Ac-KRAHHNALERKRRDHIKDSFHK(GlyMal)LRDSVP-NH₂
Ac-KRAHHNALERKRRDHIKDSFHKLRDSVP
Ac-KRAHHNALERKRRDHIKDSFSK(GlyMal)LRS₅SVP-NH₂
Ac-KRAHHNALERKRRDHIKDSFS₅KLRS₅SVP
Ac-KRAHHNALERS₅RRDS₅IKDSFHK(GlyMal)LRDSVP-NH₂
Ac-KRAHHNALERS₅RRDS₅IKDSFHKLRDSVP
Ac-KRAHHNS₅LERS₅RRDHIKDSFHK(GlyMal)LRDSVP-NH₂
Ac-KRAHHNS₅LERS₅RRDHIKDSFHKLRDSVP
Ac-KRA_{ib}HHNALERS₅RRDS₅IKDSFHKLRDSVP
Ac-KRA_{ib}HHNS₅LERS₅RRDHIKDSFHKLRDSVP
Ac-KRS₅HHNS₅LER(D-lysine)RRDHIKDSFHKLRDSVP
Ac-KRS₅HHNS₅LERA_{ib}RRDHIKDSFHKLRDSVP
Ac-KRS₅HHNS₅LERKRRDHIKDSFHK(GlyMal)LRDSVP-NH₂
Ac-KRS₅HHNS₅LERKRRDHIKDSFHKLRDSVP
Ac-KVC(StBu)ILKKATAYILS₅VQAS₅K(GlyMal)-NH₂
Ac-KVC(StBu)ILKKATAYILSVQAEK(GlyMal)-NH₂
Ac-KVCILKKATAYILSVQAS₅K(N3)-NH₂
Ac-KVCILKKATAYILSVQAEK(N3)-NH₂
Ac-KVS₅ILC(StBu)S₅ATAYILSVQAEK(GlyMal)-NH₂
Ac-KVS₅ILCS₅ATAYILSVQAEK(N3)-NH₂
Ac-KVVILC(StBu)KATAYILS₅VQAS₅K(GlyMal)-NH₂
Ac-KVVILC(StBu)KATAYILSVQAEK(GlyMal)-NH₂
Ac-KVVILCKATAYILS₅VQAS₅K(N3)-NH₂
Ac-KVVILCKATAYILSVQAEK(N3)-NH₂
Ac-RAC(StBu)ILDKATEYIQS₅MRRS₅C-NH₂
Ac-RAC(StBu)ILDKATEYIQYMRRKC-NH₂
Ac-RACILDKATEYIQS₅MRRS₅C(StBu)-NH₂
Ac-RACILDKATEYIQYMRRKC(StBu)-NH₂
Ac-RAQILC(StBu)KATEYIQS₅MRRS₅C-NH₂
Ac-RAQILC(StBu)KATEYIQS₅MRRS₅NβC-NH₂
Ac-RAQILC(StBu)KATEYIQYMRRKC-NH₂
Ac-RAQILC(StBu)KATEYIQYMRRKNβC-NH₂
Ac-RAQILCKATEYIQS₅MRRS₅C(StBu)-NH₂
Ac-RAQILCKATEYIQYMRRKC(StBu)-NH₂
Ac-RAS₅ILC(StBu)S₅ATEYIQYMRRKC-NH₂
Ac-RAS₅ILC(StBu)S₅ATEYIQYMRRKNβC-NH₂
Ac-RAS₅ILCS₅ATEYIQYMRRKC(StBu)-NH₂
Ac-SRA_{ib}QILCQATEYIQS₅N_{L}RRS₅N
Ac-SRAQILC(StBu)KATEYIQS₅N_{L}RRS₅NβC-NH₂
Ac-SRAQILCKATEYIQS₅N_{L}RRS₅N
Ac-SRAQILCKATEYIQYN_{L}R
Ac-SRAQILCKATEYIQYN_{L}RRKN
Ac-SRAQILCQATEYIQS₅N_{L}RRS₅N
Ac-SRAS₅ILC(StBu)S₅ATEYIQYN_{L}RRKNβC-NH₂
Ac-SRAS₅ILCSATEYIQYN_{L}RRKN
Ac-WβADKRAHHNALERKRRDHIKDSFHK(GlyMal)LRDSV-NH₂
Ac-WβADKRAHHNALERKRRDHIKDSFHK(N3)LRDSV-NH₂
Ac-WβADKRAHHNALERKRRDHIKDSFHSLK(GlyMal)DSV-NH₂
Ac-WβADKRAHHNALERKRRDHIKDSFHSLK(N3)DSV-NH₂
Ac-WβADKRAHHNALERKRRDHIKDSFS₅K(GlyMal)LRS₅SV-NH₂
Ac-WβADKRAHHNALERKRRDHIKDSFS₅K(N3)LRS₅SV-NH₂
Ac-WβADKRAHHNALERKRRDHIKDSFS₅SLK(GlyMal)S₅SV-NH₂
Ac-WβADKRAHHNALERKRRDHIKDSFS₅SLK(N3)S₅SV-NH₂
Ac-WβADKRAHHNALERS₅RRDS₅IKDSFHK(GlyMal)LRDSV-NH₂
Ac-WβADKRAHHNALERS₅RRDS₅IKDSFHK(N3)LRDSV-NH₂
Ac-WβADKRAHHNALERS₅RRDS₅IKDSFHSLK(GlyMal)DSV-NH₂
Ac-WβADKRAHHNALERS₅RRDS₅IKDSFHSLK(N3)DSV-NH₂
Ac-WβADKRAHHNS₅LERS₅RRDHIKDSFHK(GlyMal)LRDSV-NH₂
Ac-WβADKRAHHNS₅LERS₅RRDHIKDSFHK(N3)LRDSV-NH₂
Ac-WβADKRAHHNS₅LERS₅RRDHIKDSFHSLK(GlyMal)DSV-NH₂
Ac-WβADKRAHHNS₅LERS₅RRDHIKDSFHSLK(N3)DSV-NH₂
Ac-WβADKRS₅HHNS₅LERKRRDHIKDSFHK(GlyMal)LRDSV-NH₂
Ac-WβADKRSHHNS₅LERKRRDHIKDSFHK(N3)LRDSV-NH₂
Ac-WβADKRSHHNS₅LERKRRDHIKDSFHSLK(GlyMal)DSV-NH₂
Ac-WβADKRSHHNS₅LERKRRDHIKDSFHSLK(N3)DSV-NH₂
Ac-WβKRAHHNALERKRRDHIKDSFHK(GlyMal)LRDSV-NH₂
Ac-WβKRAHHNALERKRRDHIKDSFS₅K(GlyMal)LRS₅SV-NH₂
Ac-WβKRAHHNALERS₅RRDS₅IKDSFHK(GlyMal)LRDSV-NH₂
Ac-WβKRAHHNS₅LERS₅RRDHIKDSFHK(GlyMal)LRDSV-NH₂
Ac-WβKRSHHNS₅LERKRRDHIKDSFHK(GlyMal)LRDSV-NH₂
Ac-WβNVKRRTHNS₅LERS₅RRNELKRSFFALK(GlyMal)DQI-NH₂
Ac-WβNVKRRTHNS₅LERS₅RRNELKRSFFALK(N3)DQI-NH₂
Ac-WβNVKRRTHNS₅LERS₅RRNELKRSFFK(GlyMal)LRDQI-NH₂
Ac-WβNVKRRTHNS₅LERS₅RRNELKRSFFK(N3)LRDQI-NH₂
Ac-WβNVKRRTHNVLERQRRNELKRSFFALK(GlyMal)DQI-NH₂
Ac-WβNVKRRTHNVLERQRRNELKRSFFALK(N3)DQI-NH₂
Ac-WβNVKRRTHNVLERQRRNELKRSFFK(GlyMal)LRDQI-NH₂
Ac-WβNVKRRTHNVLERQRRNELKRSFFK(N3)LRDQI-NH₂
Ac-WβNVKRRTHNVLERQRRNELKRSFSALK(GlyMal)S₅QI-NH₂
Ac-WβNVKRRTHNVLERQRRNELKRSFS₅ALK(N3)S₅QI-NH₂
Ac-WβNVKRRTHNVLERQRRNELKRSFS₅K(GlyMal)LRSQI-NH₂
Ac-WβNVKRRTHNVLERQRRNELKRSFS₅K(N3)LRS₅QI-NH₂
Ac-WβNVKRRTHNVLERS₅RRNS₅LKRSFFALK(GlyMal)DQI-NH₂
Ac-WβNVKRRTHNVLERS₅RRNS₅LKRSFFALK(N3)DQI-NH₂
Ac-WβNVKRRTHNVLERS₅RRNS₅LKRSFFK(GlyMal)LRDQI-NH₂
Ac-WβNVKRRTHNVLERS₅RRNS₅LKRSFFK(N3)LRDQI-NH₂
Ac-WβNVKRS₅THNS₅LERQRRNELKRSFFALK(GlyMal)DQI-NH₂
Ac-WβNVKRS₅THNS₅LERQRRNELKRSFFALK(N3)DQI-NH₂
Ac-WβNVKRS₅THNS₅LERQRRNELKRSFFK(GlyMal)LRDQI-NH₂
Ac-WβNVKRS₅THNS₅LERQRRNELKRSFFK(N3)LRDQI-NH₂
Ac-KRAHHNALERKRRDHIKDSFHKLRDSVP-NH₂
Ac-SRAQILCKATEYIQYNRRKN-NH₂
Ac-PRFQSAADKRAHHNALERKRRDHIKDSFHKLRDSVP-NH₂
Ac-QSAADKRAHHNALERKRRDHIKDSFHKLRDSVP-NH₂
Ac-IEVESDADKRAHHNALERKRRDHIKDSFHKLRDSVP-NH₂
Ac-PRSSDTEENVKRAHHNALERKRRDHIKDSFHKLRDSVP-NH₂
Ac-TEENVKRAHHNALERKRRDHIKDSFHKLRDSVP-NH₂
Ac-KSKKNNSSKRAHHNALERKRRDHIKDSFHKLRDSVP-NH₂
Ac-KNNSSKRAHHNALERKRRDHIKDSFHKLRDSVP-NH₂
Ac-PRFQSAADKRS₅HHNS₅LERKRRDHIKDSFHKLRDSVP-NH₂
Ac-PRFQSAS₅DKRS₅HHNALERKRRDHIKDSFHKLRDSVP-NH₂
Ac-PRS₅FQSS₅DKRAHHNALERKRRDHIKDSFHKLRDSVP-NH₂
Ac-SRAQILCKATEYIQYN_{L}RRKN-NH₂
Ac-SRAQILCKATEYIQYNRRKNHTHQQDIDDLK-NH₂
Ac-SRAQILCKATEYIQYNRRKNHTLISE-NH₂
Ac-SRAQILCKATEYIQYN_{L}RRKLHTHE-NH₂
Ac-SRA_{ib}QILCQATEYIQS₅N_{L}RRS₅LHTHE-NH₂
Ac-KRAHHNALERKRRDHIKDSFHKLRDSVP-NH₂
Ac-KRS₅HHNS₅LERKRRDHIKDSFHKLRDSVP-NH₂
Ac-KRS₅HANS₅LERKRLDHIKDSFHKLRDSVP-NH₂
Ac-KRS₅HANS₅LERKRTDHIKDSFHKLRDSVP-NH₂
Ac-KKSHSNS₅LARKRRDHIKDSFHKLRDSVP-NH₂
Ac-KRSHHNS₅LNRKRRDHIKDSFHKLRDSVP-NH₂
Ac-PRFQSA(S5)DKR(S5)HHNALERKRRDHIKDSFHK
(GlyMal)LRDSVP-NH₂
Ac-SR(Aib)QILCQATEYIQ(S5)(Nle)RR(S5)LHTHE-NH₂
Ac-PRFQSA(S5)DKR(S5)HHNALERKRRDHIKDSFHK
(GlyMal)LRDSVP-NH₂
Ac-SRAQILCKATEYIQYLR(S5)KIH(S5)LE-NH₂
wherein
Ac is acetyl;
A_{ib} is 2-aminoisobutyric acid;
N_{L} is norleucine;
β prior to an amino acid represents that amino acid is a p amino acid;
GlyMal is glycylmaleimide;
StBu is tert-butylsulfenyl;
K(N3) is azidolysine; and
S₅ is (S)-2-(4′-pentenyl)alanine.

31. A pharmaceutical composition comprising a therapeutically effective amount of the polypeptide construct of any one of claims 1-29 or the polypeptide of claim 30 and a pharmaceutically acceptable excipient.

32. A method of treating disease in a subject in need thereof comprising administering to the subject a therapeutically effective amount of the polypeptide construct of any one of claims 1-29, the polypeptide of claim 30, or the pharmaceutical composition of claim 31.


SEQUENCE LISTING (536 sequences)

SEQ ID NO: 1 - protein, 27 aa, Homo sapiens
KRAHHNALERKRRDHIKDSFHLRDSVP

SEQ ID NO: 2 - protein, 35 aa, Homo sapiens
PRFQSAADKRAHHNALERKRRDHIKDSFHLRDSVP

SEQ ID NO: 3 - protein, 25 aa, Homo sapiens
KRHHNLERKRRDHIKDSFHLRDSVP

SEQ ID NO: 4 - protein, 24 aa, Homo sapiens
KRHHNLERRRDHIKDSFHLRDSVP

SEQ ID NO: 5 - protein, 33 aa, Homo sapiens
PRFQSADKRHHNALERKRRDHIKDSFHLRDSVP

SEQ ID NO: 6 - protein, 36 aa, Homo sapiens
QKTVKPIPAEGIKSNPSKRHRDRLNTELDRLASLLP

SEQ ID NO: 7 - protein, 28 aa, Homo sapiens
MYRSTKGASKARRDQINAEIRNLKELLP

SEQ ID NO: 8 - protein, 36 aa, Homo sapiens
PLQKQRPAVGAEKSNPSKRHRDRLNAELDHLASLLP

SEQ ID NO: 9 - protein, 36 aa, Homo sapiens
SGPCLQAQRKEKSRNAARSRRGKENLEFFELAKLLP

SEQ ID NO: 10 - protein, 36 aa, Homo sapiens
TYQNLQALRKEKSRDAARSRRGKENFEFYELAKLLP

SEQ ID NO: 11 - protein, 28 aa, Homo sapiens
MKEKSKNAARTRREKENSEFYELAKLLP

SEQ ID NO: 12 - protein, 28 aa, Homo sapiens
MKEKSKNAAKTRREKENGEFYELAKLLP

SEQ ID NO: 13 - protein, 36 aa, Homo sapiens
KKRSSSERRKEKSRDAARCRRSKETEVFYELAHELP

SEQ ID NO: 14 - protein, 36 aa, Homo sapiens
KKKISSERRKEKSRDAARSRRSKESEVFYELAHQLP

SEQ ID NO: 15 - protein, 36 aa, Homo sapiens
RARSTTELRKEKSRDAARSRRSQETEVLYQLAHTLP

SEQ ID NO: 16 - protein, 36 aa, Homo sapiens
SHKRKGSPCDTLASSTEKRRREQENKYLEELAELLS

SEQ ID NO: 17 - protein, 36 aa, Homo sapiens
RKECPDQLGPSPKRNTEKRNREQENKYIEELAELIF

SEQ ID NO: 18 - protein, 36 aa, Homo sapiens
RKLPCDTPGQGLTCSGEKRRREQESKYIEELAELIS

SEQ ID NO: 19 - protein, 36 aa, Homo sapiens
TVAEGPSSCLRRNVISERERRKRMSLSCERLRALLP

SEQ ID NO: 20 - protein, 36 aa, Homo sapiens
GPQGGRSQRRERHNRMERDRRRRIRICCDELNLLVP

SEQ ID NO: 21 - protein, 36 aa, Homo sapiens
SEFEKNKKISLLHSSKEKLRRERIKYCCEQLRTLLP

SEQ ID NO: 22 - protein, 36 aa, Homo sapiens
DKLKERKRTPVSHKVIEKRRRDRINRCLNELGKTVP

SEQ ID NO: 23 - protein, 36 aa, Homo sapiens
PSSSQMQARKKHRGIIEKRRRDRINSSLSELRRLVP

SEQ ID NO: 24 - protein, 36 aa, Homo sapiens
TTSSQILARKRRRGIIEKRRRDRINNSLSELRRLVP

SEQ ID NO: 25 - protein, 36 aa, Homo sapiens
TTTSQIMARKKRRGIIEKRRRDRINNSLSELRRLVP

SEQ ID NO: 26 - protein, 36 aa, Homo sapiens
RSEDSKETYKLPHRLIEKKRRDRINECIAQLKDLLP

SEQ ID NO: 27 - protein, 36 aa, Homo sapiens
KRDDTKDTYKLPHRLIEKKRRDRINECIAQLKDLLP

SEQ ID NO: 28 - protein, 36 aa, Homo sapiens
DRAENRDGPKMLKPLVEKRRRDRINRSLEELRLLLL

SEQ ID NO: 29 - protein, 36 aa, Homo sapiens
DGWETRGDRKARKPLVEKKRRARINESLQELRLLLA

SEQ ID NO: 30 - protein, 36 aa, Homo sapiens
ELLSPKEKNRLRKPVVEKMRRDRINSSIEQLKLLLE

SEQ ID NO: 31 - protein, 21 aa, Homo sapiens
MEKKRRARINVSLEQLKSLLE

SEQ ID NO: 32 - protein, 36 aa, Homo sapiens
RAGDAAELRKSLKPLLEKRRRARINQSLSQLKGLIL

SEQ ID NO: 33 - protein, 36 aa, Homo sapiens
KPKTASEHRKSSKPIMEKRRRARINESLSQLKTLIL

SEQ ID NO: 34 - protein, 36 aa, Homo sapiens
KPRSAAEHRKSSKPVMEKRRRARINESLAQLKTLIL

SEQ ID NO: 35 - protein, 36 aa, Homo sapiens
EEDDKDKAKRVSRNKSEKKRRDQFNVLIKELGSMLP

SEQ ID NO: 36 - protein, 36 aa, Homo sapiens
DEDEKDRAKRASRNKSEKKRRDQFNVLIKELSSMLP

SEQ ID NO: 37 - protein, 36 aa, Homo sapiens
SSADKERLARENHSEIERRRRNKMTAYITELSDMVP

SEQ ID NO: 38 - protein, 36 aa, Homo sapiens
EHQVKMKAFREAHSQTEKRRRDKMNNLIEELSAMIP

SEQ ID NO: 39 - protein, 36 aa, Homo sapiens
TKKQHRKKNRETHNAVERHRKKKINAGINRIGELIP

SEQ ID NO: 40 - protein, 36 aa, Homo sapiens
PRTTRDEKRRAQHNEVERRRRDKINNWIVQLSKIIP

SEQ ID NO: 41 - protein, 36 aa, Homo sapiens
TRTPRDERRRAQHNEVERRRRDKINNWIVQLSKIIP

SEQ ID NO: 42 - protein, 36 aa, Homo sapiens
RALAKERQKKDNHNLIERRRRFNINDRIKELGMLIP

SEQ ID NO: 43 - protein, 36 aa, Homo sapiens
RALAKERQKKDNHNLIERRRRYNINYRIKELGTLIP

SEQ ID NO: 44 - protein, 36 aa, Homo sapiens
RALAKERQKKDNHNLIERRRRFNINDRIKELGTLIP

SEQ ID NO: 45 - protein, 36 aa, Homo sapiens
KALLKERQKKDNHNLIERRRRFNINDRIKELGTLIP

SEQ ID NO: 46 - protein, 36 aa, Homo sapiens
ASAQSRGEKRTAHNAIEKRYRSSINDKIIELKDLVV

SEQ ID NO: 47 - protein, 36 aa, Homo sapiens
LEPPKEGERRTTHNIIEKRYRSSINDKIIELKDLVM

SEQ ID NO: 48 - protein, 36 aa, Homo sapiens
KKRPGGIGTREVHNKLEKNRRAHLKECFETLKRNIP

SEQ ID NO: 49 - protein, 36 aa, Homo sapiens
QAPGAQDSGRSVHNELEKRRRAQLKRCLERLKQQMP

SEQ ID NO: 50 - protein, 36 aa, Homo sapiens
GLVRKAPNNRSSHNELEKHRRAKLRLYLEQLKQLVP

SEQ ID NO: 51 - protein, 36 aa, Homo sapiens
KSKKNNSSSRSTHNEMEKNRRAHLRLCLEKLKGLVP

SEQ ID NO: 52 - protein, 36 aa, Homo sapiens
GSSNTSTANRSTHNELEKNRRAHLRLCLERLKVLIP

SEQ ID NO: 53 - protein, 36 aa, Homo sapiens
YKESYKDRRRRAHTQAEQKRRDAIKRGYDDLQTIVP

SEQ ID NO: 54 - protein, 36 aa, Homo sapiens
KNVAALKNRQMKHISAEQKRRFNIKMCFDMLNSLIS

SEQ ID NO: 55 - protein, 36 aa, Homo sapiens
PDSNKTENRRITHISAEQKRRFNIKLGFDTLHGLVS

SEQ ID NO: 56 - protein, 36 aa, Homo sapiens
ENLQLVLERRRVANAKERERIKNLNRGFARLKALVP

SEQ ID NO: 57 - protein, 36 aa, Homo sapiens
PRFQSAADKRAHHNALERKRRDHIKDSFHSLRDSVP

SEQ ID NO: 58 - protein, 36 aa, Homo sapiens
SSDTEDVTKRKNHNFLERKRRNDLRSRFLALRDQVP

SEQ ID NO: 59 - protein, 36 aa, Homo sapiens
SSDTEENVKRRTHNVLERQRRNELKRSFFALRDQIP

SEQ ID NO: 60 - protein, 36 aa, Homo sapiens
NSDSEDSERRRNHNILERQRRNDLRSSFLTLRDHVP

SEQ ID NO: 61 - protein, 36 aa, Homo sapiens
QKAEREKERRVANNARERLRVRDINEAFKELGRMCQ

SEQ ID NO: 62 - protein, 36 aa, Homo sapiens
QKIEREKERRMANNARERLRVRDINEAFKELGRMCQ

SEQ ID NO: 63 - protein, 36 aa, Homo sapiens
QKAEREKERRMANNARERLRVRDINEAFKELGRMVQ

SEQ ID NO: 64 - protein, 36 aa, Homo sapiens
SLPQQQPAAVARRNERERNRVKLVNLGFATLREHVP

SEQ ID NO: 65 - protein, 36 aa, Homo sapiens
AETGGGAAAVARRNERERNRVKLVNLGFQALRQHVP

SEQ ID NO: 66 - protein, 36 aa, Homo sapiens
CEYSYGPAFTRKRNERERQRVKCVNEGYAQLRHHLP

SEQ ID NO: 67 - protein, 36 aa, Homo sapiens
LDSAFEPAFLRKRNERERQRVRCVNEGYARLRDHLP

SEQ ID NO: 68 - protein, 36 aa, Homo sapiens
YEYPFEPAFIQKRNERERQRVKCVNEGYARLRGHLP

SEQ ID NO: 69 - protein, 36 aa, Homo sapiens
QRDQERRIRREIANSNERRRMQSINAGFQSLKTLIP

SEQ ID NO: 70 - protein, 36 aa, Homo sapiens
RSSRLGSGQRQSASEREKLRMRTLARALHELRRFLP

SEQ ID NO: 71 - protein, 36 aa, Homo sapiens
ARTGPAGGQRQSASEREKLRMRTLARALHELRRFLP

SEQ ID NO: 72 - protein, 36 aa, Homo sapiens
TKVRMSVQRRRKASEREKLRMRTLADALHTLRNYLP

SEQ ID NO: 73 - protein, 36 aa, Homo sapiens
AKEEQQQQLRRKINSRERKRMQDLNLAMDALREVIL

SEQ ID NO: 74 - protein, 36 aa, Homo sapiens
MTEPELQQLRLKINSRERKRMHDLNIAMDGLREVMP

SEQ ID NO: 75 - protein, 36 aa, Homo sapiens
LSEQDLQQLRLKINGRERKRMHDLNLAMDGLREVMP

SEQ ID NO: 76 - protein, 36 aa, Homo sapiens
KKSKEQKALRLNINARERRRMHDLNDALDELRAVIP

SEQ ID NO: 77 - protein, 36 aa, Homo sapiens
RRPREQRSLRLSINARERRRMHDLNDALDGLRAVIP

SEQ ID NO: 78 - protein, 36 aa, Homo sapiens
GRRDSSIQRRLESNERERQRMHKLNNAFQALREVIP

SEQ ID NO: 79 - protein, 36 aa, Homo sapiens
QVNGVQKQRRLAANARERRRMHGLNHAFDQLRNVIP

SEQ ID NO: 80 - protein, 35 aa, Homo sapiens
RLESAARRRLAANARERRRMQGLNTAFDRLRRVVP

SEQ ID NO: 81 - protein, 36 aa, Homo sapiens
EIKALQQTRRLLANARERTRVHTISAAFEALRKQVP

SEQ ID NO: 82 - protein, 36 aa, Homo sapiens
TVQRIKKTRRLKANNRERNRMHNLNAALDALREVLP

SEQ ID NO: 83 - protein, 36 aa, Homo sapiens
LLHSLRRSRRVKANDRERNRMHNLNAALDALRSVLP

SEQ ID NO: 84 - protein, 36 aa, Homo sapiens
ALSKQRRSRRKKANDRERNRMHNLNSALDALRGVLP

SEQ ID NO: 85 - protein, 36 aa, Homo sapiens
ARLERFRARRVKANARERTRMHGLNDALDNLRRVMP

SEQ ID NO: 86 - protein, 36 aa, Homo sapiens
ARLERFKLRRMKANARERNRMHGLNAALDNLRKVVP

SEQ ID NO: 87 - protein, 36 aa, Homo sapiens
ARLERSKLRRQKANARERNRMHDLNAALDNLRKVVP

SEQ ID NO: 88 - protein, 36 aa, Homo sapiens
LRLERVKFRRQEANARERNRMHGLNDALDNLRKVVP

SEQ ID NO: 89 - protein, 36 aa, Homo sapiens
KRKSTTMDRRKAATMRERRRLKKVNQAFETLKRCTT

SEQ ID NO: 90 - protein, 36 aa, Homo sapiens
KRKTTNADRRKAATMRERRRLSKVNEAFETLKRCTS

SEQ ID NO: 91 - protein, 36 aa, Homo sapiens
KRKSAPTDRRKAATLRERRRLKKINEAFEALKRRTV

SEQ ID NO: 92 - protein, 36 aa, Homo sapiens
KRKSVSVDRRRAATLREKRRLKKVNEAFEALKRSTL

SEQ ID NO: 93 - protein, 36 aa, Homo sapiens
ARPVRSKARRMAANVRERKRILDYNEAFNALRRALR

SEQ ID NO: 94 - protein, 36 aa, Homo sapiens
RKRVITYAQRQAANIRERKRMFNLNEAFDQLRRKVP

SEQ ID NO: 95 - protein, 36 aa, Homo sapiens
RSEAELQQLRQAANVRERRRMQSINDAFEGLRSHIP

SEQ ID NO: 96 - protein, 36 aa, Homo sapiens
RRRRATAKYRTAHATRERIRVEAFNLAFAELRKLLP

SEQ ID NO: 97 - protein, 36 aa, Homo sapiens
RRRRATAKYRSAHATRERIRVEAFNLAFAELRKLLP

SEQ ID NO: 98 - protein, 30 aa, Homo sapiens
MTRKIFTNTRERWRQQNVNSAFAKLRKLIP

SEQ ID NO: 99 - protein, 36 aa, Homo sapiens
GHQPQKVARRVFTNSRERWRQQNVNGAFAELRKLLP

SEQ ID NO: 100 - protein, 36 aa, Homo sapiens
DGPHTKVVRRIFTNSRERWRQQNVNGAFAELRKLIP

SEQ ID NO: 101 - protein, 36 aa, Homo sapiens
ALGGRLGRRKGSGPKKERRRTESINSAFAELRECIP

SEQ ID NO: 102 - protein, 36 aa, Homo sapiens
LGGPRPVKRRGTANRKERRRTQSINSAFAELRECIP

SEQ ID NO: 103 - protein, 36 aa, Homo sapiens
GLALGRSEASPENAARERSRVRTLRQAFLALQAALP

SEQ ID NO: 104 - protein, 36 aa, Homo sapiens
GSRSGSGRPAAANAARERSRVQTLRHAFLELQRTLP

SEQ ID NO: 105 - protein, 36 aa, Homo sapiens
SAAECKQSQRNAANARERARMRVLSKAFSRLKTSLP

SEQ ID NO: 106 - protein, 36 aa, Homo sapiens
VSQEGKQVQRNAANARERARMRVLSKAFSRLKTTLP

SEQ ID NO: 107 - protein, 36 aa, Homo sapiens
GRPGREPRQRHTANARERDRTNSVNTAFTALRTLIP

SEQ ID NO: 108 - protein, 36 aa, Homo sapiens
AGPVVVVRQRQAANARERDRTQSVNTAFTALRTLIP

SEQ ID NO: 109 - protein, 36 aa, Homo sapiens
QSYEELQTQRVMANVRERQRTQSLNEAFAALRKIIP

SEQ ID NO: 110 - protein, 36 aa, Homo sapiens
QSFEELQSQRILANVRERQRTQSLNEAFAALRKIIP

SEQ ID NO: 111 - protein, 18 aa, Homo sapiens
SRAQILKATEYIQYRRKN

SEQ ID NO: 112 - protein, 29 aa, Homo sapiens
SRAQILKATEYIQYRRKNHTHQQDIDDLK

SEQ ID NO: 113 - protein, 16 aa, Homo sapiens
SRAQILKATEYIQRRN

SEQ ID NO: 114 - protein, 15 aa, Homo sapiens
SRQILQATEYIQRRN

SEQ ID NO: 115 - protein, 19 aa, Homo sapiens
SRQILQATEYIQRRLHTHE

SEQ ID NO: 116 - protein, 31 aa, Homo sapiens
DKLSVLRLSVSYLRAKSFFDVALKSSPTERN

SEQ ID NO: 117 - protein, 31 aa, Homo sapiens
SYLHIMSLACIYTRKGVFFAGGTPLAGPTGL

SEQ ID NO: 118 - protein, 31 aa, Homo sapiens
DKLSVLRLSVSYLRVKSFFQVVQEQSSRQPA

SEQ ID NO: 119 - protein, 31 aa, Homo sapiens
DKASIVRLSVTYLRLRRFAALGAPPWGLRAA

SEQ ID NO: 120 - protein, 31 aa, Homo sapiens
DKASIIRLTISYLKMRDFANQGDPPWNLRME

SEQ ID NO: 121 - protein, 31 aa, Homo sapiens
DKASIIRLTTSYLKMRVVFPEGLGEAWGHSS

SEQ ID NO: 122 - protein, 31 aa, Homo sapiens
DKASIIRLTTSYLKMRAVFPEGLGDAWGQPS

SEQ ID NO: 123 - protein, 31 aa, Homo sapiens
DKASIMRLAISFLRTHKLLSSVCSENESEAE

SEQ ID NO: 124 - protein, 31 aa, Homo sapiens
DKASVMRLTISYLRVRKLLDAGDLDIEDDMK

SEQ ID NO: 125 - protein, 31 aa, Homo sapiens
DKASIMRLTISYLRMHRLCAAGEWNQVGAGG

SEQ ID NO: 126 - protein, 31 aa, Homo sapiens
DKCKILKKTVDQIQLMKRMEQEKSTTDDDVQ

SEQ ID NO: 127 - protein, 31 aa, Homo sapiens
DKCAILKETVKQIRQIKEQEKAAAANIDEVQ

SEQ ID NO: 128 - protein, 31 aa, Homo sapiens
DKCAILKETVRQIRQIKEQGKTISNDDDVQK

SEQ ID NO: 129 - protein, 31 aa, Homo sapiens
DMASVLEMSVQFLRLASALGPSQEQHAILAS

SEQ ID NO: 130 - protein, 31 aa, Homo sapiens
DKATTLQWTTAFLKYIQERHGDSLKKEFESV

SEQ ID NO: 131 - protein, 31 aa, Homo sapiens
DAASVLEATVDYVKYIREKISPAVMAQITEA

SEQ ID NO: 132 - protein, 31 aa, Homo sapiens
EKAEILEMTVQYLRALHSADFPRGREKAELL

SEQ ID NO: 133 - protein, 31 aa, Homo sapiens
EKAEVLQMTVDHLKMLHATGGTGFFDARALA

SEQ ID NO: 134 - protein, 31 aa, Homo sapiens
EKAEILQMTVDHLKMLHTAGGKGYFDAHALA

SEQ ID NO: 135 - protein, 31 aa, Homo sapiens
EKAEILQMTVDHLKMLQATGGKGYFDAHALA

SEQ ID NO: 136 - protein, 31 aa, Homo sapiens
EKAVVLELTLKHVKALTNLIDQQQQKIIALQ

SEQ ID NO: 137 - protein, 31 aa, Homo sapiens
EKAVVLELTLKHLKALTALTEQQHQKIIALQ

SEQ ID NO: 138 - protein, 31 aa, Homo sapiens
EKAEILEFAVGYLRERSRVEPPGVPRSPVQD

SEQ ID NO: 139 - protein, 31 aa, Homo sapiens
ENAEVLELTVRRVQGVLRGRAREREQLQAEA

SEQ ID NO: 140 - protein, 31 aa, Homo sapiens
EKADILEMAVSYLKHSKAFVAAAGPKSLHQD

SEQ ID NO: 141 - protein, 31 aa, Homo sapiens
EKADILELSVKYMRSLQNSLQGLWPVPRGAE

SEQ ID NO: 142 - protein, 31 aa, Homo sapiens
EKADVLEMTVRFLQELPASSWPTAAPLPCDS

SEQ ID NO: 143 - protein, 31 aa, Homo sapiens
EKADILEMTVKHLRNLQRAQMTAALSTDPSV

SEQ ID NO: 144 - protein, 31 aa, Homo sapiens
EKADILEMTVRHLRSLRRVQVTAALSADPAV

SEQ ID NO: 145 - protein, 31 aa, Homo sapiens
DKSTVLQKSIDFLRKHKEITAQSDASEIRQD

SEQ ID NO: 146 - protein, 31 aa, Homo sapiens
DKTTVLEKVIGFLQKHNEVSAQTEICDIQQD

SEQ ID NO: 147 - protein, 31 aa, Homo sapiens
DKLTILRMAVSHMKSLRGTGNTSTDGSYKPS

SEQ ID NO: 148 - protein, 31 aa, Homo sapiens
DKLTVLRMAVQHLRSLKGLTNSYVGSNYRPS

SEQ ID NO: 149 - protein, 31 aa, Homo sapiens
SKNMILDQAFKYITELKRQNDELLLNGGNNE

SEQ ID NO: 150 - protein, 31 aa, Homo sapiens
SKGGILSKACDYIQELRQSNHRLSEELQGLD

SEQ ID NO: 151 - protein, 31 aa, Homo sapiens
SKGGILSKACDYIRELRQTNQRMQETFKEAE

SEQ ID NO: 152 - protein, 31 aa, Homo sapiens
NKGTILKASVDYIRRMQKDLQKSRELENHSR

SEQ ID NO: 153 - protein, 31 aa, Homo sapiens
NKGTILKASVEYIKWLQKEQQRARELEHRQK

SEQ ID NO: 154 - protein, 31 aa, Homo sapiens
NKGTILKASVDYIRKLQREQQRAKELENRQK

SEQ ID NO: 155 - protein, 31 aa, Homo sapiens
NKGTILKASVDYIRKLQKEQQRSKDLESRQR

SEQ ID NO: 156 - protein, 31 aa, Homo sapiens
NKSAVLRKAIDYIRFLQHSNQKLKQENLSLR

SEQ ID NO: 157 - protein, 31 aa, Homo sapiens
HKSGVLRKAIDYIKYLQQVNHKLRQENMVLK

SEQ ID NO: 158 - protein, 31 aa, Homo sapiens
SNLSVLRTALRYIQSLKRKEKEYEHEMERLA

SEQ ID NO: 159 - protein, 31 aa, Homo sapiens
TTLSLLRRARMHIQKLEDQEQRARQLKERLR

SEQ ID NO: 160 - protein, 31 aa, Homo sapiens
TTLSLLKRAKVHIKKLEEQDRRALSIKEQLQ

SEQ ID NO: 161 - protein, 31 aa, Homo sapiens
TTLSLLTKAKLHIKKLEDCDRKAVHQIDQLQ

SEQ ID NO: 162 - protein, 31 aa, Homo sapiens
TTLGLLNKAKAHIKKLEEAERKSQHQLENLE

SEQ ID NO: 163 - protein, 31 aa, Homo sapiens
SKAIVLQKTIDYIQFLHKEKKKQEEEVSTLR

SEQ ID NO: 164 - protein, 31 aa, Homo sapiens
SHAITLQKTVEYITKLQQERGQMQEEARRLR

SEQ ID NO: 165 - protein, 31 aa, Homo sapiens
SKATTLQKTAEYILMLQQERAGLQEEAQQLR

SEQ ID NO: 166 - protein, 31 aa, Homo sapiens
SKVDILKGATEYIQVLSDLLEGAKDSKKQDP

SEQ ID NO: 167 - protein, 31 aa, Homo sapiens
SRAQILDKATEYIQYMRRKNHTHQQDIDDLK

SEQ ID NO: 168 - protein, 31 aa, Homo sapiens
PKVVILSKALEYLQALVGAEKRMATEKRQLR

SEQ ID NO: 169 - protein, 31 aa, Homo sapiens
PKVVILKKATAYILSVQAEEQKLISEEDLLR

SEQ ID NO: 170 - protein, 31 aa, Homo sapiens
AKVVILKKATEYVHSLQAEEHQLLLEKEKLQ

SEQ ID NO: 171 - protein, 31 aa, Homo sapiens
TKLLILHQAVSVILNLEQQVRERNLNPKAAC

SEQ ID NO: 172 - protein, 31 aa, Homo sapiens
TKLLILHQAVAVILSLEQQVRERNLNPKAAC

SEQ ID NO: 173 - protein, 31 aa, Homo sapiens; identical to SEQ ID NO: 172

SEQ ID NO: 174 - protein, 31 aa, Homo sapiens
SKVETLRSAVEYIRALQQLLDEHDAVSAAFQ

SEQ ID NO: 175 - protein, 31 aa, Homo sapiens
SKVETLRSAVEYIRALQRLLAEHDAVRNALA

SEQ ID NO: 176 - protein, 31 aa, Homo sapiens
SKVETLRAAIKYINYLQSLLYPDKAETKNNP

SEQ ID NO: 177 - protein, 31 aa, Homo sapiens
SKVETLRAAIDYIKHLQELLERQAWGLEGAA

SEQ ID NO: 178 - protein, 31 aa, Homo sapiens
SKVETLRAAIRYIKYLQELLSSAPDGSTPPA

SEQ ID NO: 179 - protein, 31 aa, Homo sapiens
SKAAILQQTAEYIFSLEQEKTRLLQQNTQLK

SEQ ID NO: 180 - protein, 31 aa, Homo sapiens
TKIETLRLAIRYIGHLSAVLGLSEESLQRRC

SEQ ID NO: 181 - protein, 31 aa, Homo sapiens
TKIETLRLAIRYIGHLSAVLGLSEESLQCRR

SEQ ID NO: 182 - protein, 31 aa, Homo sapiens
TKIQTLKYTIKYIGELTDLLNRGREPRAQSA

SEQ ID NO: 183 - protein, 31 aa, Homo sapiens
SKIATLLLARNYILLLGSSLQELRRALGEGA

SEQ ID NO: 184 - protein, 31 aa, Homo sapiens
SKIATLLLARNYILMLTNSLEEMKRLVSEIY

SEQ ID NO: 185 - protein, 31 aa, Homo sapiens
SKIATLLLARNYILMLTSSLEEMKRLVGEIY

SEQ ID NO: 186 - protein, 31 aa, Homo sapiens
SKIATLLLAKNYILMQAQALEEMRRLVAYLN

SEQ ID NO: 187 - protein, 31 aa, Homo sapiens
SKIATLLLAKNYILMQAQALDEMRRLVAFLN

SEQ ID NO: 188 - protein, 31 aa, Homo sapiens
SKIETLTLAKNYIKSLTATILTMSSSRLPGL

SEQ ID NO: 189 - protein, 31 aa, Homo sapiens
SKYETLQMAQIYINALSELLQTPSGGEQPPP

SEQ ID NO: 190 - protein, 31 aa, Homo sapiens
SKYETLQMALSYIMALTRILAEAERFGSERD

SEQ ID NO: 191 - protein, 31 aa, Homo sapiens
SKLAILRIACNYILSLARLADLDYSADHSNL

SEQ ID NO: 192 - protein, 31 aa, Homo sapiens
TKIETLRFAHNYIWALTETLRLADHCGGGGG

SEQ ID NO: 193 - protein, 31 aa, Homo sapiens
TKIETLRFAYNYIWALAETLRLADQGLPGGG

SEQ ID NO: 194 - protein, 31 aa, Homo sapiens
TKIETLRFAHNYIWALTQTLRIADHSLYALE

SEQ ID NO: 195 - protein, 31 aa, Homo sapiens
SKIETLRLARNYIWALSEVLETGQTPEGKGF

SEQ ID NO: 196 - protein, 31 aa, Homo sapiens
SKIETLRLAKNYIWALSEILRSGKSPDLVSF

SEQ ID NO: 197 - protein, 31 aa, Homo sapiens
SKIETLRLAKNYIWALSEILRSGKRPDLVSY

SEQ ID NO: 198 - protein, 31 aa, Homo sapiens
SKIETLRLAKNYIWALSEILRIGKRPDLLTF

SEQ ID NO: 199 - protein, 31 aa, Homo sapiens
PKVEILRNAIRYIESLQELLREQVENYYSLP

SEQ ID NO: 200 - protein, 31 aa, Homo sapiens
PKVEILRNAIRYIEGLQALLRDQDAAPPGAA

SEQ ID NO: 201 - protein, 31 aa, Homo sapiens
PKVEILRSAISYIERLQDLLHRLDQQEKMQE

SEQ ID NO: 202 - protein, 31 aa, Homo sapiens
PKVEILRSAIQYIERLQALLSSLNQEERDLR

SEQ ID NO: 203 - protein, 31 aa, Homo sapiens
SKIATLRRAIHRIAALSLVLRASPAPRGPCG

SEQ ID NO: 204 - protein, 29 aa, Homo sapiens
SRIETLRLAIVYISFMTELLESCEKKESG

SEQ ID NO: 205 - protein, 31 aa, Homo sapiens
SKVDTLRLAIGYINFLSELVQADLPLRGGGA

SEQ ID NO: 206 - protein, 22 aa, Homo sapiens
SKIEILRLAICYISYLNHVLDV

SEQ ID NO: 207 - protein, 22 aa, Homo sapiens
SKIEILRLAICYISYLNHVLDV

SEQ ID NO: 208 - protein, 31 aa, Homo sapiens
SKNETLRLAMRYINFLVKVLGEQSLQQTGVA

SEQ ID NO: 209 - protein, 31 aa, Homo sapiens
SKNEVLRLAMKYIGFLVRLLRDQAAALAAGP

SEQ ID NO: 210 - protein, 31 aa, Homo sapiens
SKNEILRLAMKYINFLAKLLNDQEEEGTQRA

SEQ ID NO: 211 - protein, 31 aa, Homo sapiens
SKIKTLRLATSYIAYLMDVLAKDAQSGDPEA

SEQ ID NO: 212 - protein, 31 aa, Homo sapiens
SKIKTLRLATSYIAYLMDLLAKDDQNGEAEA

SEQ ID NO: 213 - protein, 31 aa, Homo sapiens
SKLDVLVLAASYIAHLTRTLGHELPGPAWPP

SEQ ID NO: 214 - protein, 31 aa, Homo sapiens
SKLDVLLLATTYIAHLTRSLQDDAEAPADAG

SEQ ID NO: 215 - protein, 31 aa, Homo sapiens
SKLDTLRLASSYIAHLRQLLQEDRYENGYVH

SEQ ID NO: 216 - protein, 31 aa, Homo sapiens
SKLDTLRLASSYIAHLRQILANDKYENGYIH

SEQ ID NO: 217 - protein, 31 aa, Homo sapiens
SKIETLRLASSYISHLGNVLLAGEACGDGQP

SEQ ID NO: 218 - protein, 31 aa, Homo sapiens
SKIETVRLASSYIAHLANVLLLGDSADDGQP

SEQ ID NO: 219 - protein, 31 aa, Homo sapiens
SKIQTLKLAARYIDFLYQVLQSDELDSKMAS

SEQ ID NO: 220 - protein, 31 aa, Homo sapiens
SKIQTLKLAARYIDFLYQVLQSDEMDNKMTS

SEQ ID NO: 221 - protein, 53 aa, Homo sapiens
MYRSTKGASKARRDQINAEIRNLKELLPLAEADKVRLSYLHIMSLACIYTRKG

SEQ ID NO: 222 - protein, 54 aa, Homo sapiens
PAEGIKSNPSKRHRDRLNTELDRLASLLPFPQDVINKLDKLSVLRLSVSYLRAK

SEQ ID NO: 223 - protein, 54 aa, Homo sapiens
AVGAEKSNPSKRHRDRLNAELDHLASLLPFPPDIISKLDKLSVLRLSVSYLRVK

SEQ ID NO: 224 - protein, 54 aa, Homo sapiens
QRKEKSRNAARSRRGKENLEFFELAKLLPLPGAISSQLDKASIVRLSVTYLRLR

SEQ ID NO: 225 - protein, 54 aa, Homo sapiens
LRKEKSRDAARSRRGKENFEFYELAKLLPLPAAITSQLDKASIIRLTISYLKMR

SEQ ID NO: 226 - protein, 53 aa, Homo sapiens
MKEKSKNAARTRREKENSEFYELAKLLPLPSAITSQLDKASIIRLTTSYLKMR

SEQ ID NO: 227 - protein, 53 aa, Homo sapiens
MKEKSKNAAKTRREKENGEFYELAKLLPLPSAITSQLDKASIIRLTTSYLKMR

SEQ ID NO: 228 - protein, 54 aa, Homo sapiens
RRKEKSRDAARCRRSKETEVFYELAHELPLPHSVSSHLDKASIMRLAISFLRTH

SEQ ID NO: 229 - protein, 54 aa, Homo sapiens
RRKEKSRDAARSRRSKESEVFYELAHQLPLPHNVSSHLDKASVMRLTISYLRVR

SEQ ID NO: 230 - protein, 54 aa, Homo sapiens
LRKEKSRDAARSRRSQETEVLYQLAHTLPFARGVSAHLDKASIMRLTISYLRMH

SEQ ID NO: 231 - protein, 58 aa, Homo sapiens
PCDTLASSTEKRRREQENKYLEELAELLSANISDIDSLSVKPDKCKILKKTVDQIQLM

SEQ ID NO: 232 - protein, 58 aa, Homo sapiens
LGPSPKRNTEKRNREQENKYIEELAELIFANFNDIDNFNFKPDKCAILKETVKQIRQI

SEQ ID NO: 233 - protein, 58 aa, Homo sapiens
PGQGLTCSGEKRRREQESKYIEELAELISANLSDIDNFNVKPDKCAILKETVRQIRQI

SEQ ID NO: 234 - protein, 52 aa, Homo sapiens
SCLRRNVISERERRKRMSLSCERLRALLPQFDGRREDMASVLEMSVQFLRLA

SEQ ID NO: 235 - protein, 51 aa, Homo sapiens
QRRERHNRMERDRRRRIRICCDELNLLVPFCNAETDKATTLQWTTAFLKYI

SEQ ID NO: 236 - protein, 52 aa, Homo sapiens
KISLLHSSKEKLRRERIKYCCEQLRTLLPYVKGRKNDAASVLEATVDYVKYI

SEQ ID NO: 237 - protein, 56 aa, Homo sapiens
RTPVSHKVIEKRRRDRINRCLNELGKTVPMALAKQSSGKLEKAEILEMTVQYLRAL

SEQ ID NO: 238 - protein, 56 aa, Homo sapiens
ARKKHRGIIEKRRRDRINSSLSELRRLVPTAFEKQGSSKLEKAEVLQMTVDHLKML

SEQ ID NO: 239 - protein, 56 aa, Homo sapiens
ARKRRRGIIEKRRRDRINNSLSELRRLVPSAFEKQGSAKLEKAEILQMTVDHLKML

SEQ ID NO: 240 - protein, 56 aa, Homo sapiens
ARKKRRGIIEKRRRDRINNSLSELRRLVPTAFEKQGSAKLEKAEILQMTVDHLKML

SEQ ID NO: 241 - protein, 56 aa, Homo sapiens
TYKLPHRLIEKKRRDRINECIAQLKDLLPEHLKLTTLGHLEKAVVLELTLKHVKAL

SEQ ID NO: 242 - protein, 56 aa, Homo sapiens
TYKLPHRLIEKKRRDRINECIAQLKDLLPEHLKLTTLGHLEKAVVLELTLKHLKAL

SEQ ID NO: 243 - protein, 58 aa, Homo sapiens
GPKMLKPLVEKRRRDRINRSLEELRLLLLERTRDQNLRNPKLEKAEILEFAVGYLRER

SEQ ID NO: 244 - protein, 53 aa, Homo sapiens
DRKARKPLVEKKRRARINESLQELRLLLAGAEVQAKLENAEVLELTVRRVQGV

SEQ ID NO: 245 - protein, 57 aa, Homo sapiens
KNRLRKPVVEKMRRDRINSSIEQLKLLLEQEFARHQPNSKLEKADILEMAVSYLKHS

SEQ ID NO: 246 - protein, 49 aa, Homo sapiens
MEKKRRARINVSLEQLKSLLEKHYSHQIRKRKLEKADILELSVKYMRSL

SEQ ID NO: 247 - protein, 58 aa, Homo sapiens
LRKSLKPLLEKRRRARINQSLSQLKGLILPLLGRENSNCSKLEKADVLEMTVRFLQEL

SEQ ID NO: 248 - protein, 58 aa, Homo sapiens
HRKSSKPIMEKRRRARINESLSQLKTLILDALKKDSSRHSKLEKADILEMTVKHLRNL

SEQ ID NO: 249 - protein, 58 aa, Homo sapiens
HRKSSKPVMEKRRRARINESLAQLKTLILDALRKESSRHSKLEKADILEMTVRHLRSL

SEQ ID NO: 250 - protein, 51 aa, Homo sapiens
AKRVSRNKSEKKRRDQFNVLIKELGSMLPGNARKMDKSTVLQKSIDFLRKH

SEQ ID NO: 251 - protein, 51 aa, Homo sapiens
AKRASRNKSEKKRRDQFNVLIKELSSMLPGNTRKMDKTTVLEKVIGFLQKH

SEQ ID NO: 252 - protein, 54 aa, Homo sapiens
LARENHSEIERRRRNKMTAYITELSDMVPTCSALARKPDKLTILRMAVSHMKSL

SEQ ID NO: 253 - protein, 54 aa, Homo sapiens
AFREAHSQTEKRRRDKMNNLIEELSAMIPQCNPMARKLDKLTVLRMAVQHLRSL

SEQ ID NO: 254 - protein, 52 aa, Homo sapiens
KNRETHNAVERHRKKKINAGINRIGELIPCSPALKQSKNMILDQAFKYITEL

SEQ ID NO: 255 - protein, 56 aa, Homo sapiens
KRRAQHNEVERRRRDKINNWIVQLSKIIPDCSMESTKSGQSKGGILSKACDYIQEL

SEQ ID NO: 256 - protein, 56 aa, Homo sapiens
RRRAQHNEVERRRRDKINNWIVQLSKIIPDCNADNSKTGASKGGILSKACDYIREL

SEQ ID NO: 257 - protein, 54 aa, Homo sapiens
QKKDNHNLIERRRRFNINDRIKELGMLIPKANDLDVRWNKGTILKASVDYIRRM

SEQ ID NO: 258 - protein, 54 aa, Homo sapiens
QKKDNHNLIERRRRYNINYRIKELGTLIPKSNDPDMRWNKGTILKASVEYIKWL

SEQ ID NO: 259 - protein, 54 aa, Homo sapiens
QKKDNHNLIERRRRFNINDRIKELGTLIPKSNDPDMRWNKGTILKASVDYIRKL

SEQ ID NO: 260 - protein, 54 aa, Homo sapiens
QKKDNHNLIERRRRFNINDRIKELGTLIPKSSDPEMRWNKGTILKASVDYIRKL

SEQ ID NO: 261 - protein, 51 aa, Homo sapiens
EKRTAHNAIEKRYRSSINDKIIELKDLVVGTEAKLNKSAVLRKAIDYIRFL

SEQ ID NO: 262 - protein, 51 aa, Homo sapiens
ERRTTHNIIEKRYRSSINDKIIELKDLVMGTDAKMHKSGVLRKAIDYIKYL

SEQ ID NO: 263 - protein, 52 aa, Homo sapiens
GTREVHNKLEKNRRAHLKECFETLKRNIPNVDDKKTSNLSVLRTALRYIQSL

SEQ ID NO: 264 - protein, 53 aa, Homo sapiens
SGRSVHNELEKRRRAQLKRCLERLKQQMPLGADCARYTTLSLLRRARMHIQKL

SEQ ID NO: 265 - protein, 53 aa, Homo sapiens
NNRSSHNELEKHRRAKLRLYLEQLKQLVPLGPDSTRHTTLSLLKRAKVHIKKL

SEQ ID NO: 266 - protein, 53 aa, Homo sapiens
SSRSTHNEMEKNRRAHLRLCLEKLKGLVPLGPESSRHTTLSLLTKAKLHIKKL

SEQ ID NO: 267 - protein, 53 aa, Homo sapiens
ANRSTHNELEKNRRAHLRLCLERLKVLIPLGPDCTRHTTLGLLNKAKAHIKKL

SEQ ID NO: 268 - protein, 59 aa, Homo sapiens
RRRRAHTQAEQKRRDAIKRGYDDLQTIVPTCQQQDFSIGSQKLSKAIVLQKTIDYIQFL

SEQ ID NO: 269 - protein, 51 aa, Homo sapiens
NRQMKHISAEQKRRFNIKMCFDMLNSLISNNSKLTSHAITLQKTVEYITKL

SEQ ID NO: 270 - protein, 55 aa, Homo sapiens
NRRITHISAEQKRRFNIKLGFDTLHGLVSTLSAQPSLKVSKATTLQKTAEYILML

SEQ ID NO: 271 - protein, 53 aa, Homo sapiens
ERRRVANAKERERIKNLNRGFARLKALVPFLPQSRKPSKVDILKGATEYIQVL

SEQ ID NO: 272 - protein, 52 aa, Homo sapiens
DKRAHHNALERKRRDHIKDSFHSLRDSVPSLQGEKASRAQILDKATEYIQYM

SEQ ID NO: 273 - protein, 53 aa, Homo sapiens
TKRKNHNFLERKRRNDLRSRFLALRDQVPTLASCSKAPKVVILSKALEYLQAL

SEQ ID NO: 274 - protein, 53 aa, Homo sapiens
VKRRTHNVLERQRRNELKRSFFALRDQIPELENNEKAPKVVILKKATAYILSV

SEQ ID NO: 275 - protein, 53 aa, Homo sapiens
ERRRNHNILERQRRNDLRSSFLTLRDHVPELVKNEKAAKVVILKKATEYVHSL

SEQ ID NO: 276 - protein, 54 aa, Homo sapiens
ERRVANNARERLRVRDINEAFKELGRMCQLHLNSEKPQTKLLILHQAVSVILNL

SEQ ID NO: 277 - protein, 54 aa, Homo sapiens
ERRMANNARERLRVRDINEAFKELGRMCQLHLKSEKPQTKLLILHQAVAVILSL

SEQ ID NO: 278 - protein, 54 aa, Homo sapiens
ERRMANNARERLRVRDINEAFKELGRMVQLHLKSDKPQTKLLILHQAVAVILSL

SEQ ID NO: 279 - protein, 53 aa, Homo sapiens
AAVARRNERERNRVKLVNLGFATLREHVPNGAANKKMSKVETLRSAVEYIRAL

SEQ ID NO: 280 - protein, 53 aa, Homo sapiens
AAVARRNERERNRVKLVNLGFQALRQHVPHGGASKKLSKVETLRSAVEYIRAL

SEQ ID NO: 281 - protein, 53 aa, Homo sapiens
AFTRKRNERERQRVKCVNEGYAQLRHHLPEEYLEKRLSKVETLRAAIKYINYL

SEQ ID NO: 282 - protein, 53 aa, Homo sapiens
AFLRKRNERERQRVRCVNEGYARLRDHLPRELADKRLSKVETLRAAIDYIKHL

SEQ ID NO: 283 - protein, 53 aa, Homo sapiens
AFIQKRNERERQRVKCVNEGYARLRGHLPGALAEKRLSKVETLRAAIRYIKYL

SEQ ID NO: 284 - protein, 52 aa, Homo sapiens
IRREIANSNERRRMQSINAGFQSLKTLIPHTDGEKLSKAAILQQTAEYIFSL

SEQ ID NO: 285 - protein, 55 aa, Homo sapiens
GQRQSASEREKLRMRTLARALHELRRFLPPSVAPAGQSLTKIETLRLAIRYIGHL

SEQ ID NO: 286 - protein, 55 aa, Homo sapiens
GQRQSASEREKLRMRTLARALHELRRFLPPSLAPAGQSLTKIETLRLAIRYIGHL

SEQ ID NO: 287 - protein, 55 aa, Homo sapiens
QRRRKASEREKLRMRTLADALHTLRNYLPPVYSQRGQPLTKIQTLKYTIKYIGEL

SEQ ID NO: 288 - protein, 60 aa, Homo sapiens
QLRRKINSRERKRMQDLNLAMDALREVILPYSAAHCQGAPGRKLSKIATLLLARNYILLL

SEQ ID NO: 289 - protein, 55 aa, Homo sapiens
QLRLKINSRERKRMHDLNIAMDGLREVMPYAHGPSVRKLSKIATLLLARNYILML

SEQ ID NO: 290 - protein, 55 aa, Homo sapiens
QLRLKINGRERKRMHDLNLAMDGLREVMPYAHGPSVRKLSKIATLLLARNYILML

SEQ ID NO: 291 - protein, 55 aa, Homo sapiens
ALRLNINARERRRMHDLNDALDELRAVIPYAHSPSVRKLSKIATLLLAKNYILMQ

SEQ ID NO: 292 - protein, 55 aa, Homo sapiens
SLRLSINARERRRMHDLNDALDGLRAVIPYAHSPSVRKLSKIATLLLAKNYILMQ

SEQ ID NO: 293 - protein, 53 aa, Homo sapiens
QRRLESNERERQRMHKLNNAFQALREVIPHVRADKKLSKIETLTLAKNYIKSL

SEQ ID NO: 294 - protein, 53 aa, Homo sapiens
QRRLAANARERRRMHGLNHAFDQLRNVIPSFNNDKKLSKYETLQMAQIYINAL

SEQ ID NO: 295 - protein, 53 aa, Homo sapiens
RRRLAANARERRRMQGLNTAFDRLRRVVPQWGQDKKLSKYETLQMALSYIMAL

SEQ ID NO: 296 - protein, 53 aa, Homo sapiens
TRRLLANARERTRVHTISAAFEALRKQVPCYSYGQKLSKLAILRIACNYILSL

SEQ ID NO: 297 - protein, 53 aa, Homo sapiens
TRRLKANNRERNRMHNLNAALDALREVLPTFPEDAKLTKIETLRFAHNYIWAL

SEQ ID NO: 298 - protein, 53 aa, Homo sapiens
SRRVKANDRERNRMHNLNAALDALRSVLPSFPDDTKLTKIETLRFAYNYIWAL

SEQ ID NO: 299 - protein, 53 aa, Homo sapiens
SRRKKANDRERNRMHNLNSALDALRGVLPTFPDDAKLTKIETLRFAHNYIWAL

SEQ ID NO: 300 - protein, 53 aa, Homo sapiens
ARRVKANARERTRMHGLNDALDNLRRVMPCYSKTQKLSKIETLRLARNYIWAL

SEQ ID NO: 301 - protein, 53 aa, Homo sapiens
LRRMKANARERNRMHGLNAALDNLRKVVPCYSKTQKLSKIETLRLAKNYIWAL

SEQ ID NO: 302 - protein, 53 aa, Homo sapiens
LRRQKANARERNRMHDLNAALDNLRKVVPCYSKTQKLSKIETLRLAKNYIWAL

SEQ ID NO: 303 - protein, 53 aa, Homo sapiens
FRRQEANARERNRMHGLNDALDNLRKVVPCYSKTQKLSKIETLRLAKNYIWAL

SEQ ID NO: 304 - protein, 52 aa, Homo sapiens
DRRKAATMRERRRLKKVNQAFETLKRCTTTNPNQRLPKVEILRNAIRYIESL

SEQ ID NO: 305 - protein, 52 aa, Homo sapiens
DRRKAATMRERRRLSKVNEAFETLKRCTSSNPNQRLPKVEILRNAIRYIEGL

SEQ ID NO: 306 - protein, 52 aa, Homo sapiens
DRRKAATLRERRRLKKINEAFEALKRRTVANPNQRLPKVEILRSAISYIERL

SEQ ID NO: 307 - protein, 52 aa, Homo sapiens
DRRRAATLREKRRLKKVNEAFEALKRSTLLNPNQRLPKVEILRSAIQYIERL

SEQ ID NO: 308 - protein, 53 aa, Homo sapiens
ARRMAANVRERKRILDYNEAFNALRRALRHDLGGKRLSKIATLRRAIHRIAAL

SEQ ID NO: 309 - protein, 53 aa, Homo sapiens
AQRQAANIRERKRMFNLNEAFDQLRRKVPTFAYEKRLSRIETLRLAIVYISFM

SEQ ID NO: 310 - protein, 53 aa, Homo sapiens
QLRQAANVRERRRMQSINDAFEGLRSHIPTLPYEKRLSKVDTLRLAIGYINFL

SEQ ID NO: 311 - protein, 53 aa, Homo sapiens
KYRTAHATRERIRVEAFNLAFAELRKLLPTLPPDKKLSKIEILRLAICYISYL

SEQ ID NO: 312 - protein, 53 aa, Homo sapiens
KYRSAHATRERIRVEAFNLAFAELRKLLPTLPPDKKLSKIEILRLAICYISYL

SEQ ID NO: 313 - protein, 53 aa, Homo sapiens
TRKIFTNTRERWRQQNVNSAFAKLRKLIPTHPPDKKLSKNETLRLAMRYINFL

SEQ ID NO: 314 - protein, 53 aa, Homo sapiens
ARRVFTNSRERWRQQNVNGAFAELRKLLPTHPPDRKLSKNEVLRLAMKYIGFL

SEQ ID NO: 315 - protein, 53 aa, Homo sapiens
VRRIFTNSRERWRQQNVNGAFAELRKLIPTHPPDKKLSKNEILRLAMKYINFL

SEQ ID NO: 316 - protein, 53 aa, Homo sapiens
RRKGSGPKKERRRTESINSAFAELRECIPNVPADTKLSKIKTLRLATSYIAYL

SEQ ID NO: 317 - protein, 53 aa, Homo sapiens
KRRGTANRKERRRTQSINSAFAELRECIPNVPADTKLSKIKTLRLATSYIAYL

SEQ ID NO: 318 - protein, 53 aa, Homo sapiens
EASPENAARERSRVRTLRQAFLALQAALPAVPPDTKLSKLDVLVLAASYIAHL

SEQ ID NO: 319 - protein, 53 aa, Homo sapiens
RPAAANAARERSRVQTLRHAFLELQRTLPSVPPDTKLSKLDVLLLATTYIAHL

SEQ ID NO: 320 - protein, 53 aa, Homo sapiens
SQRNAANARERARMRVLSKAFSRLKTSLPWVPPDTKLSKLDTLRLASSYIAHL

SEQ ID NO: 321 - protein, 53 aa, Homo sapiens
VQRNAANARERARMRVLSKAFSRLKTTLPWVPPDTKLSKLDTLRLASSYIAHL

SEQ ID NO: 322 - protein, 53 aa, Homo sapiens
RQRHTANARERDRTNSVNTAFTALRTLIPTEPADRKLSKIETLRLASSYISHL

SEQ ID NO: 323 - protein, 53 aa, Homo sapiens
RQRQAANARERDRTQSVNTAFTALRTLIPTEPVDRKLSKIETVRLASSYIAHL

SEQ ID NO: 324 - protein, 52 aa, Homo sapiens
TQRVMANVRERQRTQSLNEAFAALRKIIPTLPSDKLSKIQTLKLAARYIDFL

SEQ ID NO: 325 - protein, 52 aa, Homo sapiens
SQRILANVRERQRTQSLNEAFAALRKIIPTLPSDKLSKIQTLKLAARYIDFL

SEQ ID NO: 326 - protein, 19 aa, Homo sapiens
  MISC_FEATURE (1)..(1): Xaa is turnlike (any of A,C,D,E,G,H,K,N,Q,R,S, T)
  MISC_FEATURE (2)..(2): Xaa is polar (any of C,D,E,H,K,N,Q,R,S,T)
  MISC_FEATURE (3)..(3): Xaa is any natural amino acid
  MISC_FEATURE (4)..(4): Xaa is any natural amino acid
  MISC_FEATURE (5)..(5): Xaa is any natural amino acid
  MISC_FEATURE (6)..(6): Xaa is any natural amino acid
  MISC_FEATURE (7)..(7): Xaa is any natural amino acid
  MISC_FEATURE (8)..(8): Xaa is any natural amino acid
  MISC_FEATURE (9)..(9): Xaa is turnlike (any of A,C,D,E,G,H,K,N,Q,R,S, T)
  MISC_FEATURE (10)..(10): Xaa is any natural amino acid
  MISC_FEATURE (11)..(11): Xaa is any natural amino acid
  MISC_FEATURE (12)..(12): Xaa is hydrophobic (any of A,C,F,G,H,I,K,L,M,R, T,V,W,Y)
  MISC_FEATURE (13)..(13): Xaa is any natural amino acid
  MISC_FEATURE (14)..(14): Xaa is any natural amino acid
  MISC_FEATURE (15)..(15): Xaa is aliphatic (I,L,V)
  MISC_FEATURE (16)..(16): Xaa is turnlike (any of A,C,D,E,G,H,K,N,Q,R,S,T ))
  MISC_FEATURE (17)..(17): Xaa is any natural amino acid
  MISC_FEATURE (18)..(18): Xaa is any natural amino acid
  MISC_FEATURE (19)..(19): Xaa is hydrophobic (any of A,C,F,G,H,I,K,L,M,R, T,V,W,Y)
XXXXXXXXXXXXXXXXXXX

SEQ ID NO: 327 - protein, 26 aa, Homo sapiens
  MISC_FEATURE (1)..(1): Xaa is turnlike (any of A,C,D,E,G,H,K,N,Q,R,S, T)
  MISC_FEATURE (2)..(2): Xaa is any natural amino acid
  MISC_FEATURE (3)..(3): Xaa is any natural amino acid
  MISC_FEATURE (4)..(4): Xaa is turnlike (any of A,C,D,E,G,H,K,N,Q,R,S, T)
  MISC_FEATURE (5)..(5): Xaa is any natural amino acid
  MISC_FEATURE (6)..(6): Xaa is any natural amino acid
  MISC_FEATURE (7)..(7): Xaa is hydrophobic (any of A,C,F,G,H,I,K,L,M,R, T,V,W,Y)
  MISC_FEATURE (9)..(9): Xaa is positive (any of H,K,R)
  MISC_FEATURE (10)..(10): Xaa is any natural amino
  MISC_FEATURE (12)..(12): Xaa is hydrophobic (any of A,C,F,G,H,I,K,L,M,R, T,V,W,Y)
  MISC_FEATURE (13)..(13): Xaa is turnlike (any of A,C,D,E,G,H,K,N,Q,R,S,T )
  MISC_FEATURE (14)..(14): Xaa is turnlike (any of A,C,D,E,G,H,K,N,Q,R,S,T )
  MISC_FEATURE (15)..(15): Xaa is hydrophobic (any of A,C,F,G,H,I,K,L,M,R, T,V,W,Y)
  MISC_FEATURE (16)..(16): Xaa is polar (any of C,D,E,H,K,N,Q,R,S,T)
  MISC_FEATURE (17)..(17): Xaa is any natural amino acid
  MISC_FEATURE (18)..(18): Xaa is any natural amino acid
  MISC_FEATURE (19)..(19): Xaa is hydrophobic (any of A,C,F,G,H,I,K,L,M,R, T,V,W,Y)
  MISC_FEATURE (20)..(20): Xaa is any natural amino acid
  MISC_FEATURE (21)..(21): Xaa is turnlike (any of A,C,D,E,G,H,K,N,Q,R,S,T )
  MISC_FEATURE (23)..(23): Xaa is turnlike (any of A,C,D,E,G,H,K,N,Q,R,S,T )
  MISC_FEATURE (24)..(24): Xaa is turnlike (any of A,C,D,E,G,H,K,N,Q,R,S,T )
  MISC_FEATURE (25)..(25): Xaa is hydrophobic (any of A,C,F,G,H,I,K,L,M,R, T,V,W,Y)
  MISC_FEATURE (26)..(26): Xaa is aliphatic (any of I,L,V)
XXXXXXXEXXRXXXXXXXXXXLXXXX

SEQ ID NO: 328 - protein, 29 aa, Homo sapiens
  MISC_FEATURE (1)..(1): Xaa is turnlike (any of A,C,D,E,G,H,K,N,Q,R,S,T )
  MISC_FEATURE (2)..(2): Xaa is hydrophobic (any of A,C,F,G,H,I,K,L,M,R, T,V,W,Y)
  MISC_FEATURE (3)..(3): Xaa is is positive (any of H,K,R)
  MISC_FEATURE (4)..(4): Xaa is any natural amino acid
  MISC_FEATURE (5)..(5): Xaa is turnlike (any of A,C,D,E,G,H,K,N,Q,R,S,T )
  MISC_FEATURE (6)..(6): Xaa is turnlike (any of A,C,D,E,G,H,K,N,Q,R,S,T )
  MISC_FEATURE (7)..(7): Xaa is small (any of A,C,D,G,N,P,S,T,V)
  MISC_FEATURE (8)..(8): Xaa is any natural amino acid
  MISC_FEATURE (9)..(9): Xaa is hydrophobic (any of A,C,F,G,H,I,K,L,M,R, T,V,W,Y)
  MISC_FEATURE (11)..(11): Xaa is positive (any of H,K,R)
  MISC_FEATURE (12)..(12): Xaa is polar (any of C,D,E,H,K,N,Q,R,S,T)
  MISC_FEATURE (14)..(14): Xaa is hydrophobic (any of A,C,F,G,H,I,K,L,M,R, T,V,W,Y)
  MISC_FEATURE (15)..(15): Xaa is polar (any of C,D,E,H,K,N,Q,R,S,T)
  MISC_FEATURE (16)..(16): Xaa is polar (any of C,D,E,H,K,N,Q,R,S,T)
  MISC_FEATURE (17)..(17): Xaa is aliphatic (and of I,L,V)
  MISC_FEATURE (18)..(18): Xaa is small (any of A,C,D,G,N,P,S,T,V)
  MISC_FEATURE (19)..(19): Xaa is turnlike (any of A,C,D,E,G,H,K,N,Q,R,S,T )
  MISC_FEATURE (20)..(20): Xaa is turnlike (any of A,C,D,E,G,H,K,N,Q,R,S,T )
  MISC_FEATURE (21)..(21): Xaa is hydrophobic (any of A,C,F,G,H,I,K,L,M,R, T,V,W,Y)
  MISC_FEATURE (22)..(22): Xaa is turnlike (any of A,C,D,E,G,H,K,N,Q,R,S,T )
  MISC_FEATURE (23)..(23): Xaa is turnlike (any of A,C,D,E,G,H,K,N,Q,R,S,T )
  MISC_FEATURE (25)..(25): Xaa is turnlike (any of A,C,D,E,G,H,K,N,Q,R,S,T )
  MISC_FEATURE (26)..(26): Xaa is polar (any of C,D,E,H,K,N,Q,R,S,T)
  MISC_FEATURE (27)..(27): Xaa is hydrophobic (any of A,C,F,G,H,I,K,L,M,R, T,V,W,Y)
  MISC_FEATURE (28)..(28): Xaa is aliphatic (any of I,L,V)
  MISC_FEATURE (29)..(29): Xaa is small (any of A,C,D,G,N,P,S,T,V)
XXXXXXXXXEXXRXXXXXXXXXXLXXXXX

SEQ ID NO: 329 - protein, 9 aa, Homo sapiens
  MISC_FEATURE (1)..(1): Xaa is hydrophobic (any of A,C,F,G,H,I,K,L,M,R, T,V,W,Y)
  MISC_FEATURE (2)..(2): Xaa is hydrophobic (any of A,C,F,G,H,I,K,L,M,R, T,V,W,Y)
  MISC_FEATURE (3)..(3): Xaa is any natural amino acid
  MISC_FEATURE (4)..(4): Xaa is any natural amino acid
  MISC_FEATURE (5)..(5): Xaa is small (any of A,C,D,G,N,P,S,T,V)
  MISC_FEATURE (6)..(6): Xaa is any natural amino acid
  MISC_FEATURE (7)..(7): Xaa is any natural amino acid
  MISC_FEATURE (8)..(8): Xaa is any natural amino acid
  MISC_FEATURE (9)..(9): Xaa is hydrophobic (any of A,C,F,G,H,I,K,L,M,R, T,V,W,Y)
XXXXXXXXX

SEQ ID NO: 330 - protein, 31 aa, Homo sapiens
  MISC_FEATURE (1)..(1): Xaa is any natural amino acid
  MISC_FEATURE (2)..(2): Xaa is any natural amino acid
  MISC_FEATURE (3)..(3): Xaa is any natural amino acid
  MISC_FEATURE (4)..(4): Xaa is any natural amino acid
  MISC_FEATURE (5)..(5): Xaa is any natural amino acid
  MISC_FEATURE (6)..(6): Xaa is any natural amino acid
  MISC_FEATURE (7)..(7): Xaa is any natural amino acid
  MISC_FEATURE (8)..(8): Xaa is any natural amino acid
  MISC_FEATURE (9)..(9): Xaa is any natural amino acid
  MISC_FEATURE (10)..(10): Xaa is turnlike (any of A,C,D,E,G,H,K,N,Q,R,S,T )
  MISC_FEATURE (11)..(11): Xaa is polar (any of C,D,E,H,K,N,Q,R,S,T)
  MISC_FEATURE (12)..(12): Xaa is any natural amino acid
  MISC_FEATURE (13)..(13): Xaa is any natural amino acid
  MISC_FEATURE (14)..(14): Xaa is any natural amino acid
  MISC_FEATURE (15)..(15): Xaa is any natural amino acid
  MISC_FEATURE (16)..(16): Xaa is any natural amino acid
  MISC_FEATURE (17)..(17): Xaa is any natural amino acid
  MISC_FEATURE (18)..(18): Xaa is turnlike (any of A,C,D,E,G,H,K,N,Q,R,S,T )
  MISC_FEATURE (19)..(19): Xaa is any natural amino acid
  MISC_FEATURE (20)..(20): Xaa is any natural amino acid
  MISC_FEATURE (21)..(21): Xaa is hydrophobic (any of A,C,F,G,H,I,K,L,M,R, T,V,W,Y)
  MISC_FEATURE (22)..(22): Xaa is any natural amino acid
  MISC_FEATURE (23)..(23): Xaa is any natural amino acid
  MISC_FEATURE (24)..(24): Xaa is aliphatic (any of I,L,V)
  MISC_FEATURE (25)..(25): Xaa is turnlike (any of A,C,D,E,G,H,K,N,Q,R,S,T )
  MISC_FEATURE (26)..(26): Xaa is any natural amino acid
  MISC_FEATURE (27)..(27): Xaa is any natural amino acid
  MISC_FEATURE (28)..(28): Xaa is hydrophobic (any of A,C,F,G,H,I,K,L,M,R, T,V,W,Y)
  MISC_FEATURE (29)..(29): Xaa is any natural amino acid
  MISC_FEATURE (30)..(30): Xaa is any natural amino acid
  MISC_FEATURE (31)..(31): Xaa is any natural amino acid
XXXXXXXXXXXXXXXXXXXXXXXXXXXXXXX

SEQ ID NO: 331 - protein, 31 aa, Homo sapiens
  MISC_FEATURE (1)..(1): Xaa is any natural amino acid
  MISC_FEATURE (2)..(2): Xaa is any natural amino acid
  MISC_FEATURE (3)..(3): Xaa is turnlike (any of A,C,D,E,G,H,K,N,Q,R,S,T )
  MISC_FEATURE (4)..(4): Xaa is any natural amino acid
  MISC_FEATURE (5)..(5): Xaa is any natural amino acid
  MISC_FEATURE (6)..(6): Xaa is turnlike (any of A,C,D,E,G,H,K,N,Q,R,S,T )
  MISC_FEATURE (7)..(7): Xaa is any natural amino acid
  MISC_FEATURE (8)..(8): Xaa is any natural amino acid
  MISC_FEATURE (9)..(9): Xaa is hydrophobic (any of A,C,F,G,H,I,K,L,M,R, T,V,W,Y)
  MISC_FEATURE (11)..(11): Xaa is positive (any of H,K,R)
  MISC_FEATURE (12)..(12): Xaa is any natural amino acid
  MISC_FEATURE (14)..(14): Xaa is hydrophobic (any of A,C,F,G,H,I,K,L,M,R, T,V,W,Y)
  MISC_FEATURE (15)..(15): Xaa is turnlike (any of A,C,D,E,G,H,K,N,Q,R,S,T )
  MISC_FEATURE (16)..(16): Xaa is turnlike (any of A,C,D,E,G,H,K,N,Q,R,S,T )
  MISC_FEATURE (17)..(17): Xaa is hydrophobic (any of A,C,F,G,H,I,K,L,M,R, T,V,W,Y)
  MISC_FEATURE (18)..(18): Xaa is polar (any of C,D,E,H,K,N,Q,R,S,T)
  MISC_FEATURE (19)..(19): Xaa is any natural amino acid
  MISC_FEATURE (20)..(20): Xaa is any natural amino acid
  MISC_FEATURE (21)..(21): Xaa is hydrophobic (any of A,C,F,G,H,I,K,L,M,R, T,V,W,Y)
  MISC_FEATURE (22)..(22): Xaa is any natural amino acid
  MISC_FEATURE (23)..(23): Xaa is turnlike (any of A,C,D,E,G,H,K,N,Q,R,S,T )
  MISC_FEATURE (25)..(25): Xaa is turnlike (any of A,C,D,E,G,H,K,N,Q,R,S,T )
  MISC_FEATURE (26)..(26): Xaa is turnlike (any of A,C,D,E,G,H,K,N,Q,R,S,T )
  MISC_FEATURE (27)..(27): Xaa is hydrophobic (any of A,C,F,G,H,I,K,L,M,R, T,V,W,Y)
  MISC_FEATURE (28)..(28): Xaa is aliphatic (any of I,L,V)
  MISC_FEATURE (29)..(29): Xaa is any natural amino acid
  MISC_FEATURE (30)..(30): Xaa is any natural amino acid
  MISC_FEATURE (31)..(31): Xaa is any natural amino acid
XXXXXXXXXEXXRXXXXXXXXXXLXXXXXXX

SEQ ID NO: 332 - protein, 31 aa, Homo sapiens; identical to SEQ ID NO: 331
  MISC_FEATURE (1)..(1): Xaa is turnlike (any of A,C,D,E,G,H,K,N,Q,R,S,T )
  MISC_FEATURE (2)..(2): Xaa is hydrophobic (any of A,C,F,G,H,I,K,L,M,R, T,V,W,Y)
  MISC_FEATURE (3)..(3): Xaa is positive (any of H,K,R)
  MISC_FEATURE (4)..(4): Xaa is any natural amino acid
  MISC_FEATURE (5)..(5): Xaa is turnlike (any of A,C,D,E,G,H,K,N,Q,R,S,T )
  MISC_FEATURE (6)..(6): Xaa is turnlike (any of A,C,D,E,G,H,K,N,Q,R,S,T )
  MISC_FEATURE (7)..(7): Xaa is small (any of A,C,D,G,N,P,S,T,V)
  MISC_FEATURE (8)..(8): Xaa is any natural amino acid
  MISC_FEATURE (9)..(9): Xaa is hydrophobic (any of A,C,F,G,H,I,K,L,M,R, T,V,W,Y)
  MISC_FEATURE (11)..(11): Xaa is positive (any of H,K,R)
  MISC_FEATURE (12)..(12): Xaa is polar (any of C,D,E,H,K,N,Q,R,S,T)
  MISC_FEATURE (14)..(14): Xaa is hydrophobic (any of A,C,F,G,H,I,K,L,M,R, T,V,W,Y)
  MISC_FEATURE (15)..(15): Xaa is polar (any of C,D,E,H,K,N,Q,R,S,T)
  MISC_FEATURE (16)..(16): Xaa is polar (any of C,D,E,H,K,N,Q,R,S,T)
  MISC_FEATURE (17)..(17): Xaa is aliphatic (any of I,L,V)
  MISC_FEATURE (18)..(18): Xaa is small (any of A,C,D,G,N,P,S,T,V)
  MISC_FEATURE (19)..(19): Xaa is turnlike (any of A,C,D,E,G,H,K,N,Q,R,S,T )
  MISC_FEATURE (20)..(20): Xaa is turnlike (any of A,C,D,E,G,H,K,N,Q,R,S,T )
  MISC_FEATURE (21)..(21): Xaa is hydrophobic (any of A,C,F,G,H,I,K,L,M,R, T,V,W,Y)
  MISC_FEATURE (22)..(22): Xaa is turnlike (any of A,C,D,E,G,H,K,N,Q,R,S,T )
  MISC_FEATURE (23)..(23): Xaa is turnlike (any of A,C,D,E,G,H,K,N,Q,R,S,T )
  MISC_FEATURE (25)..(25): Xaa is turnlike (any of A,C,D,E,G,H,K,N,Q,R,S,T )
  MISC_FEATURE (26)..(26): Xaa is polar (any of C,D,E,H,K,N,Q,R,S,T)
  MISC_FEATURE (27)..(27): Xaa is hydrophobic (any of A,C,F,G,H,I,K,L,M,R, T,V,W,Y)
  MISC_FEATURE (28)..(28): Xaa is aliphatic (any of I,L,V)
  MISC_FEATURE (29)..(29): Xaa is small (any of A,C,D,G,N,P,S,T,V)
  MISC_FEATURE (30)..(30): Xaa is is any natural amino acid
  MISC_FEATURE (31)..(31): Xaa is is any natural amino acid

SEQ ID NO: 333 - protein, 31 aa, Homo sapiens
  MISC_FEATURE (1)..(1): Xaa is turnlike (any of A,C,D,E,G,H,K,N,Q,R,S,T )
  MISC_FEATURE (2)..(2): Xaa is polar (any of C,D,E,H,K,N,Q,R,S,T)
  MISC_FEATURE (3)..(3): Xaa is positive (any of H,K,R)
  MISC_FEATURE (4)..(4): Xaa is any natural amino acid
  MISC_FEATURE (5)..(5): Xaa is turnlike (any of A,C,D,E,G,H,K,N,Q,R,S,T )
  MISC_FEATURE (6)..(6): Xaa is turnlike (any of A,C,D,E,G,H,K,N,Q,R,S,T )
  MISC_FEATURE (7)..(7): Xaa is small (any of A,C,D,G,N,P,S,T,V)
  MISC_FEATURE (8)..(8): Xaa is turnlike (any of A,C,D,E,G,H,K,N,Q,R,S,T )
  MISC_FEATURE (9)..(9): Xaa is hydrophobic (any of A,C,F,G,H,I,K,L,M,R, T,V,W,Y)
  MISC_FEATURE (11)..(11): Xaa is positive (any of H,K,R)
  MISC_FEATURE (12)..(12): Xaa is polar (any of C,D,E,H,K,N,Q,R,S,T)
  MISC_FEATURE (14)..(14): Xaa is hydrophobic (any of A,C,F,G,H,I,K,L,M,R, T,V,W,Y)
  MISC_FEATURE (15)..(15): Xaa is polar (any of C,D,E,H,K,N,Q,R,S,T)
  MISC_FEATURE (16)..(16): Xaa is polar (any of C,D,E,H,K,N,Q,R,S,T)
  MISC_FEATURE (17)..(17): Xaa is aliphatic (any of I,L,V)
  MISC_FEATURE (19)..(19): Xaa is turnlike (any of A,C,D,E,G,H,K,N,Q,R,S,T )
  MISC_FEATURE (20)..(20): Xaa is small (any of A,C,D,G,N,P,S,T,V)
  MISC_FEATURE (21)..(21): Xaa is hydrophobic (any of A,C,F,G,H,I,K,L,M,R, T,V,W,Y)
  MISC_FEATURE (22)..(22): Xaa is turnlike (any of A,C,D,E,G,H,K,N,Q,R,S,T )
  MISC_FEATURE (23)..(23): Xaa is polar (any of C,D,E,H,K,N,Q,R,S,T)
  MISC_FEATURE (25)..(25): Xaa is polar (any of C,D,E,H,K,N,Q,R,S,T)
  MISC_FEATURE (26)..(26): Xaa is polar (any of C,D,E,H,K,N,Q,R,S,T)
  MISC_FEATURE (27)..(27): Xaa is hydrophobic (any of A,C,F,G,H,I,K,L,M,R, T,V,W,Y)
  MISC_FEATURE (28)..(28): Xaa is aliphatic (any of I,L,V)
  MISC_FEATURE (30)..(30): Xaa is hydrophobic (any of A,C,F,G,H,I,K,L,M,R, T,V,W,Y)
  MISC_FEATURE (31)..(31): Xaa is hydrophobic (any of A,C,F,G,H,I,K,L,M,R, T,V,W,Y)
XXXXXXXXXEXXRXXXXNXXXXXLXXXXPXX

SEQ ID NO: 334 - protein, 20 aa, Homo sapiens
  MISC_FEATURE (1)..(1): Xaa is any natural amino acid
  MISC_FEATURE (2)..(2): Xaa is any natural amino acid
  MISC_FEATURE (3)..(3): Xaa is any natural amino acid
  MISC_FEATURE (4)..(4): Xaa is any natural amino acid
  MISC_FEATURE (5)..(5): Xaa is any natural amino acid
  MISC_FEATURE (6)..(6): Xaa is any natural amino acid
  MISC_FEATURE (7)..(7): Xaa is any natural amino acid
  MISC_FEATURE (8)..(8): Xaa is any natural amino acid
  MISC_FEATURE (9)..(9): Xaa is hydrophobic (any of A,C,F,G,H,I,K,L,M,R, T,V,W,Y)
  MISC_FEATURE (10)..(10): Xaa is hydrophobic (any of A,C,F,G,H,I,K,L,M,R, T,V,W,Y)
  MISC_FEATURE (11)..(11): Xaa is any natural amino acid
  MISC_FEATURE (12)..(12): Xaa is any natural amino acid
  MISC_FEATURE (13)..(13): Xaa is small (any of A,C,D,G,N,P,S,T,V)
  MISC_FEATURE (14)..(14): Xaa is any natural amino acid
  MISC_FEATURE (15)..(15): Xaa is any natural amino acid
  MISC_FEATURE (16)..(16): Xaa is any natural amino acid
  MISC_FEATURE (17)..(17): Xaa is hydrophobic (any of A,C,F,G,H,I,K,L,M,R, T,V,W,Y)
  MISC_FEATURE (18)..(18): Xaa is any natural amino acid
  MISC_FEATURE (19)..(19): Xaa is any natural amino acid
  MISC_FEATURE (20)..(20): Xaa is any natural amino acid
XXXXXXXXXXXXXXXXXXXX

SEQ ID NO: 335 - protein, 20 aa, Homo sapiens
  MISC_FEATURE (1)..(1): Xaa is turnlike (any of A,C,D,E,G,H,K,N,Q,R,S,T )
  MISC_FEATURE (2)..(2): Xaa is any natural amino acid
  MISC_FEATURE (3)..(3): Xaa is polar (any of C,D,E,H,K,N,Q,R,S,T)
  MISC_FEATURE (4)..(4): Xaa is any natural amino acid
  MISC_FEATURE (5)..(5): Xaa is polar (any of C,D,E,H,K,N,Q,R,S,T)
  MISC_FEATURE (6)..(6): Xaa is positive (any of H,K,R)
  MISC_FEATURE (7)..(7): Xaa is hydrophobic (any of A,C,F,G,H,I,K,L,M,R, T,V,W,Y)
  MISC_FEATURE (8)..(8): Xaa is any natural amino acid
  MISC_FEATURE (9)..(9): Xaa is hydrophobic (any of A,C,F,G,H,I,K,L,M,R, T,V,W,Y)
  MISC_FEATURE (11)..(11): Xaa is polar (any of C,D,E,H,K,N,Q,R,S,T)
  MISC_FEATURE (12)..(12): Xaa is any natural amino acid
  MISC_FEATURE (13)..(13): Xaa is small (any of A,C,D,G,N,P,S,T,V)
  MISC_FEATURE (14)..(14): Xaa is hydrophobic (any of A,C,F,G,H,I,K,L,M,R, T,V,W,Y)
  MISC_FEATURE (15)..(15): Xaa is turnlike (any of A,C,D,E,G,H,K,N,Q,R,S,T )
  MISC_FEATURE (16)..(16): Xaa is aromatic (any of F,H,W,Y)
  MISC_FEATURE (17)..(17): Xaa is aliphatic (any of I,L,V)
  MISC_FEATURE (18)..(18): Xaa is any natural amino acid
  MISC_FEATURE (19)..(19): Xaa is any natural amino acid
  MISC_FEATURE (20)..(20): Xaa is hydrophobic (any of A,C,F,G,H,I,K,L,M,R, T,V,W,Y)
XXXXXXXXXLXXXXXXXXXX

SEQ ID NO: 336 - protein, 20 aa, Homo sapiens
  MISC_FEATURE (1)..(1): Xaa is small (any of A,C,D,G,N,P,S,T,V)
  MISC_FEATURE (2)..(2): Xaa is turnlike (any of A,C,D,E,G,H,K,N,Q,R,S,T )
  MISC_FEATURE (3)..(3): Xaa is positive (any of H,K,R)
  MISC_FEATURE (4)..(4): Xaa is hydrophobic (any of A,C,F,G,H,I,K,L,M,R, T,V,W,Y)
  MISC_FEATURE (5)..(5): Xaa is small (any of A,C,D,G,N,P,S,T,V)
  MISC_FEATURE (7)..(7): Xaa is hydrophobic (any of A,C,F,G,H,I,K,L,M,R, T,V,W,Y)
  MISC_FEATURE (8)..(8): Xaa is turnlike (any of A,C,D,E,G,H,K,N,Q,R,S,T )
  MISC_FEATURE (9)..(9): Xaa is hydrophobic (any of A,C,F,G,H,I,K,L,M,R, T,V,W,Y)
  MISC_FEATURE (11)..(11): Xaa is polar (any of C,D,E,H,K,N,Q,R,S,T)
  MISC_FEATURE (12)..(12): Xaa is hydrophobic (any of A,C,F,G,H,I,K,L,M,R, T,V,W,Y)
  MISC_FEATURE (13)..(13): Xaa is small (any of A,C,D,G,N,P,S,T,V)
  MISC_FEATURE (14)..(14): Xaa is hydrophobic (any of A,C,F,G,H,I,K,L,M,R, T,V,W,Y)
  MISC_FEATURE (15)..(15): Xaa is polar (any of C,D,E,H,K,N,Q,R,S,T)
  MISC_FEATURE (16)..(16): Xaa is aromatic (any of F,H,W,Y)
  MISC_FEATURE (17)..(17): Xaa is aliphatic (any of I,L,V)
  MISC_FEATURE (18)..(18): Xaa is turnlike (any of A,C,D,E,G,H,K,N,Q,R,S,T )
  MISC_FEATURE (19)..(19): Xaa is any natural amino acid
  MISC_FEATURE (20)..(20): Xaa is aliphatic (any of I,L,V)
XXXXXKXXXLXXXXXXXXXX

SEQ ID NO: 337 - protein, 20 aa, Homo sapiens
  MISC_FEATURE (1)..(1): Xaa is small (any of A,C,D,G,N,P,S,T,V)
  MISC_FEATURE (2)..(2): Xaa is polar (any of C,D,E,H,K,N,Q,R,S,T)
  MISC_FEATURE (3)..(3): Xaa is positive (any of H,K,R)
  MISC_FEATURE (4)..(4): Xaa is hydrophobic (any of A,C,F,G,H,I,K,L,M,R, T,V,W,Y)
  MISC_FEATURE (5)..(5): Xaa is small (any of A,C,D,G,N,P,S,T,V)
  MISC_FEATURE (7)..(7): Xaa is hydrophobic (any of A,C,F,G,H,I,K,L,M,R, T,V,W,Y)
  MISC_FEATURE (8)..(8): Xaa is polar (any of C,D,E,H,K,N,Q,R,S,T)
  MISC_FEATURE (9)..(9): Xaa is hydrophobic (any of A,C,F,G,H,I,K,L,M,R, T,V,W,Y)
  MISC_FEATURE (11)..(11): Xaa is charged (any of D,E,H,K,R)
  MISC_FEATURE (12)..(12): Xaa is hydrophobic (any of A,C,F,G,H,I,K,L,M,R, T,V,W,Y)
  MISC_FEATURE (13)..(13): Xaa is tiny (any of A,G,S)
  MISC_FEATURE (14)..(14): Xaa is hydrophobic (any of A,C,F,G,H,I,K,L,M,R, T,V,W,Y)
  MISC_FEATURE (15)..(15): Xaa is polar (any of C,D,E,H,K,N,Q,R,S,T)
  MISC_FEATURE (17)..(17): Xaa is aliphatic (any of I,L,V)
  MISC_FEATURE (18)..(18): Xaa is turnlike (any of A,C,D,E,G,H,K,N,Q,R,S,T )
  MISC_FEATURE (19)..(19): Xaa is hydrophobic (any of A,C,F,G,H,I,K,L,M,R, T,V,W,Y)
XXXXXKXXXLXXXXXYXXXL

SEQ ID NO: 338 - protein, 19 aa, Artificial Sequence
  synthetic
  MISC_FEATURE (1)..(1): Xaa is acetyl-R
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
XAQILCKATEYIQXMRRXN

SEQ ID NO: 339 - protein, 19 aa, Artificial Sequence
  synthetic
  MISC_FEATURE (1)..(1): Xaa is acetyl-R
XAQILCKATEYIQYMRRKN

SEQ ID NO: 340 - protein, 19 aa, Artificial Sequence
  synthetic
  MISC_FEATURE (1)..(1): Xaa is acetyl-R
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
XAXILCXATEYIQYMRRKN

SEQ ID NO: 341 - protein, 24 aa, Artificial Sequence
  synthetic
  MISC_FEATURE (1)..(1): Xaa is acetyl-H
XNALERKRRDHIKDSFHKLRDSVP

SEQ ID NO: 342 - protein, 28 aa, Artificial Sequence
  synthetic
  MISC_FEATURE (1)..(1): Xaa is acetyl-K
  MISC_FEATURE (22)..(22): Xaa is K(GlyMal) (glycylmaleimide-lysine)
  misc_feature (28)..(28): Xaa is P-NH2
XRAHHNALERKRRDHIKDSFHXLRDSVX

SEQ ID NO: 343 - protein, 28 aa, Artificial Sequence
  synthetic
  MISC_FEATURE (1)..(1): Xaa is acetyl-K
XRAHHNALERKRRDHIKDSFHKLRDSVP

SEQ ID NO: 344 - protein, 28 aa, Artificial Sequence
  synthetic
  MISC_FEATURE (1)..(1): Xaa is acetyl-K
  MISC_FEATURE (21)..(21): Xaa is S5 ((S)-2-(4′-pentenyl)alanine)
  MISC_FEATURE (22)..(22): Xaa is K(GlyMal) (glycylmaleimide-lysine)
  MISC_FEATURE (25)..(25): Xaa is S5 ((S)-2-(4′-pentenyl)alanine)
  MISC_FEATURE (28)..(28): Xaa is P-NH2
XRAHHNALERKRRDHIKDSFXXLRXSVX

SEQ ID NO: 345 - protein, 28 aa, Artificial Sequence
  synthetic
  MISC_FEATURE (1)..(1): Xaa is acetyl-K
  MISC_FEATURE (21)..(21): Xaa is S5 ((S)-2-(4′-pentenyl)alanine)
  MISC_FEATURE (26)..(26): Xaa is S5 ((S)-2-(4′-pentenyl)alanine)
XRAHHNALERKRRDHIKDSFXKLRSXVP

SEQ ID NO: 346 - protein, 28 aa, Artificial Sequence
  synthetic
  MISC_FEATURE (1)..(1): Xaa is acetyl-K
  MISC_FEATURE (11)..(11): Xaa is S5 ((S)-2-(4′-pentenyl)alanine)
  MISC_FEATURE (15)..(15): Xaa is S5 ((S)-2-(4′-pentenyl)alanine)
  MISC_FEATURE (22)..(22): Xaa is K(GlyMal) (glycylmaleimide-lysine)
  MISC_FEATURE (28)..(28): Xaa is P-NH2
XRAHHNALERXRRDXIKDSFHXLRDSVX

SEQ ID NO: 347 - protein, 28 aa, Artificial Sequence
  synthetic
  MISC_FEATURE (1)..(1): Xaa is acetyl-K
  MISC_FEATURE (11)..(11): Xaa is S5 ((S)-2-(4′-pentenyl)alanine)
  MISC_FEATURE (15)..(15): Xaa is S5 ((S)-2-(4′-pentenyl)alanine)
XRAHHNALERXRRDXIKDSFHKLRDSVP

SEQ ID NO: 348 - protein, 28 aa, Artificial Sequence
  synthetic
  MISC_FEATURE (1)..(1): Xaa is acetyl-K
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
  MISC_FEATURE (22)..(22): Xaa is K(GlyMal) (glycylmaleimide-lysine)
  MISC_FEATURE (28)..(28): Xaa is P-NH2
XRAHHNXLERXRRDHIKDSFHXLRDSVX

SEQ ID NO: 349 - protein, 28 aa, Artificial Sequence
  synthetic
  MISC_FEATURE (1)..(1): Xaa is acetyl-K
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
XRAHHNXLERXRRDHIKDSFHKLRDSVP

SEQ ID NO: 350 - protein, 28 aa, Artificial Sequence
  synthetic
  MISC_FEATURE (1)..(1): Xaa is acetyl-K
  MISC_FEATURE (3)..(3): Xaa is Aib (2-aminoisobutyric acid)
  MISC_FEATURE (11)..(11): Xaa is S5 ((S)-2-(4′-pentenyl)alanine)
  MISC_FEATURE (15)..(15): Xaa is S5 ((S)-2-(4′-pentenyl)alanine)
XRXHHNALERXRRDXIKDSFHKLRDSVP

SEQ ID NO: 351 - protein, 28 aa, Artificial Sequence
  synthetic
  MISC_FEATURE (1)..(1): Xaa is acetyl-K
  MISC_FEATURE (3)..(3): Xaa is Aib (2-aminoisobutyric acid)
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
XRXHHNXLERXRRDHIKDSFHKLRDSVP

SEQ ID NO: 352 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (11)..(11): Xaa is D-lysine
XRXHHNXLERXRRDHIKDSFHKLRDSVP

SEQ ID NO: 353 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (11)..(11): Xaa is Aib (2-aminoisobutyric acid)
XRXHHNXLERXRRDHIKDSFHKLRDSVP

SEQ ID NO: 354 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (22)..(22): Xaa is K(GlyMal) (glycylmaleimide-lysine)
  MISC_FEATURE (28)..(28): Xaa is P-NH2
XRXHHNXLERKRRDHIKDSFHXLRDSVX

SEQ ID NO: 355 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
XRXHHNXLERKRRDHIKDSFHKLRDSVP

SEQ ID NO: 356 - protein, 19 aa, Artificial Sequence
  synthetic
  MISC_FEATURE (1)..(1): Xaa is acetyl-K
  MISC_FEATURE (3)..(3): Xaa is C(StBu) (tert-butylsulfenyl-cysteine)
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is K(GlyMal)-NH2 (glycylmaleimide-lysine(NH 2))
XVXILKKATAYILXVQAXX

SEQ ID NO: 357 - protein, 19 aa, Artificial Sequence
  synthetic
  MISC_FEATURE (1)..(1): Xaa is acetyl-K
  MISC_FEATURE (3)..(3): Xaa is C(StBu) (tert-butylsulfenyl-cysteine)
  MISC_FEATURE (19)..(19): Xaa is K(GlyMal)-NH2 ((glycylmaleimide)lysine- NH2)
XVXILKKATAYILSVQAEX

SEQ ID NO: 358 - protein, 19 aa, Artificial Sequence
  synthetic
  MISC_FEATURE (1)..(1): Xaa is acetyl-K
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is K(N3)-NH2 (azidolysine-NH2)
XVCILKKATAYILXVQAXX

SEQ ID NO: 359 - protein, 19 aa, Artificial Sequence
  synthetic
  MISC_FEATURE (1)..(1): Xaa is acetyl-K
  MISC_FEATURE (19)..(19): Xaa is K(N3)-NH2 (azidolysine-NH2)
XVCILKKATAYILSVQAEX

SEQ ID NO: 360 - protein, 19 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (6)..(6): Xaa is C(StBu) (tert-butylsulfenyl-cysteine)
  MISC_FEATURE (7)..(7): Xaa is S5 ((S)-2-(4′-pentenyl)alanine)
  MISC_FEATURE (19)..(19): Xaa is K(GlyMal)-NH2 ((glycylmaleimide)lysine- NH2)
XVXILXXATAYILSVQAEX

SEQ ID NO: 361 - protein, 19 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (19)..(19): Xaa is K(N3)-NH2 (azidolysine-NH2)
XVXILCXATAYILSVQAEX

SEQ ID NO: 362 - protein, 19 aa, Artificial Sequence
  synthetic
  MISC_FEATURE (1)..(1): Xaa is acetyl-K
  MISC_FEATURE (6)..(6): Xaa is C(StBu) (tert-butylsulfenyl-cysteine)
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is K(GlyMal)-NH2 ((glycylmaleimide)lysine- NH2)
XVVILXKATAYILXVQAXX

SEQ ID NO: 363 - protein, 19 aa, Artificial Sequence
  synthetic
  MISC_FEATURE (1)..(1): Xaa is acetyl-K
  MISC_FEATURE (6)..(6): Xaa is C(StBu) (tert-butylsulfenyl-cysteine)
  MISC_FEATURE (19)..(19): Xaa is K(GlyMal)-NH2 ((glycylmaleimide)lysine- NH2)
XVVILXKATAYILSVQAEX

SEQ ID NO: 364 - protein, 19 aa, Artificial Sequence
  synthetic
  MISC_FEATURE (1)..(1): Xaa is acetyl-K
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is K(N3)-NH2 (azidolysine-NH2)
XVVILCKATAYILXVQAXX

SEQ ID NO: 365 - protein, 19 aa, Artificial Sequence
  synthetic
  MISC_FEATURE (1)..(1): Xaa is acetyl-K
  MISC_FEATURE (19)..(19): Xaa is K(N3)-NH2 (azidolysine-NH2)
XVVILCKATAYILSVQAEX

SEQ ID NO: 366 - protein, 19 aa, Artificial Sequence
  synthetic
  MISC_FEATURE (1)..(1): Xaa is acetyl-R
  MISC_FEATURE (3)..(3): Xaa is C(StBu) (tert-butylsulfenyl-cysteine)
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  misc_feature (19)..(19): Xaa is C-NH2
XAXILDKATEYIQXMRRXX

SEQ ID NO: 367 - protein, 19 aa, Artificial Sequence
  synthetic
  MISC_FEATURE (1)..(1): Xaa is acetyl-R
  MISC_FEATURE (3)..(3): Xaa is C(StBu) (tert-butylsulfenyl-cysteine)
  MISC_FEATURE (19)..(19): Xaa is C-NH2
XAXILDKATEYIQYMRRKX

SEQ ID NO: 368 - protein, 19 aa, Artificial Sequence
  synthetic
  MISC_FEATURE (1)..(1): Xaa is acetyl-R
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is C(StBu)-NH2 ((tert-butylsulfenyl) cysteine-NH2)
XACILDKATEYIQXMRRXX

SEQ ID NO: 369 - protein, 19 aa, Artificial Sequence
  synthetic
  MISC_FEATURE (1)..(1): Xaa is acetyl-R
  MISC_FEATURE (19)..(19): Xaa is C(StBu)-NH2 ((tert-butylsulfenyl) cysteine-NH2)
XACILDKATEYIQYMRRKX

SEQ ID NO: 370 - protein, 19 aa, Artificial Sequence
  synthetic
  MISC_FEATURE (1)..(1): Xaa is acetyl-R
  MISC_FEATURE (6)..(6): Xaa is C(StBu) (tert-butylsulfenyl-cysteine)
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is C-NH2
XAQILXKATEYIQXMRRXX

SEQ ID NO: 371 - protein, 20 aa, Artificial Sequence
  synthetic
  MISC_FEATURE (1)..(1): Xaa is acetyl-R
  MISC_FEATURE (6)..(6): Xaa is C(StBu) (tert-butylsulfenyl-cysteine)
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (20)..(20): Xaa is beta-C-NH2
XAQILXKATEYIQXMRRXNX

SEQ ID NO: 372 - protein, 19 aa, Artificial Sequence
  synthetic
  MISC_FEATURE (1)..(1): Xaa is acetyl-R
  MISC_FEATURE (6)..(6): Xaa is C(StBu) (tert-butylsulfenyl-cysteine)
  MISC_FEATURE (19)..(19): Xaa is C-NH2
XAQILXKATEYIQYMRRKX

SEQ ID NO: 373 - protein, 20 aa, Artificial Sequence
  synthetic
  MISC_FEATURE (1)..(1): Xaa is acetyl-R
  MISC_FEATURE (6)..(6): Xaa is C(StBu) (tert-butylsulfenyl-cysteine)
  MISC_FEATURE (20)..(20): Xaa is beta-C-NH2
XAQILXKATEYIQYMRRKNX

SEQ ID NO: 374 - protein, 19 aa, Artificial Sequence
  synthetic
  MISC_FEATURE (1)..(1): Xaa is acetyl-R
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (19)..(19): Xaa is C(StBu)-NH2 ((tert-butylsulfenyl) cysteine-NH2)
XAQILCKATEYIQXMRRXX

SEQ ID NO: 375 - protein, 19 aa, Artificial Sequence
  synthetic
  MISC_FEATURE (1)..(1): Xaa is acetyl-R
  MISC_FEATURE (19)..(19): Xaa is C(StBu)-NH2 ((tert-butylsulfenyl) cysteine-NH2)
XAQILCKATEYIQYMRRKX

SEQ ID NO: 376 - protein, 19 aa, Artificial Sequence
  synthetic
  MISC_FEATURE (1)..(1): Xaa is acetyl-R
  MISC_FEATURE (3)..(3): Xaa is S5 ((S)-2-(4′-pentenyl)alanine)
  MISC_FEATURE (6)..(6): Xaa is C(StBu) (tert-butylsulfenyl-cysteine)
  MISC_FEATURE (7)..(7): Xaa is S5 ((S)-2-(4′-pentenyl)alanine)
  MISC_FEATURE (19)..(19): Xaa is C-NH2
XAXILXXATEYIQYMRRKX

SEQ ID NO: 377 - protein, 20 aa, Artificial Sequence
  synthetic
  MISC_FEATURE (1)..(1): Xaa is acetyl-R
  MISC_FEATURE (3)..(3): Xaa is S5 ((S)-2-(4′-pentenyl)alanine)
  MISC_FEATURE (6)..(6): Xaa is C(StBu) (tert-butylsulfenyl-cysteine)
  MISC_FEATURE (7)..(7): Xaa is S5 ((S)-2-(4′-pentenyl)alanine)
  MISC_FEATURE (20)..(20): Xaa is beta-C-NH2
XAXILXXATEYIQYMRRKNX

SEQ ID NO: 378 - protein, 19 aa, Artificial Sequence
  synthetic
  MISC_FEATURE (1)..(1): Xaa is acetyl-R
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (19)..(19): Xaa is C(StBu)-NH2 ((tert-butylsulfenyl) cysteine-NH2)
XAXILCXATEYIQYMRRKX

SEQ ID NO: 379 - protein, 20 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (15)..(15): Xaa is S5 ((S)-2-(4′-pentenyl)alanine)
  MISC_FEATURE (16)..(16): Xaa is NL (norleucine)
  MISC_FEATURE (19)..(19): Xaa is S5 ((S)-2-(4′-pentenyl)alanine)
XRXQILCQATEYIQXXRRXN

SEQ ID NO: 380 - protein, 21 aa, Artificial Sequence
  synthetic
  MISC_FEATURE (1)..(1): Xaa is acetyl-S
  MISC_FEATURE (7)..(7): Xaa is C(StBu) (tert-butylsulfenyl-cysteine)
  MISC_FEATURE (15)..(15): Xaa is S5 ((S)-2-(4′-pentenyl)alanine)
  MISC_FEATURE (16)..(16): Xaa is NL (norleucine)
  MISC_FEATURE (19)..(19): Xaa is S5 ((S)-2-(4′-pentenyl)alanine)
  MISC_FEATURE (21)..(21): Xaa is beta-C-NH2
XRAQILXKATEYIQXXRRXNX

SEQ ID NO: 381 - protein, 20 aa, Artificial Sequence
  synthetic
  MISC_FEATURE (1)..(1): Xaa is acetyl-S
  MISC_FEATURE (15)..(15): Xaa is S5 ((S)-2-(4′-pentenyl)alanine)
  MISC_FEATURE (16)..(16): Xaa is NL (norleucine)
  MISC_FEATURE (19)..(19): Xaa is S5 ((S)-2-(4′-pentenyl)alanine)
XRAQILCKATEYIQXXRRXN

SEQ ID NO: 382 - protein, 17 aa, Artificial Sequence
  synthetic
  MISC_FEATURE (1)..(1): Xaa is acetyl-S
  MISC_FEATURE (16)..(16): Xaa is NL (norleucine)
XRAQILCKATEYIQYXR

SEQ ID NO: 383 - protein, 20 aa, Artificial Sequence
  synthetic
  MISC_FEATURE (1)..(1): Xaa is acetyl-S
  MISC_FEATURE (16)..(16): Xaa is NL (norleucine)
XRAQILCKATEYIQYXRRKN

SEQ ID NO: 384 - protein, 20 aa, Artificial Sequence
  synthetic
  MISC_FEATURE (1)..(1): Xaa is acetyl-S
  MISC_FEATURE (15)..(15): Xaa is S5 ((S)-2-(4′-pentenyl)alanine)
  MISC_FEATURE (16)..(16): Xaa is NL (norleucine)
  MISC_FEATURE (19)..(19): Xaa is S5 ((S)-2-(4′-pentenyl)alanine)
XRAQILCQATEYIQXXRRXN

SEQ ID NO: 385 - protein, 21 aa, Artificial Sequence
  synthetic
  MISC_FEATURE (1)..(1): Xaa is acetyl-S
  MISC_FEATURE (4)..(4): Xaa is S5 ((S)-2-(4′-pentenyl)alanine)
  MISC_FEATURE (7)..(7): Xaa is C(StBu) (tert-butylsulfenyl-cysteine)
  MISC_FEATURE (8)..(8): Xaa is S5 ((S)-2-(4′-pentenyl)alanine)
  MISC_FEATURE (16)..(16): Xaa is NL (norleucine)
  MISC_FEATURE (21)..(21): Xaa is beta-C-NH2
XRAXILXXATEYIQYXRRKNX

SEQ ID NO: 386 - protein, 20 aa, Artificial Sequence
  synthetic
  MISC_FEATURE (1)..(1): Xaa is acetyl-S
  MISC_FEATURE (4)..(4): Xaa is S5 ((S)-2-(4′-pentenyl)alanine)
  MISC_FEATURE (8)..(8): Xaa is S5 ((S)-2-(4′-pentenyl)alanine)
  MISC_FEATURE (16)..(16): Xaa is NL (norleucine)
XRAXILCXATEYIQYXRRKN

SEQ ID NO: 387 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (25)..(25): Xaa is K(GlyMal) (glycylmaleimide-lysine)
  MISC_FEATURE (30)..(30): Xaa is V-NH2
XXDKRAHHNALERKRRDHIKDSFHXLRDSX

SEQ ID NO: 388 - protein, 30 aa, Artificial Sequence; identical to SEQ ID NO: 387
  synthetic
  MISC_FEATURE (1)..(1): Xaa is acetyl-W
  MISC_FEATURE (2)..(2): Xaa is beta-A
  MISC_FEATURE (25)..(25): Xaa is K(N3) (azidolysine)
  MISC_FEATURE (30)..(30): Xaa is V-NH2

SEQ ID NO: 389 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (27)..(27): Xaa is K(GlyMal) (glycylmaleimide-lysine)
  MISC_FEATURE (30)..(30): Xaa is V-NH2
XXDKRAHHNALERKRRDHIKDSFHSLXDSX

SEQ ID NO: 390 - protein, 30 aa, Artificial Sequence; identical to SEQ ID NO: 389
  synthetic
  MISC_FEATURE (1)..(1): Xaa is acetyl-W
  MISC_FEATURE (2)..(2): Xaa is beta-A
  MISC_FEATURE (27)..(27): Xaa is K(N3) (azidolysine)
  MISC_FEATURE (30)..(30): Xaa is V-NH2

SEQ ID NO: 391 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (24)..(24): Xaa is S5 ((S)-2-(4′-pentenyl)alanine)
  MISC_FEATURE (25)..(25): Xaa is K(GlyMal) (glycylmaleimide-lysine)
  MISC_FEATURE (28)..(28): Xaa is S5 ((S)-2-(4′-pentenyl)alanine)
  MISC_FEATURE (30)..(30): Xaa is V-NH2
XXDKRAHHNALERKRRDHIKDSFXXLRXSX

SEQ ID NO: 392 - protein, 30 aa, Artificial Sequence; identical to SEQ ID NO: 391
  synthetic
  MISC_FEATURE (1)..(1): Xaa is acetyl-W
  MISC_FEATURE (2)..(2): Xaa is beta-A
  MISC_FEATURE (24)..(24): Xaa is S5 ((S)-2-(4′-pentenyl)alanine)
  MISC_FEATURE (25)..(25): Xaa is K(N3) (azidolysine)
  MISC_FEATURE (28)..(28): Xaa is S5 ((S)-2-(4′-pentenyl)alanine)
  MISC_FEATURE (30)..(30): Xaa is V-NH2

SEQ ID NO: 393 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (24)..(24): Xaa is S5 ((S)-2-(4′-pentenyl)alanine)
  MISC_FEATURE (27)..(27): Xaa is K(GlyMal) (glycylmaleimide-lysine)
  MISC_FEATURE (28)..(28): Xaa is S5 ((S)-2-(4′-pentenyl)alanine)
  MISC_FEATURE (30)..(30): Xaa is V-NH2
XXDKRAHHNALERKRRDHIKDSFXSLXXSX

SEQ ID NO: 394 - protein, 30 aa, Artificial Sequence; identical to SEQ ID NO: 393
  synthetic
  MISC_FEATURE (1)..(1): Xaa is acetyl-W
  MISC_FEATURE (2)..(2): Xaa is beta-A
  MISC_FEATURE (24)..(24): Xaa is S5 ((S)-2-(4′-pentenyl)alanine)
  MISC_FEATURE (27)..(27): Xaa is K(N3) (azidolysine)
  MISC_FEATURE (28)..(28): Xaa is S5 ((S)-2-(4′-pentenyl)alanine)
  MISC_FEATURE (30)..(30): Xaa is V-NH2

SEQ ID NO: 395 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (25)..(25): Xaa is K(GlyMal) (glycylmaleimide-lysine)
  MISC_FEATURE (30)..(30): Xaa is V-NH2
XXDKRAHHNALERXRRDXIKDSFHXLRDSX

SEQ ID NO: 396 - protein, 30 aa, Artificial Sequence; identical to SEQ ID NO: 395
  synthetic
  MISC_FEATURE (1)..(1): Xaa is acetyl-W
  MISC_FEATURE (2)..(2): Xaa is beta-A
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (25)..(25): Xaa is K(N3) (azidolysine)
  MISC_FEATURE (30)..(30): Xaa is V-NH2

SEQ ID NO: 397 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (27)..(27): Xaa is K(GlyMal) (glycylmaleimide-lysine)
  MISC_FEATURE (30)..(30): Xaa is V-NH2
XXDKRAHHNALERXRRDXIKDSFHSLXDSX

SEQ ID NO: 398 - protein, 30 aa, Artificial Sequence; identical to SEQ ID NO: 397
  synthetic
  MISC_FEATURE (1)..(1): Xaa is acetyl-W
  MISC_FEATURE (2)..(2): Xaa is beta-A
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (27)..(27): Xaa is K(N3) (azidolysine)
  MISC_FEATURE (30)..(30): Xaa is V-NH2

SEQ ID NO: 399 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (25)..(25): Xaa is K(GlyMal) (glycylmaleimide-lysine)
  MISC_FEATURE (30)..(30): Xaa is V-NH2
XXDKRAHHNXLERXRRDHIKDSFHXLRDSX

SEQ ID NO: 400 - protein, 30 aa, Artificial Sequence; identical to SEQ ID NO: 399
  synthetic
  MISC_FEATURE (1)..(1): Xaa is acetyl-W
  MISC_FEATURE (2)..(2): Xaa is beta-A
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (25)..(25): Xaa is K(N3) (azidolysine)
  MISC_FEATURE (30)..(30): Xaa is V-NH2

SEQ ID NO: 401 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (27)..(27): Xaa is K(GlyMal) (glycylmaleimide-lysine)
  MISC_FEATURE (30)..(30): Xaa is V-NH2
XXDKRAHHNXLERXRRDHIKDSFHSLXDSX

SEQ ID NO: 402 - protein, 30 aa, Artificial Sequence; identical to SEQ ID NO: 401
  synthetic
  MISC_FEATURE (1)..(1): Xaa is acetyl-W
  MISC_FEATURE (2)..(2): Xaa is beta-A
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (27)..(27): Xaa is K(N3) (azidolysine)
  MISC_FEATURE (30)..(30): Xaa is V-NH2

SEQ ID NO: 403 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (25)..(25): Xaa is K(GlyMal) (glycylmaleimide-lysine)
  MISC_FEATURE (30)..(30): Xaa is V-NH2
XXDKRXHHNXLERKRRDHIKDSFHXLRDSX

SEQ ID NO: 404 - protein, 30 aa, Artificial Sequence; identical to SEQ ID NO: 403
  synthetic
  MISC_FEATURE (1)..(1): Xaa is acetyl-W
  MISC_FEATURE (2)..(2): Xaa is beta-A
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (25)..(25): Xaa is K(N3) (azidolysine)
  MISC_FEATURE (30)..(30): Xaa is V-NH2

SEQ ID NO: 405 - protein, 30 aa, Artificial Sequence
  synthetic
  MISC_FEATURE (1)..(1): Xaa is acetyl-W
  MISC_FEATURE (2)..(2): Xaa is beta-A
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (27)..(27): Xaa is K(GlyMal) (glycylmaleimide-lysine)
  MISC_FEATURE (30)..(30): Xaa is V-NH2
XXDKRXHHNXLERKRRDHIKDSFHSLXDSX

SEQ ID NO: 406 - protein, 30 aa, Artificial Sequence; identical to SEQ ID NO: 405
  synthetic
  MISC_FEATURE (1)..(1): Xaa is acetyl-W
  MISC_FEATURE (2)..(2): Xaa is beta-A
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (27)..(27): Xaa is K(N3) (azidolysine)
  MISC_FEATURE (30)..(30): Xaa is V-NH2

SEQ ID NO: 407 - protein, 28 aa, Artificial Sequence
  synthetic
  MISC_FEATURE (1)..(1): Xaa is acetyl-W
  MISC_FEATURE (2)..(2): Xaa is beta-K
  MISC_FEATURE (23)..(23): Xaa is K(GlyMal) (glycylmaleimide-lysine)
  MISC_FEATURE (28)..(28): Xaa is V-NH2
XXRAHHNALERKRRDHIKDSFHXLRDSX

SEQ ID NO: 408 - protein, 28 aa, Artificial Sequence
  synthetic
  MISC_FEATURE (1)..(1): Xaa is acetyl-W
  MISC_FEATURE (2)..(2): Xaa is beta-K
  MISC_FEATURE (22)..(22): Xaa is S5 ((S)-2-(4′-pentenyl)alanine),
  MISC_FEATURE (23)..(23): Xaa is K(GlyMal) (glycylmaleimide-lysine)
  MISC_FEATURE (26)..(26): Xaa is S5 ((S)-2-(4′-pentenyl)alanine)
  MISC_FEATURE (28)..(28): Xaa is V-NH2
XXRAHHNALERKRRDHIKDSFXXLRXSX

SEQ ID NO: 409 - protein, 28 aa, Artificial Sequence
  synthetic
  MISC_FEATURE (1)..(1): Xaa is acetyl-W
  MISC_FEATURE (2)..(2): Xaa is beta-K
  MISC_FEATURE (12)..(12): Xaa is S5 ((S)-2-(4′-pentenyl)alanine)
  MISC_FEATURE (16)..(16): Xaa is S5 ((S)-2-(4′-pentenyl)alanine)
  MISC_FEATURE (23)..(23): Xaa is K(GlyMal) (glycylmaleimide-lysine)
  MISC_FEATURE (28)..(28): Xaa is V-NH2
XXRAHHNALERXRRDXIKDSFHXLRDSX

SEQ ID NO: 410 - protein, 28 aa, Artificial Sequence
  synthetic
  MISC_FEATURE (1)..(1): Xaa is acetyl-W
  MISC_FEATURE (2)..(2): Xaa is beta-K
  MISC_FEATURE (8)..(8): Xaa is S5 ((S)-2-(4′-pentenyl)alanine)
  MISC_FEATURE (12)..(12): Xaa is S5 ((S)-2-(4′-pentenyl)alanine)
  MISC_FEATURE (23)..(23): Xaa is K(GlyMal) (glycylmaleimide-lysine)
  MISC_FEATURE (28)..(28): Xaa is V-NH2
XXRAHHNXLERXRRDHIKDSFHXLRDSX

SEQ ID NO: 411 - protein, 28 aa, Artificial Sequence
  synthetic
  MISC_FEATURE (1)..(1): Xaa is acetyl-W
  MISC_FEATURE (2)..(2): Xaa is beta-K
  MISC_FEATURE (4)..(4): Xaa is S5 ((S)-2-(4′-pentenyl)alanine)
  MISC_FEATURE (8)..(8): Xaa is S5 ((S)-2-(4′-pentenyl)alanine)
  MISC_FEATURE (23)..(23): Xaa is K(GlyMal) (glycylmaleimide-lysine)
  MISC_FEATURE (28)..(28): Xaa is V-NH2
XXRXHHNXLERKRRDHIKDSFHXLRDSX

SEQ ID NO: 412 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (27)..(27): Xaa is K(GlyMal) (glycylmaleimide-lysine)
  MISC_FEATURE (30)..(30): Xaa is I-NH2
XXVKRRTHNXLERXRRNELKRSFFALXDQX

SEQ ID NO: 413 - protein, 30 aa, Artificial Sequence; identical to SEQ ID NO: 412
  synthetic
  MISC_FEATURE (1)..(1): Xaa is acetyl-W
  MISC_FEATURE (2)..(2): Xaa is beta-N
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (27)..(27): Xaa is K(N3) (azidolysine)
  MISC_FEATURE (30)..(30): Xaa is I-NH2

SEQ ID NO: 414 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (25)..(25): Xaa is K(GlyMal) (glycylmaleimide-lysine)
  MISC_FEATURE (30)..(30): Xaa is I-NH2
XXVKRRTHNXLERXRRNELKRSFFXLRDQX

SEQ ID NO: 415 - protein, 30 aa, Artificial Sequence; identical to SEQ ID NO: 414
  synthetic
  MISC_FEATURE (1)..(1): Xaa is acetyl-W
  MISC_FEATURE (2)..(2): Xaa is beta-N
  MISC_FEATURE (10)..(10): Xaa is S5 ((S)-2-(4′-pentenyl)alanine)
  MISC_FEATURE (14)..(14): Xaa is S5 ((S)-2-(4′-pentenyl)alanine)
  MISC_FEATURE (25)..(25): Xaa is K(N3) (azidolysine)
  MISC_FEATURE (30)..(30): Xaa is I-NH2

SEQ ID NO: 416 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (27)..(27): Xaa is K(GlyMal) (glycylmaleimide-lysine)
  MISC_FEATURE (30)..(30): Xaa is I-NH2
XXVKRRTHNVLERQRRNELKRSFFALXDQX

SEQ ID NO: 417 - protein, 30 aa, Artificial Sequence; identical to SEQ ID NO: 416
  synthetic
  MISC_FEATURE (1)..(1): Xaa is acetyl-W
  MISC_FEATURE (2)..(2): Xaa is beta-N
  MISC_FEATURE (27)..(27): Xaa is K(N3) (azidolysine)
  MISC_FEATURE (30)..(30): Xaa is I-NH2

SEQ ID NO: 418 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (25)..(25): Xaa is K(GlyMal) (glycylmaleimide-lysine)
  MISC_FEATURE (30)..(30): Xaa is I-NH2
XXVKRRTHNVLERQRRNELKRSFFXLRDQX

SEQ ID NO: 419 - protein, 30 aa, Artificial Sequence; identical to SEQ ID NO: 418
  synthetic
  MISC_FEATURE (1)..(1): Xaa is acetyl-W
  MISC_FEATURE (2)..(2): Xaa is beta-N
  MISC_FEATURE (25)..(25): Xaa is K(N3) (azidolysine)
  MISC_FEATURE (30)..(30): Xaa is I-NH2

SEQ ID NO: 420 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (24)..(24): Xaa is S5 ((S)-2-(4′-pentenyl)alanine)
  MISC_FEATURE (27)..(27): Xaa is K(GlyMal) (glycylmaleimide-lysine)
  MISC_FEATURE (28)..(28): Xaa is S5 ((S)-2-(4′-pentenyl)alanine)
  MISC_FEATURE (30)..(30): Xaa is I-NH2
XXVKRRTHNVLERQRRNELKRSFXALXXQX

SEQ ID NO: 421 - protein, 30 aa, Artificial Sequence; identical to SEQ ID NO: 420
  synthetic
  MISC_FEATURE (1)..(1): Xaa is acetyl-W
  MISC_FEATURE (2)..(2): Xaa is beta-N
  MISC_FEATURE (24)..(24): Xaa is S5 ((S)-2-(4′-pentenyl)alanine)
  MISC_FEATURE (27)..(27): Xaa is K(N3) (azidolysine)
  MISC_FEATURE (28)..(28): Xaa is S5 ((S)-2-(4′-pentenyl)alanine)
  MISC_FEATURE (30)..(30): Xaa is I-NH2

SEQ ID NO: 422 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (24)..(24): Xaa is S5 ((S)-2-(4′-pentenyl)alanine)
  MISC_FEATURE (25)..(25): Xaa is K(GlyMal) (glycylmaleimide-lysine)
  MISC_FEATURE (28)..(28): Xaa is S5 ((S)-2-(4′-pentenyl)alanine)
  MISC_FEATURE (30)..(30): Xaa is I-NH2
XXVKRRTHNVLERQRRNELKRSFXXLRXQX

SEQ ID NO: 423 - protein, 30 aa, Artificial Sequence; identical to SEQ ID NO: 422
  synthetic
  MISC_FEATURE (1)..(1): Xaa is acetyl-W
  MISC_FEATURE (2)..(2): Xaa is beta-N
  MISC_FEATURE (24)..(24): Xaa is S5 ((S)-2-(4′-pentenyl)alanine)
  MISC_FEATURE (25)..(25): Xaa is K(N3) (azidolysine)
  MISC_FEATURE (28)..(28): Xaa is S5 ((S)-2-(4′-pentenyl)alanine)
  MISC_FEATURE (30)..(30): Xaa is I-NH2

SEQ ID NO: 424 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (27)..(27): Xaa is K(GlyMal) (glycylmaleimide-lysine)
  MISC_FEATURE (30)..(30): Xaa is I-NH2
XXVKRRTHNVLERXRRNXLKRSFFALXDQX

SEQ ID NO: 425 - protein, 30 aa, Artificial Sequence; identical to SEQ ID NO: 424
  synthetic
  MISC_FEATURE (1)..(1): Xaa is acetyl-W
  MISC_FEATURE (2)..(2): Xaa is beta-N
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (27)..(27): Xaa is K(N3) (azidolysine)
  MISC_FEATURE (30)..(30): Xaa is I-NH2

SEQ ID NO: 426 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (25)..(25): Xaa is K(GlyMal) (glycylmaleimide-lysine)
  MISC_FEATURE (30)..(30): Xaa is I-NH2
XXVKRRTHNVLERXRRNXLKRSFFXLRDQX

SEQ ID NO: 427 - protein, 30 aa, Artificial Sequence; identical to SEQ ID NO: 426
  synthetic
  MISC_FEATURE (1)..(1): Xaa is acetyl-W
  MISC_FEATURE (2)..(2): Xaa is beta-N
  MISC_FEATURE (14)..(14): Xaa is S5 ((S)-2-(4′-pentenyl)alanine)
  MISC_FEATURE (18)..(18): Xaa is S5 ((S)-2-(4′-pentenyl)alanine)
  MISC_FEATURE (25)..(25): Xaa is K(N3) (azidolysine)
  MISC_FEATURE (30)..(30): Xaa is I-NH2

SEQ ID NO: 428 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (27)..(27): Xaa is K(GlyMal) (glycylmaleimide-lysine)
  MISC_FEATURE (30)..(30): Xaa is I-NH2
XXVKRXTHNXLERQRRNELKRSFFALXDQX

SEQ ID NO: 429 - protein, 30 aa, Artificial Sequence; identical to SEQ ID NO: 428
  synthetic
  MISC_FEATURE (1)..(1): Xaa is acetyl-W
  MISC_FEATURE (2)..(2): Xaa is beta-N
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (27)..(27): Xaa is K(N3) (azidolysine)
  MISC_FEATURE (30)..(30): Xaa is I-NH2

SEQ ID NO: 430 - protein, 30 aa, Artificial Sequence
  synthetic
  MISC_FEATURE (1)..(1): Xaa is acetyl-W
  MISC_FEATURE (2)..(2): Xaa is beta-N
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (25)..(25): Xaa is K(GlyMal) (glycylmaleimide-lysine)
  MISC_FEATURE (30)..(30): Xaa is I-NH2
XXVKRXTHNXLERQRRNELKRSFFXLRDQX

SEQ ID NO: 431 - protein, 30 aa, Artificial Sequence; identical to SEQ ID NO: 430
  synthetic
  MISC_FEATURE (1)..(1): Xaa is acetyl-W
  MISC_FEATURE (2)..(2): Xaa is beta-N
  MISC_FEATURE (6)..(6): Xaa is S5 ((S)-2-(4′-pentenyl)alanine)
  MISC_FEATURE (10)..(10): Xaa is S5 ((S)-2-(4′-pentenyl)alanine)
  MISC_FEATURE (25)..(25): Xaa is K(N3) (azidolysine)
  MISC_FEATURE (30)..(30): Xaa is I-NH2

SEQ ID NO: 432 - protein, 28 aa, Artificial Sequence
  synthetic
  MISC_FEATURE (1)..(1): Xaa is acetyl-K
  MISC_FEATURE (28)..(28): Xaa is P-NH2
XRAHHNALERKRRDHIKDSFHKLRDSVX

SEQ ID NO: 433 - protein, 20 aa, Artificial Sequence
  synthetic
  MISC_FEATURE (1)..(1): Xaa is acetyl-S
  MISC_FEATURE (16)..(16): Xaa is NL (norleucine)
  MISC_FEATURE (20)..(20): Xaa is N-NH2
XRAQILCKATEYIQYXRRKX

SEQ ID NO: 434 - protein, 36 aa, Artificial Sequence
  synthetic
  MISC_FEATURE (1)..(1): Xaa is acetyl-P
  MISC_FEATURE (36)..(36): Xaa is P-NH2
XRFQSAADKRAHHNALERKRRDHIKDSFHKLRDSVX

SEQ ID NO: 435 - protein, 33 aa, Artificial Sequence
  synthetic
  MISC_FEATURE (1)..(1): Xaa is acetyl-Q
  MISC_FEATURE (33)..(33): Xaa is P-NH2
XSAADKRAHHNALERKRRDHIKDSFHKLRDSVX

SEQ ID NO: 436 - protein, 36 aa, Artificial Sequence
  synthetic
  MISC_FEATURE (1)..(1): Xaa is acetyl-I
  MISC_FEATURE (36)..(36): Xaa is P-NH2
XEVESDADKRAHHNALERKRRDHIKDSFHKLRDSVX

SEQ ID NO: 437 - protein, 38 aa, Artificial Sequence
  synthetic
  MISC_FEATURE (1)..(1): Xaa is acetyl-P
  MISC_FEATURE (38)..(38): Xaa is P-NH2
XRSSDTEENVKRAHHNALERKRRDHIKDSFHKLRDSVX

SEQ ID NO: 438 - protein, 33 aa, Artificial Sequence
  synthetic
  MISC_FEATURE (1)..(1): Xaa is acetyl-T
  MISC_FEATURE (33)..(33): Xaa is P-NH2
XEENVKRAHHNALERKRRDHIKDSFHKLRDSVX

SEQ ID NO: 439 - protein, 36 aa, Artificial Sequence
  synthetic
  MISC_FEATURE (1)..(1): Xaa is acetyl-K
  MISC_FEATURE (36)..(36): Xaa is P-NH2
XSKKNNSSKRAHHNALERKRRDHIKDSFHKLRDSVX

SEQ ID NO: 440 - protein, 33 aa, Artificial Sequence
  synthetic
  MISC_FEATURE (1)..(1): Xaa is acetyl-K
  MISC_FEATURE (33)..(33): Xaa is P-NH2
XNNSSKRAHHNALERKRRDHIKDSFHKLRDSVX

SEQ ID NO: 441 - protein, 36 aa, Artificial Sequence
  synthetic
  MISC_FEATURE (1)..(1): Xaa is acetyl-P
  MISC_FEATURE (11)..(11): Xaa is S5 ((S)-2-(4′-pentenyl)alanine)
  MISC_FEATURE (15)..(15): Xaa is S5 ((S)-2-(4′-pentenyl)alanine)
  MISC_FEATURE (36)..(36): Xaa is P-NH2
XRFQSAADKRXHHNXLERKRRDHIKDSFHKLRDSVX

SEQ ID NO: 442 - protein, 36 aa, Artificial Sequence
  synthetic
  MISC_FEATURE (1)..(1): Xaa is acetyl-P
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
  MISC_FEATURE (36)..(36): Xaa is P-NH2
XRFQSAXDKRXHHNALERKRRDHIKDSFHKLRDSVX

SEQ ID NO: 443 - protein, 36 aa, Artificial Sequence
  synthetic
  MISC_FEATURE (1)..(1): Xaa is acetyl-P
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (36)..(36): Xaa is P-NH2
XRXFQSXDKRAHHNALERKRRDHIKDSFHKLRDSVX

SEQ ID NO: 444 - protein, 31 aa, Artificial Sequence
  synthetic
  MISC_FEATURE (1)..(1): Xaa is acetyl-S
  MISC_FEATURE (16)..(16): Xaa is NL (norleucine)
  MISC_FEATURE (31)..(31): Xaa is K-NH2
XRAQILCKATEYIQYXRRKNHTHQQDIDDLX

SEQ ID NO: 445 - protein, 26 aa, Artificial Sequence
  synthetic
  MISC_FEATURE (1)..(1): Xaa is acetyl-S
  MISC_FEATURE (16)..(16): Xaa is NL (norleucine)
  MISC_FEATURE (26)..(26): Xaa is E-NH2
XRAQILCKATEYIQYXRRKNHTLISX

SEQ ID NO: 446 - protein, 24 aa, Artificial Sequence
  synthetic
  MISC_FEATURE (1)..(1): Xaa is acetyl-S
  MISC_FEATURE (16)..(16): Xaa is NL (norleucine)
  MISC_FEATURE (24)..(24): Xaa is E-NH2
XRAQILCKATEYIQYXRRKLHTHX

SEQ ID NO: 447 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (15)..(15): Xaa is S5 ((S)-2-(4′-pentenyl)alanine)
  MISC_FEATURE (16)..(16): Xaa is NL (norleucine)
  MISC_FEATURE (19)..(19): Xaa is S5 ((S)-2-(4′-pentenyl)alanine)
  MISC_FEATURE (24)..(24): Xaa is E-NH2
XRXQILCQATEYIQXXRRXLHTHX

SEQ ID NO: 448 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (28)..(28): Xaa is P-NH2
XRXHHNXLERKRRDHIKDSFHKLRDSVX

SEQ ID NO: 449 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (28)..(28): Xaa is P-NH2
XRXHANXLERKRLDHIKDSFHKLRDSVX

SEQ ID NO: 450 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (28)..(28): Xaa is P-NH2
XRXHANXLERKRTDHIKDSFHKLRDSVX

SEQ ID NO: 451 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (28)..(28): Xaa is P-NH2
XKXHSNXLARKRRDHIKDSFHKLRDSVX

SEQ ID NO: 452 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (28)..(28): Xaa is P-NH2
XRXHHNXLNRKRRDHIKDSFHKLRDSVX

SEQ ID NO: 453 - protein, 36 aa, Artificial Sequence
  synthetic
  MISC_FEATURE (1)..(1): Xaa is acetyl-P
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
  MISC_FEATURE (30)..(30): Xaa is K(GlyMal) (glycylmaleimide-lysine)
  MISC_FEATURE (36)..(36): Xaa is P-NH2
XRFQSAXDKRXHHNALERKRRDHIKDSFHXLRDSVX

SEQ ID NO: 454 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (15)..(15): Xaa is S5 ((S)-2-(4′-pentenyl)alanine)
  MISC_FEATURE (16)..(16): Xaa is Nle (norleucine)
  MISC_FEATURE (19)..(19): Xaa is S5 ((S)-2-(4′-pentenyl)alanine)
  MISC_FEATURE (24)..(24): Xaa is E-NH2
XRXQILCQATEYIQXXRRXLHTHX

SEQ ID NO: 455 - protein, 24 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQYLRXKIHXLX

SEQ ID NO: 456 - protein, 28 aa, Artificial Sequence
  synthetic
  MISC_FEATURE (1)..(1): Xaa is acetyl-L
  MISC_FEATURE (22)..(22): Xaa is K(ButMal) (butyrlmaleimide-lysine)
  MISC_FEATURE (28)..(28): Xaa is P-NH2
XRLKINSRERKRMHDLNIAMDXLREVMX

SEQ ID NO: 457 - protein, 20 aa, Artificial Sequence
  synthetic
  MISC_FEATURE (1)..(1): Xaa is acetyl-S
  MISC_FEATURE (20)..(20): Xaa is L-NH2
XKIATLCLARNYILMLTNSX

SEQ ID NO: 458 - protein, 28 aa, Artificial Sequence
  synthetic
  MISC_FEATURE (1)..(1): Xaa is acetyl-R
  MISC_FEATURE (22)..(22): Xaa is K(GlyMal) (glycylmaleimide-lysine)
  MISC_FEATURE (28)..(28): Xaa is P-NH2
XREIANSNERRRMQSINAGFQXLKTLIX

SEQ ID NO: 459 - protein, 24 aa, Artificial Sequence
  synthetic
  MISC_FEATURE (1)..(1): Xaa is acetyl-S
  MISC_FEATURE (24)..(24): Xaa is E-NH2
XKAAILCQTAEYIFRLRRKIHTLX

SEQ ID NO: 460 - DNA, 20 bp, Artificial Sequence
  synthetic
ttttctcacattgcccctgt

SEQ ID NO: 461 - DNA, 20 bp, Artificial Sequence
  synthetic
tcaatgctgtaccaggcaaa

SEQ ID NO: 462 - DNA, 19 bp, Artificial Sequence
  synthetic
gccgcactggaatttatcc

SEQ ID NO: 463 - DNA, 20 bp, Artificial Sequence
  synthetic
aggagcctcacagatgcagt

SEQ ID NO: 464 - DNA, 20 bp, Artificial Sequence
  synthetic
tgactgctaacgtgcgaaac

SEQ ID NO: 465 - DNA, 20 bp, Artificial Sequence
  synthetic
gtcaagaggaggatgcggta

SEQ ID NO: 466 - DNA, 20 bp, Artificial Sequence
  synthetic
gagtttccggaagcaaaaca

SEQ ID NO: 467 - DNA, 18 bp, Artificial Sequence
  synthetic
gcggaggccatctttctt

SEQ ID NO: 468 - DNA, 19 bp, Artificial Sequence
  synthetic
taaacgtgggcacacaacc

SEQ ID NO: 469 - DNA, 20 bp, Artificial Sequence
  synthetic
taggtggcgtgactctgatg

SEQ ID NO: 470 - DNA, 20 bp, Artificial Sequence
  synthetic
gtgataggcagcagagctga

SEQ ID NO: 471 - DNA, 18 bp, Artificial Sequence
  synthetic
tgtacgcacgtggtggtc

SEQ ID NO: 472 - DNA, 20 bp, Artificial Sequence
  synthetic
accctgcagttccttctgtg

SEQ ID NO: 473 - DNA, 20 bp, Artificial Sequence
  synthetic
gaaaacacttccctccagca

SEQ ID NO: 474 - protein, 20 aa, Artificial Sequence
  synthetic
  MISC_FEATURE (1)..(1): Xaa is acetyl-S
  MISC_FEATURE (20)..(20): Xaa is N-NH2
XRAQILCKATEYIQYBRRKX

SEQ ID NO: 475 - protein, 24 aa, Artificial Sequence
  synthetic
  MISC_FEATURE (1)..(1): Xaa is acetyl-S
  MISC_FEATURE (24)..(24): Xaa is E-NH2
XRAQILCKATEYIQYLRRKIHTLX

SEQ ID NO: 476 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (16)..(16): Xaa is NLeu (norleucine)
  MISC_FEATURE (19)..(19): Xaa is S5 ((S)-2-(4′-pentenyl)alanine)
  MISC_FEATURE (23)..(23): Xaa is S5 ((S)-2-(4′-pentenyl)alanine)
  MISC_FEATURE (24)..(24): Xaa is E-NH2
XRXQILCQATEYIQSXRRXLHTXX

SEQ ID NO: 477 - protein, 24 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQYLRXKIHXLX

SEQ ID NO: 478 - protein, 28 aa, Artificial Sequence
  synthetic
KRAHHNALERKRRDHIKDSFHKLRDSVP

SEQ ID NO: 479 - protein, 4 aa, Artificial Sequence
  synthetic
RHER

SEQ ID NO: 480 - protein, 31 aa, Artificial Sequence
  synthetic
RRKAATMRERRRLSKVNEAFETLKRCTSSNP

SEQ ID NO: 481 - protein, 4 aa, Artificial Sequence
  synthetic
RAEL

SEQ ID NO: 482 - protein, 28 aa, Artificial Sequence
  synthetic
QRVMANVRERQRTQSLNEAFAALRKIIP

SEQ ID NO: 483 - protein, 4 aa, Artificial Sequence
  synthetic
RAET

SEQ ID NO: 484 - protein, 28 aa, Artificial Sequence
  synthetic
RKEKSRDAARSRRSKESEVFYELAHQLP

SEQ ID NO: 485 - protein, 4 aa, Artificial Sequence
  synthetic
KSAR

SEQ ID NO: 486 - protein, 28 aa, Artificial Sequence
  synthetic
KRAHHNALNRKRRDHIKDSFHKLRDSVP

SEQ ID NO: 487 - protein, 4 aa, Artificial Sequence
  synthetic
RHNR

SEQ ID NO: 488 - protein, 24 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQRLRXKIRXLX

SEQ ID NO: 489 - protein, 30 aa, Artificial Sequence
  synthetic
  MISC_FEATURE (1)..(1): Xaa is acetyl-S5 (acetyl-((S)-2-(4′-pentenyl) alanine))
  MISC_FEATURE (5)..(5): Xaa is S5 ((S)-2-(4′-pentenyl)alanine)
  MISC_FEATURE (24)..(24): Xaa is K(GlyMal) (glycylmaleimide-lysine)
  MISC_FEATURE (30)..(30): Xaa is P-NH2
XDKRXHHNALERKRRDHIKDSFHXLRDSVX

SEQ ID NO: 490 - protein, 36 aa, Artificial Sequence
  synthetic
  MISC_FEATURE (1)..(1): Xaa is acetyl-P
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
  MISC_FEATURE (19)..(19): Xaa is D-lysine
  MISC_FEATURE (30)..(30): Xaa is K(GlyMal) (glycylmaleimide-lysine)
  MISC_FEATURE (36)..(36): Xaa is P-NH2
XRFQSAXDKRXHHNALERXRRDHIKDSFHXLRDSVX

SEQ ID NO: 491 - protein, 36 aa, Artificial Sequence
  synthetic
  MISC_FEATURE (1)..(1): Xaa is acetyl-P
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
  MISC_FEATURE (36)..(36): Xaa is P-NH2
XRFQSAXDKRXHHNALERKRRDHIKDSFHCLRDSVX

SEQ ID NO: 492 - protein, 37 aa, Artificial Sequence
  synthetic
  MISC_FEATURE (1)..(1): Xaa is acetyl-P
  MISC_FEATURE (12)..(12): Xaa is S5 ((S)-2-(4′-pentenyl)alanine)
  MISC_FEATURE (16)..(16): Xaa is S5 ((S)-2-(4′-pentenyl)alanine)
  MISC_FEATURE (31)..(31): Xaa is K(GlyMal) (glycylmaleimide-lysine)
  MISC_FEATURE (37)..(37): Xaa is P-NH2
XAAKRVKLDKRXHHNXLERKRRDHIKDSFHXLRDSVX

SEQ ID NO: 493 - protein, 28 aa, Artificial Sequence
  synthetic
  MISC_FEATURE (1)..(1): Xaa is acetyl-L
  MISC_FEATURE (22)..(22): Xaa is K(GlyMal) (glycylmaleimide-lysine)
  MISC_FEATURE (28)..(28): Xaa is P-NH2
XRLKINSRERKRMHDINDSFHXLRDSVX

SEQ ID NO: 494 - protein, 28 aa, Artificial Sequence
  synthetic
  MISC_FEATURE (1)..(1): Xaa is acetyl-R
  MISC_FEATURE (22)..(22): Xaa is K(GlyMal) (glycylmaleimide-lysine)
  MISC_FEATURE (28)..(28): Xaa is P-NH2
XREIANSNERRRMQSINDSFHXLRDSVX

SEQ ID NO: 495 - protein, 28 aa, Artificial Sequence
  synthetic
  MISC_FEATURE (1)..(1): Xaa is acetyl-L
  MISC_FEATURE (22)..(22): Xaa is K(GlyMal) (glycylmaleimide-lysine)
  MISC_FEATURE (28)..(28): Xaa is P-NH2
XRLKINSRERKRMHDLNIAMDXLREVMX

SEQ ID NO: 496 - protein, 28 aa, Artificial Sequence
  synthetic
  MISC_FEATURE (1)..(1): Xaa is acetyl-R
  MISC_FEATURE (22)..(22): Xaa is K(GlyMal) (glycylmaleimide-lysine)
  MISC_FEATURE (28)..(28): Xaa is S-NH2
XRKAATMRERRRLSKVNEAFEXLKRSTX

SEQ ID NO: 497 - protein, 28 aa, Artificial Sequence
  synthetic
  MISC_FEATURE (1)..(1): Xaa is acetyl-R
  MISC_FEATURE (22)..(22): Xaa is K(ButMal) (butyrlmaleimide-lysine)
  MISC_FEATURE (28)..(28): Xaa is P-NH2
XREIANSNERRRMQSINAGFQXLKTLIX

SEQ ID NO: 498 - protein, 28 aa, Artificial Sequence
  synthetic
  MISC_FEATURE (1)..(1): Xaa is acetyl-R
  MISC_FEATURE (22)..(22): Xaa is K(ButMal) (butyrlmaleimide-lysine)
  MISC_FEATURE (28)..(28): Xaa is S-NH2
XRKAATMRERRRLSKVNEAFEXLKRSTX

SEQ ID NO: 499 - protein, 36 aa, Artificial Sequence
  synthetic
  MISC_FEATURE (1)..(1): Xaa is acetyl-Q
  MISC_FEATURE (30)..(30): Xaa is K(GlyMal) (glycylmaleimide-lysine)
  MISC_FEATURE (36)..(36): Xaa is P-NH2
XRDQERRIRREIANSNERRRMQSINAGFQXLKTLIX

SEQ ID NO: 500 - protein, 36 aa, Artificial Sequence
  synthetic
  MISC_FEATURE (1)..(1): Xaa is acetyl-M
  MISC_FEATURE (30)..(30): Xaa is K(GlyMal) (glycylmaleimide-lysine)
  MISC_FEATURE (36)..(36): Xaa is P-NH2
XTEPELQQLRLKINSRERKRMHDLNIAMDXLREVMX

SEQ ID NO: 501 - protein, 36 aa, Artificial Sequence
  synthetic
  MISC_FEATURE (1)..(1): Xaa is acetyl-K
  MISC_FEATURE (30)..(30): Xaa is K(GlyMal) (glycylmaleimide-lysine)
  MISC_FEATURE (36)..(36): Xaa is S-NH2
XRKTTNADRRKAATMRERRRLSKVNEAFEXLKRCTX

SEQ ID NO: 502 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (16)..(16): Xaa is NL (norleucine)
  MISC_FEATURE (19)..(19): Xaa is S5 ((S)-2-(4′-pentenyl)alanine)
  MISC_FEATURE (23)..(23): Xaa is S5 ((S)-2-(4′-pentenyl)alanine)
  MISC_FEATURE (24)..(24): Xaa is E-NH2
XRXQILCQATEYIQSXRRXLHTXX

SEQ ID NO: 503 - protein, 24 aa, Artificial Sequence
  synthetic
  MISC_FEATURE (1)..(1): Xaa is Biotin-PEG3-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQYLRXKIHXLX

SEQ ID NO: 504 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRXQILCQATEYIQYLRXKIHXLX

SEQ ID NO: 505 - protein, 24 aa, Artificial Sequence
  synthetic
  MISC_FEATURE (1)..(1): Xaa is Diazirine-PEG3-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQYLRXKIHXLX

SEQ ID NO: 506 - protein, 24 aa, Artificial Sequence
  synthetic
  MISC_FEATURE (1)..(1): Xaa is Diazirine-(6-aminohexanoic acid)-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQYLRXKIHXLX

SEQ ID NO: 507 - protein, 24 aa, Artificial Sequence
  synthetic
  MISC_FEATURE (1)..(1): Xaa is Diazirine-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCKATEYIQYLRXKIHXLX

SEQ ID NO: 508 - protein, 24 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILKKATEYIQYLRXKIHXLX

SEQ ID NO: 509 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (19)..(19): Xaa is S5 ((S)-2-(4′-pentenyl)alanine)
  MISC_FEATURE (23)..(23): Xaa is S5 ((S)-2-(4′-pentenyl)alanine)
  MISC_FEATURE (24)..(24): Xaa is E-NH2
XRXQILCQATEYIQSLRRXIHTXX

SEQ ID NO: 510 - protein, 25 aa, Artificial Sequence
  synthetic
  MISC_FEATURE (1)..(1): Xaa is acetyl-S
  MISC_FEATURE (21)..(21): Xaa is S5 ((S)-2-(4′-pentenyl)alanine)
  MISC_FEATURE (25)..(25): Xaa is S5-NH2 ((S)-2-(4′-pentenyl)alanine-NH2)
XRAQILCKATEYIQYLRRKIXTLEX

SEQ ID NO: 511 - protein, 31 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (31)..(31): Xaa is V-NH2
XRAQILCKATEYIQYLRXKIHXLEPKKKRKX

SEQ ID NO: 512 - protein, 24 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
  MISC_FEATURE (24)..(24): Xaa is E-NH2
XRAQILCEATEYIQELRXKIEXLX

SEQ ID NO: 513 - protein, 20 aa, Artificial Sequence
  synthetic
  MISC_FEATURE (1)..(1): Xaa is Pya-Py-Py-Py-PEG3-S
  MISC_FEATURE (16)..(16): Xaa is NL (norleucine)
  MISC_FEATURE (20)..(20): Xaa is N-NH2
XRAQILCKATEYIQYXRRKX

SEQ ID NO: 514 - protein, 20 aa, Artificial Sequence
  synthetic
  MISC_FEATURE (1)..(1): Xaa is acetyl-S
  MISC_FEATURE (20)..(20): Xaa is K-NH2
XKAAILCQTAEYIFSLEQEX

SEQ ID NO: 515 - protein, 20 aa, Artificial Sequence
  synthetic
  MISC_FEATURE (1)..(1): Xaa is acetyl-P
  MISC_FEATURE (20)..(20): Xaa is L-NH2
XKVEILCNAIRYIEGLQALX

SEQ ID NO: 516 - protein, 24 aa, Artificial Sequence
  synthetic
  MISC_FEATURE (1)..(1): Xaa is acetyl-S
  MISC_FEATURE (24)..(24): Xaa is E-NH2
XKIATLCLARNYILRLRRKIHTLX

SEQ ID NO: 517 - protein, 24 aa, Artificial Sequence
  synthetic
  MISC_FEATURE (1)..(1): Xaa is acetyl-P
  MISC_FEATURE (24)..(24): Xaa is E-NH2
XKVEILCNAIRYIERLRRKIHTLX

SEQ ID NO: 518 - protein, 28 aa, Artificial Sequence
  synthetic
  MISC_FEATURE (1)..(1): Xaa is acetyl-K
  MISC_FEATURE (28)..(28): Xaa is P-NH2
XRAHHNALERKRRDHIKDSFHSLRDSVX

SEQ ID NO: 519 - protein, 20 aa, Artificial Sequence
  synthetic
  MISC_FEATURE (1)..(1): Xaa is acetyl-S
  MISC_FEATURE (20)..(20): Xaa is N-NH2
XRAQILDKATEYIQYBRRKX

SEQ ID NO: 520 - protein, 28 aa, Artificial Sequence
  synthetic
  MISC_FEATURE (1)..(1): Xaa is acetyl-K
  MISC_FEATURE (3)..(3): Xaa is S5 ((S)-2-(4′-pentenyl)alanine)
  MISC_FEATURE (7)..(7): Xaa is S5 ((S)-2-(4′-pentenyl)alanine)
  MISC_FEATURE (11)..(11): Xaa is k (D-lysine)
  MISC_FEATURE (22)..(22): Xaa is K(GlyMal) (K-glycylmaleimide)
XRXHHNXLERXRRDHIKDSFHXLRDSVP

SEQ ID NO: 521 - protein, 36 aa, Artificial Sequence
  synthetic
  MISC_FEATURE (1)..(1): Xaa is acetyl-P
  MISC_FEATURE (30)..(30): Xaa is K(GlyMal) (glycylmaleimide-lysine)
XRFQSAADKRAHHNALERKRRDHIKDSFHXLRDSVP

SEQ ID NO: 522 - protein, 36 aa, Artificial Sequence
  synthetic
  MISC_FEATURE (1)..(1): Xaa is acetyl-P
  MISC_FEATURE (7)..(7): Xaa is S5 ((S)-2-(4′-pentenyl)alanine)
  MISC_FEATURE (11)..(11): Xaa is S5 ((S)-2-(4′-pentenyl)alanine)
  MISC_FEATURE (30)..(30): Xaa is K(GlyMal) (glycylmaleimide-lysine)
XRFQSAXDKRXHHNALERKRRDHIKDSFHXLRDSVP

SEQ ID NO: 523 - protein, 31 aa, Artificial Sequence
  synthetic
  MISC_FEATURE (1)..(1): Xaa is acetyl-S
  MISC_FEATURE (16)..(16): Xaa is NL (norleucine)
XRAQILCKATEYIQYXRRKNHTHQQDIDDLK

SEQ ID NO: 524 - protein, 24 aa, Artificial Sequence
  synthetic
  MISC_FEATURE (1)..(1): Xaa is acetyl-S
  MISC_FEATURE (3)..(3): Xaa is Aib (2-aminoisobutyric acid)
  MISC_FEATURE (15)..(15): Xaa is S5 ((S)-2-(4′-pentenyl)alanine)
  MISC_FEATURE (16)..(16): Xaa is NL (norleucine)
  MISC_FEATURE (19)..(19): Xaa is S5 ((S)-2-(4′-pentenyl)alanine)
XRXQILCQATEYIQXXRRXLHTHE

SEQ ID NO: 525 - protein, 24 aa, Artificial Sequence
  synthetic
  MISC_FEATURE (1)..(1): Xaa is acetyl-S
  MISC_FEATURE (18)..(18): Xaa is S5 ((S)-2-(4′-pentenyl)alanine)
  MISC_FEATURE (22)..(22): Xaa is S5 ((S)-2-(4′-pentenyl)alanine)
XRAQILCKATEYIQYLRXKIHXLE

SEQ ID NO: 526 - DNA, 11 bp, Artificial Sequence
  synthetic
gtagcacgtgc

SEQ ID NO: 527 - DNA, 8 bp, Artificial Sequence
  synthetic
gcacgtgc

SEQ ID NO: 528 - protein, 20 aa, Artificial Sequence
  synthetic
  MISC_FEATURE (16)..(16): Xaa is Nleu (norleucine)
SRAQILCKATEYIQYXRRKN

SEQ ID NO: 529 - protein, 28 aa, Artificial Sequence
  synthetic
RREIANSNERRRMQSINAGFQKLKTLIP

SEQ ID NO: 530 - protein, 24 aa, Artificial Sequence
  synthetic
SKAAILCQTAEYIFRLRRKIHTLE

SEQ ID NO: 531 - protein, 28 aa, Artificial Sequence
  synthetic
LRLKINSRERKRMHDLNIAMDKLREVMP

SEQ ID NO: 532 - protein, 20 aa, Artificial Sequence
  synthetic
SKIATLCLARNYILMLTNSL

SEQ ID NO: 533 - DNA, 19 bp, Artificial Sequence
  synthetic
  misc_feature (1)..(1): N is IRD700-A
nccccaccacgtggtgcct

SEQ ID NO: 534 - DNA, 19 bp, Artificial Sequence
  synthetic
  misc_feature (1)..(1): N is IRD700-A
nccccaccagctggtgcct

SEQ ID NO: 535 - DNA, 19 bp, Artificial Sequence
  synthetic
  misc_feature (1)..(1): N is IRD700-A
nccccaccatatggtgcct

SEQ ID NO: 536 - protein, 28 aa, Artificial Sequence
  synthetic
LRLKINSRERKRMHDINDSFHKLRDSVP